(19) United States
(12) Patent Application Publication
(10) Pub. No.: US 2024/0368269 A1
(43) Pub. Date: Nov. 7, 2024

(54) ANTI-DLL3 ANTIBODIES AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US); TRI-INSTITUTIONAL THERAPEUTICS DISCOVERY INSTITUTE, INC., New York, NY (US)

(72) Inventors: John T. Poirier, New York, NY (US); Charles Rudin, New York, NY (US); Jason Lewis, New York, NY (US); Abdul Khan, Cedar Park, TX (US); David Andrew, Sandyhook, CT (US); Xinlei Chen, Wilton, CT (US); Ivo C. Lorenz, New York, NY (US); Kathryn M. Tully, New York, NY (US); Salomon Tendler, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US); TRI-INSTITUTIONAL THERAPEUTICS DISCOVERY INSTITUTE, INC., New York, NY (US)

(21) Appl. No.: 18/589,762

(22) Filed: Feb. 28, 2024

Related U.S. Application Data
(63) Continuation of application No. PCT/US22/42451, filed on Sep. 2, 2022.
(60) Provisional application No. 63/240,216, filed on Sep. 2, 2021.

(51) Int. Cl. C07K 16/28; A61K 51/10; A61P 35/00

(52) U.S. Cl. CPC C07K 16/28; A61K 51/1027; A61P 35/00; A61K 2121/00; C07K 2317/24; C07K 2317/31; C07K 2317/565; C07K 2317/567

(57) ABSTRACT

The presently disclosed subject matter provides antibodies or antigen-binding fragments thereof that bind to DLL3 and methods of using such antibodies or antigen-binding fragments thereof same.

### CROSS REFERENCE TO RELATED APPLICATIONS

This application is a continuation of International Application No. PCT/US22/042451 filed on Sep. 2, 2022, which claims priority to U.S. Provisional Patent Application No. 63/240,216, filed on Sep. 2, 2021, the contents of which are incorporated by reference in their entireties, and to each of which priority is claimed.

### SEQUENCE LISTING

The present application contains a Sequence Listing which has been submitted via EFS-Web and is hereby incorporated by reference in its entirety. Said Sequence Listing, created on Feb. 26, 2024, is named 0727341560.xml and is 212,467 bytes in size.

### 1. FIELD OF THE INVENTION

The presently disclosed subject matter relates to antibodies that bind to DLL3, and methods of using such antibodies.

### 2. BACKGROUND OF THE INVENTION

DLL3 is selectively expressed in high grade pulmonary neuroendocrine tumors of the lung (LU-NETs). Lu-NETs embrace a heterogeneous family of neoplasms classified into four histological variants, namely typical carcinoid (TC), atypical carcinoid (AC), large cell neuroendocrine carcinoma (LCNEC) and small cell lung carcinoma (SCLC). Increased expression of DLL3 was observed in SCLC and LCNEC patient-derived xenograft tumors and was also confirmed in primary tumors. See Saunders et al., Sci Translational Medicine (302): 302ra136 (2015). Both SCLC and pulmonary LCNEC are high-grade and poor-prognosis tumors, with higher incidence in smokers. Pulmonary LCNEC exhibits biologically aggressive behavior, similarly to SCLC. Stage by stage, survival curves of pulmonary LCNEC and SCLC overlap, and in addition, survival is lower than other NSCLCs. Prognosis is poor even in patients with potentially resectable stage I lung cancer with 5-year survival rates ranging from 27% to 67%. See Iyoda A. et al., J Thorac Cardiovasc Surg. 138:446-453 (2009).

Given the significant role for DLL3 in various diseases or disorders, antibodies that recognize DLL3, and methods of using such agents, are desired.

### 3. SUMMARY OF THE INVENTION

The presently disclosed subject matter provides antibodies or antigen-binding fragments thereof that specifically bind to DLL3, and methods of using the antibodies or antigen-binding fragments thereof.

In certain embodiments, the DLL3 antibody or an antigen-binding fragment thereof comprises a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, or SEQ ID NO: 172.

In certain embodiments, the anti-DLL3 antibody or an antigen-binding fragment thereof comprises a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, or SEQ ID NO: 173.

In certain embodiments, the anti-DLL3 antibody or an antigen-binding fragment thereof comprises (a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, or SEQ ID NO: 172; and (b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, or SEQ ID NO: 173.

In certain embodiments, the heavy chain variable region and the light chain variable region of the anti-DLL3 antibody or antigen-binding fragment thereof are selected from the group consisting of:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8;
(b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 17, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 18;
(c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 25;
(d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 34, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 35;
(e) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 42, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 43;
(f) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 52, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 53;
(g) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 60, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 61;
(h) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 66, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 67;
(i) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 76, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 77;
(j) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 83, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 84;
(k) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 92, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 93;
(l) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 102, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 103;
(m) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 108, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 109;
(n) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 108, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 113;
(o) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 119, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 120;
(p) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 126, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 127;
(q) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 131, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 132;
(r) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 141, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 142;
(s) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 147, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 148;
(t) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 153, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 154;
(u) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 157, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 158;
(v) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 163, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 164; and
(w) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 172, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 173.

In certain embodiments, the anti-DLL3 antibody or antigen-binding fragment thereof comprises a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, or SEQ ID NO: 172.

In certain embodiments, the anti-DLL3 antibody or antigen-binding fragment thereof, comprises a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, or SEQ ID NO: 173.

In certain embodiments, the DLL3 antibody or antigen-binding fragment thereof comprises a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, or SEQ ID NO: 172; and a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, or SEQ ID NO: 173.

In certain embodiments, the anti-DLL3 antibody or antigen-binding fragment thereof comprises a heavy chain variable region and a light chain variable region, wherein the heavy chain variable region and the light chain variable region are selected from the group consisting of:
(a) a heavy chain variable region comprising the amino acid sequence set forth in SEQ ID NO: 7, and a light chain variable region comprising the amino acid sequence set forth in SEQ ID NO: 8;
(b) a heavy chain variable region comprising the amino acid sequence set forth in SEQ ID NO: 17, and a light chain variable region comprising the amino acid sequence set forth in SEQ ID NO: 18;
(c) a heavy chain variable region comprising the amino acid sequence set forth in SEQ ID NO: 24, and a light chain variable region comprising the amino acid sequence set forth in SEQ ID NO: 25;
(d) a heavy chain variable region comprising the amino acid sequence set forth in SEQ ID NO: 34, and a light chain variable region comprising the amino acid sequence set forth in SEQ ID NO: 35;
(e) a heavy chain variable region comprising the amino acid sequence set forth in SEQ ID NO: 42, and a light chain variable region comprising the amino acid sequence set forth in SEQ ID NO: 43;
(f) a heavy chain variable region comprising the amino acid sequence set forth in SEQ ID NO: 52, and a light chain variable region comprising the amino acid sequence set forth in SEQ ID NO: 53;
(g) a heavy chain variable region comprising the amino acid sequence set forth in SEQ ID NO: 60, and a light chain variable region comprising the amino acid sequence set forth in SEQ ID NO: 61;
(h) a heavy chain variable region comprising the amino acid sequence set forth in SEQ ID NO: 66, and a light chain variable region comprising the amino acid sequence set forth in SEQ ID NO: 67;
(i) a heavy chain variable region comprising the amino acid sequence set forth in SEQ ID NO: 76, and a light chain variable region comprising the amino acid sequence set forth in SEQ ID NO: 77;
(j) a heavy chain variable region comprising the amino acid sequence set forth in SEQ ID NO: 83, and a light chain variable region comprising the amino acid sequence set forth in SEQ ID NO: 84;
(k) a heavy chain variable region comprising the amino acid sequence set forth in SEQ ID NO: 92, and a light chain variable region comprising the amino acid sequence set forth in SEQ ID NO: 93;
(l) a heavy chain variable region comprising the amino acid sequence set forth in SEQ ID NO: 102, and a light chain variable region comprising the amino acid sequence set forth in SEQ ID NO: 103;
(m) a heavy chain variable region comprising the amino acid sequence set forth in SEQ ID NO: 108, and a light chain variable region comprising the amino acid sequence set forth in SEQ ID NO: 109;
(n) a heavy chain variable region comprising the amino acid sequence set forth in SEQ ID NO: 108, and a light chain variable region comprising the amino acid sequence set forth in SEQ ID NO: 113;
(o) a heavy chain variable region comprising the amino acid sequence set forth in SEQ ID NO: 119, and a light chain variable region comprising the amino acid sequence set forth in SEQ ID NO: 120;
(p) a heavy chain variable region comprising the amino acid sequence set forth in SEQ ID NO: 126, and a light chain variable region comprising the amino acid sequence set forth in SEQ ID NO: 127;
(q) a heavy chain variable region comprising the amino acid sequence set forth in SEQ ID NO: 131, and a light chain variable region comprising the amino acid sequence set forth in SEQ ID NO: 132;
(r) a heavy chain variable region comprising the amino acid sequence set forth in SEQ ID NO: 141, and a light chain variable region comprising the amino acid sequence set forth in SEQ ID NO: 142;
(s) a heavy chain variable region comprising the amino acid sequence set forth in SEQ ID NO: 147, and a light chain variable region comprising the amino acid sequence set forth in SEQ ID NO: 148;
(t) a heavy chain variable region comprising the amino acid sequence set forth in SEQ ID NO: 153, and a light chain variable region comprising the amino acid sequence set forth in SEQ ID NO: 154;
(u) a heavy chain variable region comprising the amino acid sequence set forth in SEQ ID NO: 157, and a light chain variable region comprising the amino acid sequence set forth in SEQ ID NO: 158;
(v) a heavy chain variable region comprising the amino acid sequence set forth in SEQ ID NO: 163, and a light chain variable region comprising the amino acid sequence set forth in SEQ ID NO: 164; and
(w) a heavy chain variable region comprising the amino acid sequence set forth in SEQ ID NO: 172, and a light chain variable region comprising the amino acid sequence set forth in SEQ ID NO: 173.

In certain embodiments, wherein:
(a) the heavy chain variable region comprises the amino acid sequence set forth in SEQ ID NO: 7, and the light chain variable region comprises the amino acid sequence set forth in SEQ ID NO: 8;
(b) the heavy chain variable region comprises the amino acid sequence set forth in SEQ ID NO: 34, and the light chain variable region comprises the amino acid sequence set forth in SEQ ID NO: 35; or
(c) the heavy chain variable region comprises the amino acid sequence set forth in SEQ ID NO: 42, and the light chain variable region comprises the amino acid sequence set forth in SEQ ID NO: 43.

In certain embodiments, the anti-DLL3 antibody or antigen-binding fragment thereof comprises a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
(a) a heavy chain variable region CDR3 comprising the amino acid sequence set forth in SEQ ID NO: 3 and a conservative modification thereof; and a light chain variable region CDR3 comprising the amino acid sequence set forth in SEQ ID NO: 6 and a conservative modification thereof;
(b) a heavy chain variable region CDR3 comprising the amino acid sequence set forth in SEQ ID NO: 13 and a conservative modification thereof; and a light chain variable region CDR3 comprising the amino acid sequence set forth in SEQ ID NO: 16 and a conservative modification thereof;
(c) a heavy chain variable region CDR3 comprising the amino acid sequence set forth in SEQ ID NO: 22 and a conservative modification thereof; and a light chain variable region CDR3 comprising the amino acid sequence set forth in SEQ ID NO: 23 and a conservative modification thereof;
(d) a heavy chain variable region CDR3 comprising the amino acid sequence set forth in SEQ ID NO: 30 and a conservative modification thereof; and a light chain variable region CDR3 comprising the amino acid sequence set forth in SEQ ID NO: 33 and a conservative modification thereof;
(e) a heavy chain variable region CDR3 comprising the amino acid sequence set forth in SEQ ID NO: 39 and a conservative modification thereof; and a light chain variable region CDR3 comprising the amino acid sequence set forth in SEQ ID NO: 41 and a conservative modification thereof;
(f) a heavy chain variable region CDR3 comprising the amino acid sequence set forth in SEQ ID NO: 48 and a conservative modification thereof; and a light chain variable region CDR3 comprising the amino acid sequence set forth in SEQ ID NO: 51 and a conservative modification thereof;
(g) a heavy chain variable region CDR3 comprising the amino acid sequence set forth in SEQ ID NO: 56 and a conservative modification thereof; and a light chain variable region CDR3 comprising the amino acid sequence set forth in SEQ ID NO: 59 and a conservative modification thereof;
(h) a heavy chain variable region CDR3 comprising the amino acid sequence set forth in SEQ ID NO: 64 and a conservative modification thereof; and a light chain variable region CDR3 comprising the amino acid sequence set forth in SEQ ID NO: 65 and a conservative modification thereof;
(i) a heavy chain variable region CDR3 comprising the amino acid sequence set forth in SEQ ID NO: 72 and a conservative modification thereof; and a light chain variable region CDR3 comprising the amino acid sequence set forth in SEQ ID NO: 75 and a conservative modification thereof;
(j) a heavy chain variable region CDR3 comprising the amino acid sequence set forth in SEQ ID NO: 81 and a conservative modification thereof; and a light chain variable region CDR3 comprising the amino acid sequence set forth in SEQ ID NO: 82 and a conservative modification thereof;
(k) a heavy chain variable region CDR3 comprising the amino acid sequence set forth in SEQ ID NO: 89 and a conservative modification thereof; and a light chain variable region CDR3 comprising the amino acid sequence set forth in SEQ ID NO: 91 and a conservative modification thereof;
(l) a heavy chain variable region CDR3 comprising the amino acid sequence set forth in SEQ ID NO: 98 and a conservative modification thereof; and a light chain variable region CDR3 comprising the amino acid sequence set forth in SEQ ID NO: 101 and a conservative modification thereof;
(m) a heavy chain variable region CDR3 comprising the amino acid sequence set forth in SEQ ID NO: 107 and a conservative modification thereof; and a light chain variable region CDR3 comprising the amino acid sequence set forth in SEQ ID NO: 82 and a conservative modification thereof;
(n) a heavy chain variable region CDR3 comprising the amino acid sequence set forth in SEQ ID NO: 107 and a conservative modification thereof; and a light chain variable region CDR3 comprising the amino acid sequence set forth in SEQ ID NO: 112 and a conservative modification thereof;
(o) a heavy chain variable region CDR3 comprising the amino acid sequence set forth in SEQ ID NO: 116 and a conservative modification thereof; and a light chain variable region CDR3 comprising the amino acid sequence set forth in SEQ ID NO: 118 and a conservative modification thereof;
(p) a heavy chain variable region CDR3 comprising the amino acid sequence set forth in SEQ ID NO: 123 and a conservative modification thereof; and a light chain variable region CDR3 comprising the amino acid sequence set forth in SEQ ID NO: 125 and a conservative modification thereof;
(q) a heavy chain variable region CDR3 comprising the amino acid sequence set forth in SEQ ID NO: 56 and a conservative modification thereof; and a light chain variable region CDR3 comprising the amino acid sequence set forth in SEQ ID NO: 130 and a conservative modification thereof;
(r) a heavy chain variable region CDR3 comprising the amino acid sequence set forth in SEQ ID NO: 137 and a conservative modification thereof; and a light chain variable region CDR3 comprising the amino acid sequence set forth in SEQ ID NO: 140 and a conservative modification thereof;
(s) a heavy chain variable region CDR3 comprising the amino acid sequence set forth in SEQ ID NO: 145 and a conservative modification thereof; and a light chain variable region CDR3 comprising the amino acid sequence set forth in SEQ ID NO: 125 and a conservative modification thereof;
(t) a heavy chain variable region CDR3 comprising the amino acid sequence set forth in SEQ ID NO: 152 and a conservative modification thereof; and a light chain variable region CDR3 comprising the amino acid sequence set forth in SEQ ID NO: 82 and a conservative modification thereof;
(u) a heavy chain variable region CDR3 comprising the amino acid sequence set forth in SEQ ID NO: 123 and a conservative modification thereof; and a light chain variable region CDR3 comprising the amino acid sequence set forth in SEQ ID NO: 59 and a conservative modification thereof;
(v) a heavy chain variable region CDR3 comprising the amino acid sequence set forth in SEQ ID NO: 161 and a conservative modification thereof; and a light chain variable region CDR3 comprising the amino acid sequence set forth in SEQ ID NO: 162 and a conservative modification thereof; and
(w) a heavy chain variable region CDR3 comprising the amino acid sequence set forth in SEQ ID NO: 168 and a conservative modification thereof; and a light chain variable region CDR3 comprising the amino acid sequence set forth in SEQ ID NO: 171 and a conservative modification thereof.

In certain embodiments, the heavy chain variable region and light chain variable region CDR2 domains of the antibody or antigen-binding fragment thereof are selected from the group consisting of:
(a) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 5 and a conservative modification thereof;
(b) a heavy chain variable region CDR2 comprising the amino acid sequence set forth in SEQ ID NO: 12 and a conservative modification thereof; and a light chain variable region CDR2 comprising the amino acid sequence set forth in SEQ ID NO: 15 and a conservative modification thereof;
(c) a heavy chain variable region CDR2 comprising the amino acid sequence set forth in SEQ ID NO: 29 and a conservative modification thereof; and a light chain variable region CDR2 comprising the amino acid sequence set forth in SEQ ID NO: 32 and a conservative modification thereof;
(d) a heavy chain variable region CDR2 comprising the amino acid sequence set forth in SEQ ID NO: 38 and a conservative modification thereof; and a light chain variable region CDR2 comprising the amino acid sequence set forth in SEQ ID NO: 5 and a conservative modification thereof;
(e) a heavy chain variable region CDR2 comprising the amino acid sequence set forth in SEQ ID NO: 47 and a conservative modification thereof; and a light chain variable region CDR2 comprising the amino acid sequence set forth in SEQ ID NO: 50 and a conservative modification thereof;
(f) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 58 and a conservative modification thereof;
(g) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 5 and a conservative modification thereof;
(h) a heavy chain variable region CDR2 comprising the amino acid sequence set forth in SEQ ID NO: 71 and a conservative modification thereof; and a light chain variable region CDR2 comprising the amino acid sequence set forth in SEQ ID NO: 74 and a conservative modification thereof;
(i) a heavy chain variable region CDR2 comprising the amino acid sequence set forth in SEQ ID NO: 80 and a conservative modification thereof; and a light chain variable region CDR2 comprising the amino acid sequence set forth in SEQ ID NO: 58 and a conservative modification thereof;
(j) a heavy chain variable region CDR2 comprising the amino acid sequence set forth in SEQ ID NO: 88 and a conservative modification thereof; and a light chain variable region CDR2 comprising the amino acid sequence set forth in SEQ ID NO: 216 and a conservative modification thereof;
(k) a heavy chain variable region CDR2 comprising the amino acid sequence set forth in SEQ ID NO: 97 and a conservative modification thereof; and a light chain variable region CDR2 comprising the amino acid sequence set forth in SEQ ID NO: 100 and a conservative modification thereof;
(l) a heavy chain variable region CDR2 comprising the amino acid sequence set forth in SEQ ID NO: 106 and a conservative modification thereof; and a light chain variable region CDR2 comprising the amino acid sequence set forth in SEQ ID NO: 58 and a conservative modification thereof;
(m) a heavy chain variable region CDR2 comprising the amino acid sequence set forth in SEQ ID NO: 106 and a conservative modification thereof; and a light chain variable region CDR2 comprising the amino acid sequence set forth in SEQ ID NO: 5 and a conservative modification thereof;
(n) a heavy chain variable region CDR2 comprising the amino acid sequence set forth in SEQ ID NO: 115 and a conservative modification thereof; and a light chain variable region CDR2 comprising the amino acid sequence set forth in SEQ ID NO: 100 and a conservative modification thereof;
(o) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 58 and a conservative modification thereof;
(p) a heavy chain variable region CDR2 comprising the amino acid sequence set forth in SEQ ID NO: 136 and a conservative modification thereof; and a light chain variable region CDR2 comprising the amino acid sequence set forth in SEQ ID NO: 139 and a conservative modification thereof;
(q) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 146 and a conservative modification thereof;
(r) a heavy chain variable region CDR2 comprising the amino acid sequence set forth in SEQ ID NO: 136 and a conservative modification thereof; and a light chain variable region CDR2 comprising the amino acid sequence set forth in SEQ ID NO: 74 and a conservative modification thereof; and
(s) a heavy chain variable region CDR2 comprising the amino acid sequence set forth in SEQ ID NO: 167 and a conservative modification thereof; and a light chain variable region CDR2 comprising the amino acid sequence set forth in SEQ ID NO: 170 and a conservative modification thereof.

In certain embodiments, the anti-DLL3 heavy chain variable region and light chain variable region CDR1 domains of the antibody or antigen-binding fragment thereof are selected from the group consisting of:
(a) a heavy chain variable region CDR1 comprising the amino acid sequence set forth in SEQ ID NO: 1 and a conservative modification thereof; and a light chain variable region CDR1 comprising the amino acid sequence set forth in SEQ ID NO: 4 and a conservative modification thereof;
(b) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 14 and a conservative modification thereof;
(c) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 4 and a conservative modification thereof;
(d) a heavy chain variable region CDR1 comprising the amino acid sequence set forth in SEQ ID NO: 28 and a conservative modification thereof; and a light chain variable region CDR1 comprising the amino acid sequence set forth in SEQ ID NO: 31 and a conservative modification thereof;
(e) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 40 and a conservative modification thereof;
(f) a heavy chain variable region CDR1 comprising the amino acid sequence set forth in SEQ ID NO: 46 and a conservative modification thereof; and a light chain variable region CDR1 comprising the amino acid sequence set forth in SEQ ID NO: 49 and a conservative modification thereof;
(g) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 57 and a conservative modification thereof;
(h) a heavy chain variable region CDR1 comprising the amino acid sequence set forth in SEQ ID NO: 70 and a conservative modification thereof; and a light chain variable region CDR1 comprising the amino acid sequence set forth in SEQ ID NO: 73 and a conservative modification thereof;
(i) a heavy chain variable region CDR1 comprising the amino acid sequence set forth in SEQ ID NO: 87 and a conservative modification thereof; and a light chain variable region CDR1 comprising the amino acid sequence set forth in SEQ ID NO: 90 and a conservative modification thereof;
(j) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 99 and a conservative modification thereof;
(k) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 117 and a conservative modification thereof;
(l) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 124 and a conservative modification thereof;
(m) a heavy chain variable region CDR1 comprising the amino acid sequence set forth in SEQ ID NO: 135 and a conservative modification thereof; and a light chain variable region CDR1 comprising the amino acid sequence set forth in SEQ ID NO: 138 and a conservative modification thereof;
(n) a heavy chain variable region CDR1 comprising the amino acid sequence set forth in SEQ ID NO: 151 and a conservative modification thereof; and a light chain variable region CDR1 comprising the amino acid sequence set forth in SEQ ID NO: 57 and a conservative modification thereof;
(o) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 73 and a conservative modification thereof; and
(p) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 169 and a conservative modification thereof.

In certain embodiments, one or more of the CDR sequences have up to about 5 amino acid substitutions. In certain embodiments, one or more of the CDR sequences have up to about 3 amino acid substitutions.

In certain embodiments, the anti-DLL3 antibody or antigen-binding fragment thereof comprises:
(a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3;
(b) a heavy chain variable region comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13;
(c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22;
(d) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30;
(e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39;
(f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
(g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56;
(h) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64;
(i) a heavy chain variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72;
(j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81;
(k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89;
(l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98;
(m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107;
(n) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116;
(o) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123;
(p) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137;
(q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145;
(r) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152;
(s) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161;
(t) a heavy chain variable region comprising a CDR1 comprising the amino acid sequence set forth in SEQ ID NO: 96, a CDR2 comprising the amino acid sequence set forth in SEQ ID NO: 167, and a CDR3 comprising the amino acid sequence set forth in SEQ ID NO: 168.

In certain embodiments, the anti-DLL3 antibody or antigen-binding fragment thereof comprises:
(a) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
(b) a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
(c) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23;
(d) a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
(e) a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
(f) a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
(g) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
(h) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
(i) a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
(j) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
(k) a light chain variable region comprising a CDR1 comprising the amino acid sequence set forth in SEQ ID NO: 90, a CDR2 comprising the amino acid sequence set forth in SEQ ID NO: 216, and a CDR3 comprising the amino acid sequence set forth in SEQ ID NO: 91;
(l) a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
(m) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
(n) a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
(o) a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
(p) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
(q) a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
(r) a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
(s) a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59;
(t) a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162;
(u) a light chain variable region comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171.

In certain embodiments, the anti-DLL3 antibody or antigen-binding fragment thereof comprises:
(a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
(b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
(c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23;
(d) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
(e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
(f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48; and a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
(g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
(h) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
(i) a heavy chain variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
(j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
(k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a light chain variable region comprising a CDR1 comprising the amino acid sequence set forth in SEQ ID NO: 90, a CDR2 comprising the amino acid sequence set forth in SEQ ID NO: 216, and a CDR3 comprising the amino acid sequence set forth in SEQ ID NO: 91;
(l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; and a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
(m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
(n) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
(o) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116; and a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
(p) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
(q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
(r) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137; and a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
(s) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145; and a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
(t) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
(u) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59;
(v) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162;
(w) a heavy chain variable region comprising a CDR1 comprising the amino acid sequence set forth in SEQ ID NO: 96, a CDR2 comprising the amino acid sequence set forth in SEQ ID NO: 167, and a CDR3 comprising the amino acid sequence set forth in SEQ ID NO: 168; and a light chain variable region comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171.

In certain embodiments, the antibody or an antigen-binding fragment thereof of comprises:
(a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
(b) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; or
(c) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41.

In certain embodiments, the sequence of the antibody is in a light-heavy variable chain orientation (V_{L}-V_{H}). In certain embodiments, the antibody or antigen-binding fragment thereof comprises a human variable region framework region.

In certain embodiments, the antibody or antigen-binding fragment thereof is a fully human or an antigen-binding fragment thereof. In certain embodiments, the antibody or antigen-binding fragment thereof is a chimeric antibody or an antigen-binding fragment thereof. In certain embodiments, the antibody or antigen-binding fragment thereof is a humanized antibody or an antigen-binding fragment thereof. In certain embodiments, the antigen-binding fragment of the antibody is a Fab, Fab′, F(ab′)₂, variable fragment (Fv) or a single chain variable fragment (scFv).

In certain embodiments, the antigen-binding fragment of the antibody or antigen-binding fragment thereof is an scFv.

In addition, the presently disclosed subject matter provides antibodies or antigen-binding fragments thereof, which cross-compete for binding to DLL3 with any of the above-described antibody or antigen-binding fragment thereof.

The presently disclosed subject matter further provides antibodies or antigen-binding fragments thereof, which binds to the same epitope region on DLL3 with any of the above-described antibody or antigen-binding fragment thereof.

The presently disclosed subject matter also provides immunoconjugates comprising the antibody or antigen-binding fragment thereof disclosed herein, linked to a therapeutic agent. In certain embodiments, the therapeutic agent is a drug, a cytotoxin, or a radioactive isotope. In certain embodiments, the immunoconjugate further comprises a chelator. In certain embodiments, the chelator is selected from the group consisting of AAZTA, BAT, BARAC, BPCA, TE2A, CB-TE2A, CB0TE1A1P, CB-TE2P, MM-TE2A, DM TE-2A, CP356, DATA, DBCO, DiAmSar, DIBO, DIMA, DFO, DGO, DOTA, DOTMA, DTPA, EDTA, EGTA, EHPG, H2dedpa, H4octapa, H2azapa, H5decapa, H6phospa, HBED, SHBED, HEHA, HYNIC, LICAM, MECAM, NODASA, NODAGA, NOPO, NOTA, NETA, PEPA, PCTA, PDTA, TACN-TM, TCMC, TETA, TETMA, TRAP (PRP9), TRITA, TTHA, and derivatives thereof. In certain embodiments, the chelator is DFO. In certain embodiments, the radioactive isotope is selected from the group consisting of ⁴⁷Sc, ⁶⁷Cu, ⁹⁰Y, ¹³¹I, ¹⁴⁹Tb, ¹⁶¹Tb, ¹⁷⁷Lu, ²²⁵Ac, ²¹³Bi, ²²³Ra, ⁸⁹Zr, and ²²⁷Th. In certain embodiments, the radioactive isotope is ¹⁷⁷Lu. In certain embodiments, the radioactive isotope is ⁸⁹Zr.

Furthermore, the presently disclosed subject matter provides multi-specific molecules comprising the antibody or antigen-binding fragment thereof disclosed herein, linked to one or more functional moieties. In certain embodiments, the one or more functional moieties have a different binding specificity than the antibody or antigen binding fragment thereof.

Additionally, the presently disclosed subject matter provides compositions comprising the antibody or antigen-binding fragment thereof disclosed herein, the immunoconjugate disclosed herein, or the multi-specific molecule disclosed herein. In certain embodiments, the composition is a pharmaceutical composition that further comprises a pharmaceutically acceptable carrier.

In addition, the presently disclosed subject matter provides nucleic acids encoding the antibody or antigen-binding fragment thereof disclosed herein, vectors comprising such nucleic acid molecules, and host cells comprising such vectors.

The presently disclosed subject matter provides methods for detecting DLL3 in a cell, a tissue, or a blood sample. In certain embodiments, the method comprises: contacting a cell, a tissue, or a blood sample with the antibody or antigen-binding fragment thereof disclosed herein, wherein the antibody or antigen-binding fragment thereof comprises a detectable label; and determining the amount of the labeled antibody or antigen-binding fragment thereof bound to the cell, tissue, blood sample by measuring the amount of detectable label associated with the cell, tissue, or blood sample, wherein the amount of bound antibody or antigen-binding fragment thereof indicates the amount of DLL3 in the cell, tissue, or blood sample.

Furthermore, the presently disclosed subject matter provides methods of treating or ameliorating a disease or disorder in a subject. In certain embodiments, the method comprises administering to the subject an antibody or antigen-binding fragment thereof, the immunoconjugate thereof, the multi-specific molecule, or the composition disclosed herein. In certain embodiments, the disease or disorder expresses DLL3. In certain embodiments, the disease or disorder is associated with overexpression of DLL3. In certain embodiments, the disease or disorder is tumor. In certain embodiments, the tumor is cancer. In certain embodiments, the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma. In certain embodiments, the neuroendocrine tumors of the lung are selected from the group consisting of pulmonary neuroendocrine cancer (including typical carcinoid tumors, and atypical carcinoid tumors), large cell neuroendocrine carcinoma, and small-cell lung cancer.

Furthermore, the presently disclosed subject matter provides kits for treating or ameliorating a disease or disorder in a subject, comprising the antibody or antigen-binding fragment thereof, the immunoconjugate thereof, the multi-specific molecule thereof, or the composition disclosed herein. In certain embodiments, the kit further comprises written instructions for using the antibody or antigen-binding fragment thereof, the immunoconjugate thereof, the multi-specific molecule thereof, or the composition thereof disclosed herein for treating or ameliorating a disease or disorder in a subject.

Further, the presently disclosed subject matter provides antibodies or antigen-binding fragments thereof, immunoconjugates, multi-specific molecules, or composition disclosed herein for use in treating or ameliorating a disease or disorder associated with DLL3 in a subject. In certain embodiments, the disease or disorder is a tumor. I n certain embodiments, the tumor is cancer. In certain embodiments, the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma. In certain embodiments, the neuroendocrine tumors of the lung are selected from the group consisting of pulmonary neuroendocrine cancer, large cell neuroendocrine carcinoma, and small-cell lung cancer. In certain embodiments, the subject is human.

The presently disclosed subject matter provides an immunoconjugate comprising an anti-DLL3 antibody or antigen-binding fragment thereof linked to a therapeutic agent. In certain embodiments, the anti-DLL3 antibody or antigen-binding fragment thereof comprises a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 187, SEQ ID NO: 197, SEQ ID NO: 204, or SEQ ID NO: 212. In certain embodiments, the anti-DLL3 antibody or antigen-binding fragment thereof comprises a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 188, SEQ ID NO: 198, SEQ ID NO: 205, or SEQ ID NO: 213. In certain embodiments, the anti-DLL3 antibody or antigen-binding fragment thereof comprises:
a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 187, SEQ ID NO: 197, SEQ ID NO: 204, or SEQ ID NO: 212; and
b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 188, SEQ ID NO: 198, SEQ ID NO: 205, or SEQ ID NO: 213.

In certain embodiments, the anti-DLL3 antibody or antigen-binding fragment thereof comprises a heavy chain variable region and a light chain variable region selected from the group consisting of:
a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 187, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 188;
b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 197, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 198;
c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 204, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 205; and
d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 212, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 213.

In certain embodiments, the anti-DLL3 antibody or antigen-binding fragment thereof comprises a heavy chain variable region comprising the amino acid sequence set forth in SEQ ID NO: 187, SEQ ID NO: 197, SEQ ID NO: 204, or SEQ ID NO: 212. In certain embodiments, the anti-DLL3 antibody or antigen-binding fragment thereof comprises a light chain variable region comprising the amino acid sequence set forth in SEQ ID NO: 188, SEQ ID NO: 198, SEQ ID NO: 205, or SEQ ID NO: 213.

In certain embodiments, the anti-DLL3 antibody or antigen-binding fragment thereof comprises:
a) a heavy chain variable region comprising the amino acid sequence set forth in SEQ ID NO: 187, SEQ ID NO: 197, SEQ ID NO: 204, or SEQ ID NO: 212; and
b) a light chain variable region comprising the amino acid sequence set forth in SEQ ID NO: 188, SEQ ID NO: 198, SEQ ID NO: 205, or SEQ ID NO: 213.

In certain embodiments,
a) a heavy chain variable region comprising the amino acid sequence set forth in SEQ ID NO: 187, and a light chain variable region comprising the amino acid sequence set forth in SEQ ID NO: 188;
b) a heavy chain variable region comprising the amino acid sequence set forth in SEQ ID NO: 197, and a light chain variable region comprising the amino acid sequence set forth in SEQ ID NO: 198;
c) a heavy chain variable region comprising the amino acid sequence set forth in SEQ ID NO: 204, and a light chain variable region comprising the amino acid sequence set forth in SEQ ID NO: 205; and
d) a heavy chain variable region comprising the amino acid sequence set forth in SEQ ID NO: 212, and a light chain variable region comprising the amino acid sequence set forth in SEQ ID NO: 213.

In certain embodiments, the anti-DLL3 antibody or antigen-binding fragment thereof comprises a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
a) a heavy chain variable region CDR3 comprising the amino acid sequence set forth in SEQ ID NO: 184 and a conservative modification thereof; and a light chain variable region CDR3 comprising the amino acid sequence set forth in SEQ ID NO: 186 and a conservative modification thereof;
b) a heavy chain variable region CDR3 comprising the amino acid sequence set forth in SEQ ID NO: 193 and a conservative modification thereof; and a light chain variable region CDR3 comprising the amino acid sequence set forth in SEQ ID NO: 196 and a conservative modification thereof;
c) a heavy chain variable region CDR3 comprising the amino acid sequence set forth in SEQ ID NO: 202 and a conservative modification thereof; and a light chain variable region CDR3 comprising the amino acid sequence set forth in SEQ ID NO: 203 and a conservative modification thereof; and
d) a heavy chain variable region CDR3 comprising the amino acid sequence set forth in SEQ ID NO: 210 and a conservative modification thereof; and a light chain variable region CDR3 comprising the amino acid sequence set forth in SEQ ID NO: 211 and a conservative modification thereof.

In certain embodiments, the heavy chain variable region and light chain variable region CDR2 domains are selected from the group consisting of:
a) a heavy chain variable region CDR2 comprising the amino acid sequence set forth in SEQ ID NO: 183 and a conservative modification thereof; and a light chain variable region CDR2 comprising the amino acid sequence set forth in SEQ ID NO: 50 and a conservative modification thereof;
b) a heavy chain variable region CDR2 comprising the amino acid sequence set forth in SEQ ID NO: 192 and a conservative modification thereof; and a light chain variable region CDR2 comprising the amino acid sequence set forth in SEQ ID NO: 195 and a conservative modification thereof;
c) a heavy chain variable region CDR2 comprising the amino acid sequence set forth in SEQ ID NO: 201 and a conservative modification thereof; and a light chain variable region CDR2 comprising the amino acid sequence set forth in SEQ ID NO: 5 and a conservative modification thereof; and
d) a heavy chain variable region CDR2 comprising the amino acid sequence set forth in SEQ ID NO: 209 and a conservative modification thereof; and a light chain variable region CDR2 comprising the amino acid sequence set forth in SEQ ID NO: 58 and a conservative modification thereof.

In certain embodiments, the heavy chain variable region and light chain variable region CDR1 domains are selected from the group consisting of:
a) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 185 and a conservative modification thereof;
b) a heavy chain variable region CDR1 comprising the amino acid sequence set forth in SEQ ID NO: 191 and a conservative modification thereof; and a light chain variable region CDR1 comprising the amino acid sequence set forth in SEQ ID NO: 194 and a conservative modification thereof;
c) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 4 and a conservative modification thereof; and
d) a heavy chain variable region CDR1 comprising the amino acid sequence set forth in SEQ ID NO: 208 and a conservative modification thereof; and a light chain variable region CDR1 comprising the amino acid sequence set forth in SEQ ID NO: 57 and a conservative modification thereof.

In certain embodiments, one or more of the CDR sequences have up to about 5 amino acid substitutions. In certain embodiments, one or more of the CDR sequences have up to about 3 amino acid substitutions.

In certain embodiments, the anti-DLL3 antibody or antigen-binding fragment thereof comprises:
a) a heavy chain variable region comprising a CDR1 comprising the amino acid sequence set forth in SEQ ID NO: 21, a CDR2 comprising the amino acid sequence set forth in SEQ ID NO: 183, and a CDR3 comprising the amino acid sequence set forth in SEQ ID NO: 184;
b) a heavy chain variable region comprising a CDR1 comprising the amino acid sequence set forth in SEQ ID NO: 191, a CDR2 comprising the amino acid sequence set forth in SEQ ID NO: 192, and a CDR3 comprising the amino acid sequence set forth in SEQ ID NO: 193;
c) a heavy chain variable region comprising a CDR1 comprising the amino acid sequence set forth in SEQ ID NO: 11, a CDR2 comprising the amino acid sequence set forth in SEQ ID NO: 201, and a CDR3 comprising the amino acid sequence set forth in SEQ ID NO: 202; or
d) a heavy chain variable region comprising a CDR1 comprising the amino acid sequence set forth in SEQ ID NO: 208, a CDR2 comprising the amino acid sequence set forth in SEQ ID NO: 209, and a CDR3 comprising the amino acid sequence set forth in SEQ ID NO: 210.

In certain embodiments, the anti-DLL3 antibody or antigen-binding fragment thereof comprises:
a) a light chain variable region comprising a CDR1 comprising the amino acid sequence set forth in SEQ ID NO: 185, a CDR2 comprising the amino acid sequence set forth in SEQ ID NO: 50, and a CDR3 comprising the amino acid sequence set forth in SEQ ID NO: 186;
b) a light chain variable region comprising a CDR1 comprising the amino acid sequence set forth in SEQ ID NO: 194, a CDR2 comprising the amino acid sequence set forth in SEQ ID NO: 195, and a CDR3 comprising the amino acid sequence set forth in SEQ ID NO: 196;
c) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 203; or
d) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 211.

In certain embodiments, the anti-DLL3 antibody or antigen-binding fragment thereof comprises:
a) a heavy chain variable region comprising a CDR1 comprising the amino acid sequence set forth in SEQ ID NO: 21, a CDR2 comprising the amino acid sequence set forth in SEQ ID NO: 183, and a CDR3 comprising the amino acid sequence set forth in SEQ ID NO: 184; and a light chain variable region comprising a CDR1 comprising the amino acid sequence set forth in SEQ ID NO: 185, a CDR2 comprising the amino acid sequence set forth in SEQ ID NO: 50, and a CDR3 comprising the amino acid sequence set forth in SEQ ID NO: 186;
b) a heavy chain variable region comprising a CDR1 comprising the amino acid sequence set forth in SEQ ID NO: 191, a CDR2 comprising the amino acid sequence set forth in SEQ ID NO: 192, and a CDR3 comprising the amino acid sequence set forth in SEQ ID NO: 193; and a light chain variable region comprising a CDR1 comprising the amino acid sequence set forth in SEQ ID NO: 194, a CDR2 comprising the amino acid sequence set forth in SEQ ID NO: 195, and a CDR3 comprising the amino acid sequence set forth in SEQ ID NO: 196;
c) a heavy chain variable region comprising a CDR1 comprising the amino acid sequence set forth in SEQ ID NO: 11, a CDR2 comprising the amino acid sequence set forth in SEQ ID NO: 201, and a CDR3 comprising the amino acid sequence set forth in SEQ ID NO: 202; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 203; or
d) a heavy chain variable region comprising a CDR1 comprising the amino acid sequence set forth in SEQ ID NO: 208, a CDR2 comprising the amino acid sequence set forth in SEQ ID NO: 209, and a CDR3 comprising the amino acid sequence set forth in SEQ ID NO: 210; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 211.

In certain embodiments, the antibody or antigen-binding fragment thereof binds to a DLL3 comprising the amino acid sequence set forth in SEQ ID NO: 215 or a fragment thereof. In certain embodiments, the antibody comprises a human variable region framework region. In certain embodiments, the antibody or antigen-binding fragment thereof is a fully human or an antigen-binding fragment thereof. In certain embodiments, the antibody or antigen-binding fragment thereof is a chimeric antibody or an antigen-binding fragment thereof. In certain embodiments, the antibody or antigen-binding fragment thereof is a humanized antibody or an antigen-binding fragment thereof. In certain embodiments, the antigen-binding fragment is a Fab, Fab′, F(ab′)2, variable fragment (Fv), or single chain variable region (scFv). In certain embodiments, the antigen antigen-binding fragment is an scFv. In certain embodiments, the therapeutic agent is a radioactive isotope.

In certain embodiments, the immunoconjugate further comprising a chelator. In certain embodiments, the chelator is selected from the group consisting of AAZTA, BAT, BARAC, BPCA, TE2A, CB-TE2A, CB0TE1A1P, CB-TE2P, MM-TE2A, DM TE-2A, CP356, DATA, DBCO, DiAmSar, DIBO, DIMA, DFO, DGO, DOTA, DOTMA, DTPA, EDTA, EGTA, EHPG, H2dedpa, H4octapa, H2azapa, H5decapa, H6phospa, HBED, SHBED, HEHA, HYNIC, LICAM, MECAM, NODASA, NODAGA, NOPO, NOTA, NETA, PEPA, PCTA, PDTA, TACN-TM, TCMC, TETA, TETMA, TRAP (PRP9), TRITA, TTHA, and derivatives thereof. In certain embodiments, the chelator is DFO. In certain embodiments, the radioactive isotope is selected from the group consisting of ⁴⁷Sc, ⁶⁷Cu, ⁹⁰Y, ¹³¹I, ¹⁴⁹Tb, ¹⁶¹Tb, ¹⁷⁷Lu, ²²⁵Ac, ²¹³Bi, ²²³Ra, ⁸⁹Zr, and ²²⁷Th. In certain embodiments, the radioactive isotope is ¹⁷⁷Lu. In certain embodiments, the radioactive isotope is ⁸⁹Zr.

The presently disclosed subject matter also provides a composition comprising the immunoconjugate disclosed herein. In certain embodiments, the composition is a pharmaceutical composition that further comprises a pharmaceutically acceptable carrier.

The presently disclosed subject matter further provides a method for detecting DLL3 in a whole cell, a tissue, or a blood sample, comprising: a) contacting a cell, tissue or blood sample with the immunoconjugate disclosed herein; and b) determining the amount of the immunoconjugate bound to the cell, tissue or blood sample by measuring the amount of detectable label associated with said cell or tissue, wherein the amount of bound immunoconjugate indicates the amount of DLL3 in the cell, tissue or blood sample.

The presently disclosed subject matter further provides a method of treating or ameliorating a disease or disorder associated with DLL3 in a subject, comprising administering to the subject the immunoconjugate or the composition disclosed herein. In certain embodiments, the disease or disorder is a tumor. In certain embodiments, the tumor is cancer. In certain embodiments, the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma. In certain embodiments, the neuroendocrine tumors of the lung are selected from the group consisting of pulmonary neuroendocrine cancer, large cell neuroendocrine carcinoma, and small-cell lung cancer. In certain embodiments, the subject is a human.

The presently disclosed subject matter further provides a kit for treating or ameliorating a disease or disorder in a subject, comprising the immunoconjugate or the composition disclosed herein. In certain embodiments, the kit further comprises written instructions for using the antibody or antigen-binding fragment thereof, immunoconjugate, multi-specific molecule, or composition for treating or ameliorating a disease or disorder in a subject.

Finally, the presently disclosed subject matter further provides the immunoconjugate or the composition disclosed herein for use in treating or ameliorating a disease or disorder associated with DLL3 in a subject. In certain embodiments, the disease or disorder is a tumor. In certain embodiments, the tumor is cancer. In certain embodiments, the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma. In certain embodiments, the neuroendocrine tumors of the lung are selected from the group consisting of pulmonary neuroendocrine cancer, large cell neuroendocrine carcinoma, and small-cell lung cancer. In certain embodiments, the subject is human

### 4. BRIEF DESCRIPTION OF THE DRAWINGS

The following Detailed Description, given by way of example, but not intended to limit the invention to specific embodiments described, may be understood in conjunction with the accompanying drawings.

FIGS. 1A and 1B depict the role of DLL3 in SCLC. FIG. 1A shows a schematic of the molecular mechanisms involving DLL3. FIG. 1B shows expression levels of DLL3 on cell membrane of SCLC cells.

FIGS. 2A and 2B depict that DLL3 is selectively expressed on the cell membrane in SCLC. FIG. 2A shows expression of DLL3 in different tissues. FIG. 2B shows membranous H-score. LCNEC=large cell neuroendocrine carcinoma, lung cancer.

FIG. 3 shows a schematic of the presently disclosed immunoconjugates.

FIG. 4 shows results of Fab-Zap internalization assay. RLUs=relative light units.

FIG. 5 shows a schematic of the immunoconjugates disclosed herein.

FIGS. 6A-6D depict effects of ⁸⁹Zr-DFO-BivE23. FIG. 6A shows a schematic of the bioconjugation of the E23 clone. FIG. 6B shows a schematic of the in vivo experimental setup.

FIG. 6C shows maximum intensity projections of representative images from Zr-89 immunoPET imaging of E23biv 120h post-injection in H82 SCLC model (300 μCi, 30 μg, via tail vein, n=3-4 per cohort). FIG. 6D shows biodistribution of ⁸⁹Zr-DFO-BivE23 in mice using PET/CT.

FIG. 7 shows a schematic of a dosimetry study performed to analyze the presently disclosed subject matter.

FIG. 8 shows biodistribution of ⁸⁹Zr-DFO-BivE23 in H660 male nude mice using PET/CT.

FIGS. 9A and 9B depict E23 analysis of dosimetry study. FIG. 9A shows extrapolation of human absorbed doses (ADs) from biodistribution experiments of ⁸⁹Zr-DFO-BivE23. FIG. 9B shows ^{Lu}177-DTPA-BivE23 H660 tumor model mouse dosimetry.

FIG. 10 shows a full course biodistribution study for H82 in female nude mice.

FIGS. 11A and 11B show quality control post-radiolabeling of a cell binding assay for the C8 clones. FIG. 11A shows results of the C8 IgG1 clone. FIG. 11B shows results of the C8 IgG4 clone.

FIGS. 12A-12E depict effects of ⁸⁹Zr-DFO-C8 IgG1 and ⁸⁹Zr-DFO-C8 IgG4. FIG. 12A shows a schematic of the bioconjugation of the C8 clones and of the in vivo experimental setup. FIG. 12B shows maximum intensity projections of representative images from Zr-89 immunoPET imaging of C8 IgG1 120h post-injection in H82 SCLC model (120 μCi, 30 μg, via tail vein, n=3-4 per cohort). FIG. 12C shows maximum intensity projections of representative images from Zr-89 immunoPET imaging of C8 IgG4 120h post-injection in H82 SCLC model (120 μCi, 30 μg, via tail vein, n=3-4 per cohort). FIG. 12D shows biodistribution study results of C8 IgG1 120h post-injection. FIG. 12E shows biodistribution study results of C8 IgG4 120h post-injection.

FIG. 13 shows a schematic of a dosimetry study performed to analyze the C8 IgG1 and the C8 IgG4.

FIGS. 14A and 14B show quality control data for the C8 clones. FIG. 14A shows radiochemical yield of the C8 IgG1 clone. FIG. 14B shows radiochemical yield of the C8 IgG4 clone.

FIGS. 15A and 15B show biodistribution of C8 clones in H82 female nude mice using PET/CT. FIG. 15A shows biodistribution of ⁸⁹Zr-DFO-C8 IgG1. FIG. 15B shows biodistribution of ⁸⁹Zr-DFO-C8 IgG4.

FIGS. 16A and 16B show blocking studies of C8 clones in H82 female nude mice using PET/CT. FIG. 16A shows blocking of ⁸⁹Zr-DFO-C8 IgG1. FIG. 26B shows block of ⁸⁹Zr-DFO-C8 IgG4.

FIG. 17 depicts biodistribution of control SC16 in H82 female nude mice using PET/CT at 72h.

FIGS. 18A and 18B depict C8 analysis of dosimetry study. FIG. 18A shows extrapolation of human absorbed doses (ADs) from biodistribution experiments of ⁸⁹Zr-DFO-C8 IgG1 and ⁸⁹Zr-DFO-C8 IgG4. FIG. 18B shows ^{Lu}177-DTPA-C8 IgG1 and ^{Lu}177-DTPA-C8 IgG4 in H82 tumor model mouse dosimetry.

### 5. DETAILED DESCRIPTION OF EMBODIMENTS OF THE INVENTION

The presently disclosed subject matter provides anti-DLL3 antibodies. Non-limiting embodiments of the present disclosure are described by the present specification and Examples.

For purposes of clarity of disclosure and not by way of limitation, the detailed description is divided into the following subsections:
5.1. Definitions;
5.2. DLL3;
5.3. Anti-DLL3 Antibodies;
5.4. Nucleic Acids encoding the Antibodies or Antigen-binding Fragments;
5.5. Pharmaceutical Compositions and Methods of Treatment;
5.6. Diagnostic and Prognostic Methods;
5.7. Kits; and
5.8. Exemplary Embodiments.

### 5.1 Definitions

In the description that follows, certain conventions will be followed as regards the usage of terminology. Generally, terms used herein are intended to be interpreted consistently with the meaning of those terms as they are known to those of skill in the art.

“Antibody” and “antibodies” as those terms are known in the art refer to antigen binding proteins of the immune system. The term “antibody” as referred to herein includes whole, full length antibodies having an antigen-binding region, and any fragment thereof in which the “antigen-binding fragment” or “antigen-binding region” is retained, or single chains, for example, single chain variable fragment (scFv), thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Cl q) of the classical complement system.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the presently disclosed subject matter may include amino acid residues not encoded by human germline immunoglobulin sequences (e.g., mutations introduced by random or site-specific mutagenesis in vitro or by somatic mutation in vivo).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to DLL3” is intended to refer to an antibody that binds to DLL3 (e.g., human DLL3) with a dissociation constant (K_{D}) of about 1×10⁻⁸ M or less, about 5×10⁻⁹ M or less, about 1×10⁻⁹ M or less, about 5×10⁻¹ M or less, about 1×10⁻¹ M or less, about 5×10⁻¹¹ M or less, about 1×10⁻¹¹ M or less, about 5×10⁻¹² M or less, or about 1×10⁻¹² M or less.

An “antibody that competes for binding” or “antibody that cross-competes for binding” with a reference antibody for binding to an antigen, e.g., DLL3, refers to an antibody that blocks binding of the reference antibody to the antigen (e.g., DLL3) in a competition assay by 50% or more, and conversely, the reference antibody blocks binding of the antibody to the antigen (e.g., DLL3) in a competition assay by 50% or more. An exemplary competition assay is described in “Antibodies”, Harlow and Lane (Cold Spring Harbor Press, Cold Spring Harbor, NY).

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term” an antibody which binds specifically to an antigen (e.g., a DLL3 polypeptide).”

The term “antigen-binding fragment” or “antigen-binding region” of an antibody, as used herein, refers to that region or fragment of the antibody that binds to the antigen and which confers antigen specificity to the antibody; fragments of antigen-binding proteins, for example, antibodies includes one or more fragments of an antibody that retain the ability to specifically bind to an antigen (e.g., a DLL3 polypeptide). It has been shown that the antigen-binding function of an antibody can be performed by fragments of a full-length antibody. Examples of antigen-binding fragments encompassed within the term “antibody fragments” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., Nature 1989; 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science (1988); 242:423-426; and Huston et al., Proc Natl Acad Sci (1998); 85:5879-5883. These antibody fragments are obtained using conventional techniques known to those of skill in the art, and the fragments are screened for utility in the same manner as are intact antibodies.

An “antibody” or “antigen-binding protein” is one which has been identified and separated and/or recovered from a component of its natural environment. “Synthetic antibodies” or “recombinant antibodies” are generally generated using recombinant technology or using peptide synthetic techniques known to those of skill in the art.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain.

Non-limiting examples of linkers are disclosed in Shen et al., Anal Chem (2008); 80(6):1910-1917 and WO 2014/087010, the contents of which are hereby incorporated by reference in their entireties. In certain embodiments, the linker is a G4S linker. In certain embodiments, the linker comprises or consists of the amino acid sequence set forth in SEQ ID NO: 176, which is provided below:

[SEQ ID NO: 176]
GGGGSGGGGSGGGSGGGGS

In certain embodiments, the linker comprises or consists of the amino acid sequence set forth in SEQ ID NO: 177, which is provided below:

[SEQ ID NO: 177]
GGGGSGGGGSGGGGS

In certain embodiments, the linker comprises or consists of the amino acid sequence set forth in SEQ ID NO: 178, which is provided below:

[SEQ ID NO: 178]
GGGGSGGGGSGGGGSGGGSGGGGS

In certain embodiments, the linker comprises or consists of the amino acid sequence set forth in SEQ ID NO: 179, which is provided below:

[SEQ ID NO: 179]
GGGGSGGGGSGGGGSGGGGSGGGSGGGGS

In certain embodiments, the linker comprises or consists of the amino acid sequence set forth in SEQ ID NO: 180, which is provided below:

[SEQ ID NO: 180]
GGGGS

In certain embodiments, the linker comprises or consists of the amino acid sequence set forth in SEQ ID NO: 181, which is provided below:

[SEQ ID NO: 181]
GGGGSGGGGS

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 1988; 85:5879-5883). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008; 27(6):455-51; Peter et al., J Cachexia Sarcopenia Muscle 2012 Aug. 12; Shieh et al., J Imunol 2009; 183(4):2277-85; Giomarelli et al., Thromb Haemost 2007; 97(6):955-63; Fife eta., J Clin Invst 2006; 116(8):2252-61; Brocks et al., Immunotechnology 1997; 3(3):173-84; Moosmayer et al., Ther Immunol 1995; 2(10:31-40). Agonistic scFvs having stimulatory activity have been described (see, e.g., Peter et al., J Bioi Chern 2003; 25278(38):36740-7; Xie et al., Nat Biotech 1997; 15(8):768-71; Ledbetter et al., Crit Rev Immunol 1997; 17(5-6):427-55; Ho et al., Bio Chim Biophys Acta 2003; 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “vector” refers to any genetic element, such as a plasmid, phage, transposon, cosmid, chromosome, virus, virion, etc., which is capable of replication when associated with the proper control elements and which can transfer gene sequences into cells.

Thus, the term includes cloning and expression vehicles, as well as viral vectors and plasmid vectors.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise three heavy chain and three light chain CDRs or CDR regions in the variable region. CDRs provide the majority of contact residues for the binding of the antibody to the antigen or epitope region. In certain embodiments, the CDRs are identified according to the IMGT system. In certain embodiments, the CDRs are identified using the IMGT numbering system accessible at http://www.imgt.org/IMGT_vquest/input.

The terms “isolated” denotes a degree of separation from original source or surroundings.

An “isolated antibody” is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr (2007); B 848:79-87.

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated nucleic acid encoding an antibody” (including references to a specific antibody, e.g. an anti-KLB antibody) refers to one or more nucleic acid molecules encoding antibody heavy and light chains (or fragments thereof), including such nucleic acid molecule(s) in a single vector separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “immunoconjugate” is an antibody conjugated to one or more heterologous molecule(s), including, but not limited to, a cytotoxic agent.

As used herein, the term “derivative” refers to a compound that is derived from some other compound and maintains its general structure. For example, but without any limitation, trichloromethane (chloroform) is a derivative of methane.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition, and the form and effective concentration of the cells administered.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters; guinea pigs; rabbits; dogs; cats; sheep; pigs; goats; cattle; horses; and non-human primates such as apes and monkeys.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, antibodies of the presently disclosed subject matter are used to delay development of a disease or to slow the progression of a disease, e.g., a tumor, e.g., a tumor associated with DLL3.

The terms “comprises”, “comprising”, and are intended to have the broad meaning ascribed to them in U.S. Patent Law and can mean “includes”, “including” and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As described herein, any concentration range, percentage range, ratio range or integer range is to be understood to include the value of any integer within the recited range and, when appropriate, fractions thereof (such as one tenth and one hundredth of an integer), unless otherwise indicated.

Other aspects of the presently disclosed subject matter are described in the following disclosure and are within the ambit of the presently disclosed subject matter.

### 5.2 DLL3

DLL3 is selectively expressed in high grade pulmonary neuroendocrine tumors of the lung (LU-NETs). Lu-NETs embrace a heterogeneous family of neoplasms classified into four histological variants, namely typical carcinoid (TC), atypical carcinoid (AC), large cell neuroendocrine carcinoma (LCNEC) and small cell lung carcinoma (SCLC). Increased expression of DLL3 was observed in SCLC and LCNEC patient-derived xenograft tumors and was also confirmed in primary tumors. See Saunders et al., Sci Translational Medicine (302): 302ra136 (2015). Both SCLC and pulmonary LCNEC are high-grade and poor-prognosis tumors, with higher incidence in smokers. Pulmonary LCNEC exhibits biologically aggressive behavior, similarly to SCLC. Stage by stage, survival curves of pulmonary LCNEC and SCLC overlap, and in addition, survival is lower than other NSCLCs. Prognosis is poor even in patients with potentially resectable stage I lung cancer with 5-year survival rates ranging from 27% to 67%. See Iyoda A. et al., J Thorac Cardiovasc Surg. 138:446-453 (2009).

Delta is one of the Drosophila ligands of Notch that activate signaling in adjacent cells. Humans have four known Notch receptors (NOTCH1 to NOTCH4), and three homologs of Delta,
termed delta-like ligands: DLL1, DLL3 and DLL4. It has been reported that unlike DLL1 and DLL4, DLL3 inhibits Notch signaling rather than activating it.

DLL3 (also known as Delta-like 3 or SCDO1) is a member of the Delta-like family of

Notch DSL ligands. Aberrant DLL3 expression (genotypic and/or phenotypic) is associated with various tumorigenic cell subpopulations such as cancer stem cells and tumor initiating cells.

In certain embodiments, the presently disclosed anti-DLL3 antibodies or antigen-binding fragments thereof bind to human DLL3. In certain embodiments, the human DLL3 comprises or consists of the amino acid sequence with a UniProt Reference No: Q9NYJ7-1 (SEQ ID NO: 182) or a fragment thereof. SEQ ID NO: 182 is provided below. In certain embodiments, the DLL3 comprises an extracellular domain, a transmembrane domain, and a cytoplasmic domain. In certain embodiments, the extracellular domain comprises or consists of amino acids 27 to 492 of SEQ ID NO: 182. In certain embodiments, the transmembrane domain comprises or consists of amino acids 493 to 513 of SEQ ID NO: 182. In certain embodiments, the cytoplasmic domain comprises or consists of amino acids 514 to 618 of SEQ ID NO: 182.

In certain embodiments, the extracellular domain of DLL3 comprises a DSL domain, an EGF-like 1 domain, an EGF-like 2 domain, an EGF-like 3 domain, an EGF-like 4 domain, and EGF-like 5 domain, and an EGF-like 6 domain. In certain embodiments, the DSL domain comprises or consists of amino acids 176 to 215 of SEQ ID NO: 182. In certain embodiments, the EGF-like 1 domain comprises or consists of amino acids 216 to 249 of SEQ ID NO: 182. In certain embodiments, the EGF-like 2 domain comprises or consists of amino acids 274 to 310 of SEQ ID NO: 182. In certain embodiments, the EGF-like 3 domain comprises or consists of amino acids 312 to 351 of SEQ ID NO: 182. In certain embodiments, the EGF-like 4 domain comprises or consists of amino acids 353 to 389 of SEQ ID NO: 182. In certain embodiments, the EGF-like 5 domain comprises or consists of amino acids 391 to 427 of SEQ ID NO: 182. In certain embodiments, the EGF-like 6 domain comprises or consists of amino acids 429 to 465 of SEQ ID NO: 182.

[SEQ ID NO: 182]
MVSPRMSGLLSQTVILALIFLPQTRPAGVFELQIHSFGPGPGPGAPRSP
CSARLPCRLFFRVCLKPGLSEEAAESPCALGAALSARGPVYTEQPGAPA
PDLPLPDGLLQVPFRDAWPGTESFIIETWREELGDQIGGPAWSLLARVA
GRRRLAAGGPWARDIQRAGAWELRFSYRARCEPPAVGTACTRLCRPRSA
PSRCGPGLRPCAPLEDECEAPLVCRAGCSPEHGFCEQPGECRCLEGWTG
PLCTVPVSTSSCLSPRGPSSATTGCLVPGPGPCDGNPCANGGSCSETPR
SFECTCPRGFYGLRCEVSGVTCADGPCFNGGLCVGGADPDSAYICHCPP
GFQGSNCEKRVDRCSLQPCRNGGLCLDLGHALRCRCRAGFAGPRCEHDL
DDCAGRACANGGTCVEGGGAHRCSCALGFGGRDCRERADPCAARPCAHG
GRCYAHFSGLVCACAPGYMGARCEFPVHPDGASALPAAPPGLRPGDPQR
YLLPPALGLLVAAGVAGAALLLVHVRRRGHSQDAGSRLLAGTPEPSVHA
LPDALNNLRTQEGSGDGPSSSVDWNRPEDVDPQGIYVISAPSIYAREVA
TPLFPPLHTGRAGQRQHLLFPYPSSILSVK

In certain embodiments, the DLL3 comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 182 or a fragment thereof.

In certain embodiments, the antigen recognizing receptor binds to EGF-like 3 domain of DLL3. In certain embodiments, the antigen recognizing receptor binds to amino acids 312 to 351 of SEQ ID NO: 182. In certain embodiments, the antigen recognizing receptor binds to EGF-like 4 domain of DLL3. In certain embodiments, the antigen recognizing receptor binds to amino acids 353 to 389 of SEQ ID NO: 182. In certain embodiments, the antigen recognizing receptor binds to EGF-like 5 domain of DLL3. In certain embodiments, the antigen recognizing receptor binds to amino acids 391 to 427 of SEQ ID NO: 182. In certain embodiments, the antigen recognizing receptor binds to EGF-like 6 domain of DLL3. In certain embodiments, the antigen recognizing receptor binds to amino acids 429 to 465 of SEQ ID NO: 182.

In certain embodiments, the anti-DLL3 antibodies or antigen-binding fragments thereof bind to a portion of human DLL3. In certain embodiments, the anti-DLL3 antibodies or antigen-binding fragments thereof bind to the extracellular domain of DLL3. In certain embodiments, the anti-DLL3 antibodies or antigen-binding fragments thereof bind to amino acids 27 to 492 of SEQ ID NO: 182.

### 5.3 Anti-DLL3 Antibodies

The antibodies of the presently disclosed subject matter are characterized by particular functional features or properties of the antibodies. For example, the antibodies bind specifically to DLL3 (e.g., bind to human DLL3).

In certain embodiments, a presently disclosed antibody or antigen-binding fragment binds to DLL3 (e.g., human DLL3) with a binding affinity, for example with a dissociation constant (K_{D}) of about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, or about 1×10⁻¹¹ M or less, about 5×10⁻¹² M or less, or about 1×10⁻¹² M or less. In certain embodiments, a presently disclosed antibody or antigen-binding fragment binds to DLL3 (e.g., human DLL3) with a binding affinity, for example with a dissociation constant (K_{D}) of about 5×10⁻⁹ M or less. In certain embodiments, a presently disclosed antibody or antigen-binding fragment binds to DLL3 (e.g., human DLL3) with a binding affinity, for example with a dissociation constant (K_{D}) of about 1×10⁻⁹ M or less.

In certain embodiments, a presently disclosed antibody or antigen-binding fragment binds to DLL3 (e.g., human DLL3) with a binding affinity, for example with a dissociation constant (K_{D}) of about 3.5×10⁻⁹ M. In certain embodiments, a presently disclosed antibody or antigen-binding fragment binds to DLL3 (e.g., human DLL3) with a binding affinity, for example with a dissociation constant (K_{D}) of about 1.5×10⁻⁹ M. In certain embodiments, a presently disclosed antibody or antigen-binding fragment binds to DLL3 (e.g., human DLL3) with a binding affinity, for example with a dissociation constant (K_{D}) of about 1×10⁻¹² M.

The heavy and light chains of a presently disclosed antibody or antigen-binding fragment can be full-length (e.g., an antibody can include at least one (e.g., one or two) complete heavy chains, and at least one (e.g., one or two) complete light chains) or can include an antigen-binding fragment (a Fab, F(ab′)₂, Fv or a single chain Fv fragment (“scFv”)). In certain embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4. In certain embodiments, the immunoglobulin isotype is IgG1 (e.g., human IgG1). The choice of antibody isotype can depend on the immune effector function that the antibody is designed to elicit. In certain embodiments, the antibody light chain constant region is chosen from, e.g., kappa or lambda, particularly kappa.

In constructing a recombinant immunoglobulin, appropriate amino acid sequences for constant regions of various immunoglobulin isotypes and methods for the production of a wide array of antibodies are known to those of skill in the art.

### 5.3.1 Single-Chain Variable Fragments (scFvs)

In certain embodiments, the presently disclosed subject matter includes antibodies or antigen-binding fragments thereof that have the scFv sequence fused to one or more constant domains to form an antibody with an Fc region of a human immunoglobulin to yield a bivalent protein, increasing the overall avidity and stability of the antibody. In addition, the Fc portion allows the direct conjugation of other molecules, including but not limited to fluorescent dyes, cytotoxins, radioisotopes etc. to the antibody for example, for use in antigen quantitation studies, to immobilize the antibody for affinity measurements, for targeted delivery of a therapeutic agent, to test for Fc-mediated cytotoxicity using immune effector cells and many other applications.

The results presented here highlight the specificity, sensitivity and utility of the presently disclosed antibodies or antigen-binding fragments in targeting a DLL3 polypeptide (e.g., a human DLL3 polypeptide).

In certain embodiments, the anti-DLL3 scFv is an scFv-Fc fusion protein or a full-length human IgG with V_{H} and V_{L} regions or CDRs selected from Table 1. In certain embodiments, the anti-DLL3 scFv comprises a V_{H} comprising the amino acid sequence set forth in SEQ ID NO: 7. An exemplary nucleotide sequence encoding the amino acid sequence of SEQ ID NO: 7 is set forth in SEQ ID NO: 9. In certain embodiments, the anti-DLL3 scFv comprises a V_{L} comprising the amino acid sequence set forth in SEQ ID NO: 8. An exemplary nucleotide sequence encoding the amino acid sequence of SEQ ID NO: 8 is set forth in SEQ ID NO: 10. SEQ ID NO: 7-10 are provided in Table 1. In certain embodiments, the scFv is designated as “J8”.

In certain embodiments, the anti-DLL3 scFv comprises a V_{H} Comprising the amino acid sequence set forth in SEQ ID NO: 7 and a V_{L} comprising the amino acid sequence set forth in SEQ ID NO: 8. In certain embodiments, the anti-DLL3 scFv comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof. SEQ ID NOs: 1-3 are provided in Table 1.

In certain embodiments, the anti-DLL3 scFv comprises a VL comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof. SEQ ID NOs: 4-6 are provided in Table 1.

In certain embodiments, the anti-DLL3 scFv comprises a VH comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof; and a VL comprising a CDR1 comprising the amino acid sequence set forth in SEQ ID NO: 4 or a conservative modification thereof, a CDR2 comprising the amino acid sequence set forth in SEQ ID NO: 5 or a conservative modification, and a CDR3 comprising the amino acid sequence set forth in SEQ ID NO: 6 or a conservative modification thereof.

In certain embodiments, the anti-DLL3 scFv comprises a VH comprising a CDR1 comprising the amino acid sequence set forth in SEQ ID NO: 1, a CDR2 comprising the amino acid sequence set forth in SEQ ID NO: 2, a V_{H} CDR3 comprising the amino acid sequence set forth in SEQ ID NO: 3; and a VL comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the anti-DLL3 scFv comprises a V_{H} comprising the amino acid sequence set forth in SEQ ID NO: 7, and a V_{L} comprising the amino acid sequence set forth in SEQ ID NO: 8. In certain embodiments, the V_{H} and V_{L} are linked via a linker. In certain embodiments, the linker comprises the amino acid sequence set forth in SEQ ID NO: 177.

In certain embodiments, the variable regions are linked one after another such that a heavy chain variable region (V_{H}) is position at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 1

| CDRs | 1 | 2 | 3 |
|---|---|---|---|
| V_{H} | GYTFTIY | NPSGGS | NDYVDYVTTDYYGMDV |
| | [SEQ ID NO: 1] | [SEQ ID NO: 2] | [SEQ ID NO: 3] |
| V_{L} | RASQGISNYLA | AASSLQS | QQYNSSPYT |
| | [SEQ ID NO: 4] | [SEQ ID NO: 5] | [SEQ ID NO: 6] |

| | |
|---|---|
| Full V_{H} | QVHLVQSGAEVKKPGASVKVSCKASGYTFTIYYIHWVRQAPGQGLEWMG |
| | IINPSGGSTSYAQKFQGRVTMTRDTSTSTVYMELSSLRSEDTAVYYCSG |
| | NDYVDYVTTDYYGMDVWGQGTTVTVSS [SEQ ID NO: 7] |
| Full V_{L} | DIQMTQSPSSLSASVGDRVTITCRASQGISNYLAWFQQKPGKAPKSLIY |
| | AASSLQSGVPSKFSGSGSGTDFTLTISSLQPEDFATYYCQQYNSSPYTF |
| | GQGTKLEIK [SEQ ID NO: 8] |
| DNA | CAGGTGCATCTGGTGCAGTCTGGGGCTGAGGTGAAGAAGCCTGGGGCCT |
| for Full V_{H} | CAGTGAAGGTTTCTTGCAAGGCATCTGGATACACCTTCACCATCTACTA |
| | TATTCACTGGGTGCGACAGGCCCCTGGACAGGGGCTTGAGTGGATGGGA |
| | ATAATCAACCCTAGTGGTGGTAGCACAAGCTACGCACAGAAGTTCCAGG |
| | GCAGAGTCACCATGACCAGGGACACGTCCACGAGCACAGTCTACATGGA |
| | GCTGAGCAGCCTGAGATCTGAGGACACGGCCGTATATTACTGTTCGGGT |
| | AATGACTACGTTGACTACGTAACTACTGACTACTACGGTATGGACGTCT |
| | GGGGCCAAGGGACCACGGTCACCGTCTCCTCA [SEQ ID NO: 9] |
| DNA | GACATCCAGATGACCCAGTCTCCATCCTCACTGTCTGCATCTGTAGGAG |
| for Full V_{L} | ATAGAGTCACCATCACTTGTCGGGCGAGTCAGGGCATTAGCAATTATTT |
| | AGCCTGGTTTCAGCAGAAACCAGGGAAAGCCCCTAAGTCCCTGATCTAT |
| | GCTGCATCCAGTTTGCAAAGTGGGGTCCCATCAAAGTTCAGCGGCAGTG |
| | GATCTGGGACAGATTTCACACTCACCATCAGCAGCCTGCAGCCTGAAGA |
| | TTTTGCAACTTATTACTGCCAACAGTATAATAGTTCCCCGTACACTTTT |
| | GGCCAGGGGACCAAGCTGGAGATCAAA [SEQ ID NO: 10] |

In certain embodiments, the anti-DLL3 scFv is an scFv-Fc fusion protein or a full-length human IgG with V_{H} and V_{L} regions or CDRs selected from Table 2. In certain embodiments, the anti-DLL3 scFv comprises a V_{H} comprising the amino acid sequence set forth in SEQ ID NO: 17, as shown in Table 2. An exemplary nucleotide sequence encoding the amino acid sequence of SEQ ID NO: 17 is set forth in SEQ ID NO: 19. In certain embodiments, the anti-DLL3 scFv comprises a V_{L} comprising the amino acid sequence set forth in SEQ ID NO: 18. An exemplary nucleotide sequence encoding the amino acid sequence of SEQ ID NO: 18 is set forth in SEQ ID NO: 20. In certain embodiments, the anti-DLL3 scFv comprises a V_{H} comprising the amino acid sequence set forth in SEQ ID NO: 17 and a V_{L} comprising the amino acid sequence set forth in SEQ ID NO: 18. SEQ ID NO: 17-20 are provided in Table 2. In certain embodiments, the scFv is designated as “L22”.

In certain embodiments, the anti-DLL3 scFv comprises a VH comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof. SEQ ID NOs: 11-13 are provided in Table 2.

In certain embodiments, the anti-DLL3 scFv comprises a VL comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification thereof. SEQ ID NOs: 14-16 are provided in Table 2.

In certain embodiments, the anti-DLL3 scFv comprises a VH comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof; and a VL comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification.

In certain embodiments, the anti-DLL3 scFv comprises a VH comprising a CDR1 comprising the amino acid sequence set forth in SEQ ID NO: 11, a CDR2 comprising the amino acid sequence set forth in SEQ ID NO: 12, a V_{H} CDR3 comprising the amino acid sequence set forth in SEQ ID NO: 13; and a VL comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

In certain embodiments, the anti-DLL3 scFv comprises a V_{H} comprising the amino acid sequence set forth in SEQ ID NO: 17, and a V_{L} comprising the amino acid sequence set forth in SEQ ID NO: 18. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 2

| CDRs | 1 | 2 | 3 |
|---|---|---|---|
| V_{H} | GFTFSSY | SGRGGD | ERGLGFDY |
| | [SEQ ID NO: 11] | [SEQ ID NO: 12] | [SEQ ID NO: 13] |
| V_{L} | RSSQSLLDSDDGNTYLD | TLSYRAS | MQHLEFPLT |
| | [SEQ ID NO: 14] | [SEQ ID NO: 15] | [SEQ ID NO: 16] |

| | |
|---|---|
| Full V_{H} | EVQLLESGGGLVQPGGSLRLSCAASGFTFSSYAMGWVRQTPGKGLEWVS |
| | TISGRGGDTYYADSVKGRFTISRDNSKNTLYLQMNSLRAEDTAIYYCAK |
| | ERGLGFDYWGQGTLVTVSS |
| | [SEQ ID NO: 17] |
| Full V_{L} | DIVMTQTPLSLPVTPGEPASISCRSSQSLLDSDDGNTYLDWYLQKPGQS |
| | PQLLIYTLSYRASGVPDRFSGSGSGTDFTLKISRVEAEDVGVYYCMQHL |
| | EFPLTFGGGTKVEIK |
| | [SEQ ID NO: 18] |
| DNA | GAGGTGCAGCTGTTGGAGTCTGGGGGAGGCTTGGTACAGCCTGGGGGGT |
| for Full V_{H} | CCCTGAGACTCTCCTGTGCAGCCTCTGGATTCACCTTTAGCAGCTATGC |
| | CATGGGCTGGGTCCGCCAGACTCCAGGGAAGGGGCTGGAGTGGGTCTCA |
| | ACTATTAGTGGTCGTGGTGGTGACACATACTACGCAGACTCCGTGAAGG |
| | GCCGGTTCACCATCTCCAGAGACAATTCCAAGAACACGCTGTATCTGCA |
| | AATGAACAGCCTGAGAGCCGAGGACACGGCCATATATTACTGTGCGAAA |
| | GAGAGGGGACTGGGGTTTGACTACTGGGGCCAGGGAACCCTGGTCACCG |
| | TCTCCTCA |
| | [SEQ ID NO: 19] |
| DNA | GATATTGTGATGACCCAGACTCCACTCTCCCTGCCCGTCACCCCTGGAG |
| for Full V_{L} | AGCCGGCCTCCATCTCCTGCAGGTCTAGTCAGAGCCTCTTGGATAGTGA |
| | TGATGGAAACACCTATTTGGACTGGTACCTGCAGAAGCCAGGGCAGTCT |
| | CCACAGCTCCTGATCTATACGCTTTCCTATCGGGCCTCTGGAGTCCCAG |
| | ACAGGTTCAGTGGCAGTGGGTCAGGCACTGATTTCACACTGAAAATCAG |
| | CAGGGTGGAGGCTGAGGATGTTGGAGTTTATTACTGCATGCAACATCTA |
| | GAGTTTCCGCTCACTTTCGGCGGAGGGACCAAGGTGGAGATCAAA |
| | [SEQ ID NO: 20] |

In certain embodiments, the anti-DLL3 scFv is an scFv-Fc fusion protein or full-length human IgG with V_{H} and V_{L} regions or CDRs selected from Table 3. In certain embodiments, the anti-DLL3 scFv comprises a V_{H} comprising the amino acid sequence set forth in SEQ ID NO: 24, as shown in Table 3. An exemplary nucleotide sequence encoding the amino acid sequence of SEQ ID NO: 24 is set forth in SEQ ID NO: 26. In certain embodiments, the anti-DLL3 scFv comprises a V_{L} comprising the amino acid sequence set forth in SEQ ID NO: 25. An exemplary nucleotide sequence encoding the amino acid sequence of SEQ ID NO: 25 is set forth in SEQ ID NO: 27. SEQ ID NO: 24-27 are provided in Table 3. In certain embodiments, the scFv is designated as “B2”.

In certain embodiments, the anti-DLL3 scFv comprises a V_{H} comprising the amino acid sequence set forth in SEQ ID NO: 24 and a V_{L} comprising the amino acid sequence set forth in SEQ ID NO: 25.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 22 or a conservative modification thereof. SEQ ID NOs: 2, 21, and 22 are provided in Table 3.

In certain embodiments, the anti-DLL3 scFv comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 23 or a conservative modification thereof. SEQ ID NOs: 4, 5, and 23 are provided in Table 3.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 22 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 23 or a conservative modification thereof.

In certain embodiments, the anti-DLL3 scFv comprises a VH comprising a CDR1 comprising the amino acid sequence set forth in SEQ ID NO: 21, a CDR2 comprising the amino acid sequence set forth in SEQ ID NO: 2, a V_{H} CDR3 comprising the amino acid sequence set forth in SEQ ID NO: 22; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23.

In certain embodiments, the anti-DLL3 scFv comprises a V_{H} comprising the amino acid sequence set forth in SEQ ID NO: 24, and a V_{L} comprising the amino acid sequence set forth in SEQ ID NO: 25. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}

TABLE 3

| CDRS | 1 | 2 | 3 |
|---|---|---|---|
| V_{H} | GYTFTSY | NPSGGS | GDYDILTDYYYNMDV |
| | [SEQ ID NO: 21] | [SEQ ID NO: 2] | [SEQ ID NO: 22] |
| V_{L} | RASQGISNYLA | AASSLQS | LQHNSYPYT |
| | [SEQ ID NO: 4] | [SEQ ID NO: 5] | [SEQ ID NO: 23] |

| | |
|---|---|
| Full V_{H} | QVQLVQSGAEVKKPGASVKVSCKASGYTFTSYYMHWVRQAPGQGLEWMG |
| | IINPSGGSTSYAQKFRGRVTMTRDTSTSTVYMELSSLRSEDTAVYYCAR |
| | GDYDILTDYYYNMDVWGQGTTVTVSS |
| | [SEQ ID NO: 24] |
| Full V_{L} | DIQMTQSPSAMSASVGDRVTITCRASQGISNYLAWFQQKPGKVPKRLIY |
| | AASSLQSGVPSRFSGSGSGTEFTLTISSLQPEDFATYYCLQHNSYPYTF |
| | GQGTKLEIK |
| | [SEQ ID NO: 25] |
| DNA | CAGGTGCAGCTGGTGCAGTCTGGGGCTGAGGTGAAGAAGCCTGGGGCCT |
| for Full V_{H} | CAGTGAAGGTTTCCTGCAAGGCATCTGGATACACCTTCACCAGCTACTA |
| | TATGCACTGGGTGCGACAGGCCCCTGGACAAGGGCTTGAGTGGATGGGA |
| | ATAATCAACCCTAGTGGTGGTAGCACAAGCTACGCACAGAAGTTCCGGG |
| | GCAGAGTCACCATGACCAGGGACACGTCCACGAGCACAGTCTACATGGA |
| | GCTGAGCAGCCTGAGATCTGAGGACACGGCCGTTTATTACTGTGCGAGA |
| | GGGGATTACGATATTTTGACGGACTACTACTACAATATGGACGTCTGGG |
| | GCCAAGGGACCACGGTCACCGTCTCCTCA |
| | [SEQ ID NO: 26] |
| DNA | GACATCCAGATGACCCAGTCTCCATCTGCCATGTCTGCATCTGTAGGAG |
| for Full V_{L} | ACAGAGTCACCATCACTTGTCGGGCGAGTCAGGGCATTAGCAATTATTT |
| | AGCCTGGTTTCAGCAGAAACCAGGGAAAGTCCCTAAGCGCCTGATCTAT |
| | GCTGCATCCAGTTTGCAAAGTGGGGTCCCATCAAGGTTCAGCGGCAGTG |
| | GATCTGGGACAGAATTCACTCTCACAATCAGCAGCCTGCAGCCTGAAGA |
| | TTTTGCAACTTATTACTGTCTACAGCATAATAGTTACCCGTACACTTTC |
| | GGCCAGGGGACCAAGCTGGAGATCAAA |
| | [SEQ ID NO: 27] |

In certain embodiments, the anti-DLL3 scFv is an scFv-Fc fusion protein or full length human IgG with V_{H} and V_{L} regions or CDRs selected from Table 4. In certain embodiments, the anti-DLL3 scFv comprises a V_{H} comprising the amino acid sequence set forth in SEQ ID NO: 34, as shown in Table 4. An exemplary nucleotide sequence encoding the amino acid sequence of SEQ ID NO: 34 is set forth in SEQ ID NO: 36. In certain embodiments, the anti-DLL3 scFv comprises a V_{L} comprising the amino acid sequence set forth in SEQ ID NO: 35. An exemplary nucleotide sequence encoding the amino acid sequence of SEQ ID NO: 35 is set forth in SEQ ID NO: 37. SEQ ID NO: 34-37 are provided in Table 4. In certain embodiments, the scFv is designated as “A18”

In certain embodiments, the anti-DLL3 scFv comprises a V_{H} comprising the amino acid sequence set forth in SEQ ID NO: 34 and a V_{L} comprising the amino acid sequence set forth in SEQ ID NO: 35. SEQ ID NOs: 34 and 35 are provided in Table 4.

In certain embodiments, the anti-DLL3 scFv comprises a VH comprising a CDR1 comprising the amino acid sequence set forth in SEQ ID NO: 28 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof. SEQ ID NOs: 28, 29, and 30 are provided in Table 4.

In certain embodiments, the anti-DLL3 scFv comprises a V_{L} comprising a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof. SEQ ID NOs: 31, 32, and 33 are provided in Table 4.

In certain embodiments, the anti-DLL3 scFv comprises a VH comprising a CDR1 comprising the amino acid sequence set forth in SEQ ID NO: 28 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof; and a V_{L} comprising a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof.

In certain embodiments, the anti-DLL3 scFv comprises a VH comprising a CDR1 comprising the amino acid sequence set forth in SEQ ID NO: 28, a CDR2 comprising the amino acid sequence set forth in SEQ ID NO: 29, a V_{H} CDR3 comprising the amino acid sequence set forth in SEQ ID NO: 30; and a V_{L} comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33.

In certain embodiments, the anti-DLL3 scFv comprises a V_{H} comprising the amino acid sequence set forth in SEQ ID NO: 34, and a V_{L} comprising the amino acid sequence set forth in SEQ ID NO: 35. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 4

| CDRs | 1 | 2 | 3 |
|---|---|---|---|
| V_{H} | GFSLSTSGM | DWDDN | IRGYSGSYDAFDI |
| | [SEQ ID NO: 28] | [SEQ ID NO: 29] | [SEQ ID NO: 30] |
| V_{L} | RSSQSLLHSNGYNYLD | LGSNRAP | MQALQTPFT |
| | [SEQ ID NO: 31] | [SEQ ID NO: 32] | [SEQ ID NO: 33] |

| | |
|---|---|
| Full V_{H} | QVTLRESGPALVKSTQTLTLTCTFSGFSLSTSGMCVSWIRQTPGKALEW |
| | LALIDWDDNKYYSTSLKTRLIISKDTSKNQVVLTMTNMEPVDTATYYCA |
| | RIRGYSGSYDAFDIWGQGTMVTVSS |
| | [SEQ ID NO: 34] |
| Full V_{L} | DIVMTQSPLSLPVTPGEPASISCRSSQSLLHSNGYNYLDWYLQKPGQSP |
| | HILLYLGSNRAPGVPDRFSGSGSGTDFTLKISRVEAEDVGVYYCMQALQ |
| | TPFTFGPGTKVDIK |
| | [SEQ ID NO: 35] |
| DNA | CAGGTCACCTTGAGGGAGTCTGGTCCTGCGCTGGTGAAATCCACACAGA |
| for Full V_{H} | CCCTCACACTGACCTGCACCTTCTCTGGGTTCTCACTCAGTACTAGTGG |
| | AATGTGTGTGAGCTGGATCCGTCAGACCCCAGGGAAGGCCCTGGAGTGG |
| | CTTGCACTCATTGATTGGGATGATAATAAATACTACAGCACATCTCTGA |
| | AGACCAGGCTCATCATCTCCAAGGACACCTCCAAAAACCAGGTGGTCCT |
| | TACAATGACCAATATGGAACCTGTGGACACAGCCACGTATTATTGTGCA |
| | CGGATACGGGGGTATAGTGGGAGCTACGATGCTTTTGATATCTGGGGCC |
| | AAGGGACAATGGTCACCGTCTCTTCA |
| | [SEQ ID NO: 36] |
| DNA | GATATTGTGATGACTCAGTCTCCACTCTCCCTGCCCGTCACCCCTGGAG |
| for Full V_{L} | AGCCGGCCTCCATCTCCTGCAGGTCTAGTCAGAGCCTCCTGCATAGTAA |
| | TGGATACAACTATTTGGATTGGTACCTGCAGAAGCCAGGGCAGTCTCCA |
| | CACATCCTGCTCTATTTGGGTTCTAATCGGGCCCCCGGGGTCCCTGACA |
| | GGTTCAGTGGCAGTGGATCAGGCACAGATTTTACACTGAAAATCAGCAG |
| | AGTGGAGGCTGAGGATGTTGGGGTTTATTACTGCATGCAAGCTCTACAA |
| | ACTCCATTCACTTTCGGCCCTGGGACCAAAGTGGATATCAAA |
| | [SEQ ID NO: 37] |

In certain embodiments, the anti-DLL3 scFv is an scFv-Fc fusion protein or a full-length human IgG with V_{H} and V_{L} regions or CDRs selected from Table 5. In certain embodiments, the anti-DLL3 scFv comprises a V_{H} comprising the amino acid sequence set forth in SEQ ID NO: 42, as shown in Table 5. An exemplary nucleotide sequence encoding the amino acid sequence of SEQ ID NO: 42 is set forth in SEQ ID NO: 44. In certain embodiments, the anti-DLL3 scFv comprises a V_{L} comprising the amino acid sequence set forth in SEQ ID NO: 43. An exemplary nucleotide sequence encoding the amino acid sequence of SEQ ID NO: 43 is set forth in SEQ ID NO: 45. SEQ ID NO: 42-45 are provided in Table 5. In certain embodiments, the scFv is designated as “E9”.

In certain embodiments, the anti-DLL3 scFv comprises a V_{H} comprising the amino acid sequence set forth in SEQ ID NO: 42 and a V_{L} comprising the amino acid sequence set forth in SEQ ID NO: 43.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof. SEQ ID NOs: 21, 38, and −39 are provided in Table 5.

In certain embodiments, the anti-DLL3 scFv comprises a V_{L} comprising a CDR1 comprising the amino acid sequence set forth in SEQ ID NO: 40 or a conservative modification thereof, a CDR2 comprising the amino acid sequence set forth in SEQ ID NO: 5 or a conservative modification thereof, and a CDR3 comprising the amino acid sequence set forth in SEQ ID NO: 41 or a conservative modification thereof. SEQ ID NOs: 40, 5, and 41 are provided in Table 5.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof; and a V_{L} comprising a CDR1 comprising the amino acid sequence set forth in SEQ ID NO: 40 or a conservative modification thereof, a CDR2 comprising the amino acid sequence set forth in SEQ ID NO: 5 or a conservative modification thereof, and a CDR3 comprising the amino acid sequence set forth in SEQ ID NO: 41 or a conservative modification thereof.

In certain embodiments, the anti-DLL3 scFv comprises a VH comprising a CDR1 comprising the amino acid sequence set forth in SEQ ID NO: 21, a CDR2 comprising the amino acid sequence set forth in SEQ ID NO: 38, a V_{H} CDR3 comprising the amino acid sequence set forth in SEQ ID NO: 39; and a V_{L} comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41.

In certain embodiments, the anti-DLL3 scFv comprises a V_{H} comprising the amino acid sequence set forth in SEQ ID NO: 42, and a V_{L} comprising the amino acid sequence set forth in SEQ ID NO: 43. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 5

| CDRs | 1 | 2 | 3 |
|---|---|---|---|
| V_{H} | GYTFTSY | NSSGGS | GYCTNGVYYDEDEDY |
| | [SEQ ID NO: 21] | [SEQ ID NO: 38] | [SEQ ID NO: 39] |
| V_{L} | RASQGISHYLA | AASSLQS | QQYNSYPLT |
| | [SEQ ID NO: 40] | [SEQ ID NO: 5] | [SEQ ID NO: 41] |

| | |
|---|---|
| Full V_{H} | QVQLVQSGAEVKKPGASVKVSCKASGYTFTSYYMHWVRQAPGQGLEWMG |
| | VINSSGGSTSYAQKFQGRVTMTRDTSTSTVYMDLSSLRSEDTAVYYCAR |
| | GYCTNGVYYDEDFDYWGQGTLVTVSS |
| | [SEQ ID NO: 42] |
| Full V_{L} | DIQMTQSPSSLSASVGDRVTITCRASQGISHYLAWFQQKPGKAPTSLIY |
| | AASSLQSGVPSKFSGSGSGTDFTLTISSLQPEDFATYYCQQYNSYPLTF |
| | GGGTKVEIK |
| | [SEQ ID NO: 43] |
| DNA | CAGGTGCAGCTGGTGCAGTCTGGGGCTGAGGTGAAGAAGCCTGGGGCCT |
| for Full V_{H} | CAGTGAAGGTTTCCTGCAAGGCATCTGGATACACCTTCACCAGCTACTA |
| | TATGCACTGGGTGCGACAGGCCCCTGGACAAGGGCTTGAGTGGATGGGA |
| | GTAATCAATTCTAGTGGTGGTAGCACAAGCTACGCACAGAAGTTCCAGG |
| | GCAGAGTCACCATGACCAGGGACACGTCCACGAGCACAGTCTACATGGA |
| | CCTGAGCAGCCTGAGATCTGAGGACACGGCCGTGTATTACTGTGCGAGA |
| | GGGTATTGTACTAATGGTGTTTACTATGATGAGGACTTTGACTACTGGG |
| | GCCAGGGAACCCTGGTCACCGTCTCCTCA |
| | [SEQ ID NO: 44] |
| DNA | GACATCCAGATGACCCAGTCTCCATCCTCACTGTCTGCATCTGTAGGAG |
| for Full V_{L} | ACAGAGTCACCATCACTTGTCGGGCGAGTCAGGGCATTAGCCATTATTT |
| | AGCCTGGTTTCAGCAGAAACCAGGGAAAGCCCCTACGTCCCTGATCTAT |
| | GCTGCATCCAGTTTGCAAAGTGGGGTCCCATCAAAGTTCAGCGGCAGTG |
| | GATCTGGGACAGATTTCACTCTCACCATCAGCAGCCTGCAGCCTGAAGA |
| | TTTTGCAACTTATTACTGCCAACAGTATAATAGTTACCCTCTCACTTTC |
| | GGCGGAGGGACCAAGGTGGAGATCAAA |
| | [SEQ ID NO: 45] |

In certain embodiments, the anti-DLL scFv is an scFv-Fc fusion protein or a full-length human IgG with V_{H} and V_{L} regions or CDRs selected from Table 6. In certain embodiments, the anti-DLL3 scFv comprises a V_{H} comprising the amino acid sequence set forth in SEQ ID NO: 52, as shown in Table 6. An exemplary nucleotide sequence encoding the amino acid sequence of SEQ ID NO: 52 is set forth in SEQ ID NO: 54. In certain embodiments, the anti-DLL3 scFv comprises a V_{L} comprising the amino acid sequence set forth in SEQ ID NO: 53. An exemplary nucleotide sequence encoding the amino acid sequence of SEQ ID NO: 53 is set forth in SEQ ID NO: 55. SEQ ID NO: 52-55 are provided in Table 6. In certain embodiments, the scFv is designated as “G3”.

In certain embodiments, the anti-DLL3 scFv comprises a V_{H} comprising the amino acid sequence set forth in SEQ ID NO: 52 and a V_{L} comprising the amino acid sequence set forth in SEQ ID NO: 53.

In certain embodiments, the anti-DLL3 scFv comprises a VH comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof. SEQ ID NOs: 46-48 are provided in Table 6.

In certain embodiments, the anti-DLL3 scFv comprises a V_{L} comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof. SEQ ID NOs: 49, 50, and 51 are provided in Table 6.

In certain embodiments, the anti-DLL3 scFv comprises a VH comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof; and a V_{L} comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof.

In certain embodiments, the anti-DLL3 scFv comprises a VH comprising a CDR1 comprising the amino acid sequence set forth in SEQ ID NO: 46, a CDR2 comprising the amino acid sequence set forth in SEQ ID NO: 47, a V_{H} CDR3 comprising the amino acid sequence set forth in SEQ ID NO: 48; and a V_{L} comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51.

In certain embodiments, the anti-DLL3 scFv comprises a V_{H} comprising the amino acid sequence set forth in SEQ ID NO: 52, and a V_{L} comprising the amino acid sequence set forth in SEQ ID NO: 53. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}

TABLE 6

| CDRs | 1 | 2 | 3 |
|---|---|---|---|
| V_{H} | GGSISSY | YTSGS | EDWESWTFDI |
| | [SEQ ID NO: 46] | [SEQ ID NO: 47] | [SEQ ID NO: 48] |
| V_{L} | RSGQSLVYSDGDTYLN | KVSNRDS | MQGTHWPLS |
| | [SEQ ID NO: 49] | [SEQ ID NO: 50] | [SEQ ID NO: 51] |

| | |
|---|---|
| Full V_{H} | QVQLQESGPGLVKPSETLFLTCTVSGGSISSYYWTWIRQPAGKGLEWIG |
| | RIYTSGSTNYNPSLKSRVTMSVDTSKNQFSLKLSSVTAADTAVYYCARE |
| | DWESWTFDIWGQGTMVTVSS |
| | [SEQ ID NO: 52] |
| Full V_{L} | DVVMTQSPLSLPVTLGQPASISCRSGQSLVYSDGDTYLNWFQQRPGQSP |
| | RRLIYKVSNRDSGVPDRFSGSGSGTDFTLKISRVEAEDVGVYYCMQGTH |
| | WPLSFGGGTKVEIK |
| | [SEQ ID NO: 53] |
| DNA | CAGGTGCAGCTGCAGGAGTCGGGCCCAGGACTGGTGAAGCCTTCGGAGA |
| for Full V_{H} | CCCTGTTCCTCACCTGCACTGTCTCTGGTGGCTCCATCAGTAGTTACTA |
| | CTGGACCTGGATCCGGCAGCCCGCCGGGAAGGGACTGGAGTGGATTGGG |
| | CGTATCTATACCAGTGGGAGCACCAACTACAACCCCTCCCTCAAGAGTC |
| | GAGTCACCATGTCAGTAGACACGTCCAAGAACCAGTTCTCCCTGAAGCT |
| | GAGCTCTGTGACCGCCGCGGACACGGCCGTGTATTACTGTGCGAGAGAG |
| | GACTGGGAATCGTGGACTTTTGATATCTGGGGCCAAGGGACAATGGTCA |
| | CCGTCTCTTCA |
| | [SEQ ID NO: 54] |
| DNA | GATGTTGTGATGACTCAGTCTCCACTCTCCCTGCCCGTCACCCTTGGAC |
| for Full V_{L} | AGCCGGCCTCCATCTCCTGCAGGTCTGGTCAAAGCCTCGTTTACAGTGA |
| | TGGAGACACCTACTTGAATTGGTTTCAGCAGAGGCCAGGCCAATCTCCA |
| | AGGCGCCTAATTTATAAGGTTTCTAACCGGGACTCTGGGGTCCCAGACA |
| | GATTCAGCGGCAGTGGGTCAGGCACTGATTTCACACTGAAAATCAGCAG |
| | GGTGGAGGCTGAGGATGTTGGGGTTTATTACTGCATGCAAGGTACACAC |
| | TGGCCGCTCTCTTTCGGCGGAGGGACCAAGGTGGAGATCAAA |
| | [SEQ ID NO: 55] |

In certain embodiments, the anti-DLL3 scFv is an scFv-Fc fusion protein or a full-length human IgG with V_{H} and V_{L} regions or CDRs selected from Table 7. In certain embodiments, the anti-DLL3 scFv comprises a V_{H} comprising the amino acid sequence set forth in SEQ ID NO: 60, as shown in Table 7. An exemplary nucleotide sequence encoding the amino acid sequence of SEQ ID NO: 60 is set forth in SEQ ID NO: 62. In certain embodiments, the anti-DLL3 scFv comprises a V_{L} comprising the amino acid sequence set forth in SEQ ID NO: 61. An exemplary nucleotide sequence encoding the amino acid sequence of SEQ ID NO: 61 is set forth in SEQ ID NO: 63. SEQ ID NO: 60-63 are provided in Table 7. In certain embodiments, the scFv is designated as “M11”.

In certain embodiments, the anti-DLL3 scFv comprises a V_{H} comprising the amino acid sequence set forth in SEQ ID NO: 60 and a V_{L} comprising the amino acid sequence set forth in SEQ ID NO: 61.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof. SEQ ID NOs: 2, 21, and 56 are provided in Table 7.

In certain embodiments, the anti-DLL3 scFv comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof. SEQ ID NOs: 57-59 are provided in Table 7.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof.

In certain embodiments, the anti-DLL3 scFv comprises a VH comprising a CDR1 comprising the amino acid sequence set forth in SEQ ID NO: 21, a CDR2 comprising the amino acid sequence set forth in SEQ ID NO: 2, a V_{H} CDR3 comprising the amino acid sequence set forth in SEQ ID NO: 56; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59.

In certain embodiments, the anti-DLL3 scFv comprises a V_{H} comprising the amino acid sequence set forth in SEQ ID NO: 60, and a V_{L} comprising the amino acid sequence set forth in SEQ ID NO: 61. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 7

| CDRs | 1 | 2 | 3 |
|---|---|---|---|
| V_{H} | GYTFTSY | NPSGGS | QLELLGNAFDI |
| | [SEQ ID NO: 21] | [SEQ ID NO: 2] | [SEQ ID NO: 56] |
| V_{L} | RASQSVSSSYLA | GASSRAT | QQYGSSPYT |
| | [SEQ ID NO: 57] | [SEQ ID NO: 58] | [SEQ ID NO: 59] |

| | |
|---|---|
| Full V_{H} | QVQLVQSGAEVKKPGASVKVSCKASGYTFTSYYIHWVRQAPGQGLEWMG |
| | IINPSGGSKSYAQKFQGRVTMTRDTSTGTVYMELSSLRSEDTAVYYCAG |
| | QLELLGNAFDIWGQGTMVTVSS |
| | [SEQ ID NO: 60] |
| Full V_{L} | EIVLTQSPGTLSLSPGERATLSCRASQSVSSSYLAWYQQKPGQAPRLLI |
| | YGASSRATGIPDRFSGSGSGTDFTLTISRLEPEDFAVYYCQQYGSSPYT |
| | FGQGTKLEIK |
| | [SEQ ID NO: 61] |
| DNA | CAGGTGCAGCTGGTGCAGTCTGGGGCTGAGGTGAAGAAGCCTGGGGCCT |
| for Full V_{H} | CAGTGAAGGTTTCCTGCAAGGCATCTGGATACACCTTCACCAGCTACTA |
| | TATACACTGGGTGCGACAGGCCCCTGGACAAGGGCTTGAGTGGATGGGA |
| | ATAATCAACCCTAGTGGTGGTAGTAAAAGCTACGCACAGAAGTTCCAGG |
| | GCAGAGTCACCATGACCAGGGACACGTCCACGGGAACAGTCTATATGGA |
| | GCTGAGCAGCCTGAGATCTGAGGACACGGCCGTGTATTACTGTGCGGGA |
| | CAACTGGAACTTTTGGGGAATGCTTTTGATATCTGGGGCCAAGGGACAA |
| | TGGTCACCGTCTCTTCA |
| | [SEQ ID NO: 62] |
| DNA | GAAATTGTGTTGACGCAGTCTCCAGGCACCCTGTCTTTGTCTCCAGGGG |
| for Full V_{L} | AAAGAGCCACCCTCTCCTGCAGGGCCAGTCAGAGTGTTAGCAGCAGCTA |
| | CTTAGCCTGGTACCAGCAGAAACCTGGCCAGGCTCCCAGGCTCCTCATC |
| | TATGGTGCATCCAGCAGGGCCACTGGCATCCCAGACAGGTTCAGTGGCA |
| | GTGGGTCTGGGACAGACTTCACTCTCACCATCAGCAGACTGGAGCCTGA |
| | AGATTTTGCAGTGTATTACTGTCAGCAGTATGGTAGCTCACCGTACACT |
| | TTTGGCCAGGGGACCAAGCTGGAGATCAAA |
| | [SEQ ID NO: 63] |

In certain embodiments, the anti-DLL3 scFv is an scFv-Fc fusion protein or a full-length human IgG with V_{H} and V_{L} regions or CDRs selected from Table 8. In certain embodiments, the anti-DLL3 scFv comprises a V_{H} comprising the amino acid sequence set forth in SEQ ID NO: 66, as shown in Table 8. An exemplary nucleotide sequence encoding the amino acid sequence of SEQ ID NO: 66 is set forth in SEQ ID NO: 68. In certain embodiments, the anti-DLL3 scFv comprises a V_{L} comprising the amino acid sequence set forth in SEQ ID NO: 67. An exemplary nucleotide sequence encoding the amino acid sequence of SEQ ID NO: 67 is set forth in SEQ ID NO: 69. SEQ ID NO: 66-69 are provided in Table 8. In certain embodiments, the scFv is designated as “024”.

In certain embodiments, the anti-DLL3 scFv comprises a V_{H} comprising the amino acid sequence set forth in SEQ ID NO: 66 and a V_{L} comprising the amino acid sequence set forth in SEQ ID NO: 67.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 64 or a conservative modification thereof. SEQ ID NOs: 21, 2, and 64 are provided in Table 8.

In certain embodiments, the anti-DLL3 scFv comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 65 or a conservative modification thereof. SEQ ID NOs: 4, 5 and 65 are provided in Table 8.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 64 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 65 or a conservative modification thereof.

In certain embodiments, the anti-DLL3 scFv comprises a VH comprising a CDR1 comprising the amino acid sequence set forth in SEQ ID NO: 21, a CDR2 comprising the amino acid sequence set forth in SEQ ID NO: 2, a V_{H} CDR3 comprising the amino acid sequence set forth in SEQ ID NO: 64; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65.

In certain embodiments, the anti-DLL3 scFv comprises a V_{H} comprising the amino acid sequence set forth in SEQ ID NO: 66, and a V_{L} comprising the amino acid sequence set forth in SEQ ID NO: 67. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 8

| CDRs | 1 | 2 | 3 |
|---|---|---|---|
| V_{H} | GYTFTSY | NPSGGS | ERQQLDYYYFAMDV |
| | [SEQ ID NO: 21] | [SEQ ID NO: 2] | [SEQ ID NO: 64] |
| V_{L} | RASQGISNYLA | AASSLQS | QQYNSYPPT |
| | [SEQ ID NO: 4] | [SEQ ID NO: 5] | [SEQ ID NO: 65] |

| | |
|---|---|
| Full V_{H} | QVQLVQSGAEVKKPGASVKVSCKASGYTFTSYYLHWVRQAPGHGLEWMA |
| | IINPSGGSTSYAQKFLGRVTMTRDTSTNTVYMELSSLRSEDTAVYYCAR |
| | ERQQLDYYYFAMDVWGQGTTVTVSS |
| | [SEQ ID NO: 66] |
| Full V_{L} | DIQMTQSPSSLSASVGDRVTITCRASQGISNYLAWFQQKPGKAPKSLIY |
| | AASSLQSGVPSKFSGSGSGTDFTLTISSLQPEDFATYYCQQYNSYPPTF |
| | GQGTKVEIK |
| | [SEQ ID NO: 67] |
| DNA | CAGGTGCAGCTGGTGCAGTCTGGGGCTGAGGTGAAGAAGCCTGGGGCCT |
| for Full V_{H} | CAGTGAAGGTTTCCTGCAAGGCATCTGGATACACCTTCACCAGCTACTA |
| | TTTGCACTGGGTGCGACAGGCCCCTGGACACGGGCTTGAGTGGATGGCA |
| | ATAATCAACCCTAGTGGTGGTAGTACAAGCTACGCACAGAAGTTCCTGG |
| | GCAGAGTCACCATGACCAGGGACACGTCCACGAACACAGTCTACATGGA |
| | GCTGAGCAGCCTGAGATCTGAGGACACGGCCGTGTATTACTGTGCGAGA |
| | GAACGGCAGCAGCTGGACTACTACTACTTCGCTATGGACGTCTGGGGCC |
| | AAGGGACCACGGTCACCGTCTCCTCA |
| | [SEQ ID NO: 68] |
| DNA | GACATCCAGATGACCCAGTCTCCATCCTCACTGTCTGCATCTGTAGGAG |
| for Full V_{L} | ACAGAGTCACCATCACTTGTCGGGCGAGTCAGGGCATTAGCAATTATTT |
| | AGCCTGGTTTCAGCAGAAACCAGGGAAAGCCCCTAAGTCCCTGATCTAT |
| | GCTGCATCCAGTTTGCAAAGTGGGGTCCCATCAAAGTTCAGCGGCAGTG |
| | GATCTGGGACAGATTTCACTCTCACCATCAGCAGCCTGCAGCCTGAAGA |
| | TTTTGCAACTTATTACTGCCAACAGTATAATAGTTACCCTCCGACGTTC |
| | GGCCAAGGGACCAAGGTGGAAATCAAA |
| | [SEQ ID NO: 69] |

In certain embodiments, the anti-DLL3 scFv is an scFv-Fc fusion protein or a full-length human IgG with V_{H} and V_{L} regions or CDRs selected from Table 9. In certain embodiments, the anti-DLL3 scFv comprises a V_{H} comprising the amino acid sequence set forth in SEQ ID NO: 76, as shown in Table 9. An exemplary nucleotide sequence encoding the amino acid sequence of SEQ ID NO: 76 is set forth in SEQ ID NO: 78. In certain embodiments, the anti-DLL3 scFv comprises a V_{L} comprising the amino acid sequence set forth in SEQ ID NO: 77. An exemplary nucleotide sequence encoding the amino acid sequence of SEQ ID NO: 77 is set forth in SEQ ID NO: 79. SEQ ID NO: 76-79 are provided in Table 9. In certain embodiments, the scFv is designated as “P4”.

In certain embodiments, the anti-DLL3 scFv comprises a V_{H} comprising the amino acid sequence set forth in SEQ ID NO: 76 and a V_{L} comprising the amino acid sequence set forth in SEQ ID NO: 77.

In certain embodiments, the anti-DLL3 scFv comprises a VH comprising a CDR1 comprising the amino acid sequence set forth in SEQ ID NO: 70 or a conservative modification thereof, a CDR2 comprising the amino acid sequence set forth in SEQ ID NO: 71 or a conservative modification thereof, and a CDR3 comprising the amino acid sequence set forth in SEQ ID NO: 72 or a conservative modification thereof. SEQ ID NOs: 70-72 are provided in Table 9.

In certain embodiments, the anti-DLL3 scFv comprises a V_{L} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 75 or a conservative modification thereof. SEQ ID NOs: 73-75 are provided in Table 9.

In certain embodiments, the anti-DLL3 scFv comprises a VH comprising a CDR1 comprising the amino acid sequence set forth in SEQ ID NO: 70 or a conservative modification thereof, a CDR2 comprising the amino acid sequence set forth in SEQ ID NO: 71 or a conservative modification thereof, and a CDR3 comprising the amino acid sequence set forth in SEQ ID NO: 72 or a conservative modification thereof; and a V_{L} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 75 or a conservative modification thereof.

In certain embodiments, the anti-DLL3 scFv comprises a VH comprising a CDR1 comprising the amino acid sequence set forth in SEQ ID NO: 70, a CDR2 comprising the amino acid sequence set forth in SEQ ID NO: 71, a V_{H} CDR3 comprising the amino acid sequence set forth in SEQ ID NO: 72; and a V_{L} comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75.

In certain embodiments, the anti-DLL3 scFv comprises a V_{H} comprising the amino acid sequence set forth in SEQ ID NO: 78, and a V_{L} comprising the amino acid sequence set forth in SEQ ID NO: 79. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 9

| CDRS | 1 | 2 | 3 |
|---|---|---|---|
| V_{H} | GFTFSFY | SSSSSY | NYDFWRGAFDI |
| | [SEQ ID NO: 70] | [SEQ ID NO: 71] | [SEQ ID NO: 72] |
| V_{L} | RASQSVSSNLA | GASTRAT | QQYNNWPTWT |
| | [SEQ ID NO: 73] | [SEQ ID NO: 74] | [SEQ ID NO: 75] |

| | |
|---|---|
| Full V_{H} | EVQLVESGGGLVKPGGSLRLSCAASGFTESFYSMNWVRQAPGKGLEWVS |
| | SISSSSSYIYYADSVKGRFTISRDNAKNSLYLQMNSLRAEDTAVYYCAR |
| | NYDFWRGAFDIWGQGTMVTVSS |
| | [SEQ ID NO: 76] |
| Full V_{L} | EIVMTQSPATLSVSPGERATLSCRASQSVSSNLAWYQQKPGQAPRLLIY |
| | GASTRATGIPARFSGSGSGTEFTLTISSLQSEDFAVYYCQQYNNWPTWT |
| | FGQGTKVEIK |
| | [SEQ ID NO: 77] |
| DNA | GAGGTGCAGCTGGTGGAGTCTGGGGGGGGCCTGGTCAAGCCTGGAGGGT |
| for Full V_{H} | CCCTGAGACTCTCCTGTGCAGCCTCTGGATTCACCTTCAGTTTCTATAG |
| | CATGAACTGGGTCCGCCAGGCTCCAGGGAAGGGGCTGGAGTGGGTCTCA |
| | TCCATTAGTAGTAGTAGTAGTTACATATACTACGCAGACTCAGTGAAGG |
| | GCCGATTCACCATCTCCAGAGACAACGCCAAGAACTCACTGTATCTGCA |
| | AATGAACAGCCTGAGAGCCGAGGACACGGCTGTGTATTACTGTGCGAGA |
| | AATTACGATTTTTGGAGAGGTGCTTTTGATATCTGGGGCCAAGGGACAA |
| | TGGTCACCGTCTCTTCA |
| | [SEQ ID NO: 78] |
| DNA | GAAATAGTGATGACGCAGTCTCCAGCCACCCTGTCTGTGTCTCCAGGGG |
| for Full V_{L} | AAAGAGCCACCCTCTCCTGCAGGGCCAGTCAGAGTGTTAGCAGCAACTT |
| | AGCCTGGTACCAGCAGAAACCTGGCCAGGCTCCCAGGCTCCTCATCTAT |
| | GGTGCATCCACCAGGGCCACTGGTATCCCAGCCAGGTTCAGTGGCAGTG |
| | GGTCTGGGACAGAGTTCACTCTCACCATCAGCAGCCTGCAGTCTGAAGA |
| | TTTTGCAGTTTATTACTGTCAGCAGTATAATAACTGGCCCACGTGGACG |
| | TTCGGCCAAGGGACCAAGGTGGAAATCAAA |
| | [SEQ ID NO: 79] |

In certain embodiments, the anti-DLL3 scFv is an scFv-Fc fusion protein or a full-length human IgG with V_{H} and V_{L} regions or CDRs selected from Table 10. In certain embodiments, the anti-DLL3 scFv comprises a V_{H} comprising the amino acid sequence set forth in SEQ ID NO: 83, as shown in Table 10. An exemplary nucleotide sequence encoding the amino acid sequence of SEQ ID NO: 83 is set forth in SEQ ID NO: 85. In certain embodiments, the anti-DLL3 scFv comprises a V_{L} comprising the amino acid sequence set forth in SEQ ID NO: 84. An exemplary nucleotide sequence encoding the amino acid sequence of SEQ ID NO: 84 is set forth in SEQ ID NO: 86. SEQ ID NO: 83-86 are provided in Table 10. In certain embodiments, the scFv is designated as “J23”.

In certain embodiments, the anti-DLL3 scFv comprises a VH comprising the amino acid sequence set forth in SEQ ID NO: 83 and a V_{L} comprising the amino acid sequence set forth in SEQ ID NO: 84.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 80 or a conservative modification thereof, and a CDR3 comprising the amino acid sequence set forth in SEQ ID NO: 81 or a conservative modification thereof. SEQ ID NOs: 21, 80, and 81 are provided in Table 10.

In certain embodiments, the anti-DLL3 scFv comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof. SEQ ID NOs: 57, 58, and 82 are provided in Table 10.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 80 or a conservative modification thereof, and a CDR3 comprising the amino acid sequence set forth in SEQ ID NO: 81 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof.

In certain embodiments, the anti-DLL3 scFv comprises a VH comprising a CDR1 comprising the amino acid sequence set forth in SEQ ID NO: 21, a CDR2 comprising the amino acid sequence set forth in SEQ ID NO: 80, a V_{H} CDR3 comprising the amino acid sequence set forth in SEQ ID NO: 81; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82.

In certain embodiments, the anti-DLL3 scFv comprises a V_{H} comprising the amino acid sequence set forth in SEQ ID NO: 83, and a V_{L} comprising the amino acid sequence set forth in SEQ ID NO: 84. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{L}.

TABLE 10

| CDRs | 1 | 2 | 3 |
|---|---|---|---|
| V_{H} | GYTFTSY | NYSGGS | QGRYSGSYLDY |
| | [SEQ ID NO: 21] | [SEQ ID NO: 80] | [SEQ ID NO: 81] |
| V_{L} | RASQSVSSSYLA | GASSRAT | QQYGSSP |
| | [SEQ ID NO: 57] | [SEQ ID NO: 58] | [SEQ ID NO: 82] |

| | |
|---|---|
| Full V_{H} | QVQLVQSGAEVKKPGASVKVSCKASGYTFTSYYMHWVRQAPGQGLEWMG |
| | IINYSGGSKSYAQKFQGRVTMTRDTSTSTVYMELSSLRSEDTGVYYCAR |
| | QGRYSGSYLDYWGQGTLVTVSS |
| | [SEQ ID NO: 83] |
| Full V_{L} | EIVLTQSPGTLSLSPGERATLSCRASQSVSSSYLAWYQQKPGQAPRLLI |
| | YGASSRATGIPDRFSGSGSGTDFTLTISRLEPEDFAVYYCQQYGSSPFG |
| | QGTKLEIK |
| | [SEQ ID NO: 84] |
| DNA | CAGGTGCAGCTGGTGCAGTCTGGGGCTGAGGTGAAGAAGCCTGGGGCCT |
| for Full V_{H} | CAGTGAAGGTTTCCTGCAAGGCATCTGGATACACCTTCACCAGCTACTA |
| | TATGCACTGGGTGCGACAGGCCCCTGGACAAGGGCTTGAGTGGATGGGA |
| | ATAATCAACTATAGTGGTGGTAGCAAAAGCTACGCACAGAAGTTCCAGG |
| | GCAGAGTCACCATGACCCGGGACACGTCCACGAGCACAGTCTACATGGA |
| | GCTGAGCAGCCTGAGATCTGAGGACACGGGCGTGTATTACTGTGCGAGA |
| | CAGGGGCGGTATAGTGGGAGCTACCTTGACTACTGGGGCCAGGGAACCC |
| | TGGTCACCGTCTCCTCA |
| | [SEQ ID NO: 85] |
| DNA | GAAATTGTGTTGACGCAGTCTCCAGGCACCCTGTCTTTGTCTCCAGGGG |
| for Full V_{L} | AAAGAGCCACCCTCTCCTGCAGGGCCAGTCAGAGTGTTAGCAGCAGCTA |
| | CTTAGCCTGGTACCAGCAGAAACCTGGCCAGGCTCCCAGGCTCCTCATC |
| | TATGGTGCATCCAGCAGGGCCACTGGCATCCCAGACAGGTTCAGTGGCA |
| | GTGGGTCTGGGACAGACTTCACTCTCACCATCAGCAGACTGGAGCCTGA |
| | AGATTTTGCAGTGTATTACTGTCAGCAGTATGGTAGCTCACCTTTTGGC |
| | CAGGGGACCAAGCTGGAGATCAAA |
| | [SEQ ID NO: 86] |

In certain embodiments, the anti-DLL3 scFv is an scFv-Fc fusion protein or a full-length human IgG with V_{H} and V_{L} regions or CDRs selected from Table 11. In certain embodiments, the anti-DLL3 scFv comprises a V_{H} comprising the amino acid sequence set forth in SEQ ID NO: 92, as shown in Table 1. An exemplary nucleotide sequence encoding the amino acid sequence of SEQ ID NO: 92 is set forth in SEQ ID NO: 94. In certain embodiments, the anti-DLL3 scFv comprises a V_{L} comprising the amino acid sequence set forth in SEQ ID NO: 93. An exemplary nucleotide sequence encoding the amino acid sequence of SEQ ID NO: 93 is set forth in SEQ ID NO: 95. SEQ ID NO: 92-95 are provided in Table 11. In certain embodiments, the scFv is designated as “K19”.

In certain embodiments, the anti-DLL3 scFv comprises a V_{H} comprising the amino acid sequence set forth in SEQ ID NO: 92 and a V_{L} comprising the amino acid sequence set forth in SEQ ID NO: 93.

In certain embodiments, the anti-DLL3 scFv comprises a VH comprising a CDR1 comprising the amino acid sequence set forth in SEQ ID NO: 87 or a conservative modification thereof, a CDR2 comprising the amino acid sequence set forth in SEQ ID NO: 88 or a conservative modification thereof, and a CDR3 comprising the amino acid sequence set forth in SEQ ID NO: 89 or a conservative modification thereof. SEQ ID NOs: 87-89 are provided in Table 11.

In certain embodiments, the anti-DDL3 scFv comprises a V_{L} comprising a CDR1 comprising the amino acid sequence set forth in SEQ ID NO: 90 or a conservative modification thereof, a CDR2 comprising the amino acid sequence set forth in SEQ ID NO: 216 or a conservative modification thereof, and a CDR3 comprising the amino acid sequence set forth in SEQ ID NO: 91 or a conservative modification thereof. SEQ ID NOs: 90, 32, and 91 are provided in Table 11.

In certain embodiments, the anti-DLL3 scFv comprises a VH comprising a CDR1 comprising the amino acid sequence set forth in SEQ ID NO: 87 or a conservative modification thereof, a CDR2 comprising the amino acid sequence set forth in SEQ ID NO: 88 or a conservative modification thereof, and a CDR3 comprising the amino acid sequence set forth in SEQ ID NO: 89 or a conservative modification thereof; and a V_{L} comprising a CDR1 comprising the amino acid sequence set forth in SEQ ID NO: 90 or a conservative modification thereof, a CDR2 comprising the amino acid sequence set forth in SEQ ID NO: 216 or a conservative modification thereof, and a CDR3 comprising the amino acid sequence set forth in SEQ ID NO: 91 or a conservative modification thereof.

In certain embodiments, the anti-DLL3 scFv comprises a VH comprising a CDR1 comprising the amino acid sequence set forth in SEQ ID NO: 87, a CDR2 comprising the amino acid sequence set forth in SEQ ID NO: 88, a V_{H} CDR3 comprising the amino acid sequence set forth in SEQ ID NO: 89; and a V_{L} comprising a CDR1 comprising the amino acid sequence set forth in SEQ ID NO: 216, a CDR2 comprising the amino acid sequence set forth in SEQ ID NO: 32, and a CDR3 comprising the amino acid sequence set forth in SEQ ID NO: 91.

In certain embodiments, the anti-DLL3 scFv comprises a V_{H} comprising the amino acid sequence set forth in SEQ ID NO: 92, and a V_{L} comprising the amino acid sequence set forth in SEQ ID NO: 93. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 11

| CDRs | 1 | 2 | 3 |
|---|---|---|---|
| V_{H} | GYTENSY | NPSGGT | DWGEQYGMDV |
| | [SEQ ID NO: 87] | [SEQ ID NO: 88] | [SEQ ID NO: 89] |
| V_{L} | RSSQSLLHSNGYNYLD | LGSNRAS | MQALQTPLT |
| | [SEQ ID NO: 90] | [SEQ ID NO: 216] | [SEQ ID NO: 91] |

| | |
|---|---|
| Full V_{H} | QVQLVQSGAEVKKPGASLKVSCKTSGYTENSYYMHWVRQAPGQGLEWMG |
| | INNPSGGTTSYAQKFQGRVTMTRDTSTSTVYMELSSLRSEDTAVYYCAR |
| | DWGEQYGMDVWGQGTTVTVSS |
| | [SEQ ID NO: 92] |
| Full V_{L} | DIVMTQSPLSLPVTPGEPASISCRSSQSLLHSNGYNYLDWYLQKPGQSP |
| | QLLIYLGSNRASGVPDRFSGSGSGTDFTLKISRVEAEDVGVYYCMQALQ |
| | TPLTFGGGTKVEIK |
| | [SEQ ID NO: 93] |
| DNA | CAGGTGCAGCTGGTGCAGTCTGGGGCTGAGGTGAAGAAGCCTGGGGCCT |
| for Full V_{H} | CACTGAAGGTTTCCTGCAAGACATCTGGATACACCTTCAACAGCTACTA |
| | TATGCACTGGGTGCGACAGGCCCCTGGACAAGGGCTTGAGTGGATGGGA |
| | ATAAACAACCCTAGTGGTGGTACCACAAGCTACGCACAGAAGTTCCAGG |
| | GCAGAGTCACCATGACCAGGGACACGTCCACGAGCACAGTCTACATGGA |
| | GCTGAGCAGCCTGAGATCTGAGGACACGGCCGTGTATTACTGTGCGAGA |
| | GATTGGGGGGAGCAGTACGGTATGGACGTCTGGGGCCAAGGGACCACGG |
| | TCACCGTCTCCTCA |
| | [SEQ ID NO: 94] |
| DNA | GATATTGTGATGACTCAGTCTCCACTCTCCCTGCCCGTCACCCCTGGAG |
| for Full V_{L} | AGCCGGCCTCCATCTCCTGCAGGTCTAGTCAGAGCCTCCTGCATAGTAA |
| | TGGATACAACTATTTGGATTGGTACCTGCAGAAGCCAGGACAGTCTCCA |
| | CAGCTCCTGATCTATTTGGGTTCTAATCGGGCCTCCGGGGTCCCTGACA |
| | GGTTCAGTGGCAGTGGATCAGGCACAGATTTTACACTGAAAATCAGCAG |
| | AGTGGAGGCTGAGGATGTTGGGGTTTATTACTGCATGCAAGCTCTACAA |
| | ACTCCGCTCACTTTCGGCGGAGGGACCAAGGTGGAGATCAAA |
| | [SEQ ID NO: 95] |

In certain embodiments, the anti-DLL3 scFv is an scFv-Fc fusion protein or a full-length human IgG with V_{H} and V_{L} regions or CDRs selected from Table 12. In certain embodiments, the anti-DLL3 scFv comprises a V_{H} comprising the amino acid sequence set forth in SEQ ID NO: 102, as shown in Table 12. An exemplary nucleotide sequence encoding the amino acid sequence of SEQ ID NO: 102 is set forth in SEQ ID NO: 104. In certain embodiments, the anti-DLL3 scFv comprises a V_{L} comprising the amino acid sequence set forth in SEQ ID NO: 103. An exemplary nucleotide sequence encoding the amino acid sequence of SEQ ID NO: 103 is set forth in SEQ ID NO: 105. SEQ ID NO: 102-105 are provided in Table 12. In certain embodiments, the scFv is designated as “N10”.

In certain embodiments, the anti-DLL3 scFv comprises a V_{H} comprising the amino acid sequence set forth in SEQ ID NO: 102 and a V_{L} comprising the amino acid sequence set forth in SEQ ID NO: 103.

In certain embodiments, the anti-DLL3 scFv comprises a VH comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof. SEQ ID NOs: 96-98 are provided in Table 12.

In certain embodiments, the anti-DLL3 scFv comprises a V_{L} comprising a CDR1 comprising the amino acid sequence set forth in SEQ ID NO: 99 or a conservative modification thereof, a CDR2 comprising the amino acid sequence set forth in SEQ ID NO: 100 or a conservative modification thereof, and a CDR3 comprising the amino acid sequence set forth in SEQ ID NO: 101 or a conservative modification thereof. SEQ ID NOs: 99-101 are provided in Table 12.

In certain embodiments, the anti-DLL3 scFv comprises a VH comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof; and a V_{L} comprising a CDR1 comprising the amino acid sequence set forth in SEQ ID NO: 99 or a conservative modification thereof, a CDR2 comprising the amino acid sequence set forth in SEQ ID NO: 100 or a conservative modification thereof, and a CDR3 comprising the amino acid sequence set forth in SEQ ID NO: 101 or a conservative modification thereof.

In certain embodiments, the anti-DLL3 scFv comprises a VH comprising a CDR1 comprising the amino acid sequence set forth in SEQ ID NO: 96, a CDR2 comprising the amino acid sequence set forth in SEQ ID NO: 97, a V_{H} CDR3 comprising the amino acid sequence set forth in SEQ ID NO: 98; and a V_{L} comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101.

In certain embodiments, the anti-DLL3 scFv comprises a V_{H} comprising the amino acid sequence set forth in SEQ ID NO: 103, and a V_{L} comprising the amino acid sequence set forth in SEQ ID NO: 103. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 12

| CDRS | 1 | 2 | 3 |
|---|---|---|---|
| V_{H} | GYTFTGY | NPHSGA | DSSGWYGDAFDI |
| | [SEQ ID NO: 96] | [SEQ ID NO: 97] | [SEQ ID NO: 98] |
| V_{L} | RSSQSLVHSDGNTYLS | KISNRFS | MQATQFPLT |
| | [SEQ ID NO: 99] | [SEQ ID NO: 100] | [SEQ ID NO: 101] |

| | |
|---|---|
| Full V_{H} | QVQLVQSGAEVKKPGASVKVSCKASGYTFTGYYMHWVRQAPGQGLEWMG |
| | WINPHSGAPNSAQKFQVRVTMTRDTSISTAYMELSRLRSDDTAVYYCAR |
| | DSSGWYGDAFDIWGQGTMVTVSS |
| | [SEQ ID NO: 102] |
| Full V_{L} | DIVMTQTPLSSPVTLGQPASISCRSSQSLVHSDGNTYLSWLQQRPGQPP |
| | RLLIYKISNRFSGVPDRFSGSGAGTDFTLKISRVEAEDVGVYYCMQATQ |
| | FPLTFGGGTKVEIK |
| | [SEQ ID NO: 103] |
| DNA | CAGGTGCAGCTGGTGCAGTCTGGGGCTGAGGTGAAGAAGCCTGGGGCCT |
| for Full V_{H} | CAGTGAAGGTCTCCTGCAAGGCTTCTGGATACACCTTCACCGGCTACTA |
| | TATGCACTGGGTGCGACAGGCCCCTGGACAAGGGCTTGAGTGGATGGGA |
| | TGGATCAACCCTCACAGTGGTGCCCCAAACTCTGCACAGAAGTTTCAGG |
| | TCAGGGTCACCATGACCAGGGACACGTCCATCAGCACAGCCTACATGGA |
| | GCTGAGCAGGCTGAGATCTGACGACACGGCCGTGTATTACTGTGCGAGG |
| | GATAGCAGCGGCTGGTACGGTGATGCTTTTGATATCTGGGGCCAAGGGA |
| | CAATGGTCACCGTCTCTTCA |
| | [SEQ ID NO: 104] |
| DNA | GATATTGTGATGACCCAGACTCCACTCTCCTCACCTGTCACCCTTGGAC |
| for Full V_{L} | AGCCGGCCTCCATCTCCTGCAGGTCTAGTCAAAGCCTCGTACACAGTGA |
| | TGGCAACACCTACTTGAGTTGGCTTCAGCAGAGGCCAGGCCAGCCTCCA |
| | AGACTCCTAATTTATAAGATTTCTAACCGGTTCTCTGGGGTCCCAGACA |
| | GATTCAGTGGCAGTGGGGCAGGGACAGATTTCACACTGAAAATCAGCAG |
| | GGTGGAAGCTGAGGATGTCGGGGTTTATTACTGCATGCAAGCTACACAA |
| | TTTCCGCTCACTTTCGGCGGAGGGACCAAGGTGGAGATCAAA |
| | [SEQ ID NO: 105] |

In certain embodiments, the anti-DLL3 scFv is an scFv-Fc fusion protein or a full-length human IgG with V_{H} and V_{L} regions or CDRs selected from Table 13. In certain embodiments, the anti-DLL3 scFv comprises a V_{H} comprising the amino acid sequence set forth in SEQ ID NO: 108, as shown in Table 13. An exemplary nucleotide sequence encoding the amino acid sequence of SEQ ID NO: 108 is set forth in SEQ ID NO: 110. In certain embodiments, the anti-DLL3 scFv comprises a V_{L} comprising the amino acid sequence set forth in SEQ ID NO: 109. An exemplary nucleotide sequence encoding the amino acid sequence of SEQ ID NO: 109 is set forth in SEQ ID NO: 111. SEQ ID NO: 108-111 are provided in Table 13. In certain embodiments, the scFv is designated as “B16-v1”.

In certain embodiments, the anti-DLL3 scFv comprises a V_{H} comprising the amino acid sequence set forth in SEQ ID NO: 108 and a V_{L} comprising the amino acid sequence set forth in SEQ ID NO: 109.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof. SEQ ID NOs: 105-107 are provided in Table 13.

In certain embodiments, the anti-DLL3 scFv comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, a V_{L} CDR3 comprising the amino acid sequence set forth in SEQ ID NO: 82 or a conservative modification thereof. SEQ ID NOs: 57, 58, and 82 are provided in Table 13.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof.

In certain embodiments, the anti-DLL3 scFv comprises a VH comprising a CDR1 comprising the amino acid sequence set forth in SEQ ID NO: 21, a CDR2 comprising the amino acid sequence set forth in SEQ ID NO: 106, a V_{H} CDR3 comprising the amino acid sequence set forth in SEQ ID NO: 107; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82.

In certain embodiments, the anti-DLL3 scFv comprises a V_{H} comprising the amino acid sequence set forth in SEQ ID NO: 108, and a V_{L} comprising the amino acid sequence set forth in SEQ ID NO: 109. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 13

| CDRs | 1 | 2 | 3 |
|---|---|---|---|
| V_{H} | GYTFTSY | DPSGGS | QGRYVGSYLDY |
| | [SEQ ID NO: 21] | [SEQ ID NO: 106] | [SEQ ID NO: 107] |
| V_{L} | RASQSVSSSYLA | GASSRAT | QQYGSSP |
| | [SEQ ID NO: 57] | [SEQ ID NO: 58] | [SEQ ID NO: 82] |

| | |
|---|---|
| Full V_{H} | QVQLVQSGAEVKKPGASVKVSCKASGYTFTSYYMHWVRQAPGQGLEWMG |
| | IIDPSGGSTSYAQKFQGRVTMTRDTSTNTVYMDLSSLRSEDTAVYYCAR |
| | QGRYVGSYLDYWGQGTLVTVSS |
| | [SEQ ID NO: 108] |
| Full V_{L} | EIVLTQSPGTLSLSPGERATLSCRASQSVSSSYLAWYQQKPGQAPRLLI |
| | YGASSRATGIPDRFSGSGSGTDFTLTISRLEPEDFAVYYCQQYGSSPFG |
| | QGTKLEIK |
| | [SEQ ID NO: 109] |
| DNA | CAGGTGCAGCTGGTGCAGTCTGGGGCTGAGGTGAAGAAGCCTGGGGCCT |
| for Full V_{H} | CAGTGAAGGTTTCCTGCAAGGCATCTGGATACACCTTCACCAGCTACTA |
| | TATGCACTGGGTGCGACAGGCCCCTGGACAAGGGCTTGAGTGGATGGGA |
| | ATAATCGACCCTAGTGGTGGTAGCACAAGCTACGCACAGAAGTTCCAGG |
| | GCAGAGTCACCATGACCAGGGACACGTCCACGAACACAGTCTACATGGA |
| | CCTGAGCAGCCTGAGATCTGAGGACACGGCCGTGTATTACTGTGCGAGA |
| | CAGGGGCGGTATGTTGGGAGCTACCTTGACTACTGGGGCCAGGGAACCC |
| | TGGTCACCGTCTCCTCA |
| | [SEQ ID NO: 110] |
| DNA | GAAATTGTGTTGACGCAGTCTCCAGGCACCCTGTCTTTGTCTCCAGGGG |
| for Full V_{L} | AAAGAGCCACCCTCTCCTGCAGGGCCAGTCAGAGTGTTAGCAGCAGCTA |
| | CTTAGCCTGGTACCAGCAGAAACCTGGCCAGGCTCCCAGGCTCCTCATC |
| | TATGGTGCATCCAGCAGGGCCACTGGCATCCCAGACAGGTTCAGTGGCA |
| | GTGGGTCTGGGACAGACTTCACTCTCACCATCAGCAGACTGGAGCCTGA |
| | AGATTTTGCAGTGTATTACTGTCAGCAGTATGGTAGCTCACCTTTTGGC |
| | CAGGGGACCAAGCTGGAGATCAAA |
| | [SEQ ID NO: 111] |

In certain embodiments, the anti-DLL3 scFv is an scFv-Fc fusion protein or a full-length human IgG with V_{H} and V_{L} regions or CDRs selected from Table 14. In certain embodiments, the anti-DLL3 scFv comprises a V_{H} comprising the amino acid sequence set forth in SEQ ID NO: 108, as shown in Table 14. An exemplary nucleotide sequence encoding the amino acid sequence of SEQ ID NO: 108 is set forth in SEQ ID NO: 110. In certain embodiments, the anti-DLL3 scFv comprises a V_{L} comprising the amino acid sequence set forth in SEQ ID NO: 113. An exemplary nucleotide sequence encoding the amino acid sequence of SEQ ID NO: 113 is set forth in SEQ ID NO: 114. In certain embodiments, the anti-DLL3 scFv comprises a V_{H} Comprising the amino acid sequence set forth in SEQ ID NO: 108 and a V_{L} comprising the amino acid sequence set forth in SEQ ID NO: 113. SEQ ID NO: 108, 113, 110, and 114 are provided in Table 14. In certain embodiments, the scFv is designated as “B16-v2”.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof. SEQ ID NOs: 21, 106, and 107 are provided in Table 14.

In certain embodiments, the anti-DLL3 scFv comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 112 or a conservative modification thereof. SEQ ID NOs: 4, 5, and 112 are provided in Table 14.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 112 or a conservative modification.

In certain embodiments, the anti-DLL3 scFv comprises a VH comprising a CDR1 comprising the amino acid sequence set forth in SEQ ID NO: 21, a CDR2 comprising the amino acid sequence set forth in SEQ ID NO: 106, a V_{H} CDR3 comprising the amino acid sequence set forth in SEQ ID NO: 107; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112.

In certain embodiments, the anti-DLL3 scFv comprises a V_{H} comprising the amino acid sequence set forth in SEQ ID NO: 108, and a V_{L} comprising the amino acid sequence set forth in SEQ ID NO: 113. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 14

| CDRs | 1 | 2 | 3 |
|---|---|---|---|
| V_{H} | GYTFTSY | DPSGGS | QGRYVGSYLDY |
| | [SEQ ID NO: 21] | [SEQ ID NO: 106] | [SEQ ID NO: 107] |
| V_{L} | RASQGISNYLA | AASSLQS | QQYNSYPYT |
| | [SEQ ID NO: 4] | [SEQ ID NO: 5] | [SEQ ID NO: 112] |

| | |
|---|---|
| Full V_{H} | QVQLVQSGAEVKKPGASVKVSCKASGYTFTSYYMHWVRQAPGQGLEWMG |
| | IIDPSGGSTSYAQKFQGRVTMTRDTSTNTVYMDLSSLRSEDTAVYYCAR |
| | QGRYVGSYLDYWGQGTLVTVSS |
| | [SEQ ID NO: 108] |
| Full V_{L} | DIQMTQSPSSLSASVGDRVTITCRASQGSINYLAWFQQKPGKAPKSVIY |
| | AASSLQSGVPSKFSGSGSGTDFTLTISSMOPEDFATYYCQQYNSYPYTF |
| | GQGTKLEIK |
| | [SEQ ID NO: 113] |
| DNA | CAGGTGCAGCTGGTGCAGTCTGGGGCTGAGGTGAAGAAGCCTGGGGCCT |
| for Full V_{H} | CAGTGAAGGTTTCCTGCAAGGCATCTGGATACACCTTCACCAGCTACTA |
| | TATGCACTGGGTGCGACAGGCCCCTGGACAAGGGCTTGAGTGGATGGGA |
| | ATAATCGACCCTAGTGGTGGTAGCACAAGCTACGCACAGAAGTTCCAGG |
| | GCAGAGTCACCATGACCAGGGACACGTCCACGAACACAGTCTACATGGA |
| | CCTGAGCAGCCTGAGATCTGAGGACACGGCCGTGTATTACTGTGCGAGA |
| | CAGGGGCGGTATGTTGGGAGCTACCTTGACTACTGGGGCCAGGGAACCC |
| | TGGTCACCGTCTCCTCA |
| | [SEQ ID NO: 110] |
| DNA | GACATCCAGATGACCCAGTCTCCATCCTCACTGTCTGCATCTGTAGGAG |
| for Full V_{L} | ACAGAGTCACCATCACTTGTCGGGCGAGTCAGGGCATTAGCAATTATTT |
| | AGCCTGGTTTCAGCAGAAACCAGGGAAAGCCCCTAAGTCCGTGATCTAT |
| | GCTGCATCCAGTTTGCAAAGTGGGGTCCCATCAAAGTTCAGCGGCAGTG |
| | GATCTGGGACAGATTTCACTCTCACCATCAGCAGCATGCAGCCTGAAGA |
| | TTTTGCAACTTATTACTGCCAACAGTATAATAGTTACCCGTACACTTTT |
| | GGCCAGGGGACCAAGCTGGAGATCAAA |
| | [SEQ ID NO: 114] |

In certain embodiments, the anti-DLL3 scFv is an scFv-Fc fusion protein or a full-length human IgG with V_{H} and V_{L} regions or CDRs selected from Table 15. In certain embodiments, the anti-DLL3 scFv comprises a V_{H} comprising the amino acid sequence set forth in SEQ ID NO: 119, as shown in Table 15. An exemplary nucleotide sequence encoding the amino acid sequence of SEQ ID NO: 119 is set forth in SEQ ID NO: 121. In certain embodiments, the anti-DLL3 scFv comprises a V_{L} comprising the amino acid sequence set forth in SEQ ID NO: 120. An exemplary nucleotide sequence encoding the amino acid sequence of SEQ ID NO: 120 is set forth in SEQ ID NO: 122. In certain embodiments, the anti-DLL3 scFv comprises a V_{H} Comprising the amino acid sequence set forth in SEQ ID NO: 119 and a V_{L} comprising the amino acid sequence set forth in SEQ ID NO: 120. SEQ ID NO: 119-122 are provided in Table 15. In certain embodiments, the scFv is designated as “E23”.

In certain embodiments, the anti-DLL3 scFv comprises a VH comprising a CDR1 comprising the amino acid sequence set forth in SEQ ID NO: 96 or a conservative modification thereof, a CDR2 comprising the amino acid sequence set forth in SEQ ID NO: 115 or a conservative modification thereof, and a CDR3 comprising the amino acid sequence set forth in SEQ ID NO: 116 or a conservative modification thereof. SEQ ID NOs: 96, 115, and 116 are provided in Table 15.

In certain embodiments, the anti-DLL3 scFv comprises a V_{L} comprising a CDR1 comprising the amino acid sequence set forth in SEQ ID NO: 117 or a conservative modification thereof, a CDR2 comprising the amino acid sequence set forth in SEQ ID NO: 100 or a conservative modification thereof, and a CDR3 comprising the amino acid sequence set forth in SEQ ID NO: 118 or a conservative modification thereof. SEQ ID NOs: 117, 100, and 118 are provided in Table 15.

In certain embodiments, the anti-DLL3 scFv comprises a VH comprising a CDR1 comprising the amino acid sequence set forth in SEQ ID NO: 96 or a conservative modification thereof, a CDR2 comprising the amino acid sequence set forth in SEQ ID NO: 115 or a conservative modification thereof, and a CDR3 comprising the amino acid sequence set forth in SEQ ID NO: 116 or a conservative modification thereof; and a V_{L} comprising a CDR1 comprising the amino acid sequence set forth in SEQ ID NO:117 or a conservative modification thereof, a CDR2 comprising the amino acid sequence set forth in SEQ ID NO: 100 or a conservative modification thereof, and a CDR3 comprising the amino acid sequence set forth in SEQ ID NO: 118 or a conservative modification.

In certain embodiments, the anti-DLL3 scFv comprises a VH comprising a CDR1 comprising the amino acid sequence set forth in SEQ ID NO: 96, a CDR2 comprising the amino acid sequence set forth in SEQ ID NO: 115, a V_{H} CDR3 comprising the amino acid sequence set forth in SEQ ID NO: 116; and a V_{L} comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118.

In certain embodiments, the anti-DLL3 scFv comprises a V_{H} comprising the amino acid sequence set forth in SEQ ID NO: 119, and a V_{L} comprising the amino acid sequence set forth in SEQ ID NO: 112. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 15

| CDRS | 1 | 2 | 3 |
|---|---|---|---|
| V_{H} | GYTFTGY | NSYSGG | DGSGWYGSYFDF |
| | [SEQ ID NO: 96] | [SEQ ID NO: 115] | [SEQ ID NO: 116] |
| V_{L} | RSSQSLVHSDGNTYL | KISNRFS | IQTTQFPLT |
| | S | [SEQ ID NO: 100] | [SEQ ID NO: 118] |
| | [SEQ ID NO: 117] | | |

| | |
|---|---|
| Full V_{H} | QVQLVQSGAEVRKPGASVKVSCKASGYTFTGYYMHWVRQAPGQGLEWMG |
| | WINSYSGGANYAQKFQDRVTMTRDTSVSTASMELSRLRSDDTAVYYCAR |
| | DGSGWYGSYFDFWGQGTLVTVSS |
| | [SEQ ID NO: 119] |
| Full V_{L} | DIVMTQTPLSSPVTLGQPASISCRSSQSLVHSDGNTYLSWLQQRPGQPP |
| | RLLIYKISNRFSGVPDRFSGSGAGTDFTLKISRVEAEDVGFYYCIQTTQ |
| | FPLTFGGGTKVEIK |
| | [SEQ ID NO: 120] |
| DNA | CAGGTGCAGCTGGTGCAGTCTGGGGCTGAGGTGAGGAAGCCTGGGGCCT |
| for Full V_{H} | CAGTGAAGGTCTCCTGCAAGGCTTCTGGATACACCTTCACCGGCTACTA |
| | TATGCACTGGGTGCGACAGGCCCCTGGACAAGGGCTTGAGTGGATGGGA |
| | TGGATCAACTCTTACAGTGGTGGCGCAAACTATGCACAGAAGTTTCAGG |
| | ACAGGGTCACCATGACCAGGGACACGTCCGTCAGCACAGCCTCCATGGA |
| | GCTGAGCAGGCTGAGATCTGACGACACGGCCGTGTATTACTGTGCGAGA |
| | GATGGCAGTGGCTGGTACGGGTCCTACTTTGACTTCTGGGGCCAGGGAA |
| | CCCTGGTCACCGTCTCCTCA |
| | [SEQ ID NO: 121] |
| DNA | GATATTGTGATGACCCAGACTCCACTCTCCTCACCTGTCACCCTTGGAC |
| for Full V_{L} | AGCCGGCCTCCATCTCCTGCAGGTCTAGTCAAAGCCTCGTACACAGTGA |
| | TGGAAACACCTACTTGAGTTGGCTTCAGCAGAGGCCAGGCCAGCCTCCA |
| | AGACTCCTAATTTATAAGATTTCTAACCGGTTCTCTGGGGTCCCAGACA |
| | GATTCAGTGGCAGTGGGGCAGGGACAGATTTCACACTGAAAATCAGCAG |
| | GGTGGAAGCTGAGGATGTCGGGTTTTATTACTGCATACAAACTACACAA |
| | TTTCCGCTCACTTTCGGCGGAGGGACCAAGGTGGAGATCAAA |
| | [SEQ ID NO: 122] |

In certain embodiments, the anti-DLL3 scFv is an scFv-Fc fusion protein or a full-length human IgG with V_{H} and V_{L} regions or CDRs selected from Table 16. In certain embodiments, the anti-DLL3 scFv comprises a V_{H} comprising the amino acid sequence set forth in SEQ ID NO: 126, as shown in Table 16. An exemplary nucleotide sequence encoding the amino acid sequence of SEQ ID NO: 126 is set forth in SEQ ID NO: 128. In certain embodiments, the anti-DLL3 scFv comprises a V_{L} comprising the amino acid sequence set forth in SEQ ID NO: 127. An exemplary nucleotide sequence encoding the amino acid sequence of SEQ ID NO: 127 is set forth in SEQ ID NO: 129. In certain embodiments, the anti-DLL3 scFv comprises a V_{H} Comprising the amino acid sequence set forth in SEQ ID NO: 126 and a V_{L} comprising the amino acid sequence set forth in SEQ ID NO: 127. SEQ ID NO: 126-129 are provided in Table 16. In certain embodiments, the scFv is designated as “F9”.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof. SEQ ID NOs: 21, 2, and 123 are provided in Table 16.

In certain embodiments, the anti-DLL3 scFv comprises a V_{L} comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 125 or a conservative modification thereof. SEQ ID NOs: 124, 58, and 125 are provided in Table 16.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof; and a V_{L} comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 125 or a conservative modification.

In certain embodiments, the anti-DLL3 scFv comprises a VH comprising a CDR1 comprising the amino acid sequence set forth in SEQ ID NO: 21, a CDR2 comprising the amino acid sequence set forth in SEQ ID NO: 2, a V_{H} CDR3 comprising the amino acid sequence set forth in SEQ ID NO: 123; and a V_{L} comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125.

In certain embodiments, the anti-DLL3 scFv comprises a V_{H} comprising the amino acid sequence set forth in SEQ ID NO: 126, and a V_{L} comprising the amino acid sequence set forth in SEQ ID NO: 127. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 16

| CDRS | 1 | 2 | 3 |
|---|---|---|---|
| V_{H} | GYTFTSY | NPSGGS | QGGAQWLVLAFDI |
| | [SEQ ID NO: 21] | [SEQ ID NO: 2] | [SEQ ID NO: 123] |
| V_{L} | RASQSVSSYLA | GASSRAT | QQYDSSPYT |
| | [SEQ ID NO: 124] | [SEQ ID NO: 58] | [SEQ ID NO: 125] |

| | |
|---|---|
| Full V_{H} | QVQLVQSGAEVKKPGASVQVSCKASGYTFTSYYMHWVRQAPGQGLEWMG |
| | IINPSGGSTSYAQKFQGRVTMTRDTSTSTVYMELSSLRSEDTAVYYCAR |
| | QGGAQWLVLAFDIWGQGTMVTVSS |
| | [SEQ ID NO: 126] |
| Full V_{L} | EIVLTQSPGTLSLSPGERATLSCRASQSVSSYLAWYQQKPGQPPRLLIY |
| | GASSRATGIPDRFSGGGSGTDFTLTISRLEPEDFAVYYCQQYDSSPYTF |
| | GQGTKLEIK |
| | [SEQ ID NO: 127] |
| DNA | CAGGTGCAGCTGGTGCAGTCTGGGGCTGAGGTGAAGAAGCCTGGGGCCT |
| for Full V_{H} | CAGTGCAGGTTTCCTGCAAGGCATCTGGATACACCTTCACCAGCTACTA |
| | TATGCACTGGGTGCGACAGGCCCCTGGACAAGGGCTTGAGTGGATGGGA |
| | ATAATCAACCCTAGTGGTGGTAGCACAAGCTACGCACAGAAGTTCCAGG |
| | GCAGAGTCACCATGACCAGGGACACGTCCACGAGCACAGTCTACATGGA |
| | GCTGAGCAGCCTGAGATCTGAGGACACGGCCGTGTATTACTGTGCGAGA |
| | CAGGGGGGAGCCCAGTGGCTGGTACTTGCTTTTGATATCTGGGGCCAAG |
| | GGACAATGGTCACCGTCTCTTCA |
| | [SEQ ID NO: 128] |
| DNA | GAAATTGTGTTGACGCAGTCTCCAGGCACCCTGTCTTTGTCTCCAGGGG |
| for Full V_{L} | AAAGAGCCACCCTCTCCTGCAGGGCCAGTCAGAGTGTTAGCAGCTACTT |
| | AGCCTGGTACCAGCAGAAACCTGGCCAGCCTCCCAGGCTCCTCATCTAT |
| | GGTGCATCCAGCAGGGCCACTGGCATCCCAGACAGGTTCAGTGGCGGTG |
| | GGTCTGGGACAGACTTCACTCTCACCATCAGCAGACTGGAGCCTGAAGA |
| | TTTTGCAGTGTATTACTGTCAGCAGTATGATAGCTCACCGTACACTTTT |
| | GGCCAGGGGACCAAGCTGGAGATCAAA |
| | [SEQ ID NO: 129] |

In certain embodiments, the anti-DLL3 scFv is an scFv-Fc fusion protein or a full-length human IgG with V_{H} and V_{L} regions or CDRs selected from Table 17. In certain embodiments, the anti-DLL3 scFv comprises a V_{H} comprising the amino acid sequence set forth in SEQ ID NO: 131, as shown in Table 17. An exemplary nucleotide sequence encoding the amino acid sequence of SEQ ID NO: 131 is set forth in SEQ ID NO: 133. In certain embodiments, the anti-DLL3 scFv comprises a V_{L} comprising the amino acid sequence set forth in SEQ ID NO: 132. An exemplary nucleotide sequence encoding the amino acid sequence of SEQ ID NO: 132 is set forth in SEQ ID NO: 134. In certain embodiments, the anti-DLL3 scFv comprises a V_{H} Comprising the amino acid sequence set forth in SEQ ID NO: 131 and a V_{L} comprising the amino acid sequence set forth in SEQ ID NO: 132. SEQ ID NO: 131-134 are provided in Table 17. In certain embodiments, the scFv is designated as “L12”.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof. SEQ ID NOs: 21, 2, and 56 are provided in Table 17.

In certain embodiments, the anti-DLL3 scFv comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 130 or a conservative modification thereof. SEQ ID NOs: 57, 58, and 130 are provided in Table 17.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 130 or a conservative modification.

In certain embodiments, the anti-DLL3 scFv comprises a VH comprising a CDR1 comprising the amino acid sequence set forth in SEQ ID NO: 21, a CDR2 comprising the amino acid sequence set forth in SEQ ID NO: 2, a V_{H} CDR3 comprising the amino acid sequence set forth in SEQ ID NO: 56; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130.

In certain embodiments, the anti-DLL3 scFv comprises a V_{H} comprising the amino acid sequence set forth in SEQ ID NO: 131, and a V_{L} comprising the amino acid sequence set forth in SEQ ID NO: 132. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 17

| CDRS | 1 | 2 | 3 |
|---|---|---|---|
| V_{H} | GYTFTSY | NPSGGS | QLELLGNAFDI |
| | [SEQ ID NO: 21] | [SEQ ID NO: 2] | [SEQ ID NO: 56] |
| V_{L} | RASQSVSSSYLA | GASSRAT | QQYGNSPYT |
| | [SEQ ID NO: 57] | [SEQ ID NO: 58] | [SEQ ID NO: 130] |

| | |
|---|---|
| Full V_{H} | QVQLVQSGAEVKKPGASVKVSCKASGYTFTSYYMHWVRQAPGQGLEWMG |
| | IINPSGGSRSYAQKFQGRVTMTRDTSTSTVYMELSSLRSEDTAVYYCAR |
| | QLELLGNAFDIWGQGTMVTVSS |
| | [SEQ ID NO: 131] |
| Full V_{L} | EIVLTQSPGTLSLSPGERATLSCRASQSVSSSYLAWYQQKPGQAPRLLI |
| | YGASSRATGIPDRESGSGSGTDFTLTISRLEPEDFAVYYCQQYGNSPYT |
| | FGQGTKLEIK |
| | [SEQ ID NO: 132] |
| DNA | CAGGTGCAGCTGGTGCAGTCTGGGGCTGAGGTGAAGAAGCCTGGGGCCT |
| for Full V_{H} | CAGTGAAGGTTTCCTGCAAGGCATCTGGATACACCTTCACCAGCTACTA |
| | TATGCACTGGGTGCGACAGGCCCCTGGACAAGGGCTTGAGTGGATGGGA |
| | ATAATCAACCCTAGTGGTGGTAGTAGAAGCTACGCACAGAAGTTCCAGG |
| | GCAGAGTCACTATGACCAGGGACACGTCCACGAGCACAGTCTACATGGA |
| | GCTGAGCAGCCTGAGATCTGAGGACACGGCCGTGTATTACTGTGCGAGA |
| | CAACTGGAACTTTTGGGGAATGCTTTTGATATCTGGGGCCAAGGGACAA |
| | TGGTCACCGTCTCTTCA |
| | [SEQ ID NO: 133] |
| DNA | GAAATTGTGTTGACGCAGTCTCCAGGCACCCTGTCTTTGTCTCCAGGGG |
| for Full V_{L} | AAAGAGCCACCCTCTCCTGCAGGGCCAGTCAGAGTGTTAGCAGCAGCTA |
| | CTTAGCCTGGTACCAGCAGAAACCTGGCCAGGCTCCCAGGCTCCTCATC |
| | TATGGTGCATCCAGCAGGGCCACTGGCATCCCAGACAGGTTCAGTGGCA |
| | GTGGGTCTGGGACAGACTTCACTCTCACCATCAGCAGACTGGAGCCTGA |
| | AGATTTTGCAGTGTATTACTGTCAGCAGTATGGTAACTCGCCGTACACT |
| | TTTGGCCAGGGGACCAAGCTGGAGATCAAA |
| | [SEQ ID NO: 134] |

In certain embodiments, the anti-DLL3 scFv is an scFv-Fc fusion protein or a full-length human IgG with V_{H} and V_{L} regions or CDRs selected from Table 18. In certain embodiments, the anti-DLL3 scFv comprises a V_{H} comprising the amino acid sequence set forth in SEQ ID NO: 141, as shown in Table 18. An exemplary nucleotide sequence encoding the amino acid sequence of SEQ ID NO: 141 is set forth in SEQ ID NO: 143. In certain embodiments, the anti-DLL3 scFv comprises a V_{L} comprising the amino acid sequence set forth in SEQ ID NO: 142. An exemplary nucleotide sequence encoding the amino acid sequence of SEQ ID NO: 142 is set forth in SEQ ID NO: 144. In certain embodiments, the anti-DLL3 scFv comprises a V_{H} Comprising the amino acid sequence set forth in SEQ ID NO: 141 and a V_{L} comprising the amino acid sequence set forth in SEQ ID NO: 142. SEQ ID NO: 141-144 are provided in Table 18. In certain embodiments, the scFv is designated as “B22”.

In certain embodiments, the anti-DLL3 scFv comprises a VH comprising a CDR1 comprising the amino acid sequence set forth in SEQ ID NO: 135 or a conservative modification thereof, a CDR2 comprising the amino acid sequence set forth in SEQ ID NO: 136 or a conservative modification thereof, and a CDR3 comprising the amino acid sequence set forth in SEQ ID NO: 137 or a conservative modification thereof. SEQ ID NOs: 135-137 are provided in Table 18.

In certain embodiments, the anti-DLL3 scFv comprises a V_{L} comprising a CDR1 comprising the amino acid sequence set forth in SEQ ID NO: 138 or a conservative modification thereof, a CDR2 comprising the amino acid sequence set forth in SEQ ID NO: 139 or a conservative modification thereof, and a CDR3 comprising the amino acid sequence set forth in SEQ ID NO: 140 or a conservative modification thereof. SEQ ID NOs: 138-140 are provided in Table 18.

In certain embodiments, the anti-DLL3 scFv comprises a VH comprising a CDR1 comprising the amino acid sequence set forth in SEQ ID NO: 135 or a conservative modification thereof, a CDR2 comprising the amino acid sequence set forth in SEQ ID NO: 136 or a conservative modification thereof, and a CDR3 comprising the amino acid sequence set forth in SEQ ID NO: 137 or a conservative modification thereof; and a V_{L} comprising a CDR1 comprising the amino acid sequence set forth in SEQ ID NO: 138 or a conservative modification thereof, a CDR2 comprising the amino acid sequence set forth in SEQ ID NO: 139 or a conservative modification thereof, and a CDR3 comprising the amino acid sequence set forth in SEQ ID NO: 140 or a conservative modification.

In certain embodiments, the anti-DLL3 scFv comprises a VH comprising a CDR1 comprising the amino acid sequence set forth in SEQ ID NO: 135, a CDR2 comprising the amino acid sequence set forth in SEQ ID NO: 136, a V_{H} CDR3 comprising the amino acid sequence set forth in SEQ ID NO: 137; and a V_{L} comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140.

In certain embodiments, the anti-DLL3 scFv comprises a V_{H} comprising the amino acid sequence set forth in SEQ ID NO: 141, and a V_{L} comprising the amino acid sequence set forth in SEQ ID NO: 142. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 18

| CDRS | 1 | 2 | 3 |
|---|---|---|---|
| V_{H} | GFTFNNF | KQDGSE | ESYFDL |
| | [SEQ ID NO: 135] | [SEQ ID NO: 136] | [SEQ ID NO: 137] |
| V_{L} | RSSTGAVTTSNYAN | GTNNRAP | ALWYSNRWV |
| | [SEQ ID NO: 138] | [SEQ ID NO: 139] | [SEQ ID NO: 140] |

| | |
|---|---|
| Full V_{H} | EVQLVESGGGLVQPGGSLRLSCAASGFTFNNFWMSWVRQAPGKGLEWVA |
| | NIKQDGSEKYYVDSVKGRFTISRDNAKNSLYLQMNSLRAEDTAVYYCAR |
| | ESYFDLWGRGTLVTVSS |
| | [SEQ ID NO: 141] |
| Full V_{L} | QAVVTQESALTTSPGETVTLTCRSSTGAVTTSNYANWVQEKPDHLFTGL |
| | IGGTNNRAPGVPARFSGSLIGDKAALTITGAQTEDEAIYFCALWYSNRW |
| | VFGGGTKLTVL |
| | [SEQ ID NO: 142] |
| DNA | GAGGTGCAGCTGGTGGAGTCTGGGGGAGGCTTGGTCCAGCCTGGGGGGT |
| for Full V_{H} | CCCTGAGACTCTCCTGTGCAGCCTCTGGATTCACCTTTAATAACTTTTG |
| | GATGAGCTGGGTCCGCCAGGCTCCAGGGAAGGGGCTGGAGTGGGTGGCC |
| | AACATAAAGCAAGATGGAAGTGAGAAATACTATGTGGACTCTGTGAAGG |
| | GCCGATTCACCATCTCCAGAGACAACGCCAAGAACTCACTGTATCTGCA |
| | AATGAACAGCCTGAGAGCCGAGGACACGGCTGTGTATTACTGTGCGAGA |
| | GAGAGTTACTTCGATCTCTGGGGCCGTGGCACCCTGGTCACTGTCTCCT |
| | CA |
| | [SEQ ID NO: 143] |
| DNA | CAGGCTGTTGTGACTCAGGAATCTGCACTCACCACATCACCTGGTGAAA |
| for Full V_{L} | CAGTCACACTCACTTGTCGCTCAAGTACTGGGGCTGTTACAACTAGTAA |
| | CTATGCCAACTGGGTCCAAGAAAAACCAGATCATTTATTCACTGGTCTA |
| | ATAGGTGGTACCAACAACCGAGCTCCAGGTGTTCCTGCCAGATTCTCAG |
| | GCTCCCTGATTGGAGACAAGGCTGCCCTCACCATCACAGGGGCACAGAC |
| | TGAGGATGAGGCAATATATTTCTGTGCTCTATGGTACAGCAACCGCTGG |
| | GTGTTCGGTGGAGGAACCAAACTGACTGTCCTA |
| | [SEQ ID NO: 144] |

In certain embodiments, the anti-DLL3 scFv is an scFv-Fc fusion protein or a full-length human IgG with V_{H} and V_{L} regions or CDRs selected from Table 19. In certain embodiments, the anti-DLL3 scFv comprises a V_{H} comprising the amino acid sequence set forth in SEQ ID NO: 147, as shown in Table 19. An exemplary nucleotide sequence encoding the amino acid sequence of SEQ ID NO: 147 is set forth in SEQ ID NO: 149. In certain embodiments, the anti-DLL3 scFv comprises a V_{L} comprising the amino acid sequence set forth in SEQ ID NO: 148. An exemplary nucleotide sequence encoding the amino acid sequence of SEQ ID NO: 148 is set forth in SEQ ID NO: 150. In certain embodiments, the anti-DLL3 scFv comprises a V_{H} Comprising the amino acid sequence set forth in SEQ ID NO: 147 and a V_{L} comprising the amino acid sequence set forth in SEQ ID NO: 148. SEQ ID NO: 147-150 are provided in Table 19. In certain embodiments, the scFv is designated as “C22”.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 145 or a conservative modification thereof. SEQ ID NOs: 21, 2, and 145 are provided in Table 19.

In certain embodiments, the anti-DLL3 scFv comprises a V_{L} comprising a CDR1 comprising the amino acid sequence set forth in SEQ ID NO: 57 or a conservative modification thereof, a CDR2 comprising the amino acid sequence set forth in SEQ ID NO: 146 or a conservative modification thereof, and a CDR3 comprising the amino acid sequence set forth in SEQ ID NO: 125 or a conservative modification thereof. SEQ ID NOs: 57, 146, and 125 are provided in Table 19.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 145 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 146 or a conservative modification thereof, and a CDR3 comprising the amino acid sequence set forth in SEQ ID NO: 125 or a conservative modification.

In certain embodiments, the anti-DLL3 scFv comprises a VH comprising a CDR1 comprising the amino acid sequence set forth in SEQ ID NO: 21, a CDR2 comprising the amino acid sequence set forth in SEQ ID NO: 2, a V_{H} CDR3 comprising the amino acid sequence set forth in SEQ ID NO: 145; and a V_{L} comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125.

In certain embodiments, the anti-DLL3 scFv comprises a V_{H} comprising the amino acid sequence set forth in SEQ ID NO: 147, and a V_{L} comprising the amino acid sequence set forth in SEQ ID NO: 148. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 19

| CDRS | 1 | 2 | 3 |
|---|---|---|---|
| V_{H} | GYTFTSY | NPSGGS | VADIVGATFDY |
| | [SEQ ID NO: 21] | [SEQ ID NO: 2] | [SEQ ID NO: 145] |
| V_{L} | RASQSVSSSYLA | GASSWAT | QQYDSSPYT |
| | [SEQ ID NO: 57] | [SEQ ID NO: 146] | [SEQ ID NO: 125] |

| | |
|---|---|
| Full V_{H} | QVQLVQSGAEVKKPGASVKVSCKASGYTFTSYYMHWVRQAPGQGLEWMG |
| | IINPSGGSTSYAQKFQGRVTMTRDTSTSTVYMELSSLRSEDTAVYYCAR |
| | VADIVGATFDYWGQGTLVTVSS |
| | [SEQ ID NO: 147] |
| Full V_{L} | EIVLTQSPGTLSLSPGERATLSCRASQSVSSSYLAWYQQKPGQAPRLLI |
| | YGASSWATGIPDRFSGSGSGTDFTLTISRLEPEDFAVYYCQQYDSSPYT |
| | FGQGTKLEIK |
| | [SEQ ID NO: 148] |
| DNA | CAGGTGCAGCTGGTGCAGTCTGGGGCTGAGGTGAAGAAGCCTGGGGCCT |
| for Full V_{H} | CAGTGAAAGTTTCCTGCAAGGCATCTGGATACACCTTCACCAGCTACTA |
| | TATGCACTGGGTGCGACAGGCCCCTGGACAAGGGCTTGAGTGGATGGGA |
| | ATAATCAACCCTAGTGGTGGTAGCACTAGCTACGCACAGAAGTTCCAGG |
| | GCAGAGTCACCATGACCAGGGACACGTCCACGAGCACAGTCTACATGGA |
| | GCTGAGCAGCCTGAGATCTGAGGACACGGCCGTGTATTACTGTGCGAGA |
| | GTGGCGGACATAGTGGGAGCTACTTTTGACTACTGGGGCCAGGGAACCC |
| | TGGTCACCGTCTCCTCA |
| | [SEQ ID NO: 149] |
| DNA | GAAATTGTGTTGACGCAGTCTCCAGGCACCCTGTCTTTGTCTCCAGGGG |
| for Full V_{L} | AAAGAGCCACCCTCTCCTGCAGGGCCAGTCAGAGTGTTAGCAGCAGCTA |
| | CTTAGCCTGGTACCAGCAGAAACCTGGCCAGGCTCCCAGGCTCCTCATC |
| | TATGGTGCATCCAGCTGGGCCACTGGCATCCCAGACAGGTTCAGTGGCA |
| | GTGGGTCTGGGACAGACTTCACTCTCACCATCAGCAGACTGGAACCTGA |
| | AGATTTTGCAGTGTATTACTGTCAGCAGTATGATAGCTCACCCTACACT |
| | TTTGGCCAGGGGACCAAGCTGGAGATCAAA |
| | [SEQ ID NO: 150] |

In certain embodiments, the anti-DLL3 scFv is an scFv-Fc fusion protein or a full-length human IgG with V_{H} and V_{L} regions or CDRs selected from Table 20. In certain embodiments, the anti-DLL3 scFv comprises a V_{H} comprising the amino acid sequence set forth in SEQ ID NO: 153, as shown in Table 20. An exemplary nucleotide sequence encoding the amino acid sequence of SEQ ID NO: 153 is set forth in SEQ ID NO: 155. In certain embodiments, the anti-DLL3 scFv comprises a V_{L} comprising the amino acid sequence set forth in SEQ ID NO: 154. An exemplary nucleotide sequence encoding the amino acid sequence of SEQ ID NO: 154 is set forth in SEQ ID NO: 156. In certain embodiments, the anti-DLL3 scFv comprises a V_{H} Comprising the amino acid sequence set forth in SEQ ID NO: 153 and a V_{L} comprising the amino acid sequence set forth in SEQ ID NO: 154. SEQ ID NO: 153-156 are provided in Table 20. In certain embodiments, the scFv is designated as “D8”.

In certain embodiments, the anti-DLL3 scFv comprises a VH comprising a CDR1 comprising the amino acid sequence set forth in SEQ ID NO: 151 or a conservative modification thereof, a CDR2 comprising the amino acid sequence set forth in SEQ ID NO: 2 or a conservative modification thereof, and a CDR3 comprising the amino acid sequence set forth in SEQ ID NO: 152 or a conservative modification thereof. SEQ ID NOs: 151, 2, and 152 are provided in Table 20.

In certain embodiments, the anti-DLL3 scFv comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof. SEQ ID NOs: 57, 58, and 82 are provided in Table 20.

In certain embodiments, the anti-DLL3 scFv comprises a VH comprising a CDR1 comprising the amino acid sequence set forth in SEQ ID NO: 151 or a conservative modification thereof, a CDR2 comprising the amino acid sequence set forth in SEQ ID NO: 2 or a conservative modification thereof, and a CDR3 comprising the amino acid sequence set forth in SEQ ID NO: 152 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification.

In certain embodiments, the anti-DLL3 scFv comprises a VH comprising a CDR1 comprising the amino acid sequence set forth in SEQ ID NO: 151, a CDR2 comprising the amino acid sequence set forth in SEQ ID NO: 2, a V_{H} CDR3 comprising the amino acid sequence set forth in SEQ ID NO: 152; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82.

In certain embodiments, the anti-DLL3 scFv comprises a V_{H} comprising the amino acid sequence set forth in SEQ ID NO: 153, and a V_{L} comprising the amino acid sequence set forth in SEQ ID NO: 154. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 20

| CDRs | 1 | 2 | 3 |
|---|---|---|---|
| V_{H} | GYTFTTY | NPSGGS | QGHYIGNYLDY |
| | [SEQ ID NO: 151] | [SEQ ID NO: 2] | [SEQ ID NO: 152] |
| V_{L} | RASQSVSSSYLA | GASSRAT | QQYGSSP |
| | [SEQ ID NO: 57] | [SEQ ID NO: 58] | [SEQ ID NO: 82] |

| | |
|---|---|
| Full V_{H} | QVQLVQSGAEVKKPGASVKVSCKASGYTFTTYYMHWVRQAPGQGLEWMG |
| | IINPSGGSTSYAQKFQGRVTMTRDTSTSTVYMELSSLRSEDTAVYYCAR |
| | QGHYIGNYLDYWGQGTLVTVSS |
| | [SEQ ID NO: 153] |
| Full V_{L} | EIVLTQSPGTLSLSPGERATLSCRASQSVSSSYLAWYQQKPGQAPRLLI |
| | YGASSRATGIPDRFSGSGSGTDFTLTISRLEPEDFAVYYCQQYGSSPFG |
| | QGTKLEIK |
| | [SEQ ID NO: 154] |
| DNA | CAGGTGCAGCTGGTGCAGTCTGGGGCTGAGGTGAAGAAGCCTGGGGCCT |
| for Full V_{H} | CAGTGAAGGTTTCCTGCAAGGCATCTGGATACACCTTCACCACCTACTA |
| | TATGCACTGGGTGCGACAGGCCCCTGGACAAGGGCTTGAGTGGATGGGA |
| | ATAATCAACCCTAGTGGTGGTAGCACAAGCTACGCACAGAAGTTCCAGG |
| | GCAGAGTCACCATGACCAGGGACACGTCCACGAGCACAGTCTACATGGA |
| | GCTGAGCAGCCTGAGATCTGAGGACACGGCCGTGTATTACTGTGCGAGA |
| | CAGGGCCATTATATTGGGAACTACCTTGACTACTGGGGCCAGGGAACCC |
| | TGGTCACCGTCTCCTCA |
| | [SEQ ID NO: 155] |
| DNA | GAAATTGTGTTGACGCAGTCTCCAGGCACCCTGTCTTTGTCTCCAGGGG |
| for Full V_{L} | AAAGAGCCACCCTCTCCTGCAGGGCCAGTCAGAGTGTTAGCAGCAGCTA |
| | CTTAGCCTGGTACCAGCAGAAACCTGGCCAGGCTCCCAGGCTCCTCATC |
| | TATGGTGCATCCAGCAGGGCCACTGGCATCCCAGACAGGTTCAGTGGCA |
| | GTGGGTCTGGGACAGACTTCACTCTCACCATCAGCAGACTGGAGCCTGA |
| | AGATTTTGCAGTGTATTACTGTCAGCAGTATGGTAGCTCACCTTTTGGC |
| | CAGGGGACCAAGCTGGAGATCAAA |
| | [SEQ ID NO: 156] |

In certain embodiments, the anti-DLL3 scFv is an scFv-Fc fusion protein or a full-length human IgG with V_{H} and V_{L} regions or CDRs selected from Table 21. In certain embodiments, the anti-DLL3 scFv comprises a V_{H} comprising the amino acid sequence set forth in SEQ ID NO: 157, as shown in Table 21. An exemplary nucleotide sequence encoding the amino acid sequence of SEQ ID NO: 157 is set forth in SEQ ID NO: 159. In certain embodiments, the anti-DLL3 scFv comprises a V_{L} comprising the amino acid sequence set forth in SEQ ID NO: 158. An exemplary nucleotide sequence encoding the amino acid sequence of SEQ ID NO: 158 is set forth in SEQ ID NO: 160. In certain embodiments, the anti-DLL3 scFv comprises a V_{H} Comprising the amino acid sequence set forth in SEQ ID NO: 157 and a V_{L} comprising the amino acid sequence set forth in SEQ ID NO: 158. SEQ ID NO: 157-160 are provided in Table 21. In certain embodiments, the scFv is designated as “G16”.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof. SEQ ID NOs: 21, 2, and 123 are provided in Table 21.

In certain embodiments, the anti-DLL3 scFv comprises a V_{L} comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof. SEQ ID NOs: 124, 58, and 59 are provided in Table 21.

In certain embodiments, the anti-DLL3 scFv comprises a VH comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof; and a V_{L} comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification.

In certain embodiments, the anti-DLL3 scFv comprises a VH comprising a CDR1 comprising the amino acid sequence set forth in SEQ ID NO: 21, a CDR2 comprising the amino acid sequence set forth in SEQ ID NO: 2, a V_{H} CDR3 comprising the amino acid sequence set forth in SEQ ID NO: 123; and a V_{L} comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59.

In certain embodiments, the anti-DLL3 scFv comprises a V_{H} comprising the amino acid sequence set forth in SEQ ID NO: 157, and a V_{L} comprising the amino acid sequence set forth in SEQ ID NO: 158. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 21

| CDRs | 1 | 2 | 3 |
|---|---|---|---|
| V_{H} | GYTFTSY | NPSGGS | QGGAQWLVLAFDI |
| | [SEQ ID NO: 21] | [SEQ ID NO: 2] | [SEQ ID NO: 123] |
| V_{L} | RASQSVSSYLA | GASSRAT | QQYGSSPYT |
| | [SEQ ID NO: 124] | [SEQ ID NO: 58] | [SEQ ID NO: 59] |

| | |
|---|---|
| Full V_{H} | QVQLVQSGAEVKKPGASVQVSCKASGYTFTSYYMHWVRQAPGQGLEWMG |
| | IINPSGGSTSYAQKFQGRVTMTRDTSTSTVYMELSSLRSEDTAVYYCAR |
| | QGGAQWLVLAFDIWGQGTMVTVSS |
| | [SEQ ID NO: 157] |
| Full V_{L} | EIVLTQSPGTLSLSPGERATLSCRASQSVSSYLAWYQQKPGQAPRLLIY |
| | GASSRATGIPDRESGGGSGTDFTLTISRLEPEDFAVYYCQQYGSSPYTF |
| | GQGTKLEIK |
| | [SEQ ID NO: 158] |
| DNA | CAGGTGCAGCTGGTGCAGTCTGGGGCTGAGGTGAAGAAGCCTGGGGCCT |
| for Full V_{H} | CAGTGCAGGTTTCCTGCAAGGCATCTGGATACACCTTCACCAGCTACTA |
| | TATGCACTGGGTGCGACAGGCCCCTGGACAAGGGCTTGAGTGGATGGGA |
| | ATAATCAACCCTAGTGGTGGTAGCACAAGCTACGCACAGAAGTTCCAGG |
| | GCAGAGTCACCATGACCAGGGACACGTCCACGAGCACAGTCTACATGGA |
| | GCTGAGCAGCCTGAGATCTGAGGACACGGCCGTGTATTACTGTGCGAGA |
| | CAGGGGGGAGCCCAGTGGCTGGTACTTGCTTTTGATATCTGGGGCCAAG |
| | GGACAATGGTCACCGTCTCTTCA |
| | [SEQ ID NO: 159] |
| DNA | GAAATTGTGTTGACGCAGTCTCCAGGCACCCTGTCTTTGTCTCCAGGGG |
| for Full V_{L} | AAAGAGCCACCCTCTCCTGCAGGGCCAGTCAGAGTGTTAGCAGCTACTT |
| | AGCCTGGTACCAGCAGAAACCTGGCCAGGCTCCCAGGCTCCTCATCTAT |
| | GGTGCATCCAGCAGGGCCACTGGCATCCCAGACAGGTTCAGTGGCGGTG |
| | GGTCTGGGACAGACTTCACTCTCACCATCAGCAGACTGGAGCCTGAAGA |
| | TTTTGCAGTGTATTACTGTCAGCAGTATGGTAGCTCACCGTACACTTTT |
| | GGCCAGGGGACCAAGCTGGAGATCAAA |
| | [SEQ ID NO: 160] |

In certain embodiments, the anti-DLL3 scFv is an scFv-Fc fusion protein or a full-length human IgG with V_{H} and V_{L} regions or CDRs selected from Table 22. In certain embodiments, the anti-DLL3 scFv comprises a V_{H} comprising the amino acid sequence set forth in SEQ ID NO: 163, as shown in Table 22. An exemplary nucleotide sequence encoding the amino acid sequence of SEQ ID NO: 163 is set forth in SEQ ID NO: 165. In certain embodiments, the anti-DLL3 scFv comprises a V_{L} comprising the amino acid sequence set forth in SEQ ID NO: 164. An exemplary nucleotide sequence encoding the amino acid sequence of SEQ ID NO: 164 is set forth in SEQ ID NO: 166. In certain embodiments, the anti-DLL3 scFv comprises a V_{H} Comprising the amino acid sequence set forth in SEQ ID NO: 163 and a V_{L} comprising the amino acid sequence set forth in SEQ ID NO: 164. SEQ ID NO: 163-166 are provided in Table 22. In certain embodiments, the scFv is designated as “F21”.

In certain embodiments, the anti-DLL3 scFv comprises a VH comprising a CDR1 comprising the amino acid sequence set forth in SEQ ID NO: 11 or a conservative modification thereof, a CDR2 comprising the amino acid sequence set forth in SEQ ID NO: 136 or a conservative modification thereof, and a CDR3 comprising the amino acid sequence set forth in SEQ ID NO: 161 or a conservative modification thereof. SEQ ID NOs: 11, 136, and 161 are provided in Table 22.

In certain embodiments, the anti-DLL3 scFv comprises a V_{L} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 162 or a conservative modification thereof. SEQ ID NOs: 73, 74, and 162 are provided in Table 22.

In certain embodiments, the anti-DLL3 scFv comprises a VH comprising a CDR1 comprising the amino acid sequence set forth in SEQ ID NO: 11 or a conservative modification thereof, a CDR2 comprising the amino acid sequence set forth in SEQ ID NO: 136 or a conservative modification thereof, and a CDR3 comprising the amino acid sequence set forth in SEQ ID NO: 161 or a conservative modification thereof; and a V_{L} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 162 or a conservative modification.

In certain embodiments, the anti-DLL3 scFv comprises a VH comprising a CDR1 comprising the amino acid sequence set forth in SEQ ID NO: 11, a CDR2 comprising the amino acid sequence set forth in SEQ ID NO: 136, a V_{H} CDR3 comprising the amino acid sequence set forth in SEQ ID NO: 161; and a V_{L} comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162.

In certain embodiments, the anti-DLL3 scFv comprises a V_{H} comprising the amino acid sequence set forth in SEQ ID NO: 163, and a V_{L} comprising the amino acid sequence set forth in SEQ ID NO: 164. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 22

| CDRS | 1 | 2 | 3 |
|---|---|---|---|
| V_{H} | GFTFSSY | KQDGSE | EWLRFGGLVY |
| | [SEQ ID NO: 11] | [SEQ ID NO: 136] | [SEQ ID NO: 161] |
| V_{L} | RASQSVSSNLA | GASTRAT | QQYNNWPLT |
| | [SEQ ID NO: 73] | [SEQ ID NO: 74] | [SEQ ID NO: 162] |

| | |
|---|---|
| Full V_{H} | EVQLVESGGGLVQPGGSLRLSCAASGFTFSSYWMSWVRQAPGKGLEWVA |
| | NIKQDGSEKYYVDSVKGRFTISRDNAKNSLYLQMNSLRAEDTAVYYCAR |
| | EWLRFGGLVYWGQGTLVTVSS |
| | [SEQ ID NO: 163] |
| Full V_{L} | EIVMTQSPATLSVSPGERATLSCRASQSVSSNLAWYQQKPGQAPRLLIY |
| | GASTRATGIPARFSGSGSGTEFTLTISSLQSEDFAVYYCQQYNNWLPTF |
| | GGGTKVEIK |
| | [SEQ ID NO: 164] |
| DNA | GAGGTGCAGCTGGTGGAGTCTGGGGGAGGCTTGGTCCAGCCTGGGGGGT |
| for Full V_{H} | CCCTGAGACTCTCCTGTGCAGCCTCTGGATTCACCTTTAGTAGCTATTG |
| | GATGAGCTGGGTCCGCCAGGCTCCAGGGAAGGGGCTGGAGTGGGTGGCC |
| | AACATAAAGCAAGATGGAAGTGAGAAATACTATGTGGACTCTGTGAAGG |
| | GCCGATTCACCATCTCCAGAGACAACGCCAAGAACTCACTGTATCTGCA |
| | AATGAACAGCCTGAGAGCCGAGGACACGGCTGTGTATTACTGTGCGAGA |
| | GAGTGGCTACGATTTGGGGGCTTAGTGTACTGGGGCCAGGGAACCCTGG |
| | TCACCGTCTCCTCA |
| | [SEQ ID NO: 165] |
| DNA | GAAATAGTGATGACGCAGTCTCCAGCCACCCTGTCTGTGTCTCCAGGGG |
| for Full V_{L} | AAAGAGCCACCCTCTCCTGCAGGGCCAGTCAGAGTGTTAGCAGCAACTT |
| | AGCCTGGTACCAGCAGAAACCTGGCCAGGCTCCCAGGCTCCTCATCTAT |
| | GGTGCATCCACCAGGGCCACTGGTATCCCAGCCAGGTTCAGTGGCAGTG |
| | GGTCTGGGACAGAGTTCACTCTCACCATCAGCAGCCTGCAGTCTGAAGA |
| | TTTTGCAGTTTATTACTGTCAGCAGTATAATAACTGGCCGCTCACTTTC |
| | GGCGGAGGGACCAAGGTGGAGATCAAA |
| | [SEQ ID NO: 166] |

In certain embodiments, the anti-DLL3 scFv is an scFv-Fc fusion protein or a full-length human IgG with V_{H} and V_{L} regions or CDRs selected from Table 23. In certain embodiments, the anti-DLL3 scFv comprises a V_{H} comprising the amino acid sequence set forth in SEQ ID NO: 172, as shown in Table 23. An exemplary nucleotide sequence encoding the amino acid sequence of SEQ ID NO: 172 is set forth in SEQ ID NO: 174. In certain embodiments, the anti-DLL3 scFv comprises a V_{L} comprising the amino acid sequence set forth in SEQ ID NO: 173. An exemplary nucleotide sequence encoding the amino acid sequence of SEQ ID NO: 173 is set forth in SEQ ID NO: 175. In certain embodiments, the anti-DLL3 scFv comprises a V_{H} Comprising the amino acid sequence set forth in SEQ ID NO: 172 and a V_{L} comprising the amino acid sequence set forth in SEQ ID NO: 173. SEQ ID NO: 172-175 are provided in Table 23. In certain embodiments, the scFv is designated as “N12”.

In certain embodiments, the anti-DLL3 scFv comprises a VH comprising a CDR1 comprising the amino acid sequence set forth in SEQ ID NO: 96 or a conservative modification thereof, a CDR2 comprising the amino acid sequence set forth in SEQ ID NO: 167 or a conservative modification thereof, and a CDR3 comprising the amino acid sequence set forth in SEQ ID NO: 168 or a conservative modification thereof. SEQ ID NOs: 96, 167, and 168 are provided in Table 23.

In certain embodiments, the anti-DLL3 scFv comprises a V_{L} comprising a CDR1 comprising the amino acid sequence set forth in SEQ ID NO: 169 or a conservative modification thereof, a CDR2 comprising the amino acid sequence set forth in SEQ ID NO: 170 or a conservative modification thereof, and a CDR3 comprising the amino acid sequence set forth in SEQ ID NO: 171 or a conservative modification thereof. SEQ ID NOs: 169-171 are provided in Table 23.

In certain embodiments, the anti-DLL3 scFv comprises a VH comprising a CDR1 comprising the amino acid sequence set forth in SEQ ID NO: 96 or a conservative modification thereof, a CDR2 comprising the amino acid sequence set forth in SEQ ID NO: 167 or a conservative modification thereof, and a CDR3 comprising the amino acid sequence set forth in SEQ ID NO: 168 or a conservative modification thereof; and a V_{L} comprising a CDR1 comprising the amino acid sequence set forth in SEQ ID NO: 169 or a conservative modification thereof, a CDR2 comprising the amino acid sequence set forth in SEQ ID NO: 170 or a conservative modification thereof, and a CDR3 comprising the amino acid sequence set forth in SEQ ID NO: 171 or a conservative modification.

In certain embodiments, the anti-DLL3 scFv comprises a VH comprising a CDR1 comprising the amino acid sequence set forth in SEQ ID NO: 96, a CDR2 comprising the amino acid sequence set forth in SEQ ID NO: 167, a V_{H} CDR3 comprising the amino acid sequence set forth in SEQ ID NO: 168; and a V_{L} comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171.

In certain embodiments, the anti-DLL3 scFv comprises a V_{H} comprising the amino acid sequence set forth in SEQ ID NO: 172, and a V_{L} comprising the amino acid sequence set forth in SEQ ID NO: 173. In certain embodiments, the V_{H} and V_{L} are linked via a linker. In certain embodiments, the linker comprises the amino acid sequence set forth in SEQ ID NO: 177.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 23

| CDRS | 1 | 2 | 3 |
|---|---|---|---|
| V_{H} | GYTFTGY | NPNSGG | DGVITSFDY |
| | [SEQ ID NO: 96] | [SEQ ID NO: 167] | [SEQ ID NO: 168] |
| V_{L} | KSSQSVLYSSNIKTYLA | WASTRES | QQYYSAPYT |
| | [SEQ ID NO: 169] | [SEQ ID NO: 170] | [SEQ ID NO: 171] |

| | |
|---|---|
| Full V_{H} | QVQLVQSGAEVKKPGASVKVSCKASGYTFTGYYMHWVRQAPGQGLEWMG |
| | WINPNSGGTNYAQKFQGRVTMTRDTSISTAYMELSRLRSDDTAVYYCAR |
| | DGVITSFDYWGQGTLVTVSS |
| | [SEQ ID NO: 172] |
| Full V_{L} | DIVMTQSPDSLAVSLGERATINCKSSQSVLYSSNIKTYLAWYQQKPGQP |
| | PKLLIYWASTRESGVPDRFSGGGSGTDFTLTITSLQAEDVAVYYCQQYY |
| | SAPYTFGQGTKLEIK |
| | [SEQ ID NO: 173] |
| DNA | CAGGTGCAGCTGGTGCAGTCTGGGGCTGAGGTGAAGAAGCCTGGGGCCT |
| for Full V_{H} | CAGTGAAGGTCTCCTGCAAGGCTTCTGGATACACTTTCACCGGCTACTA |
| | TATGCACTGGGTGCGACAGGCCCCTGGACAAGGGCTTGAGTGGATGGGA |
| | TGGATCAACCCTAACAGTGGTGGCACAAACTATGCACAGAAGTTTCAGG |
| | GCAGGGTCACCATGACCAGGGACACGTCCATCAGCACAGCCTACATGGA |
| | GCTGAGCAGGCTGAGATCTGACGACACGGCCGTGTATTACTGTGCGAGA |
| | GACGGGGTGATTACGTCTTTTGACTACTGGGGCCAGGGAACCCTGGTCA |
| | CCGTCTCCTCA |
| | [SEQ ID NO: 174] |
| DNA | GACATCGTGATGACCCAGTCTCCAGACTCCCTGGCTGTGTCTCTGGGCG |
| for Full V_{L} | AGAGGGCCACCATCAACTGCAAGTCCAGCCAGAGTGTTTTATACAGCTC |
| | CAACATTAAGACCTACTTAGCTTGGTACCAGCAGAAACCAGGACAGCCT |
| | CCTAAGCTGCTCATTTACTGGGCATCTACCCGGGAATCCGGAGTCCCTG |
| | ACCGATTCAGTGGCGGCGGGTCTGGGACAGATTTCACTCTCACCATCAC |
| | CAGCCTGCAGGCTGAAGATGTGGCAGTTTATTACTGTCAGCAGTATTAT |
| | AGTGCTCCGTACACTTTTGGCCAGGGGACCAAGCTGGAGATCAAA |
| | [SEQ ID NO: 175] |

In certain embodiments, the anti-DLL3 antibody or antigen-binding fragment thereof comprises V_{H} and V_{L} regions or CDRs selected from Table 24. In certain embodiments, the anti-DLL3 antibody or antigen-binding fragment thereof comprises a V_{H} comprising the amino acid sequence set forth in SEQ ID NO: 187, as shown in Table 24. An exemplary nucleotide sequence encoding the amino acid sequence of SEQ ID NO: 187 is set forth in SEQ ID NO: 189. In certain embodiments, the anti-DLL3 antibody or antigen-binding fragment thereof comprises a V_{L} comprising the amino acid sequence set forth in SEQ ID NO: 188. An exemplary nucleotide sequence encoding the amino acid sequence of SEQ ID NO: 188 is set forth in SEQ ID NO: 190. In certain embodiments, the anti-DLL3 antibody or antigen-binding fragment thereof comprises a V_{H} comprising the amino acid sequence set forth in SEQ ID NO: 187 and a V_{L} Comprising the amino acid sequence set forth in SEQ ID NO: 188. SEQ ID NO: 187-190 are provided in Table 24. In certain embodiments, the antibody or antigen-binding fragment thereof is designated as “G23”.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 183 or a conservative modification thereof, and a CDR3 comprising the amino acid sequence set forth in SEQ ID NO: 184 or a conservative modification thereof. SEQ ID NOs: 21, 183, and 184 are provided in Table 24.

In certain embodiments, the anti-DLL3 antibody or antigen-binding fragment thereof comprises a V_{L} comprising a CDR1 comprising the amino acid sequence set forth in SEQ ID NO: 185 or a conservative modification thereof, a CDR2 comprising the amino acid sequence set forth in SEQ ID NO: 50 or a conservative modification thereof, and a CDR3 comprising the amino acid sequence set forth in SEQ ID NO: 186 or a conservative modification thereof. SEQ ID NOs: 50, 185, and 186 are provided in Table 24.

In certain embodiments, the anti-DLL3 antibody or antigen-binding fragment thereof comprises a V_{H} comprising a CDR1 comprising the amino acid sequence set forth in SEQ ID NO: 21 or a conservative modification thereof, a CDR2 comprising the amino acid sequence set forth in SEQ ID NO: 184 or a conservative modification thereof, and a CDR3 comprising the amino acid sequence set forth in SEQ ID NO: 185 or a conservative modification thereof; and a V_{L} comprising a CDR1 comprising the amino acid sequence set forth in SEQ ID NO: 186 or a conservative modification thereof, a CDR2 comprising the amino acid sequence set forth in SEQ ID NO: 50 or a conservative modification thereof, and a CDR3 comprising the amino acid sequence set forth in SEQ ID NO: 187 or a conservative modification.

In certain embodiments, the anti-DLL3 antibody or antigen-binding fragment thereof comprises a V_{H} comprising a CDR1 comprising the amino acid sequence set forth in SEQ ID NO: 21, a CDR2 comprising the amino acid sequence set forth in SEQ ID NO: 183, and a CDR3 comprising the amino acid sequence set forth in SEQ ID NO: 184; and a V_{L} comprising a CDR1 comprising the amino acid sequence set forth in SEQ ID NO: 185, a CDR2 comprising the amino acid sequence set forth in SEQ ID NO: 50, and a CDR3 comprising the amino acid sequence set forth in SEQ ID NO: 186.

TABLE 24

| CDRs | 1 | 2 | 3 |
|---|---|---|---|
| V_{H} | GYTFTSY | DPSDGS | DREYNYYGLDV |
| | [SEQ ID NO: 21] | [SEQ ID NO: 183] | [SEQ ID NO: 184] |
| V_{L} | RSSQSLVYRDGNTYLN | KVSNRDS | MQGTHWPPT |
| | [SEQ ID NO: 185] | [SEQ ID NO: 50] | [SEQ ID NO: 186] |

| | |
|---|---|
| Full V_{H} | QVQLVQSGAEVKKPGASVKVSCKASGYTFTSYYIHWVRQAPGQGLEWMG |
| | IIDPSDGSTNYAQKFQGRVTMTRDTSTSTVYMELSSLRSEDTAVYYCAR |
| | DREYNYYGLDVWGQGTTVTVSS |
| | [SEQ ID NO: 187] |
| Full V_{L} | DVVMTQSPLSLPVTLGQPASISCRSSQSLVYRDGNTYLNWFQQRPGQSP |
| | RRLIYKVSNRDSGVPDRFRGSGSGTDFTLKISRVEAEDVGVYYCMQGTH |
| | WPPTFGQGTKVEIK |
| | [SEQ ID NO: 188] |
| DNA | CAGGTGCAGCTGGTGCAGTCTGGGGCTGAGGTGAAGAAGCCTGGGGCCT |
| for Full V_{H} | CAGTGAAGGTTTCCTGCAAGGCATCTGGATACACCTTCACCAGCTACTA |
| | TATACACTGGGTGCGACAGGCCCCTGGACAAGGGCTTGAGTGGATGGGA |
| | ATAATCGACCCAAGTGATGGTAGCACAAACTACGCACAGAAGTTCCAGG |
| | GCAGAGTCACCATGACCAGGGACACGTCCACGAGCACAGTCTACATGGA |
| | GCTGAGCAGCCTGAGATCTGAGGACACGGCCGTGTATTACTGTGCGAGA |
| | GATCGGGAATATAACTACTACGGTTTGGACGTCTGGGGCCAAGGGACCA |
| | CGGTCACCGTCTCCTCA |
| | [SEQ ID NO: 189] |
| DNA | GATGTTGTGATGACTCAGTCTCCACTCTCCCTGCCCGTCACCCTTGGAC |
| for Full V_{L} | AGCCGGCCTCCATCTCCTGCAGGTCTAGTCAAAGCCTCGTATACCGTGA |
| | TGGAAACACCTACTTGAATTGGTTTCAGCAGAGGCCAGGCCAATCTCCA |
| | AGGCGCCTAATTTATAAGGTTTCTAACCGGGACTCTGGGGTCCCAGACA |
| | GATTCCGCGGCAGTGGGTCAGGCACTGATTTCACACTGAAAATCAGCCG |
| | GGTGGAGGCTGAGGATGTTGGGGTTTATTACTGCATGCAAGGTACACAC |
| | TGGCCTCCGACGTTCGGCCAAGGGACCAAGGTGGAAATCAAA |
| | [SEQ ID NO: 190] |

In certain embodiments, the anti-DLL3 antibody or antigen-binding fragment thereof comprises V_{H} and V_{L} regions or CDRs selected from Table 25. In certain embodiments, the anti-DLL3 antibody or antigen-binding fragment thereof comprises a V_{H} comprising the amino acid sequence set forth in SEQ ID NO: 197, as shown in Table 25. An exemplary nucleotide sequence encoding the amino acid sequence of SEQ ID NO: 197 is set forth in SEQ ID NO: 199. In certain embodiments, the anti-DLL3 antibody or antigen-binding fragment thereof comprises a V_{L} comprising the amino acid sequence set forth in SEQ ID NO: 198. An exemplary nucleotide sequence encoding the amino acid sequence of SEQ ID NO: 198 is set forth in SEQ ID NO: 200. In certain embodiments, the anti-DLL3 antibody or antigen-binding fragment thereof comprises a V_{H} comprising the amino acid sequence set forth in SEQ ID NO: 197 and a V_{L} Comprising the amino acid sequence set forth in SEQ ID NO: 198. SEQ ID NO: 197-200 are provided in Table 25. In certain embodiments, the antibody or antigen-binding fragment thereof is designated as “I1”.

In certain embodiments, the anti-DLL3 antibody or antigen-binding fragment thereof comprises a V_{H} comprising a CDR1 comprising the amino acid sequence set forth in SEQ ID NO: 191 or a conservative modification thereof, a CDR2 comprising the amino acid sequence set forth in SEQ ID NO: 192 or a conservative modification thereof, and a CDR3 comprising the amino acid sequence set forth in SEQ ID NO: 193 or a conservative modification thereof. SEQ ID NOs: 191-193 are provided in Table 25.

In certain embodiments, the anti-DLL3 antibody or antigen-binding fragment thereof comprises a V_{L} comprising a CDR1 comprising the amino acid sequence set forth in SEQ ID NO: 194 or a conservative modification thereof, a CDR2 comprising the amino acid sequence set forth in SEQ ID NO: 195 or a conservative modification thereof, and a CDR3 comprising the amino acid sequence set forth in SEQ ID NO: 196 or a conservative modification thereof. SEQ ID NOs: 194-196 are provided in Table 25.

In certain embodiments, the anti-DLL3 antibody or antigen-binding fragment thereof comprises a V_{H} comprising a CDR1 comprising the amino acid sequence set forth in SEQ ID NO: 191 or a conservative modification thereof, a CDR2 comprising the amino acid sequence set forth in SEQ ID NO: 192 or a conservative modification thereof, and a CDR3 comprising the amino acid sequence set forth in SEQ ID NO: 193 or a conservative modification thereof; and a V_{L} comprising a CDR1 comprising the amino acid sequence set forth in SEQ ID NO: 194 or a conservative modification thereof, a CDR2 comprising the amino acid sequence set forth in SEQ ID NO: 195 or a conservative modification thereof, and a CDR3 comprising the amino acid sequence set forth in SEQ ID NO: 196 or a conservative modification.

In certain embodiments, the anti-DLL3 antibody or antigen-binding fragment thereof comprises a V_{H} comprising a CDR1 comprising the amino acid sequence set forth in SEQ ID NO: 191, a CDR2 comprising the amino acid sequence set forth in SEQ ID NO: 192, and a CDR3 comprising the amino acid sequence set forth in SEQ ID NO: 193; and a V_{L} comprising a CDR1 comprising the amino acid sequence set forth in SEQ ID NO: 194, a CDR2 comprising the amino acid sequence set forth in SEQ ID NO: 195, and a CDR3 comprising the amino acid sequence set forth in SEQ ID NO: 196.

TABLE 25

| CDRS | 1 | 2 | 3 |
|---|---|---|---|
| V_{H} | GFTFSNTW | IKSKSDGGTT | TQYYWNSFDY |
| | [SEQ ID NO: 191] | [SEQ ID NO: 192] | [SEQ ID NO: 193] |
| V_{L} | QASQDISNYLN | DASNLET | QQYDNLPLT |
| | [SEQ ID NO: 194] | [SEQ ID NO: 195] | [SEQ ID NO: 196] |

| | |
|---|---|
| Full V_{H} | EVQLVESGGGLVKPGGSLRLSCAASGFTFSNTWMSWVRQAPGKGLEWVG |
| | RIKSKSDGGTTDYAAPVKGRFTISRDDSKNTLYLQMNSLKTEDTAVYYC |
| | TQYYWNSFDYWGQGTLVTVSS |
| | [SEQ ID NO: 197] |
| Full V_{L} | DIQMTQSPSSLSASVGDRVTITCQASQDISNYLNWYQQKPGKAPKLLIY |
| | DASNLETGVPSRFSGSGSGTDFTFTISSLQPEDIATYYCQQYDNLPLTF |
| | GGGTKVEIK |
| | [SEQ ID NO: 198] |
| DNA | GAGGTGCAGCTGGTGGAGTCTGGGGGGGGCTTGGTAAAGCCTGGGGGGT |
| for Full V_{H} | CCCTTAGACTCTCCTGTGCAGCCTCTGGATTCACTTTCAGTAACACCTG |
| | GATGAGCTGGGTCCGCCAGGCTCCAGGGAAGGGGCTGGAGTGGGTTGGC |
| | CGTATTAAAAGCAAATCTGATGGTGGGACAACAGACTACGCTGCACCCG |
| | TGAAAGGCAGATTCACCATCTCAAGAGATGATTCAAAAAACACGCTGTA |
| | TCTGCAAATGAACAGCCTGAAAACCGAGGACACAGCCGTGTATTACTGT |
| | ACCCAGTATTATTGGAACTCCTTTGACTACTGGGGCCAGGGAACCCTGG |
| | TCACCGTCTCCTCA |
| | [SEQ ID NO: 199] |
| DNA | GACATCCAGATGACCCAGTCTCCATCCTCCCTGTCTGCATCTGTAGGAG |
| for Full V_{L} | ACAGAGTCACCATCACTTGCCAGGCGAGTCAGGACATTAGCAACTATTT |
| | AAATTGGTATCAGCAGAAACCAGGGAAAGCCCCTAAGCTCCTGATCTAC |
| | GATGCATCCAATTTGGAAACAGGGGTCCCATCAAGGTTCAGTGGAAGTG |
| | GATCTGGGACAGATTTTACTTTCACCATCAGCAGCCTGCAGCCTGAAGA |
| | TATTGCAACATATTACTGTCAACAGTATGATAATCTCCCGCTCACTTTC |
| | GGCGGAGGGACCAAGGTGGAGATCAAA |
| | [SEQ ID NO: 200] |

In certain embodiments, the anti-DLL3 antibody or antigen-binding fragment thereof comprises V_{H} and V_{L} regions or CDRs selected from Table 26. In certain embodiments, the anti-DLL3 antibody or antigen-binding fragment thereof comprises a V_{H} comprising the amino acid sequence set forth in SEQ ID NO: 204, as shown in Table 26. An exemplary nucleotide sequence encoding the amino acid sequence of SEQ ID NO: 204 is set forth in SEQ ID NO: 206. In certain embodiments, the anti-DLL3 antibody or antigen-binding fragment thereof comprises a V_{L} comprising the amino acid sequence set forth in SEQ ID NO: 205. An exemplary nucleotide sequence encoding the amino acid sequence of SEQ ID NO: 205 is set forth in SEQ ID NO: 207. In certain embodiments, the anti-DLL3 antibody or antigen-binding fragment thereof comprises a V_{H} comprising the amino acid sequence set forth in SEQ ID NO: 204 and a V_{L} Comprising the amino acid sequence set forth in SEQ ID NO: 205. SEQ ID NO: 204-207 are provided in Table 26. In certain embodiments, the antibody or antigen-binding fragment thereof is designated as “C8”

In certain embodiments, the anti-DLL3 antibody or antigen-binding fragment thereof comprises a V_{H} comprising a CDR1 comprising the amino acid sequence set forth in SEQ ID NO: 11 or a conservative modification thereof, a CDR2 comprising the amino acid sequence set forth in SEQ ID NO: 201 or a conservative modification thereof, and a CDR3 comprising the amino acid sequence set forth in SEQ ID NO: 202 or a conservative modification thereof. SEQ ID NOs: 11, 201, and 202 are provided in Table 26.

In certain embodiments, the anti-DLL3 antibody or antigen-binding fragment thereof comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 203 or a conservative modification thereof. SEQ ID NOs: 4, 5, and 203 are provided in Table 26.

In certain embodiments, the anti-DLL3 antibody or antigen-binding fragment thereof comprises a V_{H} comprising a CDR1 comprising the amino acid sequence set forth in SEQ ID NO: 11 or a conservative modification thereof, a CDR2 comprising the amino acid sequence set forth in SEQ ID NO: 201 or a conservative modification thereof, and a CDR3 comprising the amino acid sequence set forth in SEQ ID NO: 202 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 203 or a conservative modification.

In certain embodiments, the anti-DLL3 antibody or antigen-binding fragment thereof comprises a V_{H} comprising a CDR1 comprising the amino acid sequence set forth in SEQ ID NO: 11, a CDR2 comprising the amino acid sequence set forth in SEQ ID NO: 201, and a CDR3 comprising the amino acid sequence set forth in SEQ ID NO: 202; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 203.

TABLE 26

| CDRS | 1 | 2 | 3 |
|---|---|---|---|
| V_{H} | GFTFSSY | KEDGSE | DPGWAPFDY |
| | [SEQ ID NO: 11] | [SEQ ID NO: 201] | [SEQ ID NO: 202] |
| V_{L} | RASQGISNYLA | AASSLQS | QQYNSFPYT |
| | [SEQ ID NO: 4] | [SEQ ID NO: 5] | [SEQ ID NO: 203] |

| | |
|---|---|
| Full V_{H} | EVQLVESGGGLVQPGGSQRLSCAASGFTFSSYWMNWVRQAPGKGLEWVA |
| | NIKEDGSEKYYVDSVKGRFTISRDNAKNSLYLQMNSLRAEDTAVYYCAR |
| | DPGWAPFDYWGQGTLVTVSS |
| | [SEQ ID NO: 204] |
| Full V_{L} | |
| DNA | |
| for Full V_{H} | |
| | GATGAACTGGGTCCGCCAGGCTCCAGGGAAGGGGCTGGAGTGGGTGGCC |
| | AACATAAAGGAAGATGGAAGTGAGAAATACTATGTGGACTCTGTGAAGG |
| | GCCGATTCACCATCTCCAGAGACAACGCCAAGAACTCACTGTATCTGCA |
| | AATGAACAGCCTGAGAGCCGAGGACACGGCTGTGTATTACTGTGCGAGA |
| | GATCCGGGCTGGGCTCCCTTTGACTACTGGGGCCAGGGAACCCTGGTCA |
| DNA | GACATCCAGATGTCCCAGTCTCCATCCTCACTGTCTGCATCTGTAGGAG |
| for Full V_{L} | ACAGAGTCACCATCACTTGTCGGGCGAGTCAGGGCATTAGCAATTATTT |
| | AGCCTGGTTTCAGCAGAAACCAGGGAAAGCCCCTAAGTCCCTGATCTAT |
| | GCTGCATCCAGTTTGCAAAGTGGGGTCCCATCAAAGTTCAGCGGCAGTG |
| | GATCTGGGACAGATTTCACTCTCGCCATCAGCAGCCTGCAGCCTGAAGA |
| | TTTTGCAACTTATTACTGCCAACAGTATAATAGTTTCCCGTACACTTTT |
| | GGCCAGGGGACCACGCTGGAGATCAAA |
| | [SEQ ID NO: 207] |

In certain embodiments, the anti-DLL3 antibody or antigen-binding fragment thereof comprises V_{H} and V_{L} regions or CDRs selected from Table 27. In certain embodiments, the anti-DLL3 antibody or antigen-binding fragment thereof comprises a V_{H} comprising the amino acid sequence set forth in SEQ ID NO: 212, as shown in Table 27. An exemplary nucleotide sequence encoding the amino acid sequence of SEQ ID NO: 212 is set forth in SEQ ID NO: 214. In certain embodiments, the anti-DLL3 antibody or antigen-binding fragment thereof comprises a V_{L} comprising the amino acid sequence set forth in SEQ ID NO: 213. An exemplary nucleotide sequence encoding the amino acid sequence of SEQ ID NO: 213 is set forth in SEQ ID NO: 215. In certain embodiments, the anti-DLL3 antibody or antigen-binding fragment thereof comprises a V_{H} comprising the amino acid sequence set forth in SEQ ID NO: 212 and a V_{L} Comprising the amino acid sequence set forth in SEQ ID NO: 213. SEQ ID NO: 212-215 are provided in Table 27. In certain embodiments, the antibody or antigen-binding fragment thereof is designated as “O18”.

In certain embodiments, the anti-DLL3 antibody or antigen-binding fragment thereof comprises a V_{H} comprising a CDR1 comprising the amino acid sequence set forth in SEQ ID NO: 208 or a conservative modification thereof, a CDR2 comprising the amino acid sequence set forth in SEQ ID NO: 209 or a conservative modification thereof, and a CDR3 comprising the amino acid sequence set forth in SEQ ID NO: 210 or a conservative modification thereof. SEQ ID NOs: 208-210 are provided in Table 27.

In certain embodiments, the anti-DLL3 antibody or antigen-binding fragment thereof comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 211 or a conservative modification thereof. SEQ ID NOs: 57, 58, and 211 are provided in Table 27.

In certain embodiments, the anti-DLL3 antibody or antigen-binding fragment thereof comprises a V_{H} comprising a CDR1 comprising the amino acid sequence set forth in SEQ ID NO: 208 or a conservative modification thereof, a CDR2 comprising the amino acid sequence set forth in SEQ ID NO: 209 or a conservative modification thereof, and a CDR3 comprising the amino acid sequence set forth in SEQ ID NO: 210 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 211 or a conservative modification.

In certain embodiments, the anti-DLL3 antibody or antigen-binding fragment thereof comprises a V_{H} comprising a CDR1 comprising the amino acid sequence set forth in SEQ ID NO: 201 a CDR2 comprising the amino acid sequence set forth in SEQ ID NO: 209, a CDR3 comprising the amino acid sequence set forth in SEQ ID NO: 210; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 211.

TABLE 27

| CDRS | 1 | 2 | 3 |
|---|---|---|---|
| V_{H} | GGSINSY | FYSGI | IGVAGFYFDY |
| | [SEQ ID NO: 208] | [SEQ ID NO: 209] | [SEQ ID NO: 210] |
| V_{L} | RASQSVSSSYLA | GASSRAT | QQYGTSPLT |
| | [SEQ ID NO: 57] | [SEQ ID NO: 58] | [SEQ ID NO: 211] |

| | |
|---|---|
| Full V_{H} | QVQLQESGPGLVKPSETLSLTCTVSGGSINSYYWSWIRQPPGKGLEWIG |
| | YIFYSGITNYNPSLKSRVTISLDTSKNQFSLKLSSVTAADTAVYYCARI |
| | GVAGFYFDYWGQGTLVTVSS |
| | [SEQ ID NO: 212] |
| Full V_{L} | EIVLTQSPGTLSLSPGERATLSCRASQSVSSSYLAWYQQKPGQAPRLLI |
| | YGASSRATGIPDRESGSGSGTDFTLTISRLEPEDFAVYYCQQYGTSPLT |
| | FGGGTKVEIK |
| | [SEQ ID NO: 213] |
| DNA | CAGGTGCAGCTGCAGGAGTCGGGCCCAGGACTGGTGAAGCCTTCGGAGA |
| for Full V_{H} | CCCTGTCCCTCACCTGCACTGTCTCTGGTGGCTCCATCAATAGTTACTA |
| | CTGGAGCTGGATCCGGCAGCCCCCAGGGAAGGGACTGGAGTGGATTGGG |
| | TATATCTTTTACAGTGGGATCACCAACTACAACCCCTCCCTCAAGAGTC |
| | GAGTCACCATATCATTAGACACGTCCAAGAACCAGTTCTCCCTGAAGCT |
| | GAGCTCTGTGACCGCTGCGGACACGGCCGTGTATTACTGTGCGAGAATC |
| | GGCGTGGCTGGTTTTTACTTTGACTACTGGGGCCAGGGAACCCTGGTCA |
| | CCGTCTCCTCA |
| | [SEQ ID NO: 214] |
| DNA | GAAATTGTGTTGACGCAGTCTCCAGGCACCCTGTCTTTGTCTCCAGGGG |
| for Full V_{L} | AAAGAGCCACCCTCTCCTGCAGGGCCAGTCAGAGTGTTAGCAGCAGCTA |
| Full VL | CTTAGCCTGGTACCAGCAGAAACCTGGCCAGGCTCCCAGGCTCCTCATC |
| | TATGGTGCATCCAGCAGGGCCACTGGCATCCCAGACAGGTTCAGTGGCA |
| | GTGGGTCTGGGACAGACTTCACTCTCACCATCAGCAGACTGGAGCCTGA |
| | AGATTTTGCAGTGTATTACTGTCAGCAGTATGGTACCTCACCGCTCACT |
| | TTCGGCGGAGGGACCAAGGTGGAGATCAAA |
| | [SEQ ID NO: 215] |

### 5.3.2 Monoclonal Antibodies

The presently disclosed subject matter provides antibodies (e.g., human antibodies, e.g., human monoclonal antibodies) that specifically bind to DLL3 (e.g., human DLL3). The V_{H} amino acid sequences of anti-DLL3 antibodies J8, L22, B2, A18, E9, G3, M11, O24, P4, J23, K19, N10, B16-v1, B16-v2, E23, F9, L12, B22, C22, D8, G16, F21, N12, G23, I1, C8, and O18 are set forth in SEQ ID NOs: 7, 17, 24, 34, 42, 52, 60, 66, 76, 83, 92, 102, 108, 119, 126, 131, 141, 147, 153, 157, 163, 172, 187, 197, 204, and 212 respectively. The V_{L} amino acid sequences of J8, L22, B2, A18, E9, G3, M11, O24, P4, J23, K19, N10, B16-v1, B16-v2, E23, F9, L12, B22, C22, D8, G16, F21, N12, G23, I1, C8, and O18 are set forth in SEQ ID NOs: 8, 18, 25, 35, 43, 53, 61, 67, 77, 84, 93, 103, 109, 113, 120, 127, 132, 142, 148, 154, 158, 164, 173, 188, 198, 205, and 213 respectively.

Given that each of J8, L22, B2, A18, E9, G3, M11, O24, P4, J23, K19, N10, B16-v1, B16-v2, E23, F9, L12, B22, C22, D8, G16, F21, N12, G23, I1, C8, and O18 antibodies can bind to DLL3, the V_{H} and V_{L} sequences can be “mixed and matched” to create other anti-DLL3 binding molecules. DLL3 binding of such “mixed and matched” antibodies can be tested using the binding assays known in the art, including for example, ELISAs, Western blots, RIAs, Biacore analysis. Preferably, when V_{H} and V_{L} chains are mixed and matched, a V_{H} sequence from a particular V_{H}/V_{L} pairing is replaced with a structurally similar V_{H} sequence. Likewise, a V_{L} sequence from a particular V_{H}/V_{L} pairing is replaced with a structurally similar V_{L} sequence.

In certain embodiments, the presently disclosed subject matter provides an antibody or an antigen-binding fragment thereof comprising: (a) a heavy chain variable region (V_{H}) comprising an amino acid sequence selected from SEQ ID NOs: 7, 17, 24, 34, 42, 52, 60, 66, 76, 83, 92, 102, 108, 119, 126, 131, 141, 147, 153, 157, 163, 172, 187, 197, 204, and 212; and (b) a light chain variable region (V_{L}) comprising an amino acid sequence selected from SEQ ID NOs: 8, 18, 25, 35, 43, 53, 61, 67, 77, 84, 93, 103, 109, 113, 120, 127, 132, 142, 148, 154, 158, 164, 173, 188, 198, 205, and 213; wherein the antibody or antigen-binding fragment specifically binds to DLL3, e.g., human DLL3. In certain embodiments, the V_{H} and V_{L} are selected from the group consisting of:
(a) a heavy chain variable region comprising the amino acid sequence set forth in SEQ ID NO: 7, and a light chain variable region comprising the amino acid sequence set forth in SEQ ID NO: 8;
(b) a heavy chain variable region comprising the amino acid sequence set forth in SEQ ID NO: 17, and a light chain variable region comprising the amino acid sequence set forth in SEQ ID NO: 18;
(c) a heavy chain variable region comprising the amino acid sequence set forth in SEQ ID NO: 24, and a light chain variable region comprising the amino acid sequence set forth in SEQ ID NO: 25;
(d) a heavy chain variable region comprising the amino acid sequence set forth in SEQ ID NO: 34, and a light chain variable region comprising the amino acid sequence set forth in SEQ ID NO: 35;
(e) a heavy chain variable region comprising the amino acid sequence set forth in SEQ ID NO: 42, and a light chain variable region comprising the amino acid sequence set forth in SEQ ID NO: 43;
(f) a heavy chain variable region comprising the amino acid sequence set forth in SEQ ID NO: 52, and a light chain variable region comprising the amino acid sequence set forth in SEQ ID NO: 53;
(g) a heavy chain variable region comprising the amino acid sequence set forth in SEQ ID NO: 60, and a light chain variable region comprising the amino acid sequence set forth in SEQ ID NO: 61;
(h) a heavy chain variable region comprising the amino acid sequence set forth in SEQ ID NO: 66, and a light chain variable region comprising the amino acid sequence set forth in SEQ ID NO: 67;
(i) a heavy chain variable region comprising the amino acid sequence set forth in SEQ ID NO: 76, and a light chain variable region comprising the amino acid sequence set forth in SEQ ID NO: 77;
(j) a heavy chain variable region comprising the amino acid sequence set forth in SEQ ID NO: 83, and a light chain variable region comprising the amino acid sequence set forth in SEQ ID NO: 84;
(k) a heavy chain variable region comprising the amino acid sequence set forth in SEQ ID NO: 92, and a light chain variable region comprising the amino acid sequence set forth in SEQ ID NO: 93;
(l) a heavy chain variable region comprising the amino acid sequence set forth in SEQ ID NO: 102, and a light chain variable region comprising the amino acid sequence set forth in SEQ ID NO: 103;
(m) a heavy chain variable region comprising the amino acid sequence set forth in SEQ ID NO: 108, and a light chain variable region comprising the amino acid sequence set forth in SEQ ID NO: 109;
(n) a heavy chain variable region comprising the amino acid sequence set forth in SEQ ID NO: 108, and a light chain variable region comprising the amino acid sequence set forth in SEQ ID NO: 113;
(o) a heavy chain variable region comprising the amino acid sequence set forth in SEQ ID NO: 119, and a light chain variable region comprising the amino acid sequence set forth in SEQ ID NO: 120;
(p) a heavy chain variable region comprising the amino acid sequence set forth in SEQ ID NO: 126, and a light chain variable region comprising the amino acid sequence set forth in SEQ ID NO: 127;
(q) a heavy chain variable region comprising the amino acid sequence set forth in SEQ ID NO: 131, and a light chain variable region comprising the amino acid sequence set forth in SEQ ID NO: 132;
(r) a heavy chain variable region comprising the amino acid sequence set forth in SEQ ID NO: 141, and a light chain variable region comprising the amino acid sequence set forth in SEQ ID NO: 142;
(s) a heavy chain variable region comprising the amino acid sequence set forth in SEQ ID NO: 147, and a light chain variable region comprising the amino acid sequence set forth in SEQ ID NO: 148;
(t) a heavy chain variable region comprising the amino acid sequence set forth in SEQ ID NO: 153, and a light chain variable region comprising the amino acid sequence set forth in SEQ ID NO: 154;
(u) a heavy chain variable region comprising the amino acid sequence set forth in SEQ ID NO: 157, and a light chain variable region comprising the amino acid sequence set forth in SEQ ID NO: 158;
(v) a heavy chain variable region comprising the amino acid sequence set forth in SEQ ID NO: 163, and a light chain variable region comprising the amino acid sequence set forth in SEQ ID NO: 164;
(w) a heavy chain variable region comprising the amino acid sequence set forth in SEQ ID NO: 172, and a light chain variable region comprising the amino acid sequence set forth in SEQ ID NO: 173;
(x) a heavy chain variable region comprising the amino acid sequence set forth in SEQ ID NO: 187, and a light chain variable region comprising the amino acid sequence set forth in SEQ ID NO: 188;
(y) a heavy chain variable region comprising the amino acid sequence set forth in SEQ ID NO: 197, and a light chain variable region comprising the amino acid sequence set forth in SEQ ID NO: 198;
(z) a heavy chain variable region comprising the amino acid sequence set forth in SEQ ID NO: 204, and a light chain variable region comprising the amino acid sequence set forth in SEQ ID NO: 205;
(aa) a heavy chain variable region comprising the amino acid sequence set forth in SEQ ID NO: 212, and a light chain variable region comprising the amino acid sequence set forth in SEQ ID NO: 213.

In certain embodiments, the presently disclosed subject matter provides antibodies or antigen-binding fragments thereof that comprise the heavy chain and light chain CDR1s, CDR2s and CDR3s of J8, L22, B2, A18, E9, G3, M11, O24, P4, J23, K19, N10, B16-v1, B16-v2, E23, F9, L12, B22, C22, D8, G16, F21, N12, G23, I1, C8, and O18.

The amino acid sequences of the V_{H} CDR1s of J8, L22, B2, A18, E9, G3, M11, O24, P4, J23, K19, N10, B16-v1, B16-v2, E23, F9, L12, B22, C22, D8, G16, F21, N12, G23, I1, C8, and 018 are shown in SEQ ID NOs: 1, 11, 21, 28, 46, 70, 87, 96, 135, 151, 191, and 208, respectively.

The amino acid sequences of the V_{H} CDR2s of J8, L22, B2, A18, E9, G3, M11, O24, P4, J23, K19, N10, B16-v1, B16-v2, E23, F9, L12, B22, C22, D8, G16, F21, N12, G23, I1, C8, and 018 antibodies set forth in SEQ ID NOs: 2, 12, 29, 38, 47, 71, 80, 88, 97, 106, 115, 136, 167, 183, 192, 201, and 209, respectively. The amino acid sequences of the V_{H} CDR3s of J8, L22, B2, A18, E9, G3, M11, O24, P4, J23, K19, N10, B16-v1, B16-v2, E23, F9, L12, B22, C22, D8, G16, F21, N12, G23, I1, C8, and O18 set forth in SEQ ID NOs: 3, 13, 22, 30, 39, 48, 56, 64, 72, 81, 89, 98, 107, 116, 123, 137, 145, 152, 161, 168, 184, 193, 202, and 210, respectively.

The amino acid sequences of the V_{L} CDR1s of J8, L22, B2, A18, E9, G3, M11, O24, P4, J23, K19, N10, B16-v1, B16-v2, E23, F9, L12, B22, C22, D8, G16, F21, N12, G23, I1, C8, and 018 are set forth in SEQ ID NOs: 4, 14, 31, 40, 49, 57, 73, 90, 99, 117, 124, 138, 169, 185, and 194, respectively. The amino acid sequences of the V_{L} CDR2s of J8, L22, B2, A18, E9, G3, M11, O24, P4, J23, K19, N10, B16-v1, B16-v2, E23, F9, L12, B22, C22, D8, G16, F21, N12, G23, I1, C8, and O18 are set forth in SEQ ID NOs: 5, 15, 32, 50, 58, 74, 100, 139, 146, 170, 195, and 216.

The amino acid sequences of the V_{L} CDR3s of J8, L22, B2, A18, E9, G3, M11, O24, P4, J23, K19, N10, B16-v1, B16-v2, E23, F9, L12, B22, C22, D8, G16, F21, N12, G23, I1, C8, and 018 are set forth in SEQ ID NOs: 6, 16, 23, 33, 41, 51, 59, 65, 75, 82, 91, 101, 112, 118, 125, 130, 140, 162, 171, 186, 196, 203, and 211, respectively. The CDR regions are delineated using the IMGT system. In certain embodiments, the CDR regions are delineated using the IMGT numbering system accessible at http://www.imgt.org/IMGT_vquest/input.

Given that each of these antibodies or antigen-binding fragments thereof can bind to DLL3 and that antigen-binding specificity is provided primarily by the CDR1, CDR2, and CDR3 regions, the V_{H} CDR1, CDR2, and CDR3 sequences and V_{L} CDR1, CDR2, and CDR3 sequences can be “mixed and matched” (i.e., CDRs from different antibodies can be mixed and match, although each antibody must contain a V_{H} CDR1, CDR2, and CDR3 and a V_{L} CDR1, CDR2, and CDR3) to create other anti-DLL3 binding molecules. DLL3 binding of such “mixed and matched” antibodies can be tested using the binding assays described above. When V_{H} CDR sequences are mixed and matched, the CDR1, CDR2 and/or CDR3 sequence from a particular V_{H} sequence is replaced with a structurally similar CDR sequence(s). Likewise, when V_{L} CDR sequences are mixed and matched, the CDR1, CDR2 and/or CDR3 sequence from a particular V_{L} sequence preferably is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences of the antibodies or antigen-binding fragments thereof disclosed herein J8, L22, B2, A18, E9, G3, M11, O24, P4, J23, K19, N10, B16-v1, B16-v2, E23, F9, L12, B22, C22, D8, G16, F21, N12, G23, I1, C8, and O18.

In certain embodiments, the presently disclosed subject matter provides an antibody or an antigen-binding fragment thereof comprising:
(a) a heavy chain variable region CDR1 comprising an amino acid sequence selected from SEQ ID NOs: 1, 11, 21, 28, 46, 70, 87, 96, 135, 151, 191, and 208;
(b) a heavy chain variable region CDR2 comprising an amino acid sequence selected from SEQ ID NOs: 2, 12, 29, 38, 47, 71, 80, 88, 97, 106, 115, 136, 167, 183, 192, 201, and 209;
(c) a heavy chain variable region CDR3 comprising an amino acid sequence selected from SEQ ID NOs: 3, 13, 22, 30, 39, 48, 56, 64, 72, 81, 89, 98, 107, 116, 123, 137, 145, 152, 161, 168, 184, 193, 202, and 210;
(d) a light chain variable region CDR1 comprising an amino acid sequence selected from SEQ ID NOs: 4, 14, 31, 40, 49, 57, 73, 90, 99, 117, 124, 138, 169, 185, and 194;
(e) a light chain variable region CDR2 comprising an amino acid sequence selected from SEQ ID Nos 5, 15, 32, 50, 58, 74, 100, 139, 146, 170, 195, and 216; and
(f) a light chain variable region CDR3 comprising an amino acid sequence selected from SEQ ID NOs: 6, 16, 23, 33, 41, 51, 59, 65, 75, 82, 91, 101, 112, 118, 125, 130, 140, 162, 171, 186, 196, 203, and 211.

In certain embodiments, the antibody or antigen-binding fragment thereof comprises:
(a) a heavy chain variable region CDR1 comprising the amino acid sequence set forth in SEQ ID NO: 1;
(b) a heavy chain variable region CDR2 comprising the amino acid sequence set forth in SEQ ID NO: 2;
(c) a heavy chain variable region CDR3 comprising the amino acid sequence set forth in SEQ ID NO: 3;
(d) a light chain variable region CDR1 comprising the amino acid sequence set forth in SEQ ID NO: 4;
(e) a light chain variable region CDR2 comprising the amino acid sequence set forth in SEQ ID NO: 5; and
(f) a light chain variable region CDR3 comprising the amino acid sequence set forth in SEQ ID NO: 6.

In certain embodiments, the antibody or antigen-binding fragment comprises:
(a) a heavy chain variable region CDR1 comprising the amino acid sequence set forth in SEQ ID NO: 11;
(b) a heavy chain variable region CDR2 comprising the amino acid sequence set forth in SEQ ID NO: 12;
(c) a heavy chain variable region CDR3 comprising the amino acid sequence set forth in SEQ ID NO: 13;
(d) a light chain variable region CDR1 comprising the amino acid sequence set forth in SEQ ID NO: 14;
(e) a light chain variable region CDR2 comprising the amino acid sequence set forth in SEQ ID NO: 15; and
(f) a light chain variable region CDR3 comprising the amino acid sequence set forth in SEQ ID NO: 16.

In certain embodiments, the antibody or antigen-binding fragment thereof comprises:
(a) a heavy chain variable region CDR1 comprising the amino acid sequence set forth in SEQ ID NO: 21;
(b) a heavy chain variable region CDR2 comprising the amino acid sequence set forth in SEQ ID NO: 2;
(c) a heavy chain variable region CDR3 comprising the amino acid sequence set forth in SEQ ID NO: 22;
(d) a light chain variable region CDR1 comprising the amino acid sequence set forth in SEQ ID NO: 4;
(e) a light chain variable region CDR2 comprising the amino acid sequence set forth in SEQ ID NO: 5; and
(f) a light chain variable region CDR3 comprising the amino acid sequence set forth in SEQ ID NO: 23.

In certain embodiments, the antibody or antigen-binding fragment thereof comprises:
(a) a heavy chain variable region CDR1 comprising the amino acid sequence set forth in SEQ ID NO: 28;
(b) a heavy chain variable region CDR2 comprising the amino acid sequence set forth in SEQ ID NO: 29;
(c) a heavy chain variable region CDR3 comprising the amino acid sequence set forth in SEQ ID NO: 30;
(d) a light chain variable region CDR1 comprising the amino acid sequence set forth in SEQ ID NO: 31;
(e) a light chain variable region CDR2 comprising the amino acid sequence set forth in SEQ ID NO: 32; and
(f) a light chain variable region CDR3 comprising the amino acid sequence set forth in SEQ ID NO: 33.

In certain embodiments, the antibody or antigen-binding fragment thereof comprises:
(a) a heavy chain variable region CDR1 comprising the amino acid sequence set forth in SEQ ID NO: 21;
(b) a heavy chain variable region CDR2 comprising the amino acid sequence set forth in SEQ ID NO: 38;
(c) a heavy chain variable region CDR3 comprising the amino acid sequence set forth in SEQ ID NO: 39;
(d) a light chain variable region CDR1 comprising the amino acid sequence set forth in SEQ ID NO: 40;
(e) a light chain variable region CDR2 comprising the amino acid sequence set forth in SEQ ID NO: 5; and
(f) a light chain variable region CDR3 comprising the amino acid sequence set forth in SEQ ID NO: 41.

In certain embodiments, the antibody or antigen-binding fragment thereof comprises:
(a) a heavy chain variable region CDR1 comprising the amino acid sequence set forth in SEQ ID NO: 46;
(b) a heavy chain variable region CDR2 comprising the amino acid sequence set forth in SEQ ID NO: 47;
(c) a heavy chain variable region CDR3 comprising the amino acid sequence set forth in SEQ ID NO: 48;
(d) a light chain variable region CDR1 comprising the amino acid sequence set forth in SEQ ID NO: 49;
(e) a light chain variable region CDR2 comprising the amino acid sequence set forth in SEQ ID NO: 50; and
(f) a light chain variable region CDR3 comprising the amino acid sequence set forth in SEQ ID NO: 51.

In certain embodiments, the antibody or antigen-binding fragment thereof comprises:
(a) a heavy chain variable region CDR1 comprising the amino acid sequence set forth in SEQ ID NO: 21;
(b) a heavy chain variable region CDR2 comprising the amino acid sequence set forth in SEQ ID NO: 2;
(c) a heavy chain variable region CDR3 comprising the amino acid sequence set forth in SEQ ID NO: 56;
(d) a light chain variable region CDR1 comprising the amino acid sequence set forth in SEQ ID NO: 57;
(e) a light chain variable region CDR2 comprising the amino acid sequence set forth in SEQ ID NO: 58; and
(f) a light chain variable region CDR3 comprising the amino acid sequence set forth in SEQ ID NO: 59.

In certain embodiments, the antibody or antigen-binding fragment thereof comprises:
(a) a heavy chain variable region CDR1 comprising the amino acid sequence set forth in SEQ ID NO: 70;
(b) a heavy chain variable region CDR2 comprising the amino acid sequence set forth in SEQ ID NO: 71;
(c) a heavy chain variable region CDR3 comprising the amino acid sequence set forth in SEQ ID NO: 72;
(d) a light chain variable region CDR1 comprising the amino acid sequence set forth in SEQ ID NO: 73;
(e) a light chain variable region CDR2 comprising the amino acid sequence set forth in SEQ ID NO: 74; and
(f) a light chain variable region CDR3 comprising the amino acid sequence set forth in SEQ ID NO: 75.

In certain embodiments, the antibody or antigen-binding fragment thereof comprises:
(a) a heavy chain variable region CDR1 comprising the amino acid sequence set forth in SEQ ID NO: 21;
(b) a heavy chain variable region CDR2 comprising the amino acid sequence set forth in SEQ ID NO: 80;
(c) a heavy chain variable region CDR3 comprising the amino acid sequence set forth in SEQ ID NO: 81;
(d) a light chain variable region CDR1 comprising the amino acid sequence set forth in SEQ ID NO: 57;
(e) a light chain variable region CDR2 comprising the amino acid sequence set forth in SEQ ID NO: 58; and
(f) a light chain variable region CDR3 comprising the amino acid sequence set forth in SEQ ID NO: 82.

In certain embodiments, the antibody or antigen-binding fragment thereof comprises:
(a) a heavy chain variable region CDR1 comprising the amino acid sequence set forth in SEQ ID NO: 87;
(b) a heavy chain variable region CDR2 comprising the amino acid sequence set forth in SEQ ID NO: 88;
(c) a heavy chain variable region CDR3 comprising the amino acid sequence set forth in SEQ ID NO: 89;
(d) a light chain variable region CDR1 comprising the amino acid sequence set forth in SEQ ID NO: 90;
(e) a light chain variable region CDR2 comprising the amino acid sequence set forth in SEQ ID NO: 216; and
(f) a light chain variable region CDR3 comprising the amino acid sequence set forth in SEQ ID NO: 91.

In certain embodiments, the antibody or antigen-binding fragment thereof comprises:
(a) a heavy chain variable region CDR1 comprising the amino acid sequence set forth in SEQ ID NO: 96;
(b) a heavy chain variable region CDR2 comprising the amino acid sequence set forth in SEQ ID NO: 97;
(c) a heavy chain variable region CDR3 comprising the amino acid sequence set forth in SEQ ID NO: 98;
(d) a light chain variable region CDR1 comprising the amino acid sequence set forth in SEQ ID NO: 99;
(e) a light chain variable region CDR2 comprising the amino acid sequence set forth in SEQ ID NO: 100; and
(f) a light chain variable region CDR3 comprising the amino acid sequence set forth in SEQ ID NO: 101.

In certain embodiments, the antibody or antigen-binding fragment thereof comprises:
(a) a heavy chain variable region CDR1 comprising the amino acid sequence set forth in SEQ ID NO: 21;
(b) a heavy chain variable region CDR2 comprising the amino acid sequence set forth in SEQ ID NO: 106;
(c) a heavy chain variable region CDR3 comprising the amino acid sequence set forth in SEQ ID NO: 107;
(d) a light chain variable region CDR1 comprising the amino acid sequence set forth in SEQ ID NO: 57;
(e) a light chain variable region CDR2 comprising the amino acid sequence set forth in SEQ ID NO: 58; and
(f) a light chain variable region CDR3 comprising the amino acid sequence set forth in SEQ ID NO: 82.

In certain embodiments, the antibody or antigen-binding fragment thereof comprises:
(a) a heavy chain variable region CDR1 comprising the amino acid sequence set forth in SEQ ID NO: 21;
(b) a heavy chain variable region CDR2 comprising the amino acid sequence set forth in SEQ ID NO: 106;
(c) a heavy chain variable region CDR3 comprising the amino acid sequence set forth in SEQ ID NO: 107;
(d) a light chain variable region CDR1 comprising the amino acid sequence set forth in SEQ ID NO: 4;
(e) a light chain variable region CDR2 comprising the amino acid sequence set forth in SEQ ID NO: 5; and
(f) a light chain variable region CDR3 comprising the amino acid sequence set forth in SEQ ID NO: 112.

In certain embodiments, the antibody or antigen-binding fragment thereof comprises:
(a) a heavy chain variable region CDR1 comprising the amino acid sequence set forth in SEQ ID NO: 95;
(b) a heavy chain variable region CDR2 comprising the amino acid sequence set forth in SEQ ID NO: 115;
(c) a heavy chain variable region CDR3 comprising the amino acid sequence set forth in SEQ ID NO: 116;
(d) a light chain variable region CDR1 comprising the amino acid sequence set forth in SEQ ID NO: 117;
(e) a light chain variable region CDR2 comprising the amino acid sequence set forth in SEQ ID NO: 100; and
(f) a light chain variable region CDR3 comprising the amino acid sequence set forth in SEQ ID NO: 118.

In certain embodiments, the antibody or antigen-binding fragment thereof comprises:
(a) a heavy chain variable region CDR1 comprising the amino acid sequence set forth in SEQ ID NO: 21;
(b) a heavy chain variable region CDR2 comprising the amino acid sequence set forth in SEQ ID NO: 2;
(c) a heavy chain variable region CDR3 comprising the amino acid sequence set forth in SEQ ID NO: 123;
(d) a light chain variable region CDR1 comprising the amino acid sequence set forth in SEQ ID NO: 124;
(e) a light chain variable region CDR2 comprising the amino acid sequence set forth in SEQ ID NO: 58; and
(f) a light chain variable region CDR3 comprising the amino acid sequence set forth in SEQ ID NO: 125.

In certain embodiments, the antibody or antigen-binding fragment thereof comprises:
(a) a heavy chain variable region CDR1 comprising the amino acid sequence set forth in SEQ ID NO: 21;
(b) a heavy chain variable region CDR2 comprising the amino acid sequence set forth in SEQ ID NO: 2;
(c) a heavy chain variable region CDR3 comprising the amino acid sequence set forth in SEQ ID NO: 56;
(d) a light chain variable region CDR1 comprising the amino acid sequence set forth in SEQ ID NO: 57;
(e) a light chain variable region CDR2 comprising the amino acid sequence set forth in SEQ ID NO: 58; and
(f) a light chain variable region CDR3 comprising the amino acid sequence set forth in SEQ ID NO: 130.

In certain embodiments, the antibody or antigen-binding fragment thereof comprises:
(a) a heavy chain variable region CDR1 comprising the amino acid sequence set forth in SEQ ID NO: 135;
(b) a heavy chain variable region CDR2 comprising the amino acid sequence set forth in SEQ ID NO: 136;
(c) a heavy chain variable region CDR3 comprising the amino acid sequence set forth in SEQ ID NO: 137;
(d) a light chain variable region CDR1 comprising the amino acid sequence set forth in SEQ ID NO: 138;
(e) a light chain variable region CDR2 comprising the amino acid sequence set forth in SEQ ID NO: 139; and
(f) a light chain variable region CDR3 comprising the amino acid sequence set forth in SEQ ID NO: 140.

In certain embodiments, the antibody or antigen-binding fragment thereof comprises:
(a) a heavy chain variable region CDR1 comprising the amino acid sequence set forth in SEQ ID NO: 21;
(b) a heavy chain variable region CDR2 comprising the amino acid sequence set forth in SEQ ID NO: 2;
(c) a heavy chain variable region CDR3 comprising the amino acid sequence set forth in SEQ ID NO: 145;
(d) a light chain variable region CDR1 comprising the amino acid sequence set forth in SEQ ID NO: 57;
(e) a light chain variable region CDR2 comprising the amino acid sequence set forth in SEQ ID NO: 146; and
(f) a light chain variable region CDR3 comprising the amino acid sequence set forth in SEQ ID NO: 125.

In certain embodiments, the antibody or antigen-binding fragment thereof comprises:
(a) a heavy chain variable region CDR1 comprising the amino acid sequence set forth in SEQ ID NO: 151;
(b) a heavy chain variable region CDR2 comprising the amino acid sequence set forth in SEQ ID NO: 2;
(c) a heavy chain variable region CDR3 comprising the amino acid sequence set forth in SEQ ID NO: 152;
(d) a light chain variable region CDR1 comprising the amino acid sequence set forth in SEQ ID NO: 57;
(e) a light chain variable region CDR2 comprising the amino acid sequence set forth in SEQ ID NO: 58; and
(f) a light chain variable region CDR3 comprising the amino acid sequence set forth in SEQ ID NO: 82.

In certain embodiments, the antibody or antigen-binding fragment thereof comprises:
(a) a heavy chain variable region CDR1 comprising the amino acid sequence set forth in SEQ ID NO: 21;
(b) a heavy chain variable region CDR2 comprising the amino acid sequence set forth in SEQ ID NO: 2;
(c) a heavy chain variable region CDR3 comprising the amino acid sequence set forth in SEQ ID NO: 123;
(d) a light chain variable region CDR1 comprising the amino acid sequence set forth in SEQ ID NO: 124;
(e) a light chain variable region CDR2 comprising the amino acid sequence set forth in SEQ ID NO: 58; and
(f) a light chain variable region CDR3 comprising the amino acid sequence set forth in SEQ ID NO: 59.

In certain embodiments, the antibody or antigen-binding fragment thereof comprises:
(a) a heavy chain variable region CDR1 comprising the amino acid sequence set forth in SEQ ID NO: 11;
(b) a heavy chain variable region CDR2 comprising the amino acid sequence set forth in SEQ ID NO: 136;
(c) a heavy chain variable region CDR3 comprising the amino acid sequence set forth in SEQ ID NO: 161;
(d) a light chain variable region CDR1 comprising the amino acid sequence set forth in SEQ ID NO: 73;
(e) a light chain variable region CDR2 comprising the amino acid sequence set forth in SEQ ID NO: 74; and
(f) a light chain variable region CDR3 comprising the amino acid sequence set forth in SEQ ID NO: 162.

In certain embodiments, the antibody or antigen-binding fragment thereof comprises:
(a) a heavy chain variable region CDR1 comprising the amino acid sequence set forth in SEQ ID NO: 96;
(b) a heavy chain variable region CDR2 comprising the amino acid sequence set forth in SEQ ID NO: 167;
(c) a heavy chain variable region CDR3 comprising the amino acid sequence set forth in SEQ ID NO: 168;
(d) a light chain variable region CDR1 comprising the amino acid sequence set forth in SEQ ID NO: 169;
(e) a light chain variable region CDR2 comprising the amino acid sequence set forth in SEQ ID NO: 170; and
(f) a light chain variable region CDR3 comprising the amino acid sequence set forth in SEQ ID NO: 171.

In certain embodiments, the antibody or antigen-binding fragment thereof comprises:
(a) a heavy chain variable region CDR1 comprising the amino acid sequence set forth in SEQ ID NO: 21;
(b) a heavy chain variable region CDR2 comprising the amino acid sequence set forth in SEQ ID NO: 183;
(c) a heavy chain variable region CDR3 comprising the amino acid sequence set forth in SEQ ID NO: 184;
(d) a light chain variable region CDR1 comprising the amino acid sequence set forth in SEQ ID NO: 185;
(e) a light chain variable region CDR2 comprising the amino acid sequence set forth in SEQ ID NO: 50; and
(f) a light chain variable region CDR3 comprising the amino acid sequence set forth in SEQ ID NO: 186.

In certain embodiments, the antibody or antigen-binding fragment thereof comprises:
(a) a heavy chain variable region CDR1 comprising the amino acid sequence set forth in SEQ ID NO: 191;
(b) a heavy chain variable region CDR2 comprising the amino acid sequence set forth in SEQ ID NO: 192;
(c) a heavy chain variable region CDR3 comprising the amino acid sequence set forth in SEQ ID NO: 193;
(d) a light chain variable region CDR1 comprising the amino acid sequence set forth in SEQ ID NO: 194;
(e) a light chain variable region CDR2 comprising the amino acid sequence set forth in SEQ ID NO: 195; and
(f) a light chain variable region CDR3 comprising the amino acid sequence set forth in SEQ ID NO: 196.

In certain embodiments, the antibody or antigen-binding fragment thereof comprises:
(a) a heavy chain variable region CDR1 comprising the amino acid sequence set forth in SEQ ID NO: 11;
(b) a heavy chain variable region CDR2 comprising the amino acid sequence set forth in SEQ ID NO: 201;
(c) a heavy chain variable region CDR3 comprising the amino acid sequence set forth in SEQ ID NO: 202;
(d) a light chain variable region CDR1 comprising the amino acid sequence set forth in SEQ ID NO: 4;
(e) a light chain variable region CDR2 comprising the amino acid sequence set forth in SEQ ID NO: 5; and
(f) a light chain variable region CDR3 comprising the amino acid sequence set forth in SEQ ID NO: 203.

In certain embodiments, the antibody or antigen-binding fragment thereof comprises:
(a) a heavy chain variable region CDR1 comprising the amino acid sequence set forth in SEQ ID NO: 208;
(b) a heavy chain variable region CDR2 comprising the amino acid sequence set forth in SEQ ID NO: 209;
(c) a heavy chain variable region CDR3 comprising the amino acid sequence set forth in SEQ ID NO: 210;
(d) a light chain variable region CDR1 comprising the amino acid sequence set forth in SEQ ID NO: 57;
(e) a light chain variable region CDR2 comprising the amino acid sequence set forth in SEQ ID NO: 58; and
(f) a light chain variable region CDR3 comprising the amino acid sequence set forth in SEQ ID NO: 211.

The constant region/framework region of the anti-DLL3 antibodies disclosed herein can be altered, for example, by amino acid substitution, to modify the properties of the antibody (e.g., to increase or decrease one or more of: antigen binding affinity, Fc receptor binding, antibody carbohydrate, for example, glycosylation, fucosylation etc., the number of cysteine residues, effector cell function, effector cell function, complement function or introduction of a conjugation site).

In certain embodiments, a presently disclosed anti-DLL3 antibody is a fully-human antibody, e.g., any one of J8, L22, B2, A18, E9, G3, M11, O24, P4, J23, K19, N10, B16-v1, B16-v2, E23, F9, L12, B22, C22, D8, G16, F21, N12, G23, I1, C8, and 018. Fully-human mAbs, when administered to humans, causing serious side effects, including anaphylaxis and hypersensitivity reactions.

The use of phage display libraries has made it possible to select large numbers of antibody repertoires for unique and rare Abs against very defined epitopes (for more details on phage display see McCafferty et al., Phage antibodies: filamentous phage displaying antibody variable domains. Nature, 348: 552-554.) The rapid identification of human Fab or single chain Fv (scFv) fragments highly specific for tumor antigen-derived peptide-MHC complex molecules has thus become possible. In addition, by engineering full-length monoclonal antibody (mAb) using the Fab fragments, it is possible to directly generate a therapeutic human mAb, bypassing months of time-consuming work, normally needed for developing therapeutic mAbs. The presently disclosed subject matter involves the development of a fully human mAb that recognizes, for example, a human DLL3 polypeptide (e.g., a polypeptide having the amino acid sequence set forth in SEQ ID NO: 116) for cancer therapy.

### 5.3.3 Homologous Antibodies

In certain embodiments, a presently disclosed antibody or antigen-binding fragment thereof comprises heavy and light chain variable regions comprising amino acid sequences that are homologous or identical to the amino acid sequences of the antibodies described herein (e.g., J8, L22, B2, A18, E9, G3, M11, O24, P4, J23, K19, N10, B16-v1, B16-v2, E23, F9, L12, B22, C22, D8, G16, F21, N12, G23, I1, C8, and O18 antibodies), and wherein the antibodies or antigen-binding fragments thereof retain the desired functional properties of the anti-DLL3 antibodies or antigen-binding fragments thereof of the presently disclosed subject matter.

For example, the presently disclosed subject matter provides an antibody or an antigen-binding fragment thereof, comprising a heavy chain variable region and a light chain variable region, wherein:
(a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 187, SEQ ID NO: 197, SEQ ID NO: 204, or SEQ ID NO: 212;
(b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 188, SEQ ID NO: 198, SEQ ID NO: 205, or SEQ ID NO: 213.

In certain embodiments, the V_{H} and/or V_{L} amino acid sequences can be at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the sequences set forth above. An antibody having V_{H} and V_{L} regions having high (i.e., 80% or greater) homology or identity to the V_{H} and V_{L} regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

As used herein, the percent homology between two amino acid sequences is equivalent to the percent identity between the two sequences. The percent identity or homology between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent homology or identity between two amino acid sequences can be determined using the algorithm of E. Meyers and W. Miller (Comput Appl Biosci (1988); 14:11-17) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J Mol Biol (1970); 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the presently disclosed subject matter can further be used as a “query sequence” to perform a search against public databases to, for example, identify related sequences. Such searches can be performed using the XBLAST program (version 2.0) of Altschul et al., J Mol Biol (1990); 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., Nucleic Acids Res (1997); 25(17):3389-3402. When utilizing BLAST and Gapped BLAST programs, the default parameters of the respective programs (e.g., XBLAST and NBLAST) can be used.

### 5.3.4 Antibodies with Conservative Modifications

In certain embodiments, a presently disclosed antibody or an antigen-binding fragment thereof comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein (e.g., J8, L22, B2, A18, E9, G3, M11, O24, P4, J23, K19, N10, B16-v1, B16-v2, E23, F9, L12, B22, C22, D8, G16, F21, N12, G23, I1, C8, and O18 antibodies), or a conservative modification thereof, and wherein the antibodies retain the desired functional properties of the anti-DLL3 antibodies or antigen-binding fragments thereof of the presently disclosed subject matter. The presently disclosed subject matter provides an antibody or an antigen-binding fragment thereof, comprising a heavy chain variable region comprising CDR1, CDR2, and CDR3 sequences and a light chain variable region comprising CDR1, CDR2, and CDR3 sequences, wherein:
(a) the heavy chain variable region CDR3 sequence comprises an amino acid sequence selected from the amino acid sequences of SEQ ID NOs: 3, 13, 22, 30, 39, 48, 56, 64, 72, 81, 89, 98, 107, 116, 123, 137, 145, 152, 161, 168, 184, 193, 202, and 210, and conservative modifications thereof;
(b) the light chain variable region CDR3 sequence comprises an amino acid sequence selected from the amino acid sequence of SEQ ID NOs: 6, 16, 23, 33, 41, 51, 59, 65, 75, 82, 91, 101, 112, 118, 125, 130, 140, 162, 171, 186, 196, 203, and 212, and conservative modifications thereof.

In certain embodiments, the heavy chain variable region CDR3 sequence comprises an amino acid sequence selected from the amino acid sequences of SEQ ID NOs: 3, 13, 22, 30, 39, 48, 56, 64, 72, 81, 89, 98, 107, 116, 123, 137, 145, 152, 161, 168, 184, 193, 202, and 210, and conservative modifications thereof; and the light chain variable region CDR3 sequence comprises an amino acid sequence selected from the amino acid sequences of SEQ ID NOs: 6, 16, 23, 33, 41, 51, 59, 65, 75, 82, 91, 101, 112, 118, 125, 130, 140, 162, 171, 186, 196, 203, and 212, and conservative modifications thereof.

In certain embodiments, the heavy chain variable region CDR2 sequence comprises an amino acid sequence selected from the amino acid sequences of SEQ ID NOs: 2, 12, 29, 38, 47, 71, 80, 88, 97, 106, 115, 136, 167, 183, 192, 201, and 209, and conservative modifications thereof; and the light chain variable region CDR2 sequence comprises an amino acid sequence selected from the amino acid sequences of SEQ ID NOs: 5, 15, 32, 50, 58, 74, 100, 139, 146, 170, 195, and 216, and conservative modifications thereof.

In certain embodiments, the heavy chain variable region CDR1 sequence comprises an amino acid sequence selected from the amino acid sequences of SEQ ID NOs: 1, 11, 21, 28, 46, 70, 87, 96, 135, 151, 191, and 208, and conservative modifications thereof; and the light chain variable region CDR1 sequence comprises an amino acid sequence selected from the amino acid sequences of SEQ ID NOs: 4, 14, 31, 40, 49, 57, 73, 90, 99, 117, 124, 138, 169, 185, and 194, and conservative modifications thereof.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the present disclosure by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been defined in the art. Exemplary conservative amino acid substitutions are shown in Table 24. Amino acid substitutions may be introduced into an antibody of interest and the products screened for a desired activity, e.g., retained/improved antigen binding, decreased immunogenicity, or improved ADCC or CDC. In certain embodiments, a sequence disclosed herein, e.g., a CDR sequence, a V_{H} sequence or a V_{L} sequence, can have up to about one, up to about two, up to about three, up to about four, up to about five, up to about six, up to about seven, up to about eight, up to about nine or up to about ten amino acid residues that are modified and/or substituted.

TABLE 24

| Original | Exemplary conservative amino |
|---|---|
| Residue | acid Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

Non-conservative substitutions will entail exchanging a member of one of these classes for another class.

### 5.3.5 Anti-DLL3 Antibodies that Cross-compete for Binding to DLL3 with Anti-DLL3Antibodies of the Invention

The presently disclosed subject matter provides antibodies or antigen-binding fragments thereof that cross-compete with any of the disclosed anti-DLL3 antibodies for binding to DLL3 (e.g., human DLL3). For example, and not by way of limitation, the cross-competing antibodies can bind to the same epitope region, e.g., same epitope, adjacent epitope, or overlapping as any of the anti-DLL3 antibodies or antigen-binding fragments thereof of the presently disclosed subject matter. In certain embodiments, the reference antibody or reference antigen-binding fragments thereof for cross-competition studies can be any one of the anti-DLL3 antibodies or antigen-binding fragments thereof disclosed herein, e.g., J8, L22, B2, A18, E9, G3, M11, O24, P4, J23, K19, N10, B16-v1, B16-v2, E23, F9, L12, B22, C22, D8, G16, F21, N12, G23, I1, C8, and 018 antibodies.

Such cross-competing antibodies can be identified based on their ability to cross-compete with any one of the presently disclosed anti-DLL3 antibodies or antigen-binding fragments thereof in standard DLL3 binding assays. For example, Biacore analysis, ELISA assays or flow cytometry can be used to demonstrate cross-competition with the antibodies of the presently disclosed subject matter. The ability of a test antibody to inhibit the binding of, for example, any one of the presently disclosed anti-DLL3 antibodies (e.g., J8, L22, B2, A18, E9, G3, M11, O24, P4, J23, K19, N10, B16-v1, B16-v2, E23, F9, L12, B22, C22, D8, G16, F21, N12, G23, I1, C8, and O18 antibodies) to DLL3 (e.g., human DLL3) demonstrates that the test antibody can compete with any one of the presently disclosed anti-DLL3 antibodies or antigen-binding fragments thereof for binding to DLL3 (e.g., human DLL3) and thus binds to the same epitope region on DLL3 (e.g., human DLL3) as any one of the presently disclosed anti-DLL3 antibodies or antigen-binding fragments thereof. In certain embodiments, the cross-competing antibody or antigen-binding fragment thereof binds to the same epitope on DLL3 (e.g., human DLL3) as any one of the presently disclosed anti-DLL3 antibodies or antigen-binding fragments thereof.

### 5.3.6 Characterization of Antibody Binding to Antigen

Antibodies or antigen-binding fragments thereof of the presently disclosed subject can be tested for binding to DLL3 by, for example, standard ELISA. To determine if the selected anti-DLL3 antibodies bind to unique epitopes, each antibody can be biotinylated using commercially available reagents (Pierce, Rockford, IL). Competition studies using unlabeled monoclonal antibodies and biotinylated monoclonal antibodies can be performed using DLL3 coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. Anti-DLL3 human IgGs can be further tested for reactivity with DLL3 antigen by Western blotting.

In certain embodiments, the K_{D} is measured by a radiolabeled antigen binding assay (RIA). In certain embodiments, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J Mol Biol (1999); 293:865-881).

In certain embodiments, the K_{D} is measured using a BIACORE® surface plasmon resonance assay. For example, an assay using a BIACORE®-2000 or a BIACORE®-3000 (BIAcore, Inc., Piscataway, NJ)

### 5.3.7 Immunoconjugates

The presently disclosed subject provides an anti-DLL3 antibody or an antigen-binding fragment thereof, conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Non-limiting examples of cytotoxins include taxol (such as ricin, diphtheria, gelonin), cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, calecheamicin, aureastatin, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), hypomethylating agents (azacytidine and decitabine), and anti-mitotic agents (e.g., vincristine and vinblastine).

Other examples of therapeutic cytotoxins that can be conjugated to an anti-DLL3 antibody disclosed herein include duocarmycins, calicheamicins, maytansines and auristatins, and derivatives thereof. Cytotoxins can be conjugated to an anti-DLL3 antibody or an antigen-binding fragment thereof disclosed herein using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

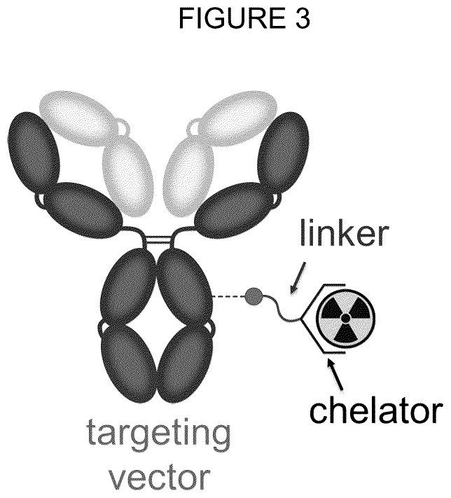

Anti-DLL3 antibodies or antigen-binding fragments thereof of the presently disclosed subject matter also can be conjugated to a radioactive isotope to generate cytotoxic radiopharmaceuticals, also referred to as radioimmunoconjugates. An exemplary graphical representation is depicted in FIG. 3. Non-limiting examples of radioactive isotopes that can be conjugated to antibodies for use diagnostically or therapeutically include ⁴⁷Sc, ⁶⁷Cu, ⁹⁰Y, ¹³¹I, ¹⁴⁹Tb, ¹⁶¹Tb, ¹⁷⁷Lu, ²²⁵Ac, ²¹³Bi, ²²³Ra, ⁸⁹Zr, and ²²⁷Th. In certain embodiments, the radioactive isotope is a ¹⁷⁷Lu radioactive isotope. In certain embodiments, the radioactive isotope is an ⁸⁹Zr radioactive isotope. Methods for preparing radioimmunoconjugates are established in the art. Examples of radioimmunoconjugates are commercially available, including Zevalin™ (IDEC Pharmaceuticals) and Bexxar™ (Corixa Pharmaceuticals), and similar methods can be used to prepare radioimmunoconjugates using the presently disclosed anti-DLL3 antibodies.

In certain embodiments, the anti-DLL3 antibodies or antigen-binding fragments thereof of the presently disclosed subject matter can be conjugated to a radioisotope to generate a radioimmunoconjugate by using a chelator. As used herein, the term “chelator” refers to a chemical compound in the form of a heterocyclic ring or surrounding structure containing a metal ion attached by coordinate bonds to at least two nonmetal ions. Non-limiting examples of chelator include 1,4,7-Triazacyclononane-1,4,7-triacetic acid (NOTA), 2,2′-(7-(1-carboxy-4-((4-isothiocyanatobenzyl)amino)-4-oxobutyl)-1,4,7-triazonane-1,4-diyl)diacetic acid (NODA), 2,2′, 2″, 2′″-(1,4,7,10-Tetraazacyclododecane-1,4,7,10-tetrayl)tetraacetic acid (DOTA), Diethylenetriamine-N,N,N′,N,N-pentaacetic acid, pentetic acid, (Carboxymethyl)imino] bis(ethylenenitrilo)-tetra-acetic acid (DTPA), 1-Hydroxy-2-pyridone; 2-Pyridinol-1-oxide (HOPO), N-(5-(3-((5-Aminopentyl)hydroxycarbamoyl) propionamido)pentyl)-3-((5-(N-hydroxyacetamido) pentyl)carbamoyl) propionohydroxamic acid (DFO), and 2-[1,4,7-Triazacyclononan-1-yl-4,7-bis(tBu-ester)]-1,5-pentanedioic acid (NODAGA). Additional exemplary chelators encompassed by the presently disclosed subject matter include AAZTA and derivatives thereof, BAT, BARAC, BPCA, TE2A, CB-TE2A, CB0TE1A1P, CB-TE2P, MM-TE2A, DM TE-2A, CP356, DATA, DBCO, DiAmSar and derivatives thereof, DIBO, DIMA, DFO, DGO, DOTA and derivatives thereof (e.g., Ac-DOTA, benzo-DOTA, dibenzo-DOTA, CB-DO2A, 3p-C-DEPA, Oxo-DO3A), DOTMA derivative thereof (e.g., benzo-DOTMA), DTPA and derivatives thereof (e.g., benzo-DTPA, dibenzo-DTPA, phenyl-DTPA, diphenyl-DTPA, benzyl-DTPA, dibenzyl-DTPA, 1B4M-DTPA, CHX-A″-DTPA), EDTA, EGTA, EHPG and derivatives thereof (e.g., 5-Cl-EHPG, 5-Br-EHPG, 5-Me-EHPG, 5t-Bu-EHPG, 5-sec-Bu-EHPG), H2dedpa, H4octapa, H2azapa, H5decapa, H6phospa, HBED and derivatives thereof, SHBED, HEHA, HYNIC, LICAM and derivatives thereof, MECAM, NODASA, NODAGA, NOPO, NOTA and derivatives thereof (e.g., benzo-NOTA), NETA, PEPA, PCTA, PDTA, TACN-TM, TCMC, TETA and derivatives thereof (e.g., benzo-TETA), TETMA and derivatives (e.g., benzo-TETMA), TRAP (PRP9), TRITA, TTHA and derivatives thereof.

The antibody conjugates of the presently disclosed subject matter can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor (TNF) or interferon-7; or, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### 5.3.7.1 Exemplary Immunoconjugate

In certain embodiments, the radioimmunoconjugate of the presently disclosed subject matter comprises an anti-DLL3 antibody or antigen-binding fragment thereof comprising a V_{H} comprising a CDR1 comprising the amino acid sequence set forth in SEQ ID NO: 11, a CDR2 comprising the amino acid sequence set forth in SEQ ID NO: 201, and a CDR3 comprising the amino acid sequence set forth in SEQ ID NO: 202; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 203. In certain embodiments, the anti-DLL3 antibody or antigen-binding fragment thereof comprises a V_{H} comprising the amino acid sequence set forth in SEQ ID NO: 204 and a V_{L} comprising the amino acid sequence set forth in SEQ ID NO: 205. In certain embodiments, the antibody or antigen-binding fragment thereof is designated as “C8”. In certain embodiments, the radioimmunoconjugate comprises a chelator. In certain embodiments, the chelator is N-(5-(3-((5-Aminopentyl)hydroxycarbamoyl)propionamido)pentyl)-3-((5-(N- hydroxyacetamido)pentyl) carbamoyl) propionohydroxamic acid (DFO). In certain embodiments, the radioimmunoconjugate comprises a radioactive isotope. In certain embodiments, the radioactive isotope is a ⁸⁹Zr radioactive isotope.

In certain embodiments, the radioimmunoconjugate of the presently disclosed subject matter comprises an anti-DLL3 antibody or antigen-binding fragment thereof comprising a V_{H} comprising a CDR1 comprising the amino acid sequence set forth in SEQ ID NO: 11, a CDR2 comprising the amino acid sequence set forth in SEQ ID NO: 201, and a CDR3 comprising the amino acid sequence set forth in SEQ ID NO: 202; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 203. In certain embodiments, the anti-DLL3 antibody or antigen-binding fragment thereof comprises a V_{H} comprising the amino acid sequence set forth in SEQ ID NO: 204 and a V_{L} comprising the amino acid sequence set forth in SEQ ID NO: 205. In certain embodiments, the antibody or antigen-binding fragment thereof is designated as “C8”. In certain embodiments, the radioimmunoconjugate comprises a chelator. In certain embodiments, the chelator is N-(5-(3-((5-Aminopentyl)hydroxycarbamoyl)propionamido)pentyl)-3-((5-(N- hydroxyacetamido)pentyl) carbamoyl) propionohydroxamic acid (DFO). In certain embodiments, the radioimmunoconjugate comprises a radioactive isotope. In certain embodiments, the radioactive isotope is a ¹¹⁷Lu radioactive isotope.

### 5.3.8 Multi-specific Molecules

The presently disclosed subject matter provides multi-specific molecules comprising an anti-DLL3 antibody, or a fragment thereof, disclosed herein. A presently disclosed or an antigen-binding fragment thereof can be derivatized or linked to one more functional molecules, e.g., one or more peptides or proteins (e.g., one or more antibodies or ligands for a receptor) to generate a multi-specific molecule that binds to two or more different binding sites or target molecules. The presently disclosed anti-DLL3 antibody or antigen-binding fragment thereof can in fact be derivatized or linked to more than one other functional molecules to generate multi-specific molecules that bind to more than two different binding sites and/or target molecules. To create a multi-specific molecule, a presently disclosed anti-DLL3 antibody or an antigen-binding fragment thereof can be functionally linked (e.g., by chemical coupling, genetic fusion, noncovalent association or otherwise) to one or more other binding molecules, such as another antibody, antibody fragment, peptide or binding mimetic, such that a bispecific molecule.

In certain embodiments, the multi-specific molecule is a bispecific molecule. In certain embodiments, the bispecific molecules comprises at least a first binding specificity for DLL3 and a second binding specificity for a second target epitope region. The second target epitope region can be a DLL3 epitope, or a non-DLL3 epitope, e.g., a different antigen. In certain embodiments, the multi-specific molecule comprises a first binding specificity for DLL3, a second binding specificity for a second target, and a third binding specificity for a third target. In certain embodiments, the second target is an antigen expressed on the surface of an immune cell (e.g., a T cell, or a human immune effector cell). In certain embodiments, the multi-specific molecule is capable of recruiting the activity of that immune effector cell by specifically binding to the effector antigen on the human immune effector cell, thereby enhancing effector function. In certain embodiments, the third target is an antigen expressed on a senescent cell.

The multi-specific molecules of the presently disclosed subject matter can be prepared by conjugating the constituent binding specificities using methods known in the art. For example, each binding specificity of the multi-specific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), 5, 5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, MA et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Conjugating agents can be SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the multi-specific molecule is a mAb×mAb, mAb×Fab, Fab×F(ab′)₂ or ligand×Fab fusion protein.

Binding of the multi-specific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a y counter or a scintillation counter or by autoradiography.

### 5.4 Nucleic Acids encoding the Antibodies or Antigen-binding Fragments

The presently disclosed subject matter provides nucleic acids encoding the anti-DLL3 antibodies or antigen-binding fragments thereof disclosed herein.

Further provided are vectors comprising the presently disclosed nucleic acids. In certain embodiments, the vector is an expression vector. The presently disclosed subject matter further provides host cells comprising the vectors disclosed herein. In certain embodiments, the host cells are T cells.

### 5.5 Pharmaceutical Compositions and Methods of Treatment

The presently disclosed subject matter provides compositions comprising a presently disclosed anti-DLL3 antibody or an antigen-binding fragment thereof, a presently disclosed immunoconjugate, or a presently disclosed multi-specific molecule. In certain embodiments, the composition is a pharmaceutical composition further comprising a pharmaceutically acceptable carrier.

The anti-DLL3 antibodies or antigen-binding fragments thereof of the presently disclosed subject matter can be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof.

Pharmaceutically acceptable carriers may further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

The presently disclosed subject matter provides various methods of using the anti-DLL3 antibodies or antigen-binding fragments thereof, the immunoconjugates, the multi-specific molecules, and the compositions disclosed herein in a therapy. The presently disclosed subject matter provides methods for treating or ameliorating a disease or disorder in a subject. In certain embodiments, the method comprises administering to the subject the presently disclosed anti-DLL3 antibodies or antigen-binding fragments thereof, immunoconjugates, multi-specific molecules, or the compositions.

In certain embodiments, the disease or disorder is associated with DLL3. In certain embodiments, the disease or disorder is associated with overexpression of DLL3. In certain embodiments, the disease or disorder is tumor. In certain embodiments, the tumor is cancer. In certain embodiments, the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma. In certain embodiments, the neuroendocrine tumors of the lung are selected from the group consisting of pulmonary neuroendocrine cancer (including typical carcinoid tumors, and atypical carcinoid tumors), large cell neuroendocrine carcinoma (LCNEC), and small-cell lung cancer. In certain embodiments, the melanoma is uveal melanoma. In certain embodiments, the breast cancer is triple negative breast cancer.

The presently disclosed anti-DLL3 antibodies or antigen-binding fragments thereof, immunoconjugates, multi-specific molecules, or the compositions may be employed in conjunction with other therapeutic agents useful in the treatment of DLL3-associated diseases or disorders. For example, the presently disclosed anti-DLL3 antibodies or antigen-binding fragments thereof, immunoconjugates, multi-specific molecules, or the compositions may be separately, sequentially or simultaneously administered with at least one additional therapeutic agent, or be conjugated to at least one additional therapeutic agent. Non-limiting examples of additional therapeutic agents include marine-derived compounds (e.g., dolastatin 10, auristatins, tasidotin, dolastatin 15 or variants thereof, monomethyl auristatin E (MNIAE), monomethyl auristatin F (MNIAF)) (see Newman & Cragg, Mar Drugs. 2017 April; 15(4): 99), vinca agents, anti-estrogen drugs, aromatase inhibitors, ovarian suppression agents, VEGF/VEGFR inhibitors, PARP inhibitors, cytostatic alkaloids, cytotoxic antibiotics, antimetabolites, endocrine/hormonal agents, bisphosphonate therapy agents and targeted biological therapy agents (e.g., therapeutic peptides described in U.S. Pat. No. 6,306,832, WO 2012007137, WO 2005000889, WO 2010096603 etc.), alkylating agents, alkyl sulfonates, amanitins, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN*) doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, folic acid analogues, purine analogs, androgens, anti-adrenals, folic acid replenisher such as frolinic acid, acegl atone, al dophosphami de glycoside, aminolevulinic acid, eniluracil, amsacrine, bestrabucil, bisantrene, edatraxate, defo famine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2- ethylhydrazide, procarbazine, PSK polysaccharide complex (JHS Natural Products, Eugene, OR), razoxane; rhizoxin; sizofiran; spirogermanium; tenuazonic acid; triaziquone; 2,2′,2″- trichlorotriethylamine; trichothecenes (especially T-2 toxin, verracurin A, roridin A and anguidine); urethan; vindesine; vemurafenib; dacarbazine; mannomustine; mitobronitol; mitolactol; pipobroman; gacytosine; arabinosi de (“Ara-C”); cyclophosphamide; thiotepa; taxoids, chloranbucil; GEMZAR′ gemcitabine; 6-thioguanine; mercaptopurine; methotrexate; platinum analogs, vinblastine, platinum; etoposide (VP- 16); ifosfamide; mitoxantrone; vincristine; NAVELBINE®vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts or solvates, acids or derivatives of any of the above. In some embodiments, the at least one additional therapeutic agent is a chemotherapeutic agent. Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, auristatins, maytansinoids, tubulysins, calicheamicins, duocarmycins, benzodiazepines, and camptothecins.

Other compatible anti-cancer agents that can be used together with the presently disclosed anti-DLL3 antibodies or antigen-binding fragments thereof, immunoconjugates, multi-specific molecules, or the compositions comprise commercially or clinically available compounds such as erlotinib (TARCEVA®, Genentech/OSI Pharm.), docetaxel (TAXOTERE®, Sanofi-Aventis), 5-FU (fiuorouracil, 5-fluorouracil, CAS No. 51-21-8), PD- 0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo- 2,3,4,6,8-pentazabicyclo [4.3.0] nona-2,7,9-triene- 9-carboxamide, CAS No. 85622-93-1, TEMODAR®, TEMODAL®, Schering Plough), tamoxifen ((Z)-2-[4-(1,2- diphenylbut-1- enyl)phenoxy]-N,N-dimethylethanamine, NOLVADEX®, ISTUBAL®, VALODEX®), and doxorubicin (ADRIAMYCIN®). Additional commercially or clinically available anti-cancer agents comprise bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ- 235 (PI3K inhibitor, Novartis), XL-147 (PI3K inhibitor, Exelixis), PTK787/ZK 222584 (Novartis), fulvestrant (FASLODEX®, Astra7eneca), leucovorin (folinic acid), rapamycin (sirolimus, RAPAMUNE®, Wyeth), lapatinib (TYKERB®, GSK572016, Glaxo Smith Kline), lonafarnib (SARAS AR™, SCH 66336, Schering Plough), sorafenib (NEXAVAR®, BAY43- 9006, Bayer Labs), gefitinib (IRESSA®, Astra7eneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); vinorelbine (NAVELBINE®); capecitabine (XELODA®, Roche), tamoxifen (including NOLVADEX®; tamoxifen citrate, FARESTON® (toremifme citrate) MEGASE® (megestrol acetate), AROMASIN® (exemestane; Pfizer), formestanie, fadrozole, RIVISOR® (vorozole), FEMARA® and ARIMIDEX® (anastrozole; Astrazeneca); dabrafmib (TAFINLAR®, GlaxoSmithKline); dasatinib (SPRYCEL®, Bristol-Myers Squibb); trametinib (MEKINIST®, GlaxoSmithKline); nilotinib (TASIGNA®, Novartis), troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rrnRH, Vinorelbine and Esperamicins and pharmaceutically acceptable salts, acids and derivatives of any of the above.

The presently disclosed anti-DLL3 antibodies or antigen-binding fragments thereof, immunoconjugates, multi-specific molecules, or the compositions may optionally be administered as a single bolus to a subject in need thereof. Alternatively, the dosing regimen may comprise multiple administrations performed at various times after the appearance of tumors. Administration can be carried out by any suitable route, including orally, intranasally, parenterally (intravenously, intramuscularly, intraperitoneally, or subcutaneously), rectally, intracranially, intratumorally, intrathecally, or topically. Administration includes self-administration and the administration by another. It is also to be appreciated that the various modes of treatment of medical conditions as described are intended to mean “substantial”, which includes total but also less than total treatment, and wherein some biologically or medically relevant result is achieved.

In certain embodiments, the presently disclosed anti-DLL3 antibodies or antigen-binding fragments thereof, immunoconjugates, multi-specific molecules, or the compositions comprise pharmaceutical formulations which may be administered to subjects in need thereof in one or more doses. Dosage regimens can be adjusted to provide the desired response (e.g., a therapeutic response).

Typically, an effective amount of the presently disclosed anti-DLL3 antibodies or antigen-binding fragments thereof, immunoconjugates, multi-specific molecules, or the compositions sufficient for achieving a therapeutic effect, range from about 0.000001 mg per kilogram body weight per day to about 10,000 mg per kilogram body weight per day. Typically, the dosage ranges are from about 0.0001 mg per kilogram body weight per day to about 100 mg per kilogram body weight per day. For administration of anti-DLL3 antibodies, the dosage ranges from about 0.0001 to 100 mg/kg, and more usually 0.01 to 5 mg/kg every week, every two weeks or every three weeks, of the subject body weight. For example, dosages can be 1 mg/kg body weight or 10 mg/kg body weight every week, every two weeks or every three weeks or within the range of 1-10 mg/kg every week, every two weeks or every three weeks. In certain embodiments, a single dosage of antibody ranges from 0.1-10,000 micrograms per kg body weight. In certain embodiments, antibody concentrations in a carrier range from 0.2 to 2000 micrograms per delivered milliliter. An exemplary treatment regime entails administration once per every two weeks or once a month or once every 3 to 6 months. Anti-DLL3 antibodies may be administered on multiple occasions. Intervals between single dosages can be hourly, daily, weekly, monthly or yearly. Intervals can also be irregular as indicated by measuring blood levels of the antibody in the subject. In some methods, dosage is adjusted to achieve a serum antibody concentration in the subject of from about 20 μg/mL to about 125 μg/mL, 100 μg/mL to about 150 μg/mL, from about 125 μg/mL to about 175 μg/mL, or from about 150 μg/mL to about 200 μg/mL.

Alternatively, anti-DLL3 antibodies can be administered as a sustained release formulation, in which case less frequent administration is required. Dosage and frequency vary depending on the half-life of the antibody in the subject. The dosage and frequency of administration can vary depending on whether the treatment is prophylactic or therapeutic. In prophylactic applications, a relatively low dosage is administered at relatively infrequent intervals over a long period of time. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

The presently disclosed anti-DLL3 antibody or antigen-binding fragment thereof can be incorporated into pharmaceutical compositions suitable for administration. The pharmaceutical compositions generally comprise recombinant or substantially purified antibody and a pharmaceutically acceptable carrier in a form suitable for administration to a subject. Pharmaceutically acceptable carriers are determined in part by the particular composition being administered, as well as by the particular method used to administer the composition. Accordingly, there is a wide variety of suitable formulations of pharmaceutical compositions for administering the antibody compositions. The pharmaceutical compositions can be formulated as sterile, substantially isotonic and in full compliance with all Good Manufacturing Practice (GMP) regulations of the U.S. Food and Drug Administration.

The terms “pharmaceutically acceptable,” “physiologically tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. The term “pharmaceutically acceptable salts and esters” means salts and esters that are pharmaceutically acceptable and have the desired pharmacological properties. Such salts include salts that can be formed where acidic protons present in the composition are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include sters formed from carboxy, sulfonyloxy, and phosphonoxy groups present in the anti-DLL3 antibody, e.g., C1-6 alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. An anti-DLL3 antibody named in this technology can be present in unsalified or unesterified form, or in salified and/or esterified form, and the naming of such anti-DLL3 antibody is intended to include both the original (unsalified and unesterified) compound and its pharmaceutically-acceptable salts and esters.

A pharmaceutical composition of the present technology is formulated to be compatible with its intended and/or suitable route of administration. The presently disclosed anti-DLL3 antibodies or antigen-binding fragments thereof or compositions can be administered by parenteral, topical, intravenous, oral, subcutaneous, intraarterial, intradermal, transdermal, rectal, intracranial, intrathecal, intraperitoneal, intranasal; or intramuscular routes, or as inhalants.

The antibodies of the present technology can be administered in the form of a depot injection or implant preparation which can be formulated in such a manner as to permit a sustained or pulsatile release of the active ingredient. Oral compositions generally include an inert diluent or an edible carrier. They can be enclosed in gelatin capsules or compressed into tablets. For the purpose of oral therapeutic administration, the anti-DLL3 antibody can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

For administration by inhalation, the anti-DLL3 antibody can be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the anti-DLL3 antibody is formulated into ointments, salves, gels, or creams as generally known in the art.

The anti-DLL3 antibody can also be prepared as pharmaceutical compositions in the form of suppositories (e.g., with conventional suppository bases such as cocoa butter and other glycerides) or retention enemas for rectal delivery.

In certain embodiments, the anti-DLL3 antibody is prepared with carriers that will protect the anti-DLL3 antibody against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

### 5.6 Diagnostic and Prognostic Methods

The presently disclosed anti-DLL3 antibodies, antigen-binding fragments thereof, multi-specific molecules, and nucleic acids encode thereof can be used for diagnostic and prognostic applications as well as use as research tools for detection of DLL3 in a biological sample, in a cell, a tissue, or a blood sample. The presently disclosed subject matter provides methods for detecting DLL3 in a cell, a tissue, or a blood sample. In certain embodiments, the method comprises: contacting a cell, a tissue, or a blood sample with the antibody, antigen-binding fragment thereof, or multi-specific molecule disclosed herein, wherein the antibody, antigen-binding fragment thereof or multi-specific molecule comprises a detectable label; and determining the amount of the labeled antibody, antigen-binding fragment thereof, or multi-specific molecule bound to the cell, tissue, or blood sample by measuring the amount of detectable label associated with the cell or tissue, wherein the amount of bound antibody, antigen-binding fragment thereof, or multi-specific molecule indicates the amount of DLL3 in the cell, tissue, or a blood sample. The cell or tissue can be any cell or tissue, including any normal, healthy, or cancerous cells and tissues. In certain embodiments, the blood sample is a peripheral blood sample.

The presently disclosed subject matter provides method for detecting a disease or disease associated with DLL3 in a subject in vivo. In certain embodiments, the method comprises (a) administering to the subject an effective amount of the presently disclosed anti-DLL3 antibody or antigen binding fragment thereof, which is configured to localize to a disease or disorder expressing DLL3 and is labeled with a radioisotope; and (b) detecting the presence of the disease or disorder in the subject by detecting radioactive levels emitted by the antibody that are higher than a reference value. In certain embodiments, the reference value is expressed as injected dose per gram (% ID/g). The reference value may be calculated by measuring the radioactive levels present in normal/control tissues, and computing the average radioactive levels present in normal/control tissues ±standard deviation. In certain embodiments, the ratio of radioactive levels between a disease/disorder and normal tissue is about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1.

In certain embodiments, the subject is diagnosed with or is suspected of having a disease or disorder that is associated with DLL3. Radioactive levels emitted by the antibody may be detected using positron emission tomography or single photon emission computed tomography.

The presently disclosed anti-DLL3 antibodies or antigen-binding fragments thereof can be used in methods known in the art relating to the localization and/or quantitation of DLL3 polypeptides (e.g., for use in measuring levels of the DLL3 protein within appropriate physiological samples, for use in diagnostic methods, for use in imaging the polypeptide, and the like). The presently disclosed anti-DLL3 antibodies or antigen-binding fragments thereof can be used to isolate a DLL3 polypeptide by standard techniques, such as affinity chromatography or immunoprecipitation. The presently disclosed anti-DLL3 antibodies or antigen-binding fragments thereof can facilitate the purification of natural immunoreactive

DLL3 proteins from biological samples, e.g., mammalian sera or cells as well as recombinantly-produced immunoreactive DLL3 proteins expressed in a host system. Moreover, anti-DLL3 antibodies of the present technology can be used to detect an immunoreactive DLL3 protein (e.g., in plasma, a cellular lysate or cell supernatant) in order to evaluate the abundance and pattern of expression of the immunoreactive polypeptide. The presently disclosed anti-DLL3 antibodies or antigen-binding fragments thereof can be used diagnostically to monitor immunoreactive DLL3 protein levels in tissue as part of a clinical testing procedure, e.g., to determine the efficacy of a given treatment regimen. As noted above, the detection can be facilitated by coupling (i.e., physically linking) the presently disclosed anti-DLL3 antibodies or antigen-binding fragments thereof to a detectable substance.

An exemplary method for detecting the presence or absence of an immunoreactive DLL3 protein in a biological sample comprises contacting a biological sample from a subject with a presently disclosed anti-DLL3 antibody or an antigen-binding fragment thereof, wherein the presence of an immunoreactive DLL3 protein is detected in the biological sample. Detection may be accomplished by means of a detectable label attached to the antibody.

The term “labeled” with regard to the anti-DLL3 antibody or antigen-binding fragment thereof is intended to encompass direct labeling of the antibody by coupling (i.e., physically linking) a detectable substance to the antibody, as well as indirect labeling of the antibody by reactivity with another compound that is directly labeled, such as a secondary antibody. Examples of indirect labeling include detection of a primary antibody using a fluorescently-labeled secondary antibody and end-labeling of a DNA probe with biotin such that it can be detected with fluorescently-labeled streptavidin.

In certain embodiments, the presently disclosed anti-DLL3 antibodies or antigen-binding fragments thereof are conjugated to one or more detectable labels. For such uses, the presently disclosed anti-DLL3 antibodies or antigen-binding fragments thereof may be detectably labeled by covalent or non-covalent attachment of a chromogenic, enzymatic, radioisotopic, isotopic, fluorescent, toxic, chemiluminescent, nuclear magnetic resonance contrast agent or other label.

Examples of suitable chromogenic labels include diaminobenzidine and 4-hydroxyazo-benzene-2-carboxylic acid. Examples of suitable enzyme labels include malate dehydrogenase, staphylococcal nuclease, A-5-steroid isomerase, yeast-alcohol dehydrogenase, a-glycerol phosphate dehydrogenase, triose phosphate isomerase, peroxidase, alkaline phosphatase, asparaginase, glucose oxidase, r3-galactosidase, ribonuclease, urease, catalase, glucose-6-phosphate dehydrogenase, glucoamylase, and acetylcholine esterase.

Examples of suitable radioisotopic labels include ³H, 1¹¹IN, ¹²⁵I ¹³¹I ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Cci, ²¹¹At, ²¹²Pb, ⁴⁷Se, ¹⁰⁹Pd, etc. In an exemplary isotope where in vivo imaging is used since it avoids the problem of dehalogenation of the ¹²⁵I or ¹³¹I labeled DLL3-binding antibodies by the liver. In addition, this isotope has a more favorable gamma emission energy for imaging. For example, ‘In coupled to monoclonal antibodies with 1-(P-isothiocyanatobenzyl)-DPTA exhibits little uptake in non-tumorous tissues, particularly the liver, and enhances specificity of tumor localization. Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include an ¹⁵²Eu label, a fluorescein label, an isothiocyanate label, a rhodamine label, a phycoerythrin label, a phycocyanin label, an allophycocyanin label, a Green Fluorescent Protein (GFP) label, an o-phthaldehyde label, and a fluorescamine label. Examples of suitable toxin labels include diphtheria toxin, ricin, and cholera toxin.

Examples of chemiluminescent labels include a luminol label, an isoluminol label, an aromatic acridinium ester label, an imidazole label, an acridinium salt label, an oxalate ester label, a luciferin label, a luciferase label, and an aequorin label. Examples of nuclear magnetic resonance contrasting agents include heavy metal nuclei such as Gd, Mn, and iron.

The presently disclosed detection methods can be used to detect an immunoreactive DLL3 protein in a biological sample in vitro as well as in vivo. Non-limiting examples of in vitro techniques for detection of an immunoreactive DLL3 protein include enzyme linked immunosorbent assays (ELISAs), Western blots, immunoprecipitations, radioimmunoassay, and immunofluorescence. Furthermore, in vivo techniques for detection of an immunoreactive DLL3 protein include introducing into a subject a labeled anti-DLL3 antibody or an antigen-binding fragment thereof. For example, the anti-DLL3 antibody or antigen-binding fragment thereof can be labeled with a radioactive marker whose presence and location in a subject can be detected by standard imaging techniques. In certain embodiments, the biological sample comprises DLL3 protein molecules from the test subject.

The presently disclosed anti-DLL3 antibodies or antigen-binding fragments thereof can be used to assay immunoreactive DLL3 protein levels in a biological sample (e.g., human plasma) using antibody-based techniques. For example, protein expression in tissues can be studied with classical immunohistological methods. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (125I, ¹²¹I, ¹³¹I), carbon (¹⁴C), sulfur (³⁵S), tritium (³H), indium (¹¹¹In), and technetium (^{99m}Tc), and fluorescent labels, such as fluorescein, rhodamine, and green fluorescent protein (GFP), as well as biotin.

In addition to assaying immunoreactive DLL3 protein levels in a biological sample, the presently disclosed anti-DLL3 antibodies or antigen-binding fragments thereof may be used for in vivo imaging of DLL3. Antibodies useful for this method include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the anti-DLL3 antibodies by labeling of nutrients for the relevant scFv clone.

The presently disclosed anti-DLL3 antibodies or antigen-binding fragments thereof, which are labeled with an appropriate detectable imaging moiety (such as a radioisotope (e.g., ¹³¹I, ¹¹¹IN ^{99m}Tc, ¹⁸F, ⁸⁹Zr), a radio-opaque substance, or a material detectable by nuclear magnetic resonance) are introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled anti-DLL3 antibody or antigen-binding fragment thereof then accumulates at the location of cells which contain the specific target polypeptide. For example, the labeled anti-DLL3 antibodies or antigen-binding fragments thereof accumulate within the subject in cells and tissues in which the DLL3 protein has localized.

Thus, the presently disclosed subject matter provides diagnostic methods of a medical condition. In certain embodiments, the method comprises: (a) assaying the expression of immunoreactive DLL3 protein by measuring binding of a presently disclosed anti-DLL3 antibody or an antigen-binding fragment thereof in cells or body fluid of an individual; and (b) comparing the amount of immunoreactive DLL3 protein present in the sample with a standard reference, wherein an increase or decrease in immunoreactive DLL3 protein levels compared to the standard is indicative of a medical condition.

Furthermore, the presently disclosed anti-DLL3 antibodies or antigen-binding fragments thereof may be used to purify immunoreactive DLL3 protein from a sample. In certain embodiments, the antibodies are immobilized on a solid support. Non-limiting examples of such solid supports include plastics such as polycarbonate, complex carbohydrates such as agarose and sepharose, acrylic resins and such as polyacrylamide and latex beads. Techniques for coupling antibodies to such solid supports are well known in the art.

The simplest method to bind the antigen to the antibody-support matrix is to collect the beads in a column and pass the antigen solution down the column. The efficiency of this method depends on the contact time between the immobilized antibody and the antigen, which can be extended by using low flow rates. The immobilized antibody captures the antigen as it flows past. Alternatively, an antigen can be contacted with the antibody-support matrix by mixing the antigen solution with the support (e.g., beads) and rotating the slurry, allowing maximum contact between the antigen and the immobilized antibody. After the binding reaction has been completed, the slurry is passed into a column for collection of the beads. The beads are washed using a suitable washing buffer and then the pure or substantially pure antigen is eluted.

An antibody or polypeptide of interest can be conjugated to a solid support, such as a bead. In addition, a first solid support such as a bead can also be conjugated, if desired, to a second solid support, which can be a second bead or other support, by any suitable means, including those disclosed herein for conjugation of a polypeptide to a support. Accordingly, any of the conjugation methods and means disclosed herein with reference to conjugation of a polypeptide to a solid support can also be applied for conjugation of a first support to a second support, where the first and second solid support can be the same or different.

Appropriate linkers, which can be cross-linking agents, for use for conjugating a polypeptide to a solid support include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl)propionic acid can be employed as a means for cleaving a polypeptide from a solid support. (Brown et al., Mol. Divers, pp, 4-12 (1995); Rothschild et al., Nucl. Acids Res., 24:351-66 (1996); and U.S. Pat. No. 5,643,722). Other cross-linking reagents are well-known in the art. (See, e.g., Wong (1991), supra; and Hermanson (1996), supra).

An antibody or polypeptide can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoroacetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, ncluding, if desired, under typical MS conditions, where a matrix, such as 3-HPA acts as an acid.

Orthogonally cleavable linkers can also be useful for binding a first solid support, e.g., a bead to a second solid support, or for binding a polypeptide of interest to a solid support. Using such linkers, a first solid support, e.g., a bead, can be selectively cleaved from a second solid support, without cleaving the polypeptide from the support; the polypeptide then can be cleaved from the bead at a later time. For example, a disulfide linker, which can be cleaved using a reducing agent, such as DTT, can be employed to bind a bead to a second solid support, and an acid cleavable bi-functional trityl group could be used to immobilize a polypeptide to the support. As desired, the linkage of the polypeptide to the solid support can be cleaved first, e.g., leaving the linkage between the first and second support intact. Trityl linkers can provide a covalent or hydrophobic conjugation and, regardless of the nature of the conjugation, the trityl group is readily cleaved in acidic conditions.

For example, a bead can be bound to a second support through a linking group which can be selected to have a length and a chemical nature such that high density binding of the beads to the solid support, or high density binding of the polypeptides to the beads, is promoted. Such a linking group can have, e.g., “tree-like” structure, thereby providing a multiplicity of functional groups per attachment site on a solid support. Examples of such linking group; include polylysine, polyglutamic acid, penta-erythrole and tris-hydroxy-aminomethane.

Noncovalent Binding Association. An antibody or polypeptide can be conjugated to a solid support, or a first solid support can also be conjugated to a second solid support, through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide or a second solid support containing a complementary binding moiety. For example, a bead coated with avidin or with streptavidin can be bound to a polypeptide having a biotin moiety incorporated therein, or to a second solid support coated with biotin or derivative of biotin, such as iminobiotin.

It should be recognized that any of the binding members disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

The presently disclosed anti-DLL3 antibodies or antigen-binding fragments thereof are useful in diagnostic methods. As such, the presently disclosed subject matter provides methods using the presently disclosed anti-DLL3 antibodies or antigen-binding fragments thereof in diagnosis of DLL3 activity in a subject. The presently disclosed anti-DLL3 antibodies or antigen-binding fragments thereof may be selected such that they have any level of epitope binding specificity and high binding affinity to a DLL3 polypeptide.

The presently disclosed anti-DLL3 antibodies or antigen-binding fragments thereof can be used to detect an immunoreactive DLL3 protein in a variety of standard assay formats. Such formats include immunoprecipitation, Western blotting, ELISA, radioimmunoassay, and immunometric assays. Biological samples can be obtained from any tissue or body fluid of a subject. In certain embodiments, the subject is at an early stage of cancer. In certain embodiments, the early stage of cancer is determined by the level or expression pattern of DLL3 protein in a sample obtained from the subject. In certain embodiments, the sample is selected from the group consisting of urine, blood, serum, plasma, saliva, amniotic fluid, cerebrospinal fluid (CSF), and biopsied body tissue.

Immunometric or sandwich assays are one format for the diagnostic methods of the present technology. Such assays use one antibody, e.g., the anti-DLL3 antibody or a population of anti-DLL3 antibodies immobilized to a solid phase, and another anti-DLL3 antibody or a population of anti-DLL3 antibodies in solution. Typically, the solution anti-DLL3 antibody or population of anti-DLL3 antibodies is labeled. If an antibody population is used, the population can contain antibodies binding to different epitope specificities within the target polypeptide. Accordingly, the same population can be used for both solid phase and solution antibody. If anti-DLL3 monoclonal antibodies are used, first and second DLL3 monoclonal antibodies having different binding specificities are used for the solid and solution phase. Solid phase (also referred to as “capture”) and solution (also referred to as “detection”) antibodies can be contacted with target antigen in either order or simultaneously. If the solid phase antibody is contacted first, the assay is referred to as being a forward assay. Conversely, if the solution antibody is contacted first, the assay is referred to as being a reverse assay. If the target is contacted with both antibodies simultaneously, the assay is referred to as a simultaneous assay. After contacting the DLL3 protein with the anti-DLL3 antibody, a sample is incubated for a period that usually varies from about 10 min to about 24 hr and is usually about 1 hr. A wash step is then performed to remove components of the sample not specifically bound to the anti-DLL3 antibody being used as a diagnostic reagent. When solid phase and solution antibodies are bound in separate steps, a wash can be performed after either or both binding steps. After washing, binding is quantified, typically by detecting a label linked to the solid phase through binding of labeled solution antibody. Usually for a given pair of antibodies or populations of antibodies and given reaction conditions, a calibration curve is prepared from samples containing known concentrations of target antigen. Concentrations of the immunoreactive DLL3 protein in samples being tested are then read by interpolation from the calibration curve (i.e., standard curve). Analyte can be measured either from the amount of labeled solution antibody bound at equilibrium or by kinetic measurements of bound labeled solution antibody at a series of time points before equilibrium is reached. The slope of such a curve is a measure of the concentration of the DLL3 protein in a sample.

Suitable supports for use in the above methods include, e.g., nitrocellulose membranes, nylon membranes, and derivatized nylon membranes, and also particles, such as agarose, a dextran-based gel, dipsticks, particulates, microspheres, magnetic particles, test tubes, microtiter wells, SEPHADEX™ (Amersham Pharmacia Biotech, Piscataway N.J.), and the like. Immobilization can be by absorption or by covalent attachment. Optionally, anti-DLL3 antibodies can be joined to a linker molecule, such as biotin for attachment to a surface bound linker, such as avidin.

In certain embodiments, the presently disclosed anti-DLL3 antibody or antigen-binding fragment thereof is conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells comprising the antigen.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). In certain embodiments, the diagnostic agents are selected from the group consisting of radioisotopes, enhancing agents for use in magnetic resonance imaging, and fluorescent compounds. Chelates may be coupled to the presently disclosed anti-DLL3 antibodies or antigen-binding fragments thereof using standard chemistries. The chelate is normally linked to the antibody by a group which enables formation of a bond to the molecule with minimal loss of immunoreactivity and minimal aggregation and/or internal cross-linking.

### 5.7 Kits

The presently disclosed subject matter provides kits for treatment or ameliorating a disease or disorder, and/or detecting DLL3. In certain embodiments, the kit comprises the anti-DLL3 antibodies or antigen-binding fragments thereof, the immunoconjugate, the multi-specific molecule, or the composition disclosed herein. In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit further comprises instructions for administering the anti-DLL3 antibodies or antigen-binding fragments thereof, the immunoconjugate, the multi-specific molecule, or the composition disclosed herein to a subject in need the treatment. The instructions can generally include information about the use of the anti-DLL3 antibodies or antigen-binding fragments thereof, the immunoconjugate, the multi-specific molecule, and the composition disclosed herein for the treatment or ameliorating a disease or disorder. In certain embodiments, the instructions include at least one of the following: description of the therapeutic agent; dosage schedule and administration for treatment and/or prevention of a tumor or neoplasm or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.8 Exemplary Embodiments

A1. In certain non-limiting embodiments, the presently disclosed subject matter provides an anti-DLL3 antibody or an antigen-binding fragment thereof, comprising a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, or SEQ ID NO: 172.

A2. In certain non-limiting embodiments, the presently disclosed subject matter provides an anti-DLL3 antibody or an antigen-binding fragment thereof, comprising a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, or SEQ ID NO: 173.

A3. In certain non-limiting embodiments, the presently disclosed subject matter provides an anti-DLL3 antibody or an antigen-binding fragment thereof, comprising:
a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, or SEQ ID NO: 172; and
b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, or SEQ ID NO: 173.

A4. In certain non-limiting embodiments, the presently disclosed subject matter provides an anti-DLL3 antibody or an antigen-binding fragment thereof, comprising a heavy chain variable region and a light chain variable region, wherein the heavy chain variable region and the light chain variable region are selected from the group consisting of:
a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO:7, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8;
b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 17, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 18;
c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 25;
d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 34, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 35;
e) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 42, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 43;
f) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 52, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 53;
g) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 60, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 61;
h) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 66, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 67;
i) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 76, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 77;
j) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 83, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 84;
k) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 92, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 93;
l) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 102, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 103;
m) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 108, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 109;
n) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 108, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 113;
o) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 119, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 120;
p) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 126, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 127;
q) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 131, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 132;
r) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 141, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 142;
s) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 147, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 148;
t) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 153, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 154;
u) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 157, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 158;
v) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 163, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 164; and
w) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 172, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 173.

A5. In certain non-limiting embodiments, the presently disclosed subject matter provides an anti-DLL3 antibody or an antigen-binding fragment thereof, comprising a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, or SEQ ID NO: 172.

A6. In certain non-limiting embodiments, the presently disclosed subject matter provides an anti-DLL3 antibody or an antigen-binding fragment thereof, comprising a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, or SEQ ID NO: 173.

A7. In certain non-limiting embodiments, the presently disclosed subject matter provides an anti-DLL3 antibody or an antigen-binding fragment thereof, comprising
a) a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, or SEQ ID NO: 172; and
b) a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, or SEQ ID NO: 173.

A8. The foregoing antibody or antigen-binding fragment thereof of any one of A1-A7, wherein
a) the heavy chain variable region comprises the amino acid sequence set forth in SEQ ID NO: 7, and the light chain variable region comprises the amino acid sequence set forth in SEQ ID NO: 8;
b) the heavy chain variable region comprises the amino acid sequence set forth in SEQ ID NO: 17, and the light chain variable region comprises the amino acid sequence set forth in SEQ ID NO: 18;
c) the heavy chain variable region comprises the amino acid sequence set forth in SEQ ID NO: 24, and the light chain variable region comprises the amino acid sequence set forth in SEQ ID NO: 25;
d) the heavy chain variable region comprises the amino acid sequence set forth in SEQ ID NO: 34, and the light chain variable region comprises the amino acid sequence set forth in SEQ ID NO: 35;
e) the heavy chain variable region comprises the amino acid sequence set forth in SEQ ID NO: 42, and the light chain variable region comprises the amino acid sequence set forth in SEQ ID NO: 43;
f) the heavy chain variable region comprises the amino acid sequence set forth in SEQ ID NO: 52, and the light chain variable region comprises the amino acid sequence set forth in SEQ ID NO: 53;
g) the heavy chain variable region comprises the amino acid sequence set forth in SEQ ID NO: 60, and the light chain variable region comprises the amino acid sequence set forth in SEQ ID NO: 61;
h) the heavy chain variable region comprises the amino acid sequence set forth in SEQ ID NO: 66, and the light chain variable region comprises the amino acid sequence set forth in SEQ ID NO: 67;
i) the heavy chain variable region comprises the amino acid sequence set forth in SEQ ID NO: 76, and the light chain variable region comprises the amino acid sequence set forth in SEQ ID NO: 77;
j) the heavy chain variable region comprises the amino acid sequence set forth in SEQ ID NO: 83, and the light chain variable region comprises the amino acid sequence set forth in SEQ ID NO: 84;
k) the heavy chain variable region comprises the amino acid sequence set forth in SEQ ID NO: 92, and the light chain variable region comprises the amino acid sequence set forth in SEQ ID NO: 93;
l) the heavy chain variable region comprises the amino acid sequence set forth in SEQ ID NO: 102, and the light chain variable region comprises the amino acid sequence set forth in SEQ ID NO: 103;
m) a heavy chain variable region comprising the amino acid sequence set forth in SEQ ID NO: 108, and a light chain variable region comprising the amino acid sequence set forth in SEQ ID NO: 109;
n) a heavy chain variable region comprising the amino acid sequence set forth in SEQ ID NO: 108, and a light chain variable region comprising the amino acid sequence set forth in SEQ ID NO: 113;
o) a heavy chain variable region comprising the amino acid sequence set forth in SEQ ID NO: 119, and a light chain variable region comprising the amino acid sequence set forth in SEQ ID NO: 120;
p) a heavy chain variable region comprising the amino acid sequence set forth in SEQ ID NO: 126, and a light chain variable region comprising the amino acid sequence set forth in SEQ ID NO: 127;
q) a heavy chain variable region comprising the amino acid sequence set forth in SEQ ID NO: 131, and a light chain variable region comprising the amino acid sequence set forth in SEQ ID NO: 132;
r) a heavy chain variable region comprising the amino acid sequence set forth in SEQ ID NO: 141, and a light chain variable region comprising the amino acid sequence set forth in SEQ ID NO: 142;
s) a heavy chain variable region comprising the amino acid sequence set forth in SEQ ID NO: 147, and a light chain variable region comprising the amino acid sequence set forth in SEQ ID NO: 148;
t) a heavy chain variable region comprising the amino acid sequence set forth in SEQ ID NO: 153, and a light chain variable region comprising the amino acid sequence set forth in SEQ ID NO: 154;
u) a heavy chain variable region comprising the amino acid sequence set forth in SEQ ID NO: 157, and a light chain variable region comprising the amino acid sequence set forth in SEQ ID NO: 158;
v) a heavy chain variable region comprising the amino acid sequence set forth in SEQ ID NO: 163, and a light chain variable region comprising the amino acid sequence set forth in SEQ ID NO: 164; or
w) a heavy chain variable region comprising the amino acid sequence set forth in SEQ ID NO: 172, and a light chain variable region comprising the amino acid sequence set forth in SEQ ID NO: 173.

A9. The foregoing antibody or antigen-binding fragment thereof of any one of A1-A7, wherein:
a) the heavy chain variable region comprises the amino acid sequence set forth in SEQ ID NO: 7, and the light chain variable region comprises the amino acid sequence set forth in SEQ ID NO: 8;
b) the heavy chain variable region comprises the amino acid sequence set forth in SEQ ID NO: 34, and the light chain variable region comprises the amino acid sequence set forth in SEQ ID NO: 35; or
c) the heavy chain variable region comprises the amino acid sequence set forth in SEQ ID NO: 42, and the light chain variable region comprises the amino acid sequence set forth in SEQ ID NO: 43.

A10. In certain non-limiting embodiments, the presently disclosed subject matter provides an anti-DLL3 antibody or an antigen-binding fragment thereof, comprising a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
a) a heavy chain variable region CDR3 comprising the amino acid sequence set forth in SEQ ID NO: 3 and a conservative modification thereof; and a light chain variable region CDR3 comprising the amino acid sequence set forth in SEQ ID NO: 6 and a conservative modification thereof;
b) a heavy chain variable region CDR3 comprising the amino acid sequence set forth in SEQ ID NO: 13 and a conservative modification thereof; and a light chain variable region CDR3 comprising the amino acid sequence set forth in SEQ ID NO: 16 and a conservative modification thereof;
c) a heavy chain variable region CDR3 comprising the amino acid sequence set forth in SEQ ID NO: 22 and a conservative modification thereof; and a light chain variable region CDR3 comprising the amino acid sequence set forth in SEQ ID NO: 23 and a conservative modification thereof;
d) a heavy chain variable region CDR3 comprising the amino acid sequence set forth in SEQ ID NO: 30 and a conservative modification thereof; and a light chain variable region CDR3 comprising the amino acid sequence set forth in SEQ ID NO: 33 and a conservative modification thereof;
e) a heavy chain variable region CDR3 comprising the amino acid sequence set forth in SEQ ID NO: 39 and a conservative modification thereof; and a light chain variable region CDR3 comprising the amino acid sequence set forth in SEQ ID NO: 41 and a conservative modification thereof;
f) a heavy chain variable region CDR3 comprising the amino acid sequence set forth in SEQ ID NO: 48 and a conservative modification thereof; and a light chain variable region CDR3 comprising the amino acid sequence set forth in SEQ ID NO: 51 and a conservative modification thereof;
g) a heavy chain variable region CDR3 comprising the amino acid sequence set forth in SEQ ID NO: 56 and a conservative modification thereof; and a light chain variable region CDR3 comprising the amino acid sequence set forth in SEQ ID NO: 59 and a conservative modification thereof;
h) a heavy chain variable region CDR3 comprising the amino acid sequence set forth in SEQ ID NO: 64 and a conservative modification thereof; and a light chain variable region CDR3 comprising the amino acid sequence set forth in SEQ ID NO: 65 and a conservative modification thereof;
i) a heavy chain variable region CDR3 comprising the amino acid sequence set forth in SEQ ID NO: 72 and a conservative modification thereof; and a light chain variable region CDR3 comprising the amino acid sequence set forth in SEQ ID NO: 75 and a conservative modification thereof;
j) a heavy chain variable region CDR3 comprising the amino acid sequence set forth in SEQ ID NO: 81 and a conservative modification thereof; and a light chain variable region CDR3 comprising the amino acid sequence set forth in SEQ ID NO: 82 and a conservative modification thereof;
k) a heavy chain variable region CDR3 comprising the amino acid sequence set forth in SEQ ID NO: 89 and a conservative modification thereof; and a light chain variable region CDR3 comprising the amino acid sequence set forth in SEQ ID NO: 91 and a conservative modification thereof;
l) a heavy chain variable region CDR3 comprising the amino acid sequence set forth in SEQ ID NO: 98 and a conservative modification thereof; and a light chain variable region CDR3 comprising the amino acid sequence set forth in SEQ ID NO: 101 and a conservative modification thereof;
m) a heavy chain variable region CDR3 comprising the amino acid sequence set forth in SEQ ID NO: 107 and a conservative modification thereof; and a light chain variable region CDR3 comprising the amino acid sequence set forth in SEQ ID NO: 82 and a conservative modification thereof;
n) a heavy chain variable region CDR3 comprising the amino acid sequence set forth in SEQ ID NO: 107 and a conservative modification thereof; and a light chain variable region CDR3 comprising the amino acid sequence set forth in SEQ ID NO: 112 and a conservative modification thereof;
o) a heavy chain variable region CDR3 comprising the amino acid sequence set forth in SEQ ID NO: 116 and a conservative modification thereof; and a light chain variable region CDR3 comprising the amino acid sequence set forth in SEQ ID NO: 118 and a conservative modification thereof;
p) a heavy chain variable region CDR3 comprising the amino acid sequence set forth in SEQ ID NO: 123 and a conservative modification thereof; and a light chain variable region CDR3 comprising the amino acid sequence set forth in SEQ ID NO: 125 and a conservative modification thereof;
q) a heavy chain variable region CDR3 comprising the amino acid sequence set forth in SEQ ID NO: 56 and a conservative modification thereof; and a light chain variable region CDR3 comprising the amino acid sequence set forth in SEQ ID NO: 130 and a conservative modification thereof;
r) a heavy chain variable region CDR3 comprising the amino acid sequence set forth in SEQ ID NO: 137 and a conservative modification thereof; and a light chain variable region CDR3 comprising the amino acid sequence set forth in SEQ ID NO: 140 and a conservative modification thereof;
s) a heavy chain variable region CDR3 comprising the amino acid sequence set forth in SEQ ID NO: 145 and a conservative modification thereof; and a light chain variable region CDR3 comprising the amino acid sequence set forth in SEQ ID NO: 125 and a conservative modification thereof;
t) a heavy chain variable region CDR3 comprising the amino acid sequence set forth in SEQ ID NO: 152 and a conservative modification thereof; and a light chain variable region CDR3 comprising the amino acid sequence set forth in SEQ ID NO: 82 and a conservative modification thereof;
u) a heavy chain variable region CDR3 comprising the amino acid sequence set forth in SEQ ID NO: 123 and a conservative modification thereof; and a light chain variable region CDR3 comprising the amino acid sequence set forth in SEQ ID NO: 59 and a conservative modification thereof;
v) a heavy chain variable region CDR3 comprising the amino acid sequence set forth in SEQ ID NO: 161 and a conservative modification thereof; and a light chain variable region CDR3 comprising the amino acid sequence set forth in SEQ ID NO: 162 and a conservative modification thereof; or
w) a heavy chain variable region CDR3 comprising the amino acid sequence set forth in SEQ ID NO: 168 and a conservative modification thereof; and a light chain variable region CDR3 comprising the amino acid sequence set forth in SEQ ID NO: 171 and a conservative modification thereof.

A11. The foregoing antibody or antigen-binding fragment thereof of A10, wherein the heavy chain variable region and light chain variable region CDR2 domains are selected from the group consisting of:
a) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 5 and a conservative modification thereof;
b) a heavy chain variable region CDR2 comprising the amino acid sequence set forth in SEQ ID NO: 12 and a conservative modification thereof; and a light chain variable region CDR2 comprising the amino acid sequence set forth in SEQ ID NO: 15 and a conservative modification thereof;
c) a heavy chain variable region CDR2 comprising the amino acid sequence set forth in SEQ ID NO: 29 and a conservative modification thereof; and a light chain variable region CDR2 comprising the amino acid sequence set forth in SEQ ID NO: 32 and a conservative modification thereof;
d) a heavy chain variable region CDR2 comprising the amino acid sequence set forth in SEQ ID NO: 38 and a conservative modification thereof; and a light chain variable region CDR2 comprising the amino acid sequence set forth in SEQ ID NO: 5 and a conservative modification thereof;
e) a heavy chain variable region CDR2 comprising the amino acid sequence set forth in SEQ ID NO: 47 and a conservative modification thereof; and a light chain variable region CDR2 comprising the amino acid sequence set forth in SEQ ID NO: 50 and a conservative modification thereof;
f) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 58 and a conservative modification thereof;
g) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 5 and a conservative modification thereof;
h) a heavy chain variable region CDR2 comprising the amino acid sequence set forth in SEQ ID NO: 71 and a conservative modification thereof; and a light chain variable region CDR2 comprising the amino acid sequence set forth in SEQ ID NO: 74 and a conservative modification thereof;
i) a heavy chain variable region CDR2 comprising the amino acid sequence set forth in SEQ ID NO: 80 and a conservative modification thereof; and a light chain variable region CDR2 comprising the amino acid sequence set forth in SEQ ID NO: 58 and a conservative modification thereof;
j) a heavy chain variable region CDR2 comprising the amino acid sequence set forth in SEQ ID NO: 88 and a conservative modification thereof; and a light chain variable region CDR2 comprising the amino acid sequence set forth in SEQ ID NO: 32 216 and a conservative modification thereof;
k) a heavy chain variable region CDR2 comprising the amino acid sequence set forth in SEQ ID NO: 97 and a conservative modification thereof; and a light chain variable region CDR2 comprising the amino acid sequence set forth in SEQ ID NO: 100 and a conservative modification thereof;
l) a heavy chain variable region CDR2 comprising the amino acid sequence set forth in SEQ ID NO: 106 and a conservative modification thereof; and a light chain variable region CDR2 comprising the amino acid sequence set forth in SEQ ID NO: 58 and a conservative modification thereof;
m) a heavy chain variable region CDR2 comprising the amino acid sequence set forth in SEQ ID NO: 106 and a conservative modification thereof; and a light chain variable region CDR2 comprising the amino acid sequence set forth in SEQ ID NO: 5 and a conservative modification thereof;
n) a heavy chain variable region CDR2 comprising the amino acid sequence set forth in SEQ ID NO: 115 and a conservative modification thereof; and a light chain variable region CDR2 comprising the amino acid sequence set forth in SEQ ID NO: 100 and a conservative modification thereof;
o) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 58 and a conservative modification thereof;
p) a heavy chain variable region CDR2 comprising the amino acid sequence set forth in SEQ ID NO: 136 and a conservative modification thereof; and a light chain variable region CDR2 comprising the amino acid sequence set forth in SEQ ID NO: 139 and a conservative modification thereof;
q) a heavy chain variable region CDR2 comprising the amino acid sequence set forth in SEQ ID NO: 2 and a conservative modification thereof; and a light chain variable region CDR2 comprising the amino acid sequence set forth in SEQ ID NO: 146 and a conservative modification thereof;
r) a heavy chain variable region CDR2 comprising the amino acid sequence set forth in SEQ ID NO: 136 and a conservative modification thereof; and a light chain variable region CDR2 comprising the amino acid sequence set forth in SEQ ID NO: 74 and a conservative modification thereof; and
s) a heavy chain variable region CDR2 comprising the amino acid sequence set forth in SEQ ID NO: 167 and a conservative modification thereof; and a light chain variable region CDR2 comprising the amino acid sequence set forth in SEQ ID NO: 170 and a conservative modification thereof.

A12. The foregoing antibody or antigen-binding fragment thereof of A10 or A11, wherein the heavy chain variable region and light chain variable region CDR1 domains are selected from the group consisting of:
a) a heavy chain variable region CDR1 comprising the amino acid sequence set forth in SEQ ID NO: 1 and a conservative modification thereof; and a light chain variable region CDR1 comprising the amino acid sequence set forth in SEQ ID NO: 4 and a conservative modification thereof;
b) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 14 and a conservative modification thereof;
c) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 4 and a conservative modification thereof;
d) a heavy chain variable region CDR1 comprising the amino acid sequence set forth in SEQ ID NO: 28 and a conservative modification thereof; and a light chain variable region CDR1 comprising the amino acid sequence set forth in SEQ ID NO: 31 and a conservative modification thereof;
e) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 40 and a conservative modification thereof;
f) a heavy chain variable region CDR1 comprising the amino acid sequence set forth in SEQ ID NO: 46 and a conservative modification thereof; and a light chain variable region CDR1 comprising the amino acid sequence set forth in SEQ ID NO: 49 and a conservative modification thereof;
g) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 57 and a conservative modification thereof;
h) a heavy chain variable region CDR1 comprising the amino acid sequence set forth in SEQ ID NO: 70 and a conservative modification thereof; and a light chain variable region CDR1 comprising the amino acid sequence set forth in SEQ ID NO: 73 and a conservative modification thereof;
i) a heavy chain variable region CDR1 comprising the amino acid sequence set forth in SEQ ID NO: 87 and a conservative modification thereof; and a light chain variable region CDR1 comprising the amino acid sequence set forth in SEQ ID NO: 90 and a conservative modification thereof;
j) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 99 and a conservative modification thereof;
k) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 117 and a conservative modification thereof;
l) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 124 and a conservative modification thereof;
m) a heavy chain variable region CDR1 comprising the amino acid sequence set forth in SEQ ID NO: 135 and a conservative modification thereof; and a light chain variable region CDR1 comprising the amino acid sequence set forth in SEQ ID NO: 138 and a conservative modification thereof;
n) a heavy chain variable region CDR1 comprising the amino acid sequence set forth in SEQ ID NO: 151 and a conservative modification thereof; and a light chain variable region CDR1 comprising the amino acid sequence set forth in SEQ ID NO: 57 and a conservative modification thereof;
o) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 73 and a conservative modification thereof; and
p) a heavy chain variable region CDR1 comprising the amino acid sequence set forth in SEQ ID NO: 96 and a conservative modification thereof; and a light chain variable region CDR1 comprising the amino acid sequence set forth in SEQ ID NO: 169 and a conservative modification thereof.

A13. The foregoing antibody or antigen-binding fragment thereof of any one of A10-A12, wherein one or more of the CDR sequences have up to about 5 amino acid substitutions.

A14. The foregoing antibody or antigen-binding fragment thereof of any one of A10-A12, wherein one or more of the CDR sequences have up to about 3 amino acid substitutions.

A15. In certain non-limiting embodiments, the presently disclosed subject matter provides an anti-DLL3 antibody or an antigen-binding fragment thereof, comprising:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; b) a heavy chain variable region comprising a CDR1 comprising the amino acid
sequence set forth in SEQ ID NO: 11, a CDR2 comprising the amino acid sequence set forth in SEQ ID NO: 12, and a CDR3 comprising the amino acid sequence set forth in SEQ ID NO: 13;
c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22;
d) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30;
e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39;
f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56;
h) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64;
i) a heavy chain variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72;
j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81;
k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89;
l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98;
m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107;
n) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116;
o) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123;
p) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137;
q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145;
r) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152;
s) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161;
t) a heavy chain variable region comprising a CDR1 comprising the amino acid sequence set forth in SEQ ID NO: 96, a CDR2 comprising the amino acid sequence set forth in SEQ ID NO: 167, and a CDR3 comprising the amino acid sequence set forth in SEQ ID NO: 168.

A16. In certain non-limiting embodiments, the presently disclosed subject matter provides an anti-DLL3 antibody or an antigen-binding fragment thereof, comprising:
a) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
b) a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
c) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23;
d) a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
e) a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
f) a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
g) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
h) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
i) a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
j) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
k) a light chain variable region comprising a CDR1 comprising the amino acid sequence set forth in SEQ ID NO: 90, a CDR2 comprising the amino acid sequence set forth in SEQ ID NO: 32216, and a CDR3 comprising the amino acid sequence set forth in SEQ ID NO: 91;
l) a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
m) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
n) a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
o) a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
p) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
q) a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
r) a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
s) a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59;
t) a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162; or
u) a light chain variable region comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171.

A17. In certain non-limiting embodiments, the presently disclosed subject matter provides an anti-DLL3 antibody or an antigen-binding fragment thereof, comprising:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23;
d) a heavy chain variable region comprising a CDR1 comprising the amino acid sequence set forth in SEQ ID NO: 28, a CDR2 comprising the amino acid sequence set forth in SEQ ID NO: 29; and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48; a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
h) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64; a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
i) a heavy chain variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a light chain variable region comprising a CDR1 comprising the amino acid sequence set forth in SEQ ID NO: 90, a CDR2 comprising the amino acid sequence set forth in SEQ ID NO: 32216, and a CDR3 comprising the amino acid sequence set forth in SEQ ID NO: 91;
l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
n) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
o) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116; and a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
p) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
r) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
s) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137; and a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
t) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145; and a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
u) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
v) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59;
w) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162; or
x) a heavy chain variable region comprising a CDR1 comprising the amino acid sequence set forth in SEQ ID NO: 96, a CDR2 comprising the amino acid sequence set forth in SEQ ID NO: 167, a CDR3 comprising the amino acid sequence set forth in SEQ ID NO: 168; and a light chain variable region comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171.

A18. The foregoing antibody or an antigen-binding fragment thereof of A17, comprising:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
b) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; or
c) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41.

A19. The foregoing antibody or antigen-binding fragment thereof of any one of A1-A18, wherein the antibody or antigen-binding fragment thereof binds to a DLL3 comprising the amino acid sequence set forth in SEQ ID NO: 215 or a fragment thereof.

A20. The foregoing antibody or antigen-binding fragment thereof of any one of A1-A19, wherein the antibody comprises a human variable region framework region.

A21. The foregoing antibody or antigen-binding fragment thereof of any one of A1-A20, which is a fully human or an antigen-binding fragment thereof.

A22. The foregoing antibody or antigen-binding fragment thereof of any one of A1-A20, which is a chimeric antibody or an antigen-binding fragment thereof.

A23. The foregoing antibody or antigen-binding fragment thereof of any one of A1-A20, which is a humanized antibody or an antigen-binding fragment thereof.

A24. The foregoing antibody or antigen-binding fragment thereof of any one of A1-A23, wherein the antigen-binding fragment is a Fab, Fab′, F(ab′)2, variable fragment (Fv), or single chain variable region (scFv).

A25. The foregoing antibody or antigen-binding fragment thereof of A24, wherein the antigen antigen-binding fragment is an scFv.

A26. In certain non-limiting embodiments, the presently disclosed subject matter provides an antibody or an antigen-binding fragment thereof, which cross-competes for binding to DLL3 with an antibody or an antigen-binding fragment thereof of any one of A1-A25.

A27. In certain non-limiting embodiments, the presently disclosed subject matter provides an antibody or an antigen-binding fragment thereof, which binds to the same epitope region on DLL3 with an antibody or an antigen-binding fragment thereof of any one of A1-A25.

B1. In certain non-limiting embodiments, the presently disclosed subject matter provides a composition comprising the antibody or antigen-binding fragment thereof of any one of A1-A27.

B2. The foregoing composition of B1, which is a pharmaceutical composition that further comprises a pharmaceutically acceptable carrier.

C1. In certain non-limiting embodiments, the presently disclosed subject matter provides an immunoconjugate comprising the antibody or antigen-binding fragment thereof of any one of A1-A27, linked to a therapeutic agent.

C2. The foregoing immunoconjugate of C1, wherein the therapeutic agent is a drug, a cytotoxin, or a radioactive isotope.

C3. The foregoing immunoconjugate of C1 or C2 further comprising a chelator.

C4. The foregoing immunoconjugate of C3, wherein the chelator is selected from the group consisting of AAZTA, BAT, BARAC, BPCA, TE2A, CB-TE2A, CB0TE1A1P, CB-TE2P, MM-TE2A, DM TE-2A, CP356, DATA, DBCO, DiAmSar, DIBO, DIMA, DFO, DGO, DOTA, DOTMA, DTPA, EDTA, EGTA, EHPG, H2dedpa, H4octapa, H2azapa, H5decapa, H6phospa, HBED, SHBED, HEHA, HYNIC, LICAM, MECAM, NODASA, NODAGA, NOPO, NOTA, NETA, PEPA, PCTA, PDTA, TACN-TM, TCMC, TETA, TETMA, TRAP (PRP9), TRITA, TTHA, and derivatives thereof.

C5. The foregoing immunoconjugate of C3 or C4, wherein the chelator is DFO.

C6. The foregoing immunoconjugate of C2-C5, wherein the radioactive isotope is selected from the group consisting of ⁴⁷Sc, ⁶⁷Cu, ⁹⁰Y, ¹³¹I, ¹⁴⁹Tb, ¹⁶¹Tb, ¹⁷⁷Lu, ²²⁵Ac, ²¹³Bi, ²²³Ra, ⁸⁹Zr, and ²²⁷Th.

C7. The foregoing immunoconjugate of C6, wherein the radioactive isotope is ¹⁷⁷Lu.

C8. The foregoing immunoconjugate of C6, wherein the radioactive isotope is ⁸⁹Zr.

D1. In certain non-limiting embodiments, the presently disclosed subject matter provides a composition comprising the immunoconjugate of any one of C1-C8.

D2. The foregoing composition of D1, which is a pharmaceutical composition that further comprises a pharmaceutically acceptable carrier.

E1. In certain non-limiting embodiments, the presently disclosed subject matter provides a multi-specific molecule comprising the antibody or antigen-binding fragment thereof of any one of A1-A27, linked to one or more functional moieties.

E2. The foregoing multi-specific molecule of E1, wherein the one or more functional moieties have a different binding specificity than the antibody or antigen binding fragment thereof.

F1. In certain non-limiting embodiments, the presently disclosed subject matter provides a composition comprising the multi-specific molecule of E1 or E2.

F2. The foregoing composition of F1, which is a pharmaceutical composition that further comprises a pharmaceutically acceptable carrier.

G1. In certain non-limiting embodiments, the presently disclosed subject matter provides a nucleic acid that encodes an antibody or antigen-binding fragment thereof of any one of A1-A27.

G2. In certain non-limiting embodiments, the presently disclosed subject matter provides a vector comprising the nucleic acid molecule of G1.

G3. In certain non-limiting embodiments, the presently disclosed subject matter provides a host cell comprising the vector of G2.

G4. In certain non-limiting embodiments, the presently disclosed subject matter provides a method for detecting DLL3 in a whole cell, a tissue, or a blood sample, comprising:
a) contacting a cell, tissue or blood sample with the antibody or antigen-binding fragment thereof of any one of A1-A27, wherein the antibody or antigen-binding fragment thereof comprises a detectable label; and
b) determining the amount of the labeled antibody or antigen-binding fragment thereof bound to the cell, tissue or blood sample by measuring the amount of detectable label associated with said cell or tissue, wherein the amount of bound antibody or antigen-binding fragment thereof indicates the amount of DLL3 in the cell, tissue or blood sample.

H1. In certain non-limiting embodiments, the presently disclosed subject matter provides a method of treating or ameliorating a disease or disorder associated with DLL3 in a subject, comprising administering to the subject the antibody or antigen-binding fragment thereof of any one of A1-A27, the immunoconjugate of any one of C1-C8, the multi-specific molecule of E1 or E2, or the composition of any one of claims B1, B2, D1, D2, F1, and F2.

H2. The foregoing method of H1, wherein the disease or disorder is a tumor.

H3. The foregoing method of H2, wherein the tumor is cancer.

H4. The foregoing method of any one of H1-H3, wherein the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma.

H5. The foregoing method of H5, wherein the neuroendocrine tumors of the lung are selected from the group consisting of pulmonary neuroendocrine cancer, large cell neuroendocrine carcinoma, and small-cell lung cancer.

H6. The foregoing method of any one of H1-H5, wherein the subject is a human.

I1. In certain non-limiting embodiments, the presently disclosed subject matter provides a kit for treating or ameliorating a disease or disorder in a subject, comprising the antibody or antigen-binding fragment thereof of any one of A1-A27, the immunoconjugate of any one of C1-C8, the multi-specific molecule of E1 or E2, or the composition of any one of claims B1, B2, D1, D2, F1, and F2.

I2. The foregoing kit of I1, wherein the kit further comprises written instructions for using the antibody or antigen-binding fragment thereof, immunoconjugate, multi-specific molecule, or composition for treating or ameliorating a disease or disorder in a subject.

J1. In certain non-limiting embodiments, the presently disclosed subject matter provides the antibody or antigen-binding fragment thereof of any one of claims A1-A27, the immunoconjugate of any one of C1-C8, the multi-specific molecule of E1 or E2, or the composition of any one of claims B1, B2, D1, D2, F1, and F2 for use in treating or ameliorating a disease or disorder associated with DLL3 in a subject.

J2. The foregoing antibody or antigen-binding fragment thereof, the foregoing immunoconjugate, the multi-specific molecule, or the foregoing composition for use in J1, wherein the disease or disorder is a tumor.

J3. The foregoing antibody or antigen-binding fragment thereof, the foregoing immunoconjugate, the foregoing multi-specific molecule, or the foregoing composition for use in J2, wherein the tumor is cancer.

J4. The foregoing antibody or antigen-binding fragment thereof, the foregoing immunoconjugate, the foregoing multi-specific molecule, or the foregoing composition for use in any one of J1-J3, wherein the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma.

J5. The foregoing antibody or antigen-binding fragment thereof, the foregoing immunoconjugate, the foregoing multi-specific molecule, or the foregoing composition for use in J4, wherein the neuroendocrine tumors of the lung are selected from the group consisting of pulmonary neuroendocrine cancer, large cell neuroendocrine carcinoma, and small-cell lung cancer.

J6. The foregoing antibody or antigen-binding fragment thereof, the foregoing immunoconjugate, the foregoing multi-specific molecule, or the foregoing composition for use in any one of J1-J5, wherein the subject is human.

K1. In certain non-limiting embodiments, the presently disclosed subject matter provides an immunoconjugate comprising an anti-DLL3 antibody or antigen-binding fragment thereof linked to a therapeutic agent, wherein the anti-DLL3 antibody or antigen-binding fragment thereof comprises a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 187, SEQ ID NO: 197, SEQ ID NO: 204, or SEQ ID NO: 212.

K2. In certain non-limiting embodiments, the presently disclosed subject matter provides an immunoconjugate comprising an anti-DLL3 antibody or antigen-binding fragment thereof linked to a therapeutic agent, wherein the anti-DLL3 antibody or antigen-binding fragment thereof comprises a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 188, SEQ ID NO: 198, SEQ ID NO: 205, or SEQ ID NO: 213.

K3. In certain non-limiting embodiments, the presently disclosed subject matter provides an immunoconjugate comprising an anti-DLL3 antibody or antigen-binding fragment thereof linked to a therapeutic agent, wherein the anti-DLL3 antibody or antigen-binding fragment thereof comprises:
a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 187, SEQ ID NO: 197, SEQ ID NO: 204, or SEQ ID NO: 212; and
b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 188, SEQ ID NO: 198, SEQ ID NO: 205, or SEQ ID NO: 213.

K4. In certain non-limiting embodiments, the presently disclosed subject matter provides an immunoconjugate comprising an anti-DLL3 antibody or antigen-binding fragment thereof linked to a therapeutic agent, wherein the anti-DLL3 antibody or antigen-binding fragment thereof comprises a heavy chain variable region and a light chain variable region selected from the group consisting of:
a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 187, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 188;
b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 197, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 198;
c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 204, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 205; and
d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 212, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 213.

K5. In certain non-limiting embodiments, the presently disclosed subject matter provides an immunoconjugate comprising an anti-DLL3 antibody or antigen-binding fragment thereof linked to a therapeutic agent, wherein the anti-DLL3 antibody or antigen-binding fragment thereof comprises a heavy chain variable region comprising the amino acid sequence set forth in SEQ ID NO: 187, SEQ ID NO: 197, SEQ ID NO: 204, or SEQ ID NO: 212.

K6. In certain non-limiting embodiments, the presently disclosed subject matter provides an immunoconjugate comprising an anti-DLL3 antibody or antigen-binding fragment thereof linked to a therapeutic agent, wherein the anti-DLL3 antibody or antigen-binding fragment thereof comprises a light chain variable region comprising the amino acid sequence set forth in SEQ ID NO: 188, SEQ ID NO: 198, SEQ ID NO: 205, or SEQ ID NO: 213.

K7. In certain non-limiting embodiments, the presently disclosed subject matter provides an immunoconjugate comprising an anti-DLL3 antibody or antigen-binding fragment thereof linked to a therapeutic agent, wherein the anti-DLL3 antibody or antigen-binding fragment thereof comprises:
a) a heavy chain variable region comprising the amino acid sequence set forth in SEQ ID NO: 187, SEQ ID NO: 197, SEQ ID NO: 204, or SEQ ID NO: 212; and
b) a light chain variable region comprising the amino acid sequence set forth in SEQ ID NO: 188, SEQ ID NO: 198, SEQ ID NO: 205, or SEQ ID NO: 213.

K8. The foregoing immunoconjugate of any one of claims K1-K7, wherein
a) a heavy chain variable region comprising the amino acid sequence set forth in SEQ ID NO: 187, and a light chain variable region comprising the amino acid sequence set forth in SEQ ID NO: 188;
b) a heavy chain variable region comprising the amino acid sequence set forth in SEQ ID NO: 197, and a light chain variable region comprising the amino acid sequence set forth in SEQ ID NO: 198;
c) a heavy chain variable region comprising the amino acid sequence set forth in SEQ ID NO: 204, and a light chain variable region comprising the amino acid sequence set forth in SEQ ID NO: 205; and
d) a heavy chain variable region comprising the amino acid sequence set forth in SEQ ID NO: 212, and a light chain variable region comprising the amino acid sequence set forth in SEQ ID NO: 213.

K9. In certain non-limiting embodiments, the presently disclosed subject matter provides an immunoconjugate comprising an anti-DLL3 antibody or antigen-binding fragment thereof linked to a therapeutic agent, wherein the anti-DLL3 antibody or antigen-binding fragment thereof comprises a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
a) a heavy chain variable region CDR3 comprising the amino acid sequence set forth in SEQ ID NO: 184 and a conservative modification thereof; and a light chain variable region CDR3 comprising the amino acid sequence set forth in SEQ ID NO: 186 and a conservative modification thereof;
b) a heavy chain variable region CDR3 comprising the amino acid sequence set forth in SEQ ID NO: 193 and a conservative modification thereof; and a light chain variable region CDR3 comprising the amino acid sequence set forth in SEQ ID NO: 196 and a conservative modification thereof;
c) a heavy chain variable region CDR3 comprising the amino acid sequence set forth in SEQ ID NO: 202 and a conservative modification thereof; and a light chain variable region CDR3 comprising the amino acid sequence set forth in SEQ ID NO: 203 and a conservative modification thereof; and
d) a heavy chain variable region CDR3 comprising the amino acid sequence set forth in SEQ ID NO: 210 and a conservative modification thereof; and a light chain variable region CDR3 comprising the amino acid sequence set forth in SEQ ID NO: 211 and a conservative modification thereof.

K10. The foregoing immunoconjugate of K9, wherein the heavy chain variable region and light chain variable region CDR2 domains are selected from the group consisting of:
a) a heavy chain variable region CDR2 comprising the amino acid sequence set forth in SEQ ID NO: 183 and a conservative modification thereof; and a light chain variable region CDR2 comprising the amino acid sequence set forth in SEQ ID NO: 50 and a conservative modification thereof;
b) a heavy chain variable region CDR2 comprising the amino acid sequence set forth in SEQ ID NO: 192 and a conservative modification thereof; and a light chain variable region CDR2 comprising the amino acid sequence set forth in SEQ ID NO: 195 and a conservative modification thereof;
c) a heavy chain variable region CDR2 comprising the amino acid sequence set forth in SEQ ID NO: 201 and a conservative modification thereof; and a light chain variable region CDR2 comprising the amino acid sequence set forth in SEQ ID NO: 5 and a conservative modification thereof; and
d) a heavy chain variable region CDR2 comprising the amino acid sequence set forth in SEQ ID NO: 209 and a conservative modification thereof; and a light chain variable region CDR2 comprising the amino acid sequence set forth in SEQ ID NO: 58 and a conservative modification thereof.

K11. The foregoing immunoconjugate of K9 or K10, wherein the heavy chain variable region and light chain variable region CDR1 domains are selected from the group consisting of:
a) a heavy chain variable region CDR1 comprising the amino acid sequence set forth in SEQ ID NO: 21 and a conservative modification thereof; and a light chain variable region CDR1 comprising the amino acid sequence set forth in SEQ ID NO: 185 and a conservative modification thereof;
b) a heavy chain variable region CDR1 comprising the amino acid sequence set forth in SEQ ID NO: 191 and a conservative modification thereof; and a light chain variable region CDR1 comprising the amino acid sequence set forth in SEQ ID NO: 194 and a conservative modification thereof;
c) a heavy chain variable region CDR1 comprising the amino acid sequence set forth in SEQ ID NO: 11 and a conservative modification thereof; and a light chain variable region CDR1 comprising the amino acid sequence set forth in SEQ ID NO: 4 and a conservative modification thereof; and
d) a heavy chain variable region CDR1 comprising the amino acid sequence set forth in SEQ ID NO: 208 and a conservative modification thereof; and a light chain variable region CDR1 comprising the amino acid sequence set forth in SEQ ID NO: 57 and a conservative modification thereof.

K12. The foregoing immunoconjugate of any one of claims K9-K11, wherein one or more of the CDR sequences have up to about 5 amino acid substitutions.

K13. The foregoing immunoconjugate of any one of claims K9-K11, wherein one or more of the CDR sequences have up to about 3 amino acid substitutions.

K14. In certain non-limiting embodiments, the presently disclosed subject matter provides an immunoconjugate comprising an anti-DLL3 antibody or antigen-binding fragment thereof linked to a therapeutic agent, wherein the anti-DLL3 antibody or antigen-binding fragment thereof comprises:
a) a heavy chain variable region comprising a CDR1 comprising the amino acid sequence set forth in SEQ ID NO: 21, a CDR2 comprising the amino acid sequence set forth in SEQ ID NO: 183, and a CDR3 comprising the amino acid sequence set forth in SEQ ID NO: 184;
b) a heavy chain variable region comprising a CDR1 comprising the amino acid sequence set forth in SEQ ID NO: 191, a CDR2 comprising the amino acid sequence set forth in SEQ ID NO: 192, and a CDR3 comprising the amino acid sequence set forth in SEQ ID NO: 193;
c) a heavy chain variable region comprising a CDR1 comprising the amino acid sequence set forth in SEQ ID NO: 11, a CDR2 comprising the amino acid sequence set forth in SEQ ID NO: 201, and a CDR3 comprising the amino acid sequence set forth in SEQ ID NO: 202; or
d) a heavy chain variable region comprising a CDR1 comprising the amino acid sequence set forth in SEQ ID NO: 208, a CDR2 comprising the amino acid sequence set forth in SEQ ID NO: 209, and a CDR3 comprising the amino acid sequence set forth in SEQ ID NO: 210.

76. An immunoconjugate comprising an anti-DLL3 antibody or antigen-binding fragment thereof linked to a therapeutic agent, wherein the anti-DLL3 antibody or antigen-binding fragment thereof comprises:
a) a light chain variable region comprising a CDR1 comprising the amino acid sequence set forth in SEQ ID NO: 185, a CDR2 comprising the amino acid sequence set forth in SEQ ID NO: 50, and a CDR3 comprising the amino acid sequence set forth in SEQ ID NO: 186;
b) a light chain variable region comprising a CDR1 comprising the amino acid sequence set forth in SEQ ID NO: 194, a CDR2 comprising the amino acid sequence set forth in SEQ ID NO: 195, and a CDR3 comprising the amino acid sequence set forth in SEQ ID NO: 196;
c) a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 203; or
d) a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 211.

K15. In certain non-limiting embodiments, the presently disclosed subject matter provides an immunoconjugate comprising an anti-DLL3 antibody or antigen-binding fragment thereof linked to a therapeutic agent, wherein the anti-DLL3 antibody or antigen-binding fragment thereof comprises:
a) a heavy chain variable region comprising a CDR1 comprising the amino acid sequence set forth in SEQ ID NO: 21, a CDR2 comprising the amino acid sequence set forth in SEQ ID NO: 183, and a CDR3 comprising the amino acid sequence set forth in SEQ ID NO: 184; and a light chain variable region comprising a CDR1 comprising the amino acid sequence set forth in SEQ ID NO: 185, a CDR2 comprising the amino acid sequence set forth in SEQ ID NO: 50, and a CDR3 comprising the amino acid sequence set forth in SEQ ID NO: 186;
b) a heavy chain variable region comprising a CDR1 comprising the amino acid sequence set forth in SEQ ID NO: 191, a CDR2 comprising the amino acid sequence set forth in SEQ ID NO: 192, and a CDR3 comprising the amino acid sequence set forth in SEQ ID NO: 193; and a light chain variable region comprising a CDR1 comprising the amino acid sequence set forth in SEQ ID NO: 194, a CDR2 comprising the amino acid sequence set forth in SEQ ID NO: 195, and a CDR3 comprising the amino acid sequence set forth in SEQ ID NO: 196;
c) a heavy chain variable region comprising a CDR1 comprising the amino acid sequence set forth in SEQ ID NO: 11, a CDR2 comprising the amino acid sequence set forth in SEQ ID NO: 201, and a CDR3 comprising the amino acid sequence set forth in SEQ ID NO: 202; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 203; or
d) a heavy chain variable region comprising a CDR1 comprising the amino acid sequence set forth in SEQ ID NO: 208, a CDR2 comprising the amino acid sequence set forth in SEQ ID NO: 209, and a CDR3 comprising the amino acid sequence set forth in SEQ ID NO: 210; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 211.

K16. The foregoing immunoconjugate of any one of claims K1-K15, wherein the antibody or antigen-binding fragment thereof binds to a DLL3 comprising the amino acid sequence set forth in SEQ ID NO: 215 or a fragment thereof.

K17. The foregoing immunoconjugate of any one of K1-K16, wherein the antibody comprises a human variable region framework region.

K18. The foregoing immunoconjugate of any one of K1-K17, wherein the antibody or antigen-binding fragment thereof is a fully human or an antigen-binding fragment thereof.

K19. The foregoing immunoconjugate of any one of K1-K17, wherein the antibody or antigen-binding fragment thereof is a chimeric antibody or an antigen-binding fragment thereof.

K20. The foregoing immunoconjugate of any one of K1-K17, wherein the antibody or antigen-binding fragment thereof is a humanized antibody or an antigen-binding fragment thereof.

K21. The foregoing immunoconjugate of any one of K1-K₂O, wherein the antigen-binding fragment is a Fab, Fab′, F(ab′)2, variable fragment (Fv), or single chain variable region (scFv).

K22. The foregoing immunoconjugate of K21, wherein the antigen antigen-binding fragment is an scFv.

K23. The foregoing immunoconjugate of claim any one of K1-K23, wherein the therapeutic agent is a radioactive isotope.

K24. The foregoing immunoconjugate of any one of K1-K23 further comprising a chelator.

K25. The foregoing immunoconjugate of K24, wherein the chelator is selected from the group consisting of AAZTA, BAT, BARAC, BPCA, TE2A, CB-TE2A, CB0TE1A1P, CB-TE2P, MM-TE2A, DM TE-2A, CP356, DATA, DBCO, DiAmSar, DIBO, DIMA, DFO, DGO, DOTA, DOTMA, DTPA, EDTA, EGTA, EHPG, H2dedpa, H4octapa, H2azapa, H5decapa, H6phospa, HBED, SHBED, HEHA, HYNIC, LICAM, MECAM, NODASA, NODAGA, NOPO, NOTA, NETA, PEPA, PCTA, PDTA, TACN-TM, TCMC, TETA, TETMA, TRAP (PRP9), TRITA, TTHA, and derivatives thereof.

K26. The foregoing immunoconjugate of K24 or K25, wherein the chelator is DFO.

K27. The foregoing immunoconjugate of any one of K23-K26, wherein the radioactive isotope is selected from the group consisting of ⁴⁷Sc, ⁶⁷Cu, ⁹⁰Y, ¹³¹I, ¹⁴⁹Tb, ¹⁶¹Tb, ¹⁷⁷Lu, ²²⁵Ac, ²¹³Bi, ²²³Ra, ⁸⁹Zr, and ²²⁷Th.

K28. The foregoing immunoconjugate of K27, wherein the radioactive isotope is ¹⁷⁷Lu.

K29. The foregoing immunoconjugate of K27, wherein the radioactive isotope is ⁸⁹Zr.

L1. In certain non-limiting embodiments, the presently disclosed subject matter provides a composition comprising the immunoconjugate of any one of claims 62-91.

L2. The composition of claim 92, which is a pharmaceutical composition that further comprises a pharmaceutically acceptable carrier.

M1. A method for detecting DLL3 in a whole cell, a tissue, or a blood sample, comprising:
a) contacting a cell, tissue or blood sample with the immunoconjugate of any one of K1-K29; and
b) determining the amount of the immunoconjugate bound to the cell, tissue or blood sample by measuring the amount of detectable label associated with said cell or tissue, wherein the amount of bound immunoconjugate indicates the amount of DLL3 in the cell, tissue or blood sample.

N1. In certain non-limiting embodiments, the presently disclosed subject matter provides a method of treating or ameliorating a disease or disorder associated with DLL3 in a subject, comprising administering to the subject the immunoconjugate of any one of K1-K29 or the composition of L1 or L2.

N2. The foregoing method of N1, wherein the disease or disorder is a tumor.

N3. The foregoing method of N2, wherein the tumor is cancer.

N4. The foregoing method of any one of N1-N3, wherein the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma.

N5. The foregoing method of N4, wherein the neuroendocrine tumors of the lung are selected from the group consisting of pulmonary neuroendocrine cancer, large cell neuroendocrine carcinoma, and small-cell lung cancer.

N6. The foregoing method of any one of N1-N5, wherein the subject is a human.

O1. In certain non-limiting embodiments, the presently disclosed subject matter provides a kit for treating or ameliorating a disease or disorder in a subject, comprising the immunoconjugate of any one of K1-K29 or the composition of L1 or L2.

O2. The foregoing kit of 01, wherein the kit further comprises written instructions for using the antibody or antigen-binding fragment thereof, immunoconjugate, multi-specific molecule, or composition for treating or ameliorating a disease or disorder in a subject.

P1. The foregoing immunoconjugate of any one of K1-K29 or the foregoing composition of L1 or L2 for use in treating or ameliorating a disease or disorder associated with DLL3 in a subject.

P2. The foregoing immunoconjugate or the foregoing composition for use in P1, wherein the disease or disorder is a tumor.

P3. The foregoing immunoconjugate or the foregoing composition for use in P2, wherein the tumor is cancer.

P4. The foregoing immunoconjugate or the foregoing composition for use in any one of P1-P3, wherein the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma.

P5. The foregoing immunoconjugate or the foregoing composition for use in P4, wherein the neuroendocrine tumors of the lung are selected from the group consisting of pulmonary neuroendocrine cancer, large cell neuroendocrine carcinoma, and small-cell lung cancer.

P6. The foregoing immunoconjugate or the foregoing composition for use in any one of P1-P5, wherein the subject is human.

### 6. EXAMPLES

The following examples are put forth so as to provide those of ordinary skill in the art with a complete disclosure and description of how to make and use the antibodies, multi-specific antibodies, compositions comprising thereof, screening, and therapeutic methods of the presently disclosed subject matter, and are not intended to limit the scope of what the inventors regard as their presently disclosed subject matter. It is understood that various other embodiments may be practiced, given the general description provided above.

### 6.1 Example 1—Generation of the Presently Disclosed Anti-DLL3 Antibodies and Scfvs

The extracellular domain (ECD) of DLL3 (GenBank accession number Q9NY J7-1) corresponding to amino acids Ala27-Ala479 with a C-terminal 6×His tag produced in HEK293T cells stably expressing full length DLL3 were used as immunogens. Ablexis AlivaMAb Kappa Mice (Ablexis, San Diego, CA) harboring a human immunoglobulin repertoire were immunized either with soluble DLL3-ECD following standard immunization techniques over a period of 3 weeks. Splenocytes and draining lymph node cells from mice with high serum titers specific for DLL3 were harvested and fused with mouse myeloma cells to generate hybridomas using electrofusion. These hybridomas were then screened to identify the presence of antibodies that bound specifically to soluble DLL3-ECD by ELISA and full-length DLL3 protein on stably expressing 293 cells by flow cytometry versus parental 293 cells. In addition, these hybridomas were screened for reactivity with HEK293 cells stably expressing cynomolgus DLL3 or mouse DLL3. Hybridomas were selected for further investigation by ranking in flow cytometry for staining intensity on 293 DLL3 transfectants along with 4° C./37° C. staining to evaluate the internalization of the antibodies.

### 6.2 Example 2—Epitope Binning of Panel of the Presently Disclosed Anti-DLL3 Monoclonal Antibodies

The panel of purified anti-DLL3 monoclonal antibodies and the reference monoclonal antibody were subjected to pairwise epitope binning on a Carterra® array surface plasmon resonance (SPR) assay platform (Carterra*Inc., Salt Lake City UT) where each monoclonal antibody was tested for the capture of Histidine-tagged DLL3 antigen (DLL3-His), and also for competing with every other antibody in the panel for the binding to DLL3-His. The antibodies were immobilized on a HC200M chip (ligand) through standard amine coupling techniques by the print array method. Then in each cycle antigen was injected across the entire array followed by a single antibody (analyte). At the end of each cycle the surface was regenerated to remove antigen and analyte before a new cycle started. As shown in the Table 25 below. These results demonstrate that the anti-DLL3-antibodies disclosed herein bind to distinct epitopes present in DLL3 protein. Accordingly, the anti-DLL3-antibodies disclosed herein can be used in combination with each other for delivering multiple therapeutic agents to tumor cells expressing DLL3

TABLE 25

| Monoclonal Antibodies | Epitope Bin |
|---|---|
| E23 | 7 |
| F9 | 4 |
| E9 | 4 |
| B22 | 2 |
| N12 | 8 |
| N10 | 7 |
| B16-v1 | 4 |
| B16-v2 | 4 |
| L22 | 8 |
| A18 | 6 |
| D8 | 2 |
| G16 | 4 |
| K19 | 5 |
| G3 | 1 |
| J23 | 2 |
| J8 | 4 |
| L12 | 4 |
| B2 | 4 |
| C22 | 9 |
| F21 | 5 |
| O24 | 4 |
| M11 | 2 |
| P4 | 5 |

### 6.3 Example 3—Binding Affinities of the Presently Disclosed Anti-DLL3 Antibodies

Binding affinities of the anti-DDL3 antibodies disclosed herein were determined by biolayer interferometry (BLI) using the Octet HTX instrument at 25° C. using PBS 0.1% BSA 0.02% Tween 20 as the binding buffer and 10 mM Glycine pH 1.7 as the regeneration buffer. The 23 purified monoclonal antibodies (5 μg/mL each) were loaded onto anti-mouse Fc sensors. Loaded sensors were dipped into a serial dilutions of Recombinant Human DLL3 Protein, (amino acids Ala27-Ala479, cat #9749-DL, R&D Systems) at a 200 nM starting concentration, with 7 serial 1:3 dilutions. The binding affinities of all the monoclonal antibodies are shown in Table 26 below.

These results demonstrate that the anti-DLL3 antibodies disclosed herein specifically bind DLL3 with high affinity.

TABLE 26

| Monoclonal antibodies | Kd (nM) |
|---|---|
| E23 | 0.511 |
| F9 | 3.010 |
| E9 | 0.300 |
| B22 | 1.840 |
| N12 | 0.467 |
| N10 | 1.940 |
| L22 | 3.550 |
| A18 | 0.236 |
| D8 | 0.438 |
| G16 | 4.880 |
| K19 | 1.890 |
| G3 | 0.908 |
| J23 | 0.998 |
| J8 | 0.001 |
| L12 | 3.100 |
| B2 | 1.460 |
| C22 | 0.431 |
| F21 | 6.120 |
| O24 | 1.280 |
| M11 | 2.760 |
| P4 | 6.500 |

Next, affinity, binding domain, and specificity of antibodies including scFv designated as J8, B2, and L22 were analyzed. Affinity, binding domain and specificity of novel human anti-DLL3 antibodies are depicted in Table 30 below.

TABLE 30

| | Affinity | | Binding | ELISA | ELISA | ELISA |
|---|---|---|---|---|---|---|
| Name | hDLL3 | | domain | hDLL3 | hDLL1 | hDLL4 |
| B2 | 3.55 | nM | EGF-like | 1.205 | 0.4 | 0.043 |
| | | | 5 and | | | |
| | | | EGF-like | | | |
| | | | 6 | | | |
| J8 | <0.001 | nM | EGF-like | 1.377 | 0.095 | 0.085 |
| | | | 5 and | | | |
| | | | EGF-like | | | |
| | | | 6 | | | |
| L22 | 1.46 | nM | EGF-like | 1.639 | 0.105 | 0.056 |
| | | | 3 and | | | |
| | | | EGF-like | | | |
| | | | 4 | | | |

### 6.4 Example 4—Screening of Novel Anti-DLL3 Antibodies E23 and C8 for Imaging and Radioimmunotherapy

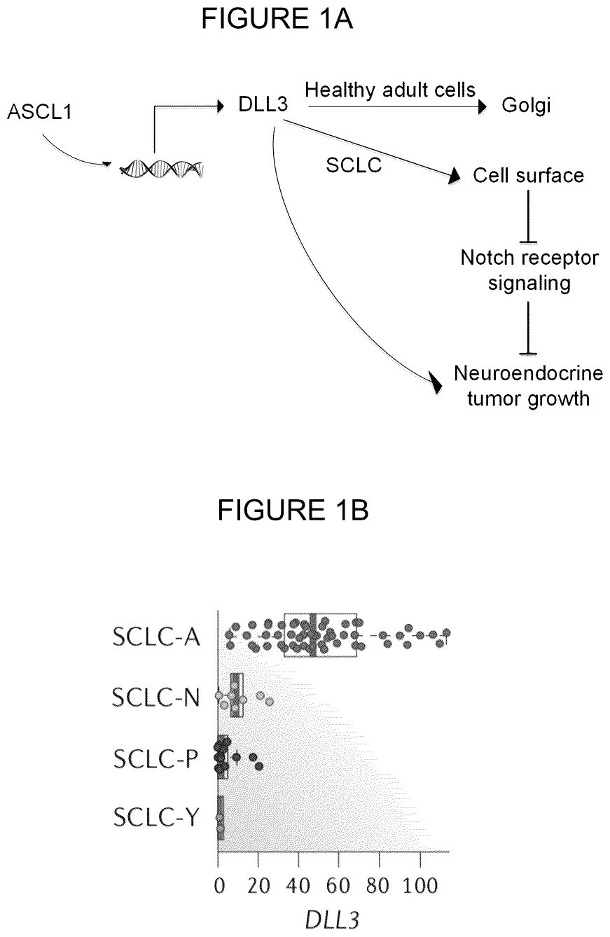

Small cell lung cancer (SCLC) is a particularly lethal form of lung cancer, accounting for roughly 15% of new lung cancer cases (Saunders et al., Sci Transl Med 2015; 7(302):302ra136). Delta-like ligand 3 (DLL3) has recently emerged as a therapeutic target in SCLC, in part because DLL3 cell surface expression is restricted almost exclusively to malignant tissue. As illustrated in FIGS. 1A and 1B, DLL3 is known to be downstream of the transcription factor ASCL1, which is incredibly important in small cell lung cancer biology. Notably, DLL3 is an excellent target for antibody-based therapeutics because in healthy adult cells it is localized intracellularly within the Golgi apparatus, while in SCLC is markedly overexpressed and aberrantly trafficked to the cell surface. Recent studies have determined that the role of DLL3 in SCLC is to inhibit Notch receptor signaling. Since Notch signaling inhibits neuroendocrine tumor growth, the net effect of DLL3 overexpression is promotion of neuroendocrine tumor growth (Saunders et al., Science translational medicine 7, no. 302 (2015); Rudin et al., Nature Reviews Cancer 19, no. 5 (2019): 289-297; Yan et al., Oncology Letters 18, no. 3 (2019): 2254-2261; and Furuta et al., Cancer science 110, no. 5 (2019): 1599-1608).

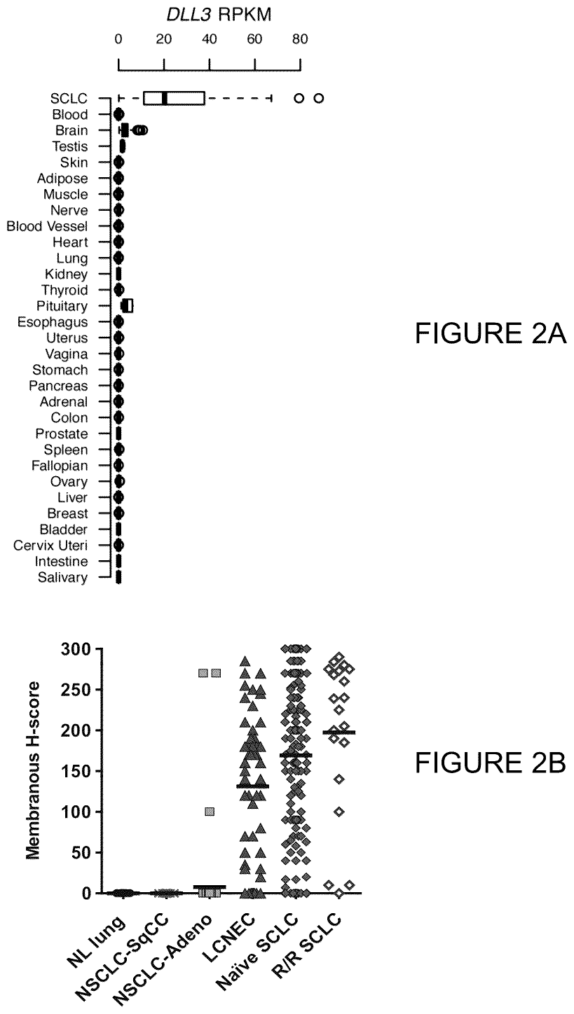

The presently disclosed subject matter is focused on DLL3 as a target for antibody-based imaging and therapies, which rely on high tumor to tissue ratios of membrane expression. As shown in FIGS. 2A and 2B, RNAseq data indicated that DLL3 is not expressed in healthy adult tissue and that and when overexpressed in small cell, its membrane H-score is significantly higher.

There are currently available two main different DLL3-targeted treatment options that have been clinically tested: i) Stemcentryx, which is an antibody-drug conjugate Rovalpituzumab Tesirine (RovaT), and ii) AMG 119, which is a bispecific T cell engager used in combination with a CAR T cell against DLL3. Importantly, RovaT failed clinical trials due to dose-limiting toxicities associated with the warhead, while the phase 1 clinical data for AMG119 have not yet been published.

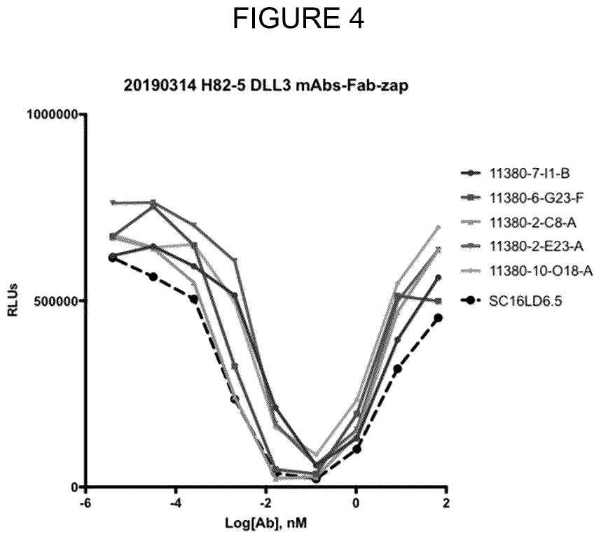

In order to generate high-affinity anti-DLL3 antibodies with high internalization for direct targeting approach and therapy, anti-DLL3 antibodies were subjected to several internalization assays including the Fab-Zap assay for determining killing abilities. As shown in FIG. 4 and in the table below, several antibodies showed internalization profile similar to Rovalpituzumab Tesirine (SC16LD6.5), which was used as control.

TABLE 28

| Well ID | Percent Max Killing |
|---|---|
| 1-N10-B (10 ug/ml) | 74.0 |
| 2-B16-A (10 ug/ml) | 80.5 |
| 2-E23-A (10 ug/ml) | 88.7 |
| 2-F9-C (10 ug/ml) | 70.0 |
| 5-B17-D (10 ug/ml) | 1.0 |
| 5-B22-A (10 ug/ml) | 61.9 |
| 5-E9-C (10 ug/ml) | 61.4 |
| 6-G23-F (10 ug/ml) | 94.0 |
| 7-I1-B (10 ug/ml) | 97.0 |
| 9-N12-F (10 ug/ml) | 88.0 |
| 10-G3-A (10 ug/ml) | 86.0 |
| 10-O18-A (10 ug/ml) | 88.0 |
| 1-A18-C (10 ug/ml) | 67.0 |
| 1-K19-D (10 ug/ml) | 71.0 |
| 2-C8-A (10 ug/ml) | 89.0 |
| 6-D8-D (10 ug/ml) | 68.0 |
| 8-G16-C (10 ug/ml) | 46.0 |
| 9-L22-A (10 ug/ml) | 82.0 |
| 10-M11-A (10 ug/ml) | 65.2 |
| 10-O24-D (10 ug/ml) | 80.3 |
| 10-P4-E (10 ug/ml) | 68.0 |
| 1-J23-A (10 ug/ml) | 59.5 |
| 2-J8-A (10 ug/ml) | 64.0 |
| 2-L12-A (10 ug/ml) | 61.0 |
| 3-B2-A (10 ug/ml) | 71.4 |
| 6-C22-A (10 ug/ml) | 69.1 |
| 9-F21-A (10 ug/ml) | 20.0 |
| SC16LD6.5 (10 ug/ml) | 96.0 |

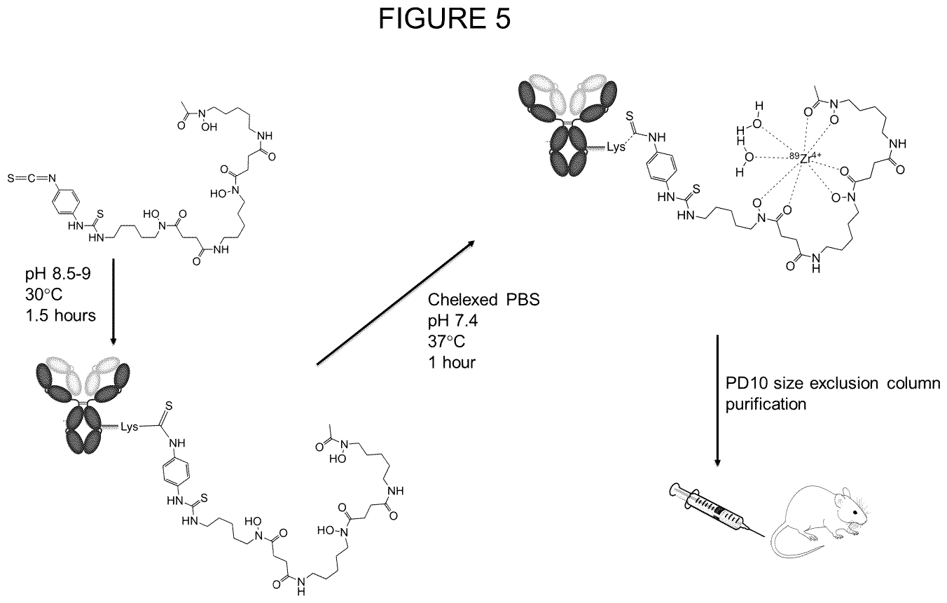

The first candidate to be tested was E23. FIG. 5 shows that a DFO chelator containing an isothiocyanate group was combined with the antibody at a pH of 8.5-9.0. The mixture was then heated to 30° C. for 1.5 h. To the reaction, Zr-89 and chelexed PBS were added and the resulting mixture was heated to 37° C. for 1 h at a pH of 7.4. The desired Zr-89 radiolabeled antibody was then isolated using a PD10 size exclusion column.

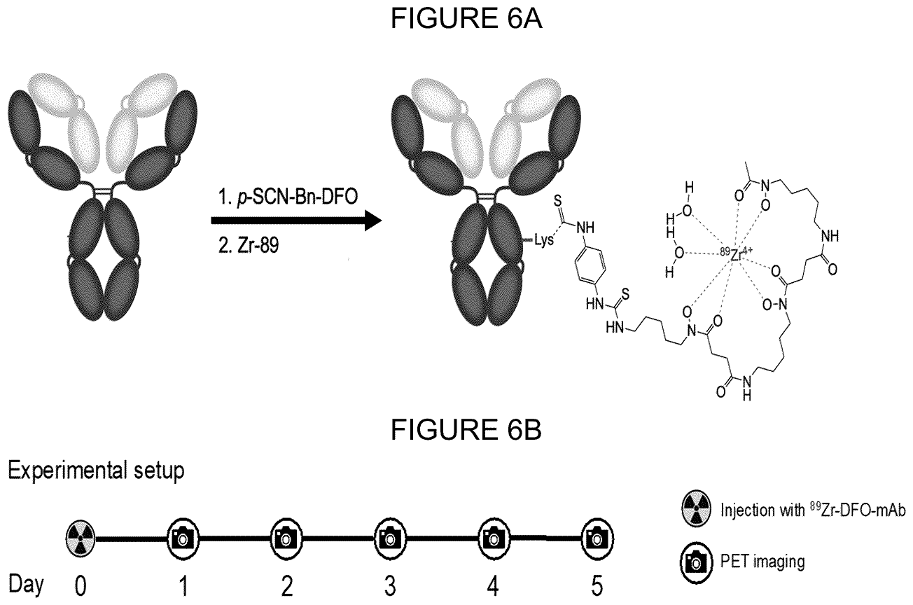

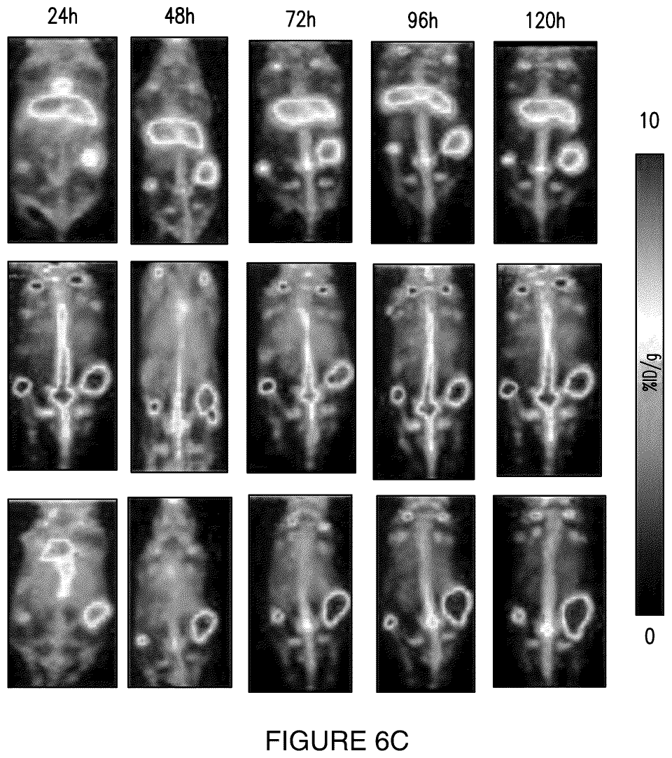

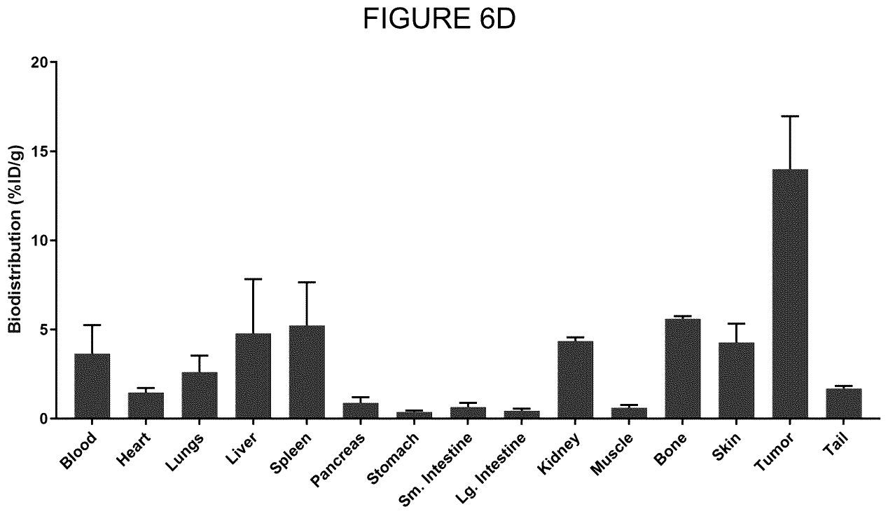

FIG. 6A shows that E23bivalent antibody was bioconjugated to p-SCN-Bn-DFO via lysine residues on the mAb, then radiolabeled with Zr-89. Extensive quality control assays showed that the antibody was stable in human serum over time and radiolabeled. To determine the properties in vivo, mice bearing subcutaneous H82 (SCLC) or H660 (NEPC) xenografts were injected with ⁸⁹Zr-DFO-E23biv radioconjugate and imaged at 24, 48, 72, 96, and 120h post-injection (FIG. 6B). As illustrated in FIGS. 6C and 6D, the tumor uptake was observed in all mice with some blood and bone uptake present as well. Further, although bone uptake was high, tumor uptake was significantly increased as compared to other tissues.

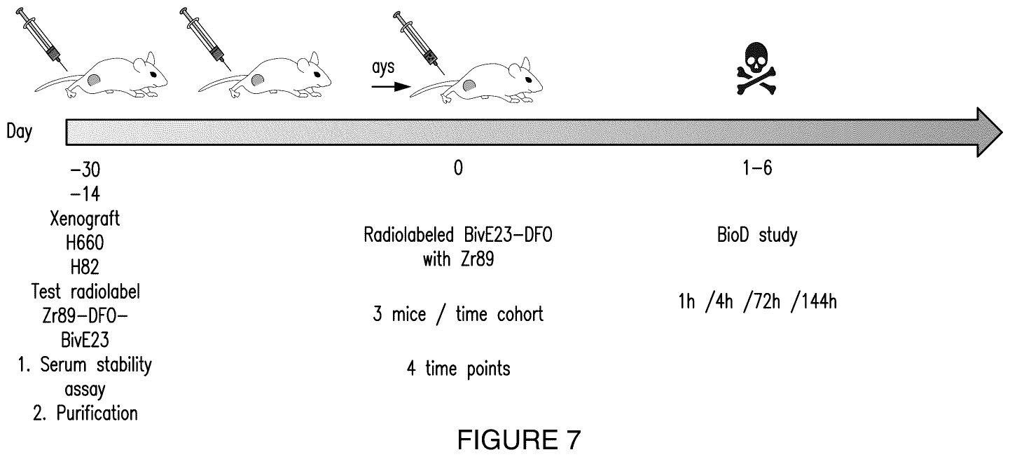

After the initial study, a full length biodistribution study with 4 time points was performed in order to evaluate the potential toxicity profile of the E23 clone. E23bivalent was bioconjugated to p-SCN-Bn-DFO via lysine residues on the mAb, then radiolabeled with Zr-89. Next, mice bearing subcutaneous H82 (SCLC) and H660 (NEPC) xenografts were injected with ⁸⁹Zr-DFO-E23biv radioconjugate and sacrificed at 1h, 4h, 72h, and 144h post-injection at which time, the most relevant organs were analyzed (FIG. 7). The dosages used was 2 μg and 20 μCi in the H660 model.

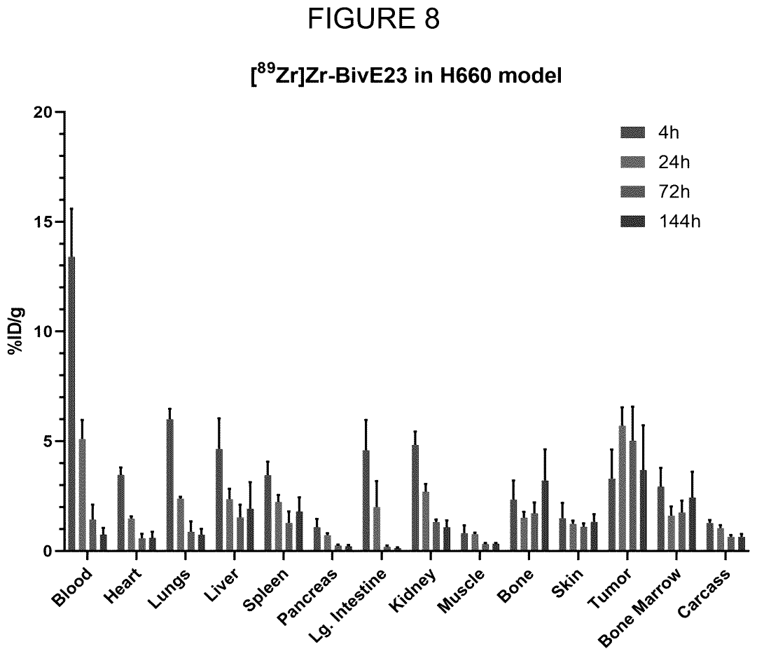

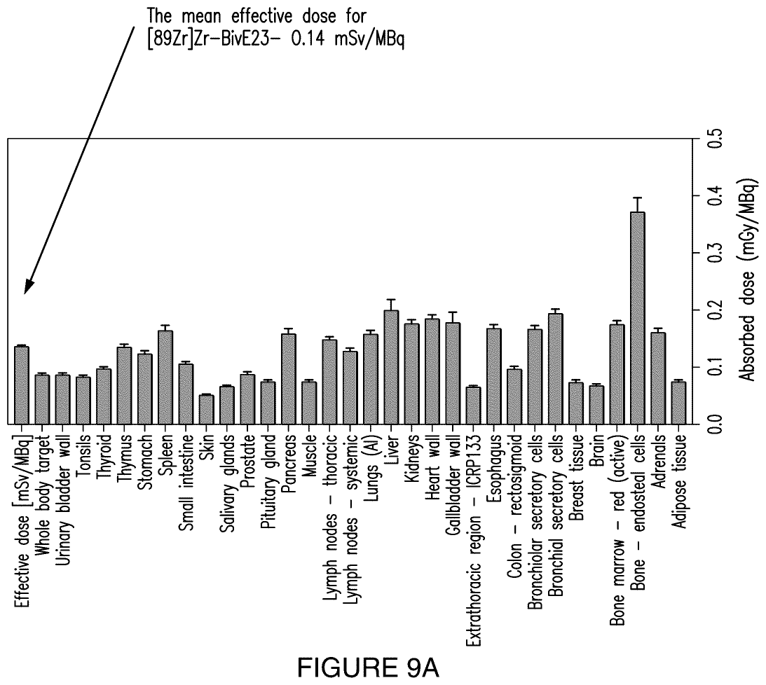

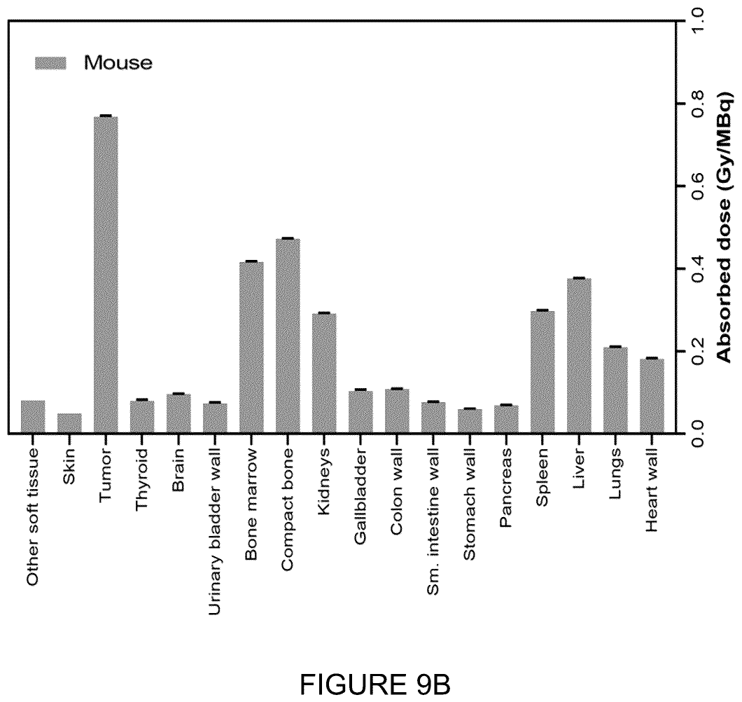

The results showed that the tumor uptake for the H600 NEPC tumor model was around 5% Injected dose/gram, indicating that the E23 cleared out rapidly from most non-tumor organs (FIG. 8). Further, the E23 dosimetry analysis extrapolated to humans also showed that, compared to other antibodies radiolabeled with Zr89, the E23 showed a low mean effective dose (FIGS. 9A and 9B). These results indicate that E23 does not cause serious harm to the entire body since it is comparable to other FDA-approved antibodies (e.g., [⁸⁹Zr]Zr-Trastuzumab=0.48 mSv/MBq; [⁸⁹Zr]Zr-HJ591=0.36 mSv/MBq; [⁸⁹Zr]Zr-IAB2 minibody=0.41 mSv/MBq; and [89Zr]Zr-Cetuximab=0.41 mSv/MBq). In addition, the E23 dosimetry analysis extrapolated to the therapeutic radioisotope Lu177 in male mice showed that the therapeutic index (TI) was around 2 (TI=2) for liver and bone marrow, which are the two most important dose-limiting organs (FIGS. 9A and 9B). To compare, SC16 had a TI of around 4 for Liver and even higher for bone marrow.

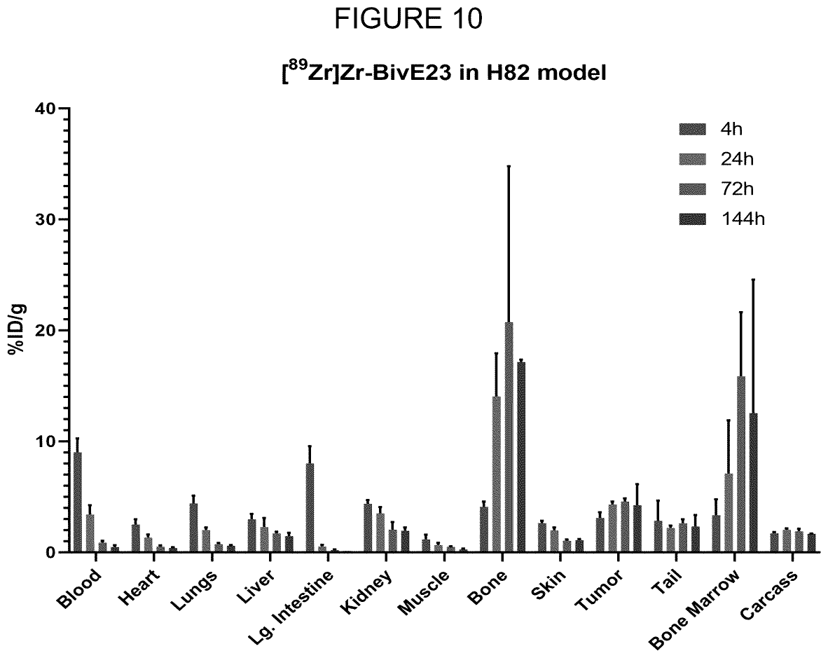

Next, the same experiments were performed in the H82 tumor model using 5 μg and 50 μCi/mouse. However, biodistribution results indicated accumulation in bone (FIG. 10). One possible explanation for this data is that the chelator may have fallen off the antibody causing the Zr89 radioisotope to accumulate into the bone. Overall, E23 can be used as an imaging agent, while the therapeutic index showed a limitation for its usage as a therapeutic agent.

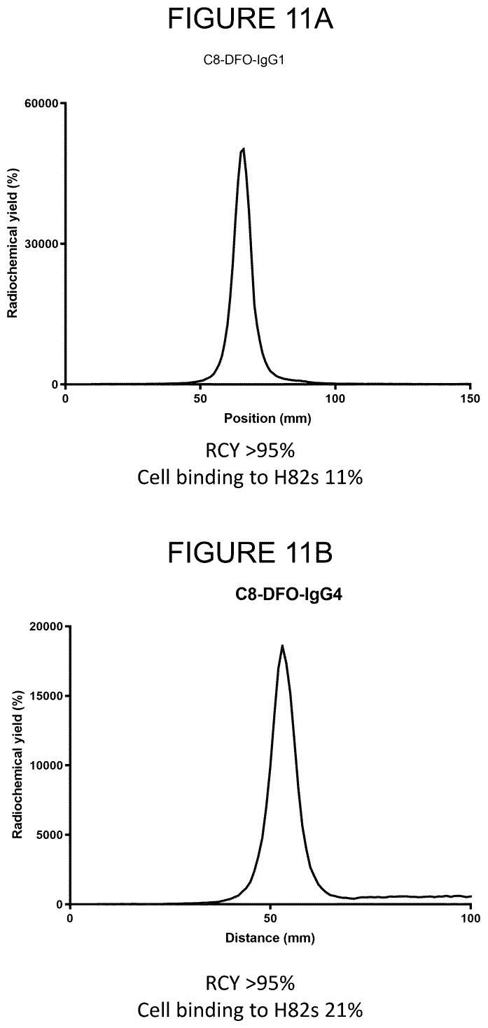

Next, another candidate (C8) was tested. C8 was prepared as IgG1 and IgG4. Quality control analysis showed that both C8 IgG1 and C8 IgG4 had good binding to H82 tumor cells (FIGS. 11A and 11B).

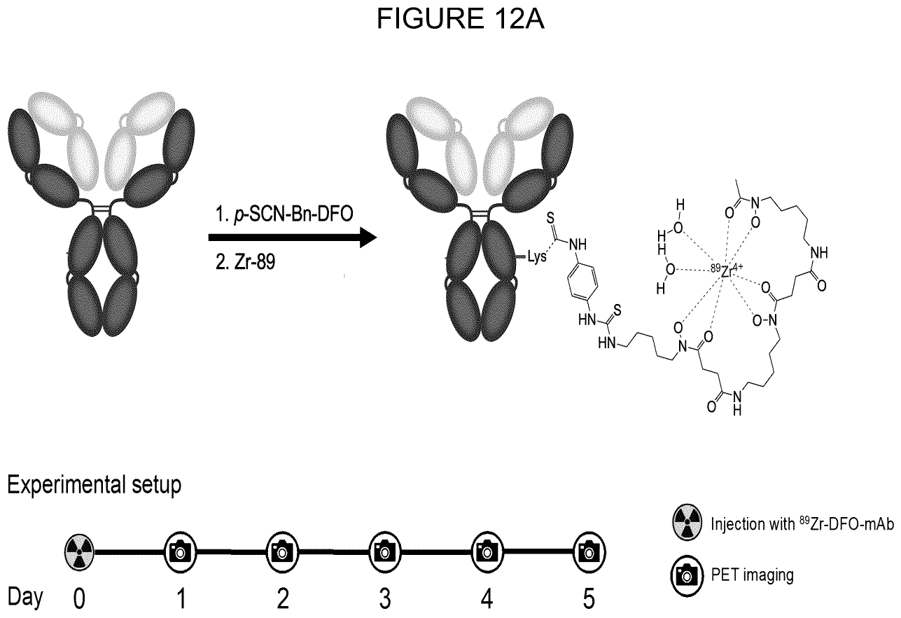

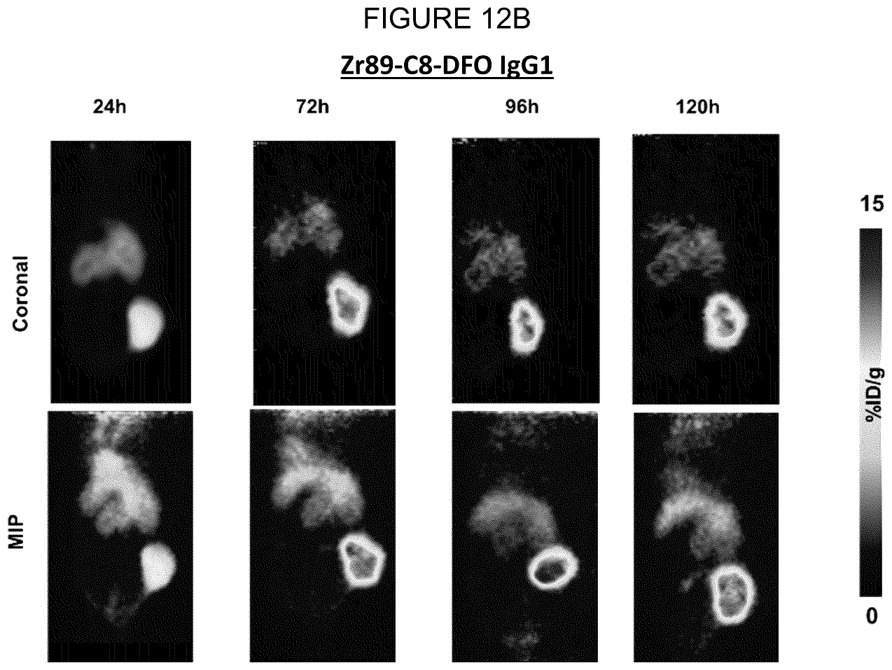

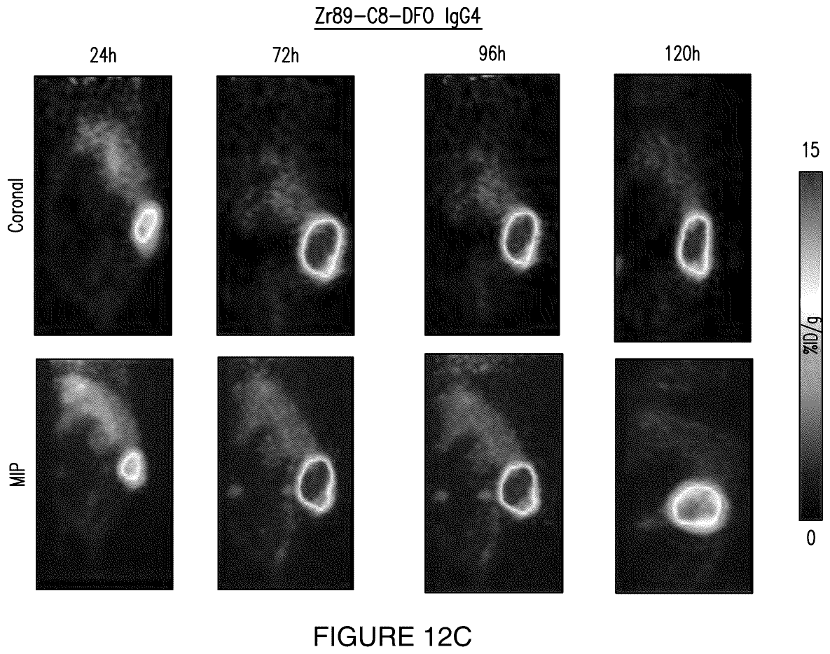

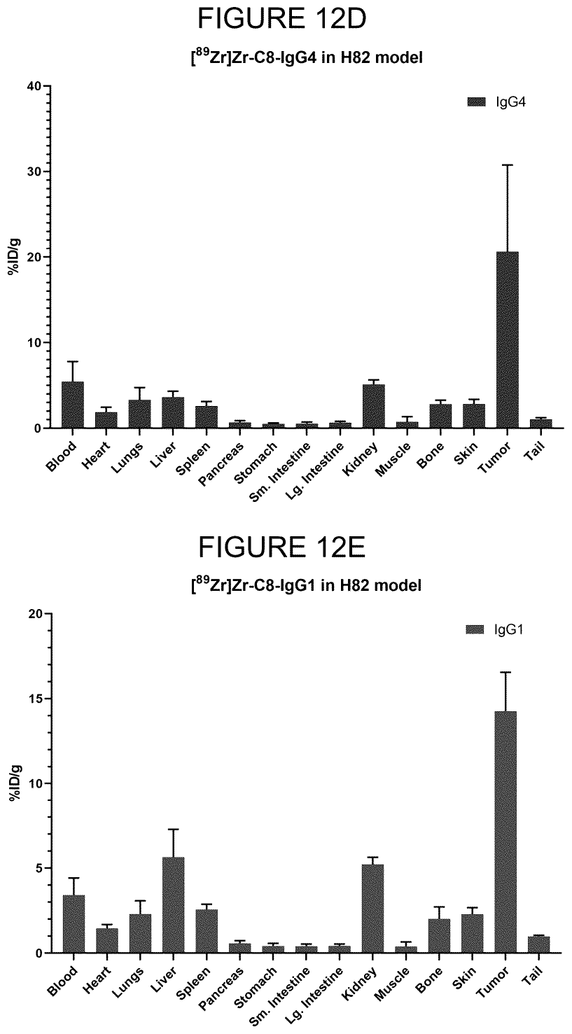

C8 IgG1 and C8 IgG4 were bioconjugated to p-SCN-Bn-DFO via lysine residues on the mAb, then radiolabeled with Zr-89 and injected into mice bearing subcutaneous H82 (SCLC) or H660 (NEPC) xenografts (FIG. 12A). As shown in FIGS. 12B-12E, C8 IgG1 clone and C8 IgG4 clone showed tumor uptake with clear delineation between tumor-to-nontumor organs after 72 hours. Notably, the tumor uptake of C8 IgG4 seemed higher compared to C8 IgG1 clone.

Importantly, the biodistribution analysis reflected the PET/CT images and showed tumor uptake around 15-20% ID/g. Similarly to the evaluation for the PET/CT images, tumor uptake was higher for the C8-IgG4 clone.

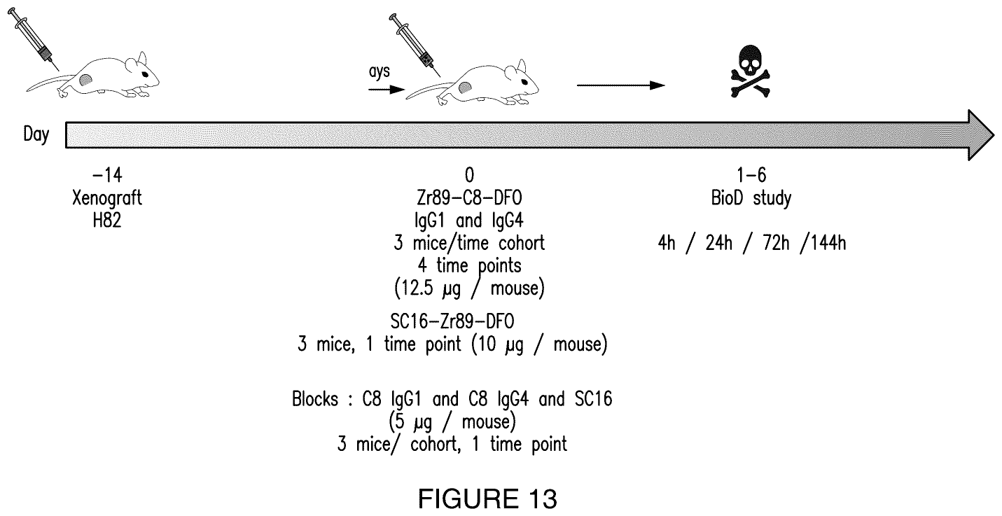

Next, a full biodistribution study in the H82 cells was performed since they grow out quicker. The experimental set up was the same as for the E23 clone, as outlined above. Briefly, C8 IgG1 and C8 IgG4 were bioconjugated to p-SCN-Bn-DFO via lysine residues on the mAb, then radiolabeled with Zr-89. Next, mice bearing subcutaneous H82 (SCLC) xenograft was injected with ⁸⁹Zr-DFO-C8 IgG1 and IgG4 radioconjugate and sacrificed at 1, 4, 72, and 144h post-injection (FIG. 13). The most relevant organs were analyzed. Further, for each cohort the blocks for C8 IgG1 and C8 IgG4 were included.

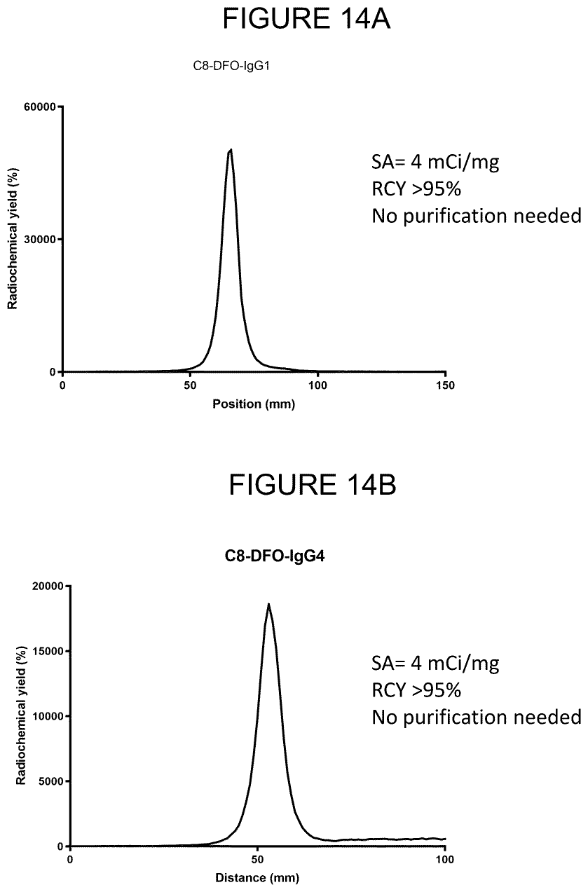

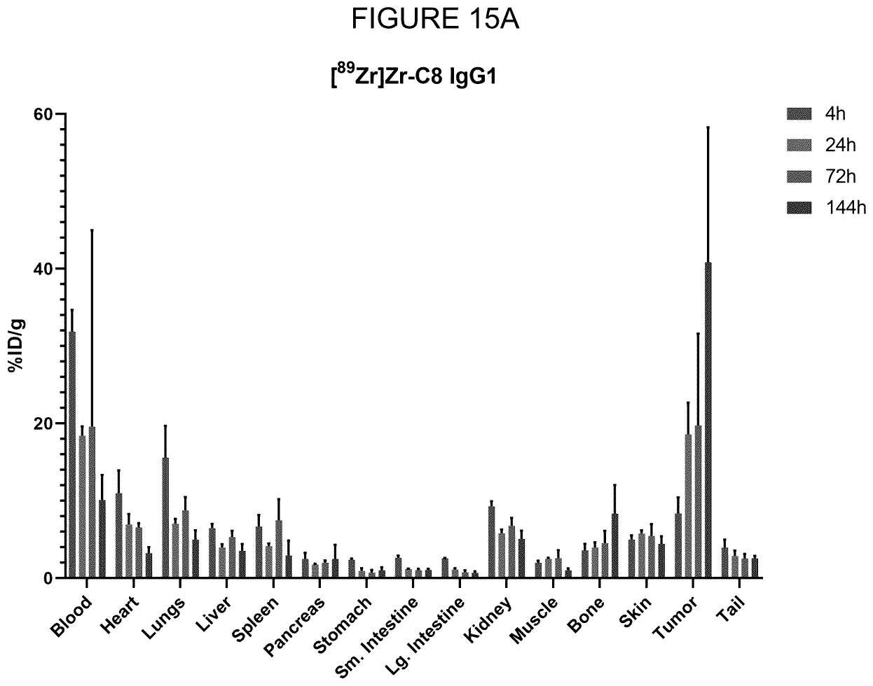

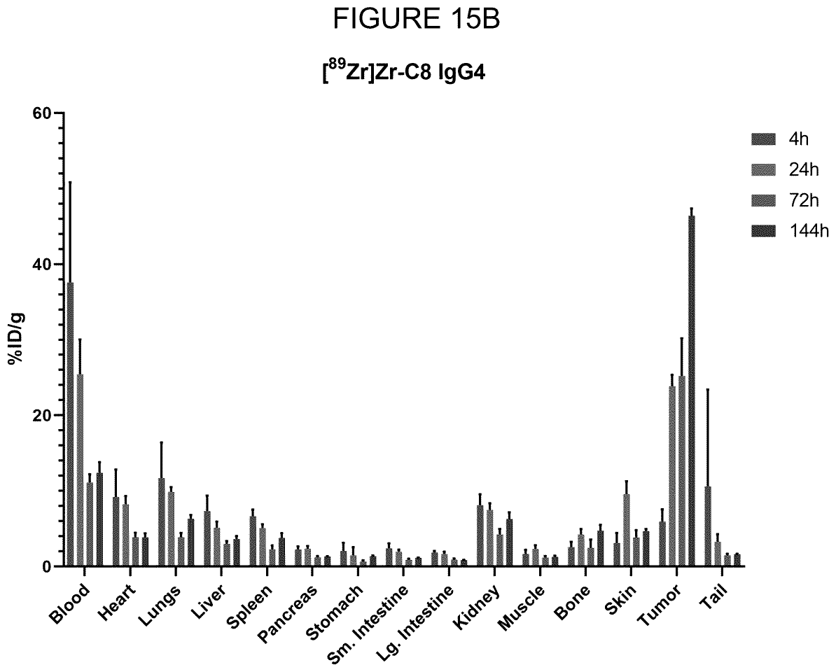

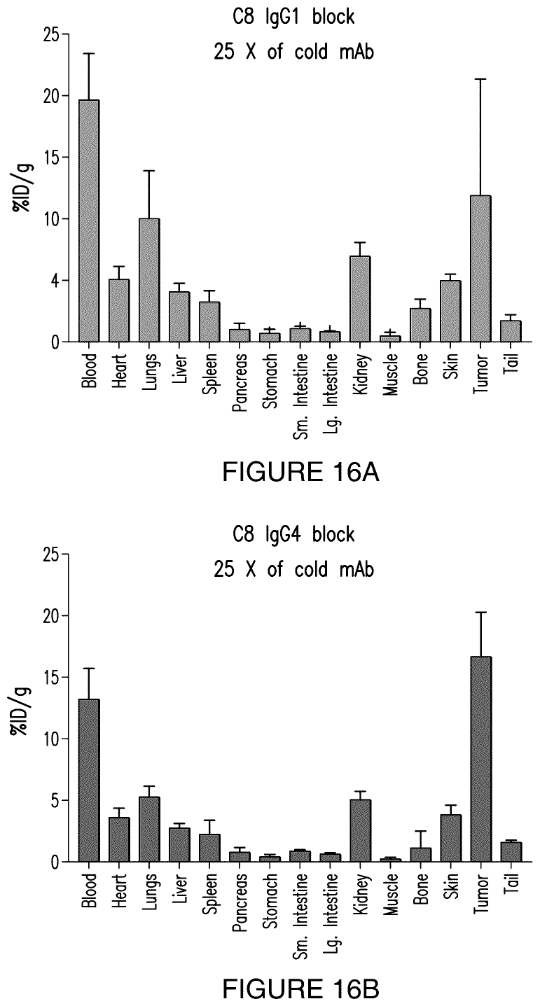

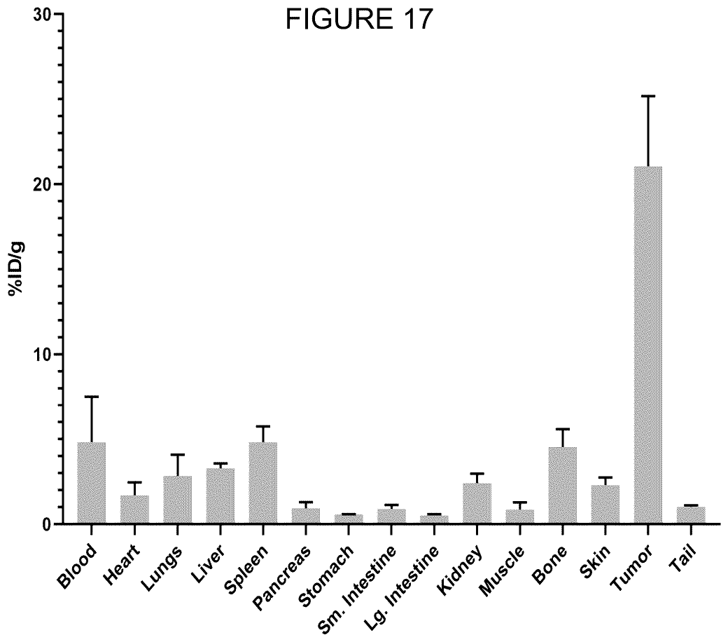

The quality control showed that the radiochemical yield was shown to be very good for both clones and no purification was needed (FIGS. 14A and 14B). Further, as shown in FIGS. 15A and 15B, the full course biodistribution study showed very high tumor uptake that was accentuated at the latest time point (i.e., 144h). The non-tumor bearing organs showed that the clones uptake decreased over time, with a higher TI compared to the E23. Further, the blocks at the 72 h also showed that the tumor uptake was lower than the C8 IgG1 and C8 IgG4 groups (FIGS. 16A and 16B). Importantly, the SC16 control group showed similar results to the previous studies (FIG. 17).

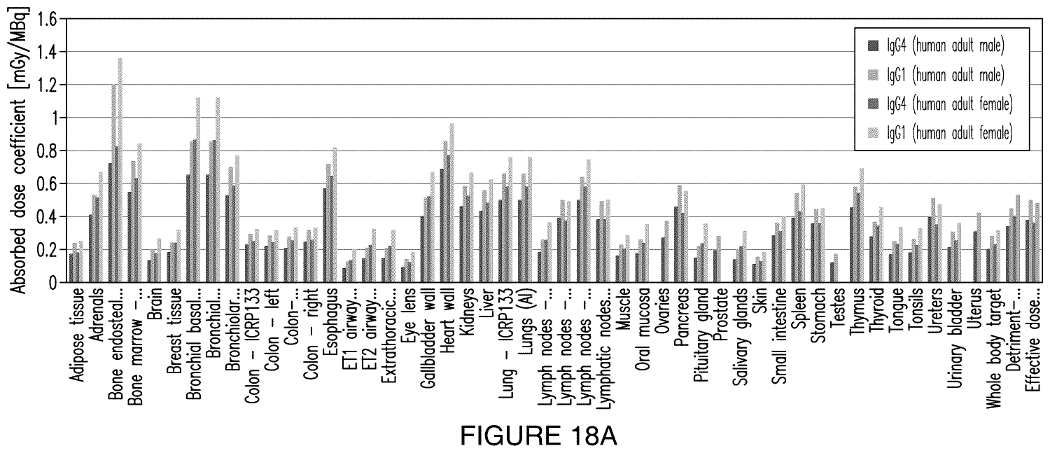

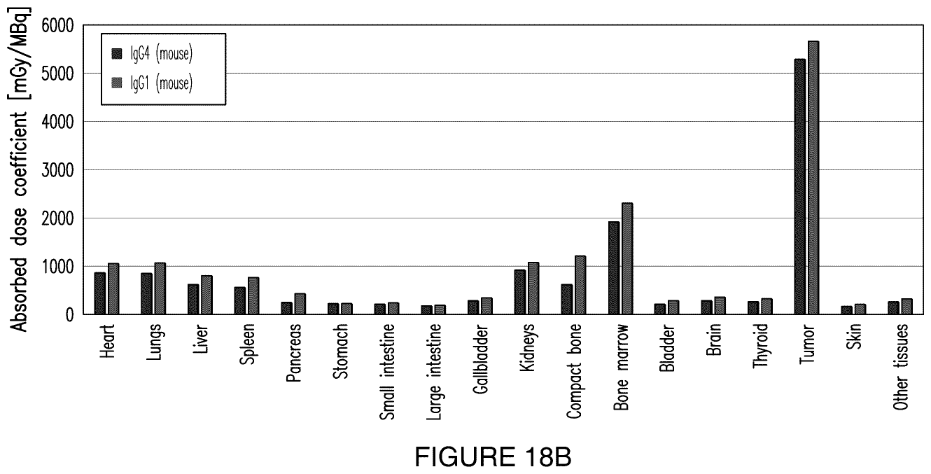

Next, the dosimetry data were extrapolated to human adults. As seen in FIGS. 18A and 18B, the mean effective dose is approx. 0.4 mGy/MBq, while the therapeutic index (TI) in mice was higher for the C8 IgG1 and C8 IgG4 compared to the E23.

In conclusion, these data demonstrate that C8 clone showed very high DLL3 targeting binding and very strong tumor to non-tumor organ delineation and is an excellent candidate for translation as both an imaging and therapeutic agent.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

The invention claimed is:

1.-108. (canceled)

109. An anti-DLL3 antibody or an antigen-binding fragment thereof, comprising:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23;
d) a heavy chain variable region comprising a CDR1 comprising the amino acid sequence set forth in SEQ ID NO: 28, a CDR2 comprising the amino acid sequence set forth in SEQ ID NO: 29; and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48; a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
h) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64; a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
i) a heavy chain variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a light chain variable region comprising a CDR1 comprising the amino acid sequence set forth in SEQ ID NO: 90, a CDR2 comprising the amino acid sequence set forth in SEQ ID NO: 216, and a CDR3 comprising the amino acid sequence set forth in SEQ ID NO: 91;
l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
n) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
o) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116; and a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
p) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
r) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
s) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137; and a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
t) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145; and a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
u) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
v) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59;
w) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162; or
x) a heavy chain variable region comprising a CDR1 comprising the amino acid sequence set forth in SEQ ID NO: 96, a CDR2 comprising the amino acid sequence set forth in SEQ ID NO: 167, a CDR3 comprising the amino acid sequence set forth in SEQ ID NO: 168; and a light chain variable region comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171.

110. The antibody or antigen-binding fragment thereof of claim 109, comprising:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, or SEQ ID NO: 172; and
(b) a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, or SEQ ID NO: 173.

111. The antibody or antigen-binding fragment thereof of claim 109, wherein the antibody or antigen-binding fragment thereof binds to a DLL3 comprising the amino acid sequence set forth in SEQ ID NO: 215 or a fragment thereof.

112. The antibody or antigen-binding fragment thereof of claim 109, wherein the antibody comprises a human variable region framework region.

113. The antibody or antigen-binding fragment thereof of claim 109, which is:
(a) a fully human or an antigen-binding fragment thereof,
(b) a chimeric antibody or an antigen-binding fragment thereof,
(c) a humanized antibody or an antigen-binding fragment thereof.

114. The antibody or antigen-binding fragment thereof of claim 109, wherein the antigen-binding fragment is a Fab, Fab′, F(ab′)2, variable fragment (Fv), or single chain variable region (scFv).

115. A multi-specific molecule comprising the antibody or antigen-binding fragment thereof of claim 109, linked to one or more functional moieties.

116. The multi-specific molecule of claim 115, wherein the one or more functional moieties have a different binding specificity than the antibody or antigen binding fragment thereof.

117. A host cell comprising a nucleic acid encoding an anti-DLL3antibody or antigen-binding fragment thereof, wherein the anti-DLL3antibody or antigen-binding fragment thereof comprises:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3;
and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13;
and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22;
and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23;
d) a heavy chain variable region comprising a CDR1 comprising the amino acid sequence set forth in SEQ ID NO: 28, a CDR2 comprising the amino acid sequence set forth in SEQ ID NO: 29; and a CDR3 comprising the amino acid sequence set forth in SEQ ID NO: 30;
and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39;
and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56;
and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
h) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64;
a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
i) a heavy chain variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72;
and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81;
and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89;
and a light chain variable region comprising a CDR1 comprising the amino acid sequence set forth in SEQ ID NO: 90, a CDR2 comprising the amino acid sequence set forth in SEQ ID NO: 216, and a CDR3 comprising the amino acid sequence set forth in SEQ ID NO: 91;
l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98;
a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
n) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
o) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116; and a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
p) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123;
and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123;
and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
r) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56;
and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
s) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137; and a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
t) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145;
and a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
u) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
v) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123;
and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59;
w) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO:
161; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162; or
x) a heavy chain variable region comprising a CDR1 comprising the amino acid sequence set forth in SEQ ID NO: 96, a CDR2 comprising the amino acid sequence set forth in SEQ ID NO: 167, a CDR3 comprising the amino acid sequence set forth in SEQ ID NO: 168;
and a light chain variable region comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171.

118. An immunoconjugate comprising an anti-DLL3 antibody or antigen-binding fragment thereof linked to a therapeutic agent, wherein the anti-DLL3 antibody or antigen-binding fragment thereof comprises:
a) a heavy chain variable region comprising a CDR1 comprising the amino acid sequence set forth in SEQ ID NO: 21, a CDR2 comprising the amino acid sequence set forth in SEQ ID NO: 183, and a CDR3 comprising the amino acid sequence set forth in SEQ ID NO: 184; and a light chain variable region comprising a CDR1 comprising the amino acid sequence set forth in SEQ ID NO: 185, a CDR2 comprising the amino acid sequence set forth in SEQ ID NO: 50, and a CDR3 comprising the amino acid sequence set forth in SEQ ID NO: 186;
b) a heavy chain variable region comprising a CDR1 comprising the amino acid sequence set forth in SEQ ID NO: 191, a CDR2 comprising the amino acid sequence set forth in SEQ ID NO: 192, and a CDR3 comprising the amino acid sequence set forth in SEQ ID NO: 193; and a light chain variable region comprising a CDR1 comprising the amino acid sequence set forth in SEQ ID NO: 194, a CDR2 comprising the amino acid sequence set forth in SEQ ID NO: 195, and a CDR3 comprising the amino acid sequence set forth in SEQ ID NO: 196;
c) a heavy chain variable region comprising a CDR1 comprising the amino acid sequence set forth in SEQ ID NO: 11, a CDR2 comprising the amino acid sequence set forth in SEQ ID NO: 201, and a CDR3 comprising the amino acid sequence set forth in SEQ ID NO: 202; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 203; or
d) a heavy chain variable region comprising a CDR1 comprising the amino acid sequence set forth in SEQ ID NO: 208, a CDR2 comprising the amino acid sequence set forth in SEQ ID NO: 209, and a CDR3 comprising the amino acid sequence set forth in SEQ ID NO: 210; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 211.

119. The immunoconjugate of claim 118, wherein the anti-DLL3 antibody or antigen-binding fragment thereof comprises:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 187, SEQ ID NO: 197, SEQ ID NO: 204, or SEQ ID NO: 212; and
(b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 188, SEQ ID NO: 198, SEQ ID NO: 205, or SEQ ID NO: 213.

120. The immunoconjugate of claim 118, wherein the antibody or antigen-binding fragment thereof binds to a DLL3 comprising the amino acid sequence set forth in SEQ ID NO: 215 or a fragment thereof.

121. The immunoconjugate of claim 118, wherein the antibody comprises a human variable region framework region.

122. The immunoconjugate of claim 118, wherein the antibody or antigen-binding fragment thereof is:
(a) a fully human or an antigen-binding fragment thereof,
(b) a chimeric antibody or an antigen-binding fragment thereof, or
(c) a humanized antibody or an antigen-binding fragment thereof.

123. The immunoconjugate of claim 118, wherein the antigen-binding fragment is a Fab, Fab′, F(ab′)2, variable fragment (Fv), or single chain variable region (scFv).

124. The immunoconjugate of claim 118, wherein the therapeutic agent is a radioactive isotope.

125. The immunoconjugate of claim 118, further comprising a chelator.

126. A pharmaceutical composition comprising:
A) an immunoconjugate comprising an anti-DLL3 antibody or antigen-binding fragment thereof linked to a therapeutic agent; and
B) a pharmaceutically acceptable carrier;
wherein the anti-DLL3 antibody or antigen-binding fragment thereof comprises:
a) a heavy chain variable region comprising a CDR1 comprising the amino acid sequence set forth in SEQ ID NO: 21, a CDR2 comprising the amino acid sequence set forth in SEQ ID NO: 183, and a CDR3 comprising the amino acid sequence set forth in SEQ ID NO: 184; and a light chain variable region comprising a CDR1 comprising the amino acid sequence set forth in SEQ ID NO: 185, a CDR2 comprising the amino acid sequence set forth in SEQ ID NO: 50, and a CDR3 comprising the amino acid sequence set forth in SEQ ID NO: 186;
b) a heavy chain variable region comprising a CDR1 comprising the amino acid sequence set forth in SEQ ID NO: 191, a CDR2 comprising the amino acid sequence set forth in SEQ ID NO: 192, and a CDR3 comprising the amino acid sequence set forth in SEQ ID NO: 193; and a light chain variable region comprising a CDR1 comprising the amino acid sequence set forth in SEQ ID NO: 194, a CDR2 comprising the amino acid sequence set forth in SEQ ID NO: 195, and a CDR3 comprising the amino acid sequence set forth in SEQ ID NO: 196;
c) a heavy chain variable region comprising a CDR1 comprising the amino acid sequence set forth in SEQ ID NO: 11, a CDR2 comprising the amino acid sequence set forth in SEQ ID NO: 201, and a CDR3 comprising the amino acid sequence set forth in SEQ ID NO: 202; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 203; or
d) a heavy chain variable region comprising a CDR1 comprising the amino acid sequence set forth in SEQ ID NO: 208, a CDR2 comprising the amino acid sequence set forth in SEQ ID NO: 209, and a CDR3 comprising the amino acid sequence set forth in SEQ ID NO: 210; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 211.

127. A method of treating or ameliorating a disease or disorder associated with DLL3 in a subject, comprising administering to the subject:
(A) an immunoconjugate comprising an anti-DLL3 antibody or antigen-binding fragment thereof linked to a therapeutic agent; or
(B) a pharmaceutical composition thereof,
wherein the anti-DLL3antibody or antigen-binding fragment thereof comprises:
a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3;
and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13;
and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22;
and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23;
d) a heavy chain variable region comprising a CDR1 comprising the amino acid sequence set forth in SEQ ID NO: 28, a CDR2 comprising the amino acid sequence set forth in SEQ ID NO: 29; and a CDR3 comprising the amino acid sequence set forth in SEQ ID NO: 30;
and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39;
and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56;
and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
h) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64;
a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
i) a heavy chain variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72;
and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81;
and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89;
and a light chain variable region comprising a CDR1 comprising the amino acid sequence set forth in SEQ ID NO: 90, a CDR2 comprising the amino acid sequence set forth in SEQ ID NO: 216, and a CDR3 comprising the amino acid sequence set forth in SEQ ID NO: 91;
l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98;
a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
n) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
o) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116; and a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
p) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123;
and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123;
and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
r) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56;
and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
s) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137; and a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
t) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145;
and a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
u) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
v) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123;
and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59;
w) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162; or
x) a heavy chain variable region comprising a CDR1 comprising the amino acid sequence set forth in SEQ ID NO: 96, a CDR2 comprising the amino acid sequence set forth in SEQ ID NO: 167, a CDR3 comprising the amino acid sequence set forth in SEQ ID NO: 168;
and a light chain variable region comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171.

128. The method of claim 125, wherein the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma.


SEQUENCE LISTING (216 sequences)

SEQ ID NO: 1 - protein, 7 aa, synthetic construct
GYTFTIY

SEQ ID NO: 2 - protein, 6 aa, synthetic construct
NPSGGS

SEQ ID NO: 3 - protein, 16 aa, synthetic construct
NDYVDYVTTDYYGMDV

SEQ ID NO: 4 - protein, 11 aa, synthetic construct
RASQGISNYLA

SEQ ID NO: 5 - protein, 7 aa, synthetic construct
AASSLQS

SEQ ID NO: 6 - protein, 9 aa, synthetic construct
QQYNSSPYT

SEQ ID NO: 7 - protein, 125 aa, synthetic construct
QVHLVQSGAEVKKPGASVKVSCKASGYTFTIYYIHWVRQAPGQGLEWMGIINPSGGSTSYAQKFQGRVTMTRDTSTSTVYMELSSLRSEDTAVYYCSGNDYVDYVTTDYYGMDVWGQGTTVTVSS

SEQ ID NO: 8 - protein, 107 aa, synthetic construct
DIQMTQSPSSLSASVGDRVTITCRASQGISNYLAWFQQKPGKAPKSLIYAASSLQSGVPSKFSGSGSGTDFTLTISSLQPEDFATYYCQQYNSSPYTFGQGTKLEIK

SEQ ID NO: 9 - DNA, 375 bp, synthetic construct
  source 1..375: mol_type = other DNA
caggtgcatctggtgcagtctggggctgaggtgaagaagcctggggcctcagtgaaggtttcttgcaaggcatctggatacaccttcaccatctactatattcactgggtgcgacaggcccctggacaggggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtatattactgttcgggtaatgactacgttgactacgtaactactgactactacggtatggacgtctggggccaagggaccacggtcaccgtctcctca

SEQ ID NO: 10 - DNA, 321 bp, synthetic construct
  source 1..321: mol_type = other DNA
gacatccagatgacccagtctccatcctcactgtctgcatctgtaggagatagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcacactcaccatcagcagcctgcagcctgaagattttgcaacttattactgccaacagtataatagttccccgtacacttttggccaggggaccaagctggagatcaaa

SEQ ID NO: 11 - protein, 7 aa, synthetic construct
GFTFSSY

SEQ ID NO: 12 - protein, 6 aa, synthetic construct
SGRGGD

SEQ ID NO: 13 - protein, 8 aa, synthetic construct
ERGLGFDY

SEQ ID NO: 14 - protein, 17 aa, synthetic construct
RSSQSLLDSDDGNTYLD

SEQ ID NO: 15 - protein, 7 aa, synthetic construct
TLSYRAS

SEQ ID NO: 16 - protein, 9 aa, synthetic construct
MQHLEFPLT

SEQ ID NO: 17 - protein, 117 aa, synthetic construct
EVQLLESGGGLVQPGGSLRLSCAASGFTFSSYAMGWVRQTPGKGLEWVSTISGRGGDTYYADSVKGRFTISRDNSKNTLYLQMNSLRAEDTAIYYCAKERGLGFDYWGQGTLVTVSS

SEQ ID NO: 18 - protein, 113 aa, synthetic construct
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQHLEFPLTFGGGTKVEIK

SEQ ID NO: 19 - DNA, 351 bp, synthetic construct
  source 1..351: mol_type = other DNA
gaggtgcagctgttggagtctgggggaggcttggtacagcctggggggtccctgagactctcctgtgcagcctctggattcacctttagcagctatgccatgggctgggtccgccagactccagggaaggggctggagtgggtctcaactattagtggtcgtggtggtgacacatactacgcagactccgtgaagggccggttcaccatctccagagacaattccaagaacacgctgtatctgcaaatgaacagcctgagagccgaggacacggccatatattactgtgcgaaagagaggggactggggtttgactactggggccagggaaccctggtcaccgtctcctca

SEQ ID NO: 20 - DNA, 339 bp, synthetic construct
  source 1..339: mol_type = other DNA
gatattgtgatgacccagactccactctccctgcccgtcacccctggagagccggcctccatctcctgcaggtctagtcagagcctcttggatagtgatgatggaaacacctatttggactggtacctgcagaagccagggcagtctccacagctcctgatctatacgctttcctatcgggcctctggagtcccagacaggttcagtggcagtgggtcaggcactgatttcacactgaaaatcagcagggtggaggctgaggatgttggagtttattactgcatgcaacatctagagtttccgctcactttcggcggagggaccaaggtggagatcaaa

SEQ ID NO: 21 - protein, 7 aa, synthetic construct
GYTFTSY

SEQ ID NO: 22 - protein, 15 aa, synthetic construct
GDYDILTDYYYNMDV

SEQ ID NO: 23 - protein, 9 aa, synthetic construct
LQHNSYPYT

SEQ ID NO: 24 - protein, 124 aa, synthetic construct
QVQLVQSGAEVKKPGASVKVSCKASGYTFTSYYMHWVRQAPGQGLEWMGIINPSGGSTSYAQKFRGRVTMTRDTSTSTVYMELSSLRSEDTAVYYCARGDYDILTDYYYNMDVWGQGTTVTVSS

SEQ ID NO: 25 - protein, 107 aa, synthetic construct
DIQMTQSPSAMSASVGDRVTITCRASQGISNYLAWFQQKPGKVPKRLIYAASSLQSGVPSRFSGSGSGTEFTLTISSLQPEDFATYYCLQHNSYPYTFGQGTKLEIK

SEQ ID NO: 26 - DNA, 372 bp, synthetic construct
  source 1..372: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccggggcagagtcaccatgaccagggacacgtccacgagcacagtctacatggagctgagcagcctgagatctgaggacacggccgtttattactgtgcgagaggggattacgatattttgacggactactactacaatatggacgtctggggccaagggaccacggtcaccgtctcctca

SEQ ID NO: 27 - DNA, 321 bp, synthetic construct
  source 1..321: mol_type = other DNA
gacatccagatgacccagtctccatctgccatgtctgcatctgtaggagacagagtcaccatcacttgtcgggcgagtcagggcattagcaattatttagcctggtttcagcagaaaccagggaaagtccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtacactttcggccaggggaccaagctggagatcaaa

SEQ ID NO: 28 - protein, 9 aa, synthetic construct
GFSLSTSGM

SEQ ID NO: 29 - protein, 5 aa, synthetic construct
DWDDN

SEQ ID NO: 30 - protein, 13 aa, synthetic construct
IRGYSGSYDAFDI

SEQ ID NO: 31 - protein, 16 aa, synthetic construct
RSSQSLLHSNGYNYLD

SEQ ID NO: 32 - protein, 7 aa, synthetic construct
LGSNRAP

SEQ ID NO: 33 - protein, 9 aa, synthetic construct
MQALQTPFT

SEQ ID NO: 34 - protein, 123 aa, synthetic construct
QVTLRESGPALVKSTQTLTLTCTFSGFSLSTSGMCVSWIRQTPGKALEWLALIDWDDNKYYSTSLKTRLIISKDTSKNQVVLTMTNMEPVDTATYYCARIRGYSGSYDAFDIWGQGTMVTVSS

SEQ ID NO: 35 - protein, 112 aa, synthetic construct
DIVMTQSPLSLPVTPGEPASISCRSSQSLLHSNGYNYLDWYLQKPGQSPHILLYLGSNRAPGVPDRFSGSGSGTDFTLKISRVEAEDVGVYYCMQALQTPFTFGPGTKVDIK

SEQ ID NO: 36 - DNA, 369 bp, synthetic construct
  source 1..369: mol_type = other DNA
caggtcaccttgagggagtctggtcctgcgctggtgaaatccacacagaccctcacactgacctgcaccttctctgggttctcactcagtactagtggaatgtgtgtgagctggatccgtcagaccccagggaaggccctggagtggcttgcactcattgattgggatgataataaatactacagcacatctctgaagaccaggctcatcatctccaaggacacctccaaaaaccaggtggtccttacaatgaccaatatggaacctgtggacacagccacgtattattgtgcacggatacgggggtatagtgggagctacgatgcttttgatatctggggccaagggacaatggtcaccgtctcttca

SEQ ID NO: 37 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacacatcctgctctatttgggttctaatcgggcccccggggtccctgacaggttcagtggcagtggatcaggcacagattttacactgaaaatcagcagagtggaggctgaggatgttggggtttattactgcatgcaagctctacaaactccattcactttcggccctgggaccaaagtggatatcaaa

SEQ ID NO: 38 - protein, 6 aa, synthetic construct
NSSGGS

SEQ ID NO: 39 - protein, 15 aa, synthetic construct
GYCTNGVYYDEDFDY

SEQ ID NO: 40 - protein, 11 aa, synthetic construct
RASQGISHYLA

SEQ ID NO: 41 - protein, 9 aa, synthetic construct
QQYNSYPLT

SEQ ID NO: 42 - protein, 124 aa, synthetic construct
QVQLVQSGAEVKKPGASVKVSCKASGYTFTSYYMHWVRQAPGQGLEWMGVINSSGGSTSYAQKFQGRVTMTRDTSTSTVYMDLSSLRSEDTAVYYCARGYCTNGVYYDEDFDYWGQGTLVTVSS

SEQ ID NO: 43 - protein, 107 aa, synthetic construct
DIQMTQSPSSLSASVGDRVTITCRASQGISHYLAWFQQKPGKAPTSLIYAASSLQSGVPSKFSGSGSGTDFTLTISSLQPEDFATYYCQQYNSYPLTFGGGTKVEIK

SEQ ID NO: 44 - DNA, 372 bp, synthetic construct
  source 1..372: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggagtaatcaattctagtggtggtagcacaagctacgcacagaagttccagggcagagtcaccatgaccagggacacgtccacgagcacagtctacatggacctgagcagcctgagatctgaggacacggccgtgtattactgtgcgagagggtattgtactaatggtgtttactatgatgaggactttgactactggggccagggaaccctggtcaccgtctcctca

SEQ ID NO: 45 - DNA, 321 bp, synthetic construct
  source 1..321: mol_type = other DNA
gacatccagatgacccagtctccatcctcactgtctgcatctgtaggagacagagtcaccatcacttgtcgggcgagtcagggcattagccattatttagcctggtttcagcagaaaccagggaaagcccctacgtccctgatctatgctgcatccagtttgcaaagtggggtcccatcaaagttcagcggcagtggatctgggacagatttcactctcaccatcagcagcctgcagcctgaagattttgcaacttattactgccaacagtataatagttaccctctcactttcggcggagggaccaaggtggagatcaaa

SEQ ID NO: 46 - protein, 7 aa, synthetic construct
GGSISSY

SEQ ID NO: 47 - protein, 5 aa, synthetic construct
YTSGS

SEQ ID NO: 48 - protein, 10 aa, synthetic construct
EDWESWTFDI

SEQ ID NO: 49 - protein, 16 aa, synthetic construct
RSGQSLVYSDGDTYLN

SEQ ID NO: 50 - protein, 7 aa, synthetic construct
KVSNRDS

SEQ ID NO: 51 - protein, 9 aa, synthetic construct
MQGTHWPLS

SEQ ID NO: 52 - protein, 118 aa, synthetic construct
QVQLQESGPGLVKPSETLFLTCTVSGGSISSYYWTWIRQPAGKGLEWIGRIYTSGSTNYNPSLKSRVTMSVDTSKNQFSLKLSSVTAADTAVYYCAREDWESWTFDIWGQGTMVTVSS

SEQ ID NO: 53 - protein, 112 aa, synthetic construct
DVVMTQSPLSLPVTLGQPASISCRSGQSLVYSDGDTYLNWFQQRPGQSPRRLIYKVSNRDSGVPDRFSGSGSGTDFTLKISRVEAEDVGVYYCMQGTHWPLSFGGGTKVEIK

SEQ ID NO: 54 - DNA, 354 bp, synthetic construct
  source 1..354: mol_type = other DNA
caggtgcagctgcaggagtcgggcccaggactggtgaagccttcggagaccctgttcctcacctgcactgtctctggtggctccatcagtagttactactggacctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagagaggactgggaatcgtggacttttgatatctggggccaagggacaatggtcaccgtctcttca

SEQ ID NO: 55 - DNA, 336 bp, synthetic construct
  source 1..336: mol_type = other DNA
gatgttgtgatgactcagtctccactctccctgcccgtcacccttggacagccggcctccatctcctgcaggtctggtcaaagcctcgtttacagtgatggagacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgctctctttcggcggagggaccaaggtggagatcaaa

SEQ ID NO: 56 - protein, 11 aa, synthetic construct
QLELLGNAFDI

SEQ ID NO: 57 - protein, 12 aa, synthetic construct
RASQSVSSSYLA

SEQ ID NO: 58 - protein, 7 aa, synthetic construct
GASSRAT

SEQ ID NO: 59 - protein, 9 aa, synthetic construct
QQYGSSPYT

SEQ ID NO: 60 - protein, 120 aa, synthetic construct
QVQLVQSGAEVKKPGASVKVSCKASGYTFTSYYIHWVRQAPGQGLEWMGIINPSGGSKSYAQKFQGRVTMTRDTSTGTVYMELSSLRSEDTAVYYCAGQLELLGNAFDIWGQGTMVTVSS

SEQ ID NO: 61 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIK

SEQ ID NO: 62 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatacactgggtgcgacaggcccctggacaagggcttgagtggatgggaataatcaaccctagtggtggtagtaaaagctacgcacagaagttccagggcagagtcaccatgaccagggacacgtccacgggaacagtctatatggagctgagcagcctgagatctgaggacacggccgtgtattactgtgcgggacaactggaacttttggggaatgcttttgatatctggggccaagggacaatggtcaccgtctcttca

SEQ ID NO: 63 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtacacttttggccaggggaccaagctggagatcaaa

SEQ ID NO: 64 - protein, 14 aa, synthetic construct
ERQQLDYYYFAMDV

SEQ ID NO: 65 - protein, 9 aa, synthetic construct
QQYNSYPPT

SEQ ID NO: 66 - protein, 123 aa, synthetic construct
QVQLVQSGAEVKKPGASVKVSCKASGYTFTSYYLHWVRQAPGHGLEWMAIINPSGGSTSYAQKFLGRVTMTRDTSTNTVYMELSSLRSEDTAVYYCARERQQLDYYYFAMDVWGQGTTVTVSS

SEQ ID NO: 67 - protein, 107 aa, synthetic construct
DIQMTQSPSSLSASVGDRVTITCRASQGISNYLAWFQQKPGKAPKSLIYAASSLQSGVPSKFSGSGSGTDFTLTISSLQPEDFATYYCQQYNSYPPTFGQGTKVEIK

SEQ ID NO: 68 - DNA, 369 bp, synthetic construct
  source 1..369: mol_type = other DNA
caggtgcagctggtgcagtctggggctgaggtgaagaagcctggggcctcagtgaaggtttcctgcaaggcatctggatacaccttcaccagctactatttgcactgggtgcgacaggcccctggacacgggcttgagtggatggcaataatcaaccctagtggtggtagtacaagctacgcacagaagttcctgggcagagtcaccatgaccagggacacgtccacgaacacagtctacatggagctgagcagcctgagatctgaggacacggccgtgtattactgtgcgagagaacggcagcagctggactactactacttcgctatggacgtctggggccaagggaccacggtcaccgtctcctca

SEQ ID NO: 69 - DNA, 321 bp, synthetic construct
  source 1..321: mol_type = other DNA
gacatccagatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataatagttaccctccgacgttcggccaagggaccaaggtggaaatcaaa

SEQ ID NO: 70 - protein, 7 aa, synthetic construct
GFTFSFY

SEQ ID NO: 71 - protein, 6 aa, synthetic construct
SSSSSY

SEQ ID NO: 72 - protein, 11 aa, synthetic construct
NYDFWRGAFDI

SEQ ID NO: 73 - protein, 11 aa, synthetic construct
RASQSVSSNLA

SEQ ID NO: 74 - protein, 7 aa, synthetic construct
GASTRAT

SEQ ID NO: 75 - protein, 10 aa, synthetic construct
QQYNNWPTWT

SEQ ID NO: 76 - protein, 120 aa, synthetic construct
EVQLVESGGGLVKPGGSLRLSCAASGFTFSFYSMNWVRQAPGKGLEWVSSISSSSSYIYYADSVKGRFTISRDNAKNSLYLQMNSLRAEDTAVYYCARNYDFWRGAFDIWGQGTMVTVSS

SEQ ID NO: 77 - protein, 108 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPTWTFGQGTKVEIK

SEQ ID NO: 78 - DNA, 360 bp, synthetic construct
  source 1..360: mol_type = other DNA
gaggtgcagctggtggagtctggggggggcctggtcaagcctggagggtccctgagactctcctgtgcagcctctggattcaccttcagtttctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagaaattacgatttttggagaggtgcttttgatatctggggccaagggacaatggtcaccgtctcttca

SEQ ID NO: 79 - DNA, 324 bp, synthetic construct
  source 1..324: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcccacgtggacgttcggccaagggaccaaggtggaaatcaaa

SEQ ID NO: 80 - protein, 6 aa, synthetic construct
NYSGGS

SEQ ID NO: 81 - protein, 11 aa, synthetic construct
QGRYSGSYLDY

SEQ ID NO: 82 - protein, 7 aa, synthetic construct
QQYGSSP

SEQ ID NO: 83 - protein, 120 aa, synthetic construct
QVQLVQSGAEVKKPGASVKVSCKASGYTFTSYYMHWVRQAPGQGLEWMGIINYSGGSKSYAQKFQGRVTMTRDTSTSTVYMELSSLRSEDTGVYYCARQGRYSGSYLDYWGQGTLVTVSS

SEQ ID NO: 84 - protein, 106 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFGQGTKLEIK

SEQ ID NO: 85 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaactatagtggtggtagcaaaagctacgcacagaagttccagggcagagtcaccatgacccgggacacgtccacgagcacagtctacatggagctgagcagcctgagatctgaggacacgggcgtgtattactgtgcgagacaggggcggtatagtgggagctaccttgactactggggccagggaaccctggtcaccgtctcctca

SEQ ID NO: 86 - DNA, 318 bp, synthetic construct
  source 1..318: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttttggccaggggaccaagctggagatcaaa

SEQ ID NO: 87 - protein, 7 aa, synthetic construct
GYTFNSY

SEQ ID NO: 88 - protein, 6 aa, synthetic construct
NPSGGT

SEQ ID NO: 89 - protein, 10 aa, synthetic construct
DWGEQYGMDV

SEQ ID NO: 90 - protein, 16 aa, synthetic construct
RSSQSLLHSNGYNYLD

SEQ ID NO: 91 - protein, 9 aa, synthetic construct
MQALQTPLT

SEQ ID NO: 92 - protein, 119 aa, synthetic construct
QVQLVQSGAEVKKPGASLKVSCKTSGYTFNSYYMHWVRQAPGQGLEWMGINNPSGGTTSYAQKFQGRVTMTRDTSTSTVYMELSSLRSEDTAVYYCARDWGEQYGMDVWGQGTTVTVSS

SEQ ID NO: 93 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 94 - DNA, 357 bp, synthetic construct
  source 1..357: mol_type = other DNA
caggtgcagctggtgcagtctggggctgaggtgaagaagcctggggcctcactgaaggtttcctgcaagacatctggatacaccttcaacagctactatatgcactgggtgcgacaggcccctggacaagggcttgagtggatgggaataaacaaccctagtggtggtaccacaagctacgcacagaagttccagggcagagtcaccatgaccagggacacgtccacgagcacagtctacatggagctgagcagcctgagatctgaggacacggccgtgtattactgtgcgagagattggggggagcagtacggtatggacgtctggggccaagggaccacggtcaccgtctcctca

SEQ ID NO: 95 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccaggacagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggagatcaaa

SEQ ID NO: 96 - protein, 7 aa, synthetic construct
GYTFTGY

SEQ ID NO: 97 - protein, 6 aa, synthetic construct
NPHSGA

SEQ ID NO: 98 - protein, 12 aa, synthetic construct
DSSGWYGDAFDI

SEQ ID NO: 99 - protein, 16 aa, synthetic construct
RSSQSLVHSDGNTYLS

SEQ ID NO: 100 - protein, 7 aa, synthetic construct
KISNRFS

SEQ ID NO: 101 - protein, 9 aa, synthetic construct
MQATQFPLT

SEQ ID NO: 102 - protein, 121 aa, synthetic construct
QVQLVQSGAEVKKPGASVKVSCKASGYTFTGYYMHWVRQAPGQGLEWMGWINPHSGAPNSAQKFQVRVTMTRDTSISTAYMELSRLRSDDTAVYYCARDSSGWYGDAFDIWGQGTMVTVSS

SEQ ID NO: 103 - protein, 112 aa, synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPLTFGGGTKVEIK

SEQ ID NO: 104 - DNA, 363 bp, synthetic construct
  source 1..363: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcacagtggtgccccaaactctgcacagaagtttcaggtcagggtcaccatgaccagggacacgtccatcagcacagcctacatggagctgagcaggctgagatctgacgacacggccgtgtattactgtgcgagggatagcagcggctggtacggtgatgcttttgatatctggggccaagggacaatggtcaccgtctcttca

SEQ ID NO: 105 - DNA, 336 bp, synthetic construct
  source 1..336: mol_type = other DNA
gatattgtgatgacccagactccactctcctcacctgtcacccttggacagccggcctccatctcctgcaggtctagtcaaagcctcgtacacagtgatggcaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaagctacacaatttccgctcactttcggcggagggaccaaggtggagatcaaa

SEQ ID NO: 106 - protein, 6 aa, synthetic construct
DPSGGS

SEQ ID NO: 107 - protein, 11 aa, synthetic construct
QGRYVGSYLDY

SEQ ID NO: 108 - protein, 120 aa, synthetic construct
QVQLVQSGAEVKKPGASVKVSCKASGYTFTSYYMHWVRQAPGQGLEWMGIIDPSGGSTSYAQKFQGRVTMTRDTSTNTVYMDLSSLRSEDTAVYYCARQGRYVGSYLDYWGQGTLVTVSS

SEQ ID NO: 109 - protein, 106 aa, synthetic construct; identical to SEQ ID NO: 84

SEQ ID NO: 110 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcgaccctagtggtggtagcacaagctacgcacagaagttccagggcagagtcaccatgaccagggacacgtccacgaacacagtctacatggacctgagcagcctgagatctgaggacacggccgtgtattactgtgcgagacaggggcggtatgttgggagctaccttgactactggggccagggaaccctggtcaccgtctcctca

SEQ ID NO: 111 - DNA, 318 bp, synthetic construct; identical to SEQ ID NO: 86
  source 1..318: mol_type = other DNA

SEQ ID NO: 112 - protein, 9 aa, synthetic construct
QQYNSYPYT

SEQ ID NO: 113 - protein, 107 aa, synthetic construct
DIQMTQSPSSLSASVGDRVTITCRASQGISNYLAWFQQKPGKAPKSVIYAASSLQSGVPSKFSGSGSGTDFTLTISSMQPEDFATYYCQQYNSYPYTFGQGTKLEIK

SEQ ID NO: 114 - DNA, 321 bp, synthetic construct
  source 1..321: mol_type = other DNA
gacatccagatgacccagtctccatcctcactgtctgcatctgtaggagacagagtcaccatcacttgtcgggcgagtcagggcattagcaattatttagcctggtttcagcagaaaccagggaaagcccctaagtccgtgatctatgctgcatccagtttgcaaagtggggtcccatcaaagttcagcggcagtggatctgggacagatttcactctcaccatcagcagcatgcagcctgaagattttgcaacttattactgccaacagtataatagttacccgtacacttttggccaggggaccaagctggagatcaaa

SEQ ID NO: 115 - protein, 6 aa, synthetic construct
NSYSGG

SEQ ID NO: 116 - protein, 12 aa, synthetic construct
DGSGWYGSYFDF

SEQ ID NO: 117 - protein, 16 aa, synthetic construct
RSSQSLVHSDGNTYLS

SEQ ID NO: 118 - protein, 9 aa, synthetic construct
IQTTQFPLT

SEQ ID NO: 119 - protein, 121 aa, synthetic construct
QVQLVQSGAEVRKPGASVKVSCKASGYTFTGYYMHWVRQAPGQGLEWMGWINSYSGGANYAQKFQDRVTMTRDTSVSTASMELSRLRSDDTAVYYCARDGSGWYGSYFDFWGQGTLVTVSS

SEQ ID NO: 120 - protein, 112 aa, synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGFYYCIQTTQFPLTFGGGTKVEIK

SEQ ID NO: 121 - DNA, 363 bp, synthetic construct
  source 1..363: mol_type = other DNA
caggtgcagctggtgcagtctggggctgaggtgaggaagcctggggcctcagtgaaggtctcctgcaaggcttctggatacaccttcaccggctactatatgcactgggtgcgacaggcccctggacaagggcttgagtggatgggatggatcaactcttacagtggtggcgcaaactatgcacagaagtttcaggacagggtcaccatgaccagggacacgtccgtcagcacagcctccatggagctgagcaggctgagatctgacgacacggccgtgtattactgtgcgagagatggcagtggctggtacgggtcctactttgacttctggggccagggaaccctggtcaccgtctcctca

SEQ ID NO: 122 - DNA, 336 bp, synthetic construct
  source 1..336: mol_type = other DNA
gatattgtgatgacccagactccactctcctcacctgtcacccttggacagccggcctccatctcctgcaggtctagtcaaagcctcgtacacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcgggttttattactgcatacaaactacacaatttccgctcactttcggcggagggaccaaggtggagatcaaa

SEQ ID NO: 123 - protein, 13 aa, synthetic construct
QGGAQWLVLAFDI

SEQ ID NO: 124 - protein, 11 aa, synthetic construct
RASQSVSSYLA

SEQ ID NO: 125 - protein, 9 aa, synthetic construct
QQYDSSPYT

SEQ ID NO: 126 - protein, 122 aa, synthetic construct
QVQLVQSGAEVKKPGASVQVSCKASGYTFTSYYMHWVRQAPGQGLEWMGIINPSGGSTSYAQKFQGRVTMTRDTSTSTVYMELSSLRSEDTAVYYCARQGGAQWLVLAFDIWGQGTMVTVSS

SEQ ID NO: 127 - protein, 107 aa, synthetic construct
EIVLTQSPGTLSLSPGERATLSCRASQSVSSYLAWYQQKPGQPPRLLIYGASSRATGIPDRFSGGGSGTDFTLTISRLEPEDFAVYYCQQYDSSPYTFGQGTKLEIK

SEQ ID NO: 128 - DNA, 366 bp, synthetic construct
  source 1..366: mol_type = other DNA
caggtgcagctggtgcagtctggggctgaggtgaagaagcctggggcctcagtgcaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagacaggggggagcccagtggctggtacttgcttttgatatctggggccaagggacaatggtcaccgtctcttca

SEQ ID NO: 129 - DNA, 321 bp, synthetic construct
  source 1..321: mol_type = other DNA
gaaattgtgttgacgcagtctccaggcaccctgtctttgtctccaggggaaagagccaccctctcctgcagggccagtcagagtgttagcagctacttagcctggtaccagcagaaacctggccagcctcccaggctcctcatctatggtgcatccagcagggccactggcatcccagacaggttcagtggcggtgggtctgggacagacttcactctcaccatcagcagactggagcctgaagattttgcagtgtattactgtcagcagtatgatagctcaccgtacacttttggccaggggaccaagctggagatcaaa

SEQ ID NO: 130 - protein, 9 aa, synthetic construct
QQYGNSPYT

SEQ ID NO: 131 - protein, 120 aa, synthetic construct
QVQLVQSGAEVKKPGASVKVSCKASGYTFTSYYMHWVRQAPGQGLEWMGIINPSGGSRSYAQKFQGRVTMTRDTSTSTVYMELSSLRSEDTAVYYCARQLELLGNAFDIWGQGTMVTVSS

SEQ ID NO: 132 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGNSPYTFGQGTKLEIK

SEQ ID NO: 133 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagtagaagctacgcacagaagttccagggcagagtcactatgaccagggacacgtccacgagcacagtctacatggagctgagcagcctgagatctgaggacacggccgtgtattactgtgcgagacaactggaacttttggggaatgcttttgatatctggggccaagggacaatggtcaccgtctcttca

SEQ ID NO: 134 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtaactcgccgtacacttttggccaggggaccaagctggagatcaaa

SEQ ID NO: 135 - protein, 7 aa, synthetic construct
GFTFNNF

SEQ ID NO: 136 - protein, 6 aa, synthetic construct
KQDGSE

SEQ ID NO: 137 - protein, 6 aa, synthetic construct
ESYFDL

SEQ ID NO: 138 - protein, 14 aa, synthetic construct
RSSTGAVTTSNYAN

SEQ ID NO: 139 - protein, 7 aa, synthetic construct
GTNNRAP

SEQ ID NO: 140 - protein, 9 aa, synthetic construct
ALWYSNRWV

SEQ ID NO: 141 - protein, 115 aa, synthetic construct
EVQLVESGGGLVQPGGSLRLSCAASGFTFNNFWMSWVRQAPGKGLEWVANIKQDGSEKYYVDSVKGRFTISRDNAKNSLYLQMNSLRAEDTAVYYCARESYFDLWGRGTLVTVSS

SEQ ID NO: 142 - protein, 109 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSNRWVFGGGTKLTVL

SEQ ID NO: 143 - DNA, 345 bp, synthetic construct
  source 1..345: mol_type = other DNA
gaggtgcagctggtggagtctgggggaggcttggtccagcctggggggtccctgagactctcctgtgcagcctctggattcacctttaataacttttggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagagagagttacttcgatctctggggccgtggcaccctggtcactgtctcctca

SEQ ID NO: 144 - DNA, 327 bp, synthetic construct
  source 1..327: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccgctgggtgttcggtggaggaaccaaactgactgtccta

SEQ ID NO: 145 - protein, 11 aa, synthetic construct
VADIVGATFDY

SEQ ID NO: 146 - protein, 7 aa, synthetic construct
GASSWAT

SEQ ID NO: 147 - protein, 120 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARVADIVGATFDYWGQGTLVTVSS

SEQ ID NO: 148 - protein, 108 aa, synthetic construct
EIVLTQSPGTLSLSPGERATLSCRASQSVSSSYLAWYQQKPGQAPRLLIYGASSWATGIPDRFSGSGSGTDFTLTISRLEPEDFAVYYCQQYDSSPYTFGQGTKLEIK

SEQ ID NO: 149 - DNA, 360 bp, synthetic construct
  source 1..360: mol_type = other DNA
caggtgcagctggtgcagtctggggctgaggtgaagaagcctggggcctcagtgaaagtttcctgcaaggcatctggatacaccttcaccagctactatatgcactgggtgcgacaggcccctggacaagggcttgagtggatgggaataatcaaccctagtggtggtagcactagctacgcacagaagttccagggcagagtcaccatgaccagggacacgtccacgagcacagtctacatggagctgagcagcctgagatctgaggacacggccgtgtattactgtgcgagagtggcggacatagtgggagctacttttgactactggggccagggaaccctggtcaccgtctcctca

SEQ ID NO: 150 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagctgggccactggcatcccagacaggttcagtggcagtgggtctgggacagacttcactctcaccatcagcagactggaacctgaagattttgcagtgtattactgtcagcagtatgatagctcaccctacacttttggccaggggaccaagctggagatcaaa

SEQ ID NO: 151 - protein, 7 aa, synthetic construct
GYTFTTY

SEQ ID NO: 152 - protein, 11 aa, synthetic construct
QGHYIGNYLDY

SEQ ID NO: 153 - protein, 120 aa, synthetic construct
QVQLVQSGAEVKKPGASVKVSCKASGYTFTTYYMHWVRQAPGQGLEWMGIINPSGGSTSYAQKFQGRVTMTRDTSTSTVYMELSSLRSEDTAVYYCARQGHYIGNYLDYWGQGTLVTVSS

SEQ ID NO: 154 - protein, 106 aa, synthetic construct; identical to SEQ ID NO: 84

SEQ ID NO: 155 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccacctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagacagggccattatattgggaactaccttgactactggggccagggaaccctggtcaccgtctcctca

SEQ ID NO: 156 - DNA, 318 bp, synthetic construct; identical to SEQ ID NO: 86
  source 1..318: mol_type = other DNA

SEQ ID NO: 157 - protein, 122 aa, synthetic construct; identical to SEQ ID NO: 126

SEQ ID NO: 158 - protein, 107 aa, synthetic construct
EIVLTQSPGTLSLSPGERATLSCRASQSVSSYLAWYQQKPGQAPRLLIYGASSRATGIPDRFSGGGSGTDFTLTISRLEPEDFAVYYCQQYGSSPYTFGQGTKLEIK

SEQ ID NO: 159 - DNA, 366 bp, synthetic construct; identical to SEQ ID NO: 128
  source 1..366: mol_type = other DNA

SEQ ID NO: 160 - DNA, 321 bp, synthetic construct
  source 1..321: mol_type = other DNA
gaaattgtgttgacgcagtctccaggcaccctgtctttgtctccaggggaaagagccaccctctcctgcagggccagtcagagtgttagcagctacttagcctggtaccagcagaaacctggccaggctcccaggctcctcatctatggtgcatccagcagggccactggcatcccagacaggttcagtggcggtgggtctgggacagacttcactctcaccatcagcagactggagcctgaagattttgcagtgtattactgtcagcagtatggtagctcaccgtacacttttggccaggggaccaagctggagatcaaa

SEQ ID NO: 161 - protein, 10 aa, synthetic construct
EWLRFGGLVY

SEQ ID NO: 162 - protein, 9 aa, synthetic construct
QQYNNWPLT

SEQ ID NO: 163 - protein, 119 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAREWLRFGGLVYWGQGTLVTVSS

SEQ ID NO: 164 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 165 - DNA, 357 bp, synthetic construct
  source 1..357: mol_type = other DNA
gaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagagagtggctacgatttgggggcttagtgtactggggccagggaaccctggtcaccgtctcctca

SEQ ID NO: 166 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgctcactttcggcggagggaccaaggtggagatcaaa

SEQ ID NO: 167 - protein, 6 aa, synthetic construct
NPNSGG

SEQ ID NO: 168 - protein, 9 aa, synthetic construct
DGVITSFDY

SEQ ID NO: 169 - protein, 17 aa, synthetic construct
KSSQSVLYSSNIKTYLA

SEQ ID NO: 170 - protein, 7 aa, synthetic construct
WASTRES

SEQ ID NO: 171 - protein, 9 aa, synthetic construct
QQYYSAPYT

SEQ ID NO: 172 - protein, 118 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDGVITSFDYWGQGTLVTVSS

SEQ ID NO: 173 - protein, 113 aa, synthetic construct
DIVMTQSPDSLAVSLGERATINCKSSQSVLYSSNIKTYLAWYQQKPGQPPKLLIYWASTRESGVPDRFSGGGSGTDFTLTITSLQAEDVAVYYCQQYYSAPYTFGQGTKLEIK

SEQ ID NO: 174 - DNA, 354 bp, synthetic construct
  source 1..354: mol_type = other DNA
caggtgcagctggtgcagtctggggctgaggtgaagaagcctggggcctcagtgaaggtctcctgcaaggcttctggatacactttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagacggggtgattacgtcttttgactactggggccagggaaccctggtcaccgtctcctca

SEQ ID NO: 175 - DNA, 339 bp, synthetic construct
  source 1..339: mol_type = other DNA
gacatcgtgatgacccagtctccagactccctggctgtgtctctgggcgagagggccaccatcaactgcaagtccagccagagtgttttatacagctccaacattaagacctacttagcttggtaccagcagaaaccaggacagcctcctaagctgctcatttactgggcatctacccgggaatccggagtccctgaccgattcagtggcggcgggtctgggacagatttcactctcaccatcaccagcctgcaggctgaagatgtggcagtttattactgtcagcagtattatagtgctccgtacacttttggccaggggaccaagctggagatcaaa

SEQ ID NO: 176 - protein, 19 aa, synthetic construct
GGGGSGGGGSGGGSGGGGS

SEQ ID NO: 177 - protein, 15 aa, synthetic construct
GGGGSGGGGSGGGGS

SEQ ID NO: 178 - protein, 24 aa, synthetic construct
GGGGSGGGGSGGGGSGGGSGGGGS

SEQ ID NO: 179 - protein, 29 aa, synthetic construct
GGGGSGGGGSGGGGSGGGGSGGGSGGGGS

SEQ ID NO: 180 - protein, 5 aa, synthetic construct
GGGGS

SEQ ID NO: 181 - protein, 10 aa, synthetic construct
GGGGSGGGGS

SEQ ID NO: 182 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 183 - protein, 6 aa, synthetic construct
DPSDGS

SEQ ID NO: 184 - protein, 11 aa, synthetic construct
DREYNYYGLDV

SEQ ID NO: 185 - protein, 16 aa, synthetic construct
RSSQSLVYRDGNTYLN

SEQ ID NO: 186 - protein, 9 aa, synthetic construct
MQGTHWPPT

SEQ ID NO: 187 - protein, 120 aa, synthetic construct
QVQLVQSGAEVKKPGASVKVSCKASGYTFTSYYIHWVRQAPGQGLEWMGIIDPSDGSTNYAQKFQGRVTMTRDTSTSTVYMELSSLRSEDTAVYYCARDREYNYYGLDVWGQGTTVTVSS

SEQ ID NO: 188 - protein, 112 aa, synthetic construct
DVVMTQSPLSLPVTLGQPASISCRSSQSLVYRDGNTYLNWFQQRPGQSPRRLIYKVSNRDSGVPDRFRGSGSGTDFTLKISRVEAEDVGVYYCMQGTHWPPTFGQGTKVEIK

SEQ ID NO: 189 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatacactgggtgcgacaggcccctggacaagggcttgagtggatgggaataatcgacccaagtgatggtagcacaaactacgcacagaagttccagggcagagtcaccatgaccagggacacgtccacgagcacagtctacatggagctgagcagcctgagatctgaggacacggccgtgtattactgtgcgagagatcgggaatataactactacggtttggacgtctggggccaagggaccacggtcaccgtctcctca

SEQ ID NO: 190 - DNA, 336 bp, synthetic construct
  source 1..336: mol_type = other DNA
gatgttgtgatgactcagtctccactctccctgcccgtcacccttggacagccggcctccatctcctgcaggtctagtcaaagcctcgtataccgtgatggaaacacctacttgaattggtttcagcagaggccaggccaatctccaaggcgcctaatttataaggtttctaaccgggactctggggtcccagacagattccgcggcagtgggtcaggcactgatttcacactgaaaatcagccgggtggaggctgaggatgttggggtttattactgcatgcaaggtacacactggcctccgacgttcggccaagggaccaaggtggaaatcaaa

SEQ ID NO: 191 - protein, 8 aa, synthetic construct
GFTFSNTW

SEQ ID NO: 192 - protein, 10 aa, synthetic construct
IKSKSDGGTT

SEQ ID NO: 193 - protein, 10 aa, synthetic construct
TQYYWNSFDY

SEQ ID NO: 194 - protein, 11 aa, synthetic construct
QASQDISNYLN

SEQ ID NO: 195 - protein, 7 aa, synthetic construct
DASNLET

SEQ ID NO: 196 - protein, 9 aa, synthetic construct
QQYDNLPLT

SEQ ID NO: 197 - protein, 119 aa, synthetic construct
EVQLVESGGGLVKPGGSLRLSCAASGFTFSNTWMSWVRQAPGKGLEWVGRIKSKSDGGTTDYAAPVKGRFTISRDDSKNTLYLQMNSLKTEDTAVYYCTQYYWNSFDYWGQGTLVTVSS

SEQ ID NO: 198 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 199 - DNA, 357 bp, synthetic construct
  source 1..357: mol_type = other DNA
gaggtgcagctggtggagtctggggggggcttggtaaagcctggggggtcccttagactctcctgtgcagcctctggattcactttcagtaacacctggatgagctgggtccgccaggctccagggaaggggctggagtgggttggccgtattaaaagcaaatctgatggtgggacaacagactacgctgcacccgtgaaaggcagattcaccatctcaagagatgattcaaaaaacacgctgtatctgcaaatgaacagcctgaaaaccgaggacacagccgtgtattactgtacccagtattattggaactcctttgactactggggccagggaaccctggtcaccgtctcctca

SEQ ID NO: 200 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctcccgctcactttcggcggagggaccaaggtggagatcaaa

SEQ ID NO: 201 - protein, 6 aa, synthetic construct
KEDGSE

SEQ ID NO: 202 - protein, 9 aa, synthetic construct
DPGWAPFDY

SEQ ID NO: 203 - protein, 9 aa, synthetic construct
QQYNSFPYT

SEQ ID NO: 204 - protein, 118 aa, synthetic construct
EVQLVESGGGLVQPGGSQRLSCAASGFTFSSYWMNWVRQAPGKGLEWVANIKEDGSEKYYVDSVKGRFTISRDNAKNSLYLQMNSLRAEDTAVYYCARDPGWAPFDYWGQGTLVTVSS

SEQ ID NO: 205 - protein, 107 aa, synthetic construct
DIQMSQSPSSLSASVGDRVTITCRASQGISNYLAWFQQKPGKAPKSLIYAASSLQSGVPSKFSGSGSGTDFTLAISSLQPEDFATYYCQQYNSFPYTFGQGTTLEIK

SEQ ID NO: 206 - DNA, 354 bp, synthetic construct
  source 1..354: mol_type = other DNA
gaggtgcagctggtggagtctgggggaggcttggtccagcctggggggtcccagagactctcctgtgcagcctctggattcacctttagtagctattggatgaactgggtccgccaggctccagggaaggggctggagtgggtggccaacataaaggaagatggaagtgagaaatactatgtggactctgtgaagggccgattcaccatctccagagacaacgccaagaactcactgtatctgcaaatgaacagcctgagagccgaggacacggctgtgtattactgtgcgagagatccgggctgggctccctttgactactggggccagggaaccctggtcaccgtctcctca

SEQ ID NO: 207 - DNA, 321 bp, synthetic construct
  source 1..321: mol_type = other DNA
gacatccagatgtcccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcgccatcagcagcctgcagcctgaagattttgcaacttattactgccaacagtataatagtttcccgtacacttttggccaggggaccacgctggagatcaaa

SEQ ID NO: 208 - protein, 7 aa, synthetic construct
GGSINSY

SEQ ID NO: 209 - protein, 5 aa, synthetic construct
FYSGI

SEQ ID NO: 210 - protein, 10 aa, synthetic construct
IGVAGFYFDY

SEQ ID NO: 211 - protein, 9 aa, synthetic construct
QQYGTSPLT

SEQ ID NO: 212 - protein, 118 aa, synthetic construct
QVQLQESGPGLVKPSETLSLTCTVSGGSINSYYWSWIRQPPGKGLEWIGYIFYSGITNYNPSLKSRVTISLDTSKNQFSLKLSSVTAADTAVYYCARIGVAGFYFDYWGQGTLVTVSS

SEQ ID NO: 213 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGTSPLTFGGGTKVEIK

SEQ ID NO: 214 - DNA, 354 bp, synthetic construct
  source 1..354: mol_type = other DNA
caggtgcagctgcaggagtcgggcccaggactggtgaagccttcggagaccctgtccctcacctgcactgtctctggtggctccatcaatagttactactggagctggatccggcagcccccagggaagggactggagtggattgggtatatcttttacagtgggatcaccaactacaacccctccctcaagagtcgagtcaccatatcattagacacgtccaagaaccagttctccctgaagctgagctctgtgaccgctgcggacacggccgtgtattactgtgcgagaatcggcgtggctggtttttactttgactactggggccagggaaccctggtcaccgtctcctca

SEQ ID NO: 215 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtacctcaccgctcactttcggcggagggaccaaggtggagatcaaa

SEQ ID NO: 216 - protein, 7 aa, synthetic construct
LGSNRAS